(19) United States
(12) Patent Application Publication
(10) Pub. No.: US 2023/0266342 A1
(43) Pub. Date: Aug. 24, 2023

(54) Biomarkers for Predicting Multiple Sclerosis Disease Progression

(71) Applicant: Octave Bioscience, Inc., Menlo Park, CA (US)

(72) Inventors: Michael Justin Becich, San Francisco, CA (US); Victor Michael Gehman, Burlingame, CA (US); Ferhan Qureshi, Fremont, CA (US); William A. Hagstrom, Santa Barbara, CA (US); Fatima Rubio da Costa, Mountain View, CA (US); Fujun Zhang, Menlo Park, CA (US)

(21) Appl. No.: 18/024,149

(22) PCT Filed: Sep. 3, 2021
(86) PCT No.: PCT/US2021/049155

§ 371 Date: Mar. 1, 2023

Related U.S. Application Data
(60) Provisional application No. 63/074,768, filed on Sep. 4, 2020.
(60) Provisional application No. 63/148,906, filed on Feb. 12, 2021.

(51) Int. Cl. G01N 33/68; G16B 25/10; G16H 50/30

(52) U.S. Cl. CPC G01N 33/6896; G16B 25/10; G16H 50/30; G01N 2800/285; G01N 2800/60

(57) ABSTRACT

Disclosed herein are methods for analyzing quantitative expression values of biomarkers of a biomarker panel for determining multiple sclerosis disease activity (e.g., multiple sclerosis disease progression) in a human subject. Further disclosed herein are kits for measuring quantitative expression values of the markers as well as computer systems and software embodiments of predictive models for determining multiple sclerosis disease activity (e.g., multiple sclerosis disease progression) in human subjects based on the quantitative expression values of the markers.

### CROSS REFERENCE TO RELATED APPLICATIONS

This application claims the benefit of and priority to U.S. Provisional Patent Application No. 63/074,768, filed Sep. 4, 2020, and U.S. Provisional Patent Application No. 63/148,906, filed Feb. 12, 2021, the entire disclosure of each of which is hereby incorporated by reference in its entirety for all purposes.

### SUMMARY

Generally, MRI scans are typically performed to determine MS disease progression in a subject. However, MM scans are expensive and slow to perform. Higher-frequency measurement of the state of a patient's MS would allow for more nimble clinical management. Disclosed herein are methods for predicting multiple sclerosis disease activity (e.g., multiple sclerosis disease progression) using multivariate biomarker panels that analyze quantitative expression levels of biomarkers in samples obtained from the subject. Samples, such as samples obtained through blood draws, are simpler, faster, and cheaper than MRIs. Thus, analyzing expression levels of biomarkers, in conjunction with MRI volumetrics or just the biomarkers alone, in samples obtained from the subject can enable earlier detection and monitoring of MS disease progression.

Additionally disclosed herein are non-transitory computer readable mediums for predicting multiple sclerosis disease activity (e.g., multiple sclerosis disease progression) using multivariate biomarker panels. Additionally disclosed herein are kits containing a set of reagents for determining expression levels of multivariate biomarkers that are informative for predicting multiple sclerosis disease activity (e.g., multiple sclerosis disease progression). Additionally disclosed herein are systems for predicting multiple sclerosis disease activity (e.g., multiple sclerosis disease progression) using multivariate biomarker panels.

The advantages of a multivariate biomarker panel for detecting multiple sclerosis disease activity (e.g., multiple sclerosis disease progression) include the following:
Improved sensitivity: A multi-biomarker test improves performance (area under the curve (AUC (also referred to herein as AUROC), accuracy), especially by eliminating false negatives as that individual biomarkers are unable to detect
Detecting silent progression: A multi-biomarker test would enable detection of subclinical progression that manifests through radiographic atrophy, but does not manifest in worsening symptoms.
Specificity: Individual biomarkers are often differentially expressed in other neurologic conditions. A multi-biomarker test would help differentiate multiple sclerosis specific disease progression.
Predictive Power: Multivariate models incorporating shifts in biomarker levels identify patients heading towards increasing or decreasing active lesions (w/stronger performance than individual biomarkers alone).

Disclosed herein is a method for predicting multiple sclerosis disease progression in a subject, the method comprising: obtaining or having obtained a dataset comprising expression levels of a plurality of biomarkers, wherein the plurality of biomarkers comprise one or more biomarkers in at least one group selected from group 1, group 2, and group 3, wherein group 1 comprises one or more of biomarker 1, biomarker 2, biomarker 3, biomarker 4, biomarker 5, biomarker 6, biomarker 7, and biomarker 8, wherein biomarker 1 is GFAP, NEFL, OPN, CXCL9, MOG, or CHI3L1, wherein biomarker 2 is CDCP1, IL-18BP, IL-18, GFAP, or MSR1, wherein biomarker 3 is MOG, CADM3, KLK6, BCAN, OMG, or GFAP, wherein biomarker 4 is CXCL13, NOS3, or MMP-2, wherein biomarker 5 is OPG, TFF3, or ENPP2, wherein biomarker 6 is APLP1, SEZ6L, BCAN, DPP6, NCAN, or KLK6, wherein biomarker 7 is VCAN, TINAGL1, CANT1, NECTIN2, MMP-9, or NPDC1, wherein biomarker 8 is NEFL, MOG, CADM3, or GFAP, and wherein group 2 comprises one or more of biomarker 9, biomarker 10, biomarker 11, biomarker 12, biomarker 13, biomarker 14, biomarker 15, biomarker 16, and biomarker 17, wherein biomarker 9 is CXCL9, CXCL10, IL-12B, CXCL11, or GFAP, wherein biomarker 10 is TNFRSF10A, TNFRSF11A, SPON2, CHI3L1, or IFI30, wherein biomarker 11 is CCL20, CCL3, or TWEAK, wherein biomarker 12 is TNFSF13B, CXCL16, ALCAM, or IL-18, wherein biomarker 13 is OPN, OMD, MEPE, or GFAP, wherein biomarker 14 is SERPINA9, TNFRSF9, or CNTN4, wherein biomarker 15 is CD6, CD5, CRTAM, CD244, or TNFRSF9, wherein biomarker 16 is FLRT2, DDR1, NTRK2, CDH6, MMP-2, wherein biomarker 17 is CNTN2, DPP6, GDNFR-alpha-3, or SCARF2, and wherein group 3 comprises one or more of biomarker 18, biomarker 19, biomarker 20, and biomarker 21, wherein biomarker 18 is COL4A1, IL-6, Notch 3, or PCDH17, wherein biomarker 19 is GH, GH2, or IGFBP-1, wherein biomarker 20 is IL-12B, IL12A, or CXCL9, and wherein biomarker 21 is PRTG, NTRK2, NTRK3, or CNTN4, and generating a prediction of multiple sclerosis disease progression by applying a predictive model to the expression levels of the plurality of biomarkers.

In various embodiments, the plurality of biomarkers comprise each biomarker in group 1, wherein biomarker 1 is GFAP, wherein biomarker 2 is CDCP1, wherein biomarker 3 is MOG, wherein biomarker 4 is CXCL13, wherein biomarker 5 is OPG, wherein biomarker 6 is APLP1, wherein biomarker 7 is VCAN, and wherein biomarker 8 is NEFL. In various embodiments, a performance of the predictive model is characterized by a correlation coefficient (R²) of at least 0.31. In various embodiments, a performance of the predictive model is characterized by an AUROC of at least 0.77. In various embodiments, a performance of the predictive model is characterized by a PPV of at least 0.19.

In various embodiments, the plurality of biomarkers further comprise each biomarker in group 2, wherein biomarker 9 is CXCL9, wherein biomarker 10 is TNFRSF10A, wherein biomarker 11 is CCL20, wherein biomarker 12 is TNFSF13B, wherein biomarker 13 is OPN, wherein biomarker 14 is SERPINA9, wherein biomarker 15 is CD6, wherein biomarker 16 is FLRTs, and wherein biomarker 17 is CNTN2. In various embodiments, a performance of the predictive model is characterized by a correlation coefficient (R²) of at least 0.35. In various embodiments, a performance of the predictive model is characterized by an AUROC of at least 0.76. In various embodiments, a performance of the predictive model is characterized by a PPV of at least 0.19.

In various embodiments, the plurality of biomarkers further comprise each biomarker in group 3, wherein biomarker 18 is COL4A1, wherein biomarker 19 is GH, wherein biomarker 20 is IL-12B, and wherein biomarker 21 is PRTG. In various embodiments, a performance of the predictive model is characterized by a correlation coefficient (R²) of at least 0.36. In various embodiments, a performance of the predictive model is characterized by an AUROC of at least 0.74. In various embodiments, a performance of the predictive model is characterized by a PPV of at least 0.19.

In various embodiments, the plurality of biomarkers comprises one or more biomarkers in group 1, wherein the one or more biomarkers in group 1 comprises GFAP. In various embodiments, a performance of the predictive model is characterized by an AUROC of at least 0.70. In various embodiments, a performance of the predictive model is characterized by an AUROC between 0.72 and 0.78. In various embodiments, a performance of the predictive model is characterized by a Pearson's R² coefficient of at least 0.10. In various embodiments, a performance of the predictive model is characterized by an Pearson's R² coefficient between 0.11 and 0.22.

In various embodiments, the plurality of biomarkers does not include GFAP. In various embodiments, a performance of the predictive model is characterized by an AUROC of at least 0.65. In various embodiments, a performance of the predictive model is characterized by an AUROC between 0.66 and 0.70. In various embodiments, a performance of the predictive model is characterized by a Pearson's R² coefficient of at least 0.10. In various embodiments, a performance of the predictive model is characterized by an Pearson's R² coefficient between 0.015 and 0.090.

In various embodiments, the prediction of multiple sclerosis disease progression is a measure of brain parenchymal fraction value. In various embodiments, the prediction of multiple sclerosis disease progression is a measure of expanded disability status scale (EDSS) score. In various embodiments, an expanded disability status scale (EDSS) score less than 6 indicates a mild/moderate MS disease progression and a EDSS score greater than or equal to 6 indicates a severe MS disease progression. In various embodiments, the prediction of multiple sclerosis disease progression is a measure of a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression. In various embodiments, the prediction of multiple sclerosis disease progression is a PRO measurement information system (PROMIS) score. In various embodiments, the prediction of multiple sclerosis disease progression is a Multiple Sclerosis Rating Scale, Revised (MSRS-R) score.

Additionally disclosed herein is a method for predicting multiple sclerosis disease progression in a subject, the method comprising: obtaining or having obtained a dataset comprising expression levels of a plurality of biomarkers comprising at least one of: one or more neuroaxonal integrity biomarkers selected from a group consisting of CNTN2, FLRT2, NEFL, PRTG, SERPINA9, OPG, GFAP, and TNFRSF10A; one or more neuroinflammation biomarkers selected from a group consisting of CCL20, GH, TNFRSF10A, CXCL9, CXCL13, IL-12B, CD6, and TNFSF13B; one or more immune modulation biomarkers selected from a group consisting of CDCP1, CD6, CXCL9, CXCL13, IL-12B, and TNFSF13B; or one or more myelination biomarkers selected from a group consisting of MOG, APLP1, and OPN; and generating a prediction of multiple sclerosis disease progression by applying a predictive model to the expression levels of the plurality of biomarkers. Additionally disclosed herein is a method for predicting multiple sclerosis disease progression in a subject, the method comprising: obtaining or having obtained a dataset comprising expression levels of a plurality of biomarkers comprising at least one of: one or more neuroaxonal integrity biomarkers selected from a group consisting of CNTN2, FLRT2, NEFL, PRTG, SERPINA9, OPG, GFAP, and TNFRSF10A; one or more neuroinflammation biomarkers selected from a group consisting of CCL20, GH, TNFRSF10A, CXCL9, CXCL13, IL-12B, CD6, and TNFSF13B; one or more immune modulation biomarkers selected from a group consisting of CDCP1, CD6, CXCL9, CXCL13, IL-12B, and TNFSF13B; one or more myelination biomarkers selected from a group consisting of MOG, APLP1, and OPN; or one or more cerebrovascular function biomarkers selected from a group consisting of COL4A1, VCAN, GFAP, and CD6; and generating a prediction of multiple sclerosis disease progression by applying a predictive model to the expression levels of the plurality of biomarkers.

In various embodiments, the one or more neuroaxonal integrity biomarkers comprise NEFL, OPG, and GFAP, wherein the one or more neuroinflammation biomarkers comprise CXCL13, and CXCL9, wherein the one or more immune modulation biomarkers comprise CDCP1, and wherein the one or more myelination biomarkers comprise MOG and APLP1. In various embodiments, the one or more neuroaxonal integrity biomarkers further comprise SERPINA9, FLRT2, and CNTN2, wherein the one or more neuroinflammation biomarkers further comprise CCL20, CXCL9, TNFRSF10A, and CD6, wherein the one or more immune modulation biomarkers further comprise TNFSF13B, and wherein the one or more myelination biomarkers further comprise OPN. In various embodiments, the one or more neuroaxonal integrity biomarkers further comprise PRTG, and wherein the one or more immune modulation biomarkers further comprise IL-12B.

In various embodiments, a performance of the predictive model is characterized by a correlation coefficient (R²) of at least 0.35. In various embodiments, a performance of the predictive model is characterized by an AUROC of at least 0.74. In various embodiments, a performance of the predictive model is characterized by a PPV of at least 0.17.

In various embodiments, the plurality of biomarkers comprises one or more neuroaxonal integrity biomarkers, wherein the one or more neuroaxonal integrity biomarkers comprises GFAP. In various embodiments, a performance of the predictive model is characterized by an AUROC of at least 0.70. In various embodiments, a performance of the predictive model is characterized by an AUROC between 0.72 and 0.78. In various embodiments, a performance of the predictive model is characterized by a Pearson's R² coefficient of at least 0.10. In various embodiments, a performance of the predictive model is characterized by an Pearson's R² coefficient between 0.11 and 0.22.

In various embodiments, the plurality of biomarkers does not include GFAP. In various embodiments, a performance of the predictive model is characterized by an AUROC of at least 0.65. In various embodiments, a performance of the predictive model is characterized by an AUROC between 0.66 and 0.70. In various embodiments, a performance of the predictive model is characterized by a Pearson's R² coefficient of at least 0.10. In various embodiments, a performance of the predictive model is characterized by an Pearson's R² coefficient between 0.015 and 0.090.

In various embodiments, the prediction of multiple sclerosis disease progression is a brain parenchymal fraction value. In various embodiments, the prediction of multiple sclerosis disease progression is an expanded disability status scale (EDSS) score. In various embodiments, an expanded disability status scale (EDSS) score less than 6 indicates a mild/moderate MS disease progression and a EDSS score greater than or equal to 6.0 indicates a severe MS disease progression. In various embodiments, the prediction of multiple sclerosis disease progression is a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression. In various embodiments, the prediction of multiple sclerosis disease progression is a PRO measurement information system (PROMIS) score. In various embodiments, the prediction of multiple sclerosis disease progression is a Multiple Sclerosis Rating Scale, Revised (MSRS-R) score.

Additionally disclosed herein is a method for predicting multiple sclerosis disease progression in a subject, the method comprising: obtaining or having obtained a dataset comprising expression levels of a plurality of biomarkers, wherein the plurality of biomarkers comprises two or more of: GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG; and generating a prediction of multiple sclerosis disease progression by applying a predictive model to the expression levels of the plurality of biomarkers. In various embodiments, the plurality of biomarkers comprise each of GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG.

In various embodiments, a performance of the predictive model is characterized by a correlation coefficient (R²) of at least 0.35. In various embodiments, a performance of the predictive model is characterized by an AUROC of at least 0.74. In various embodiments, a performance of the predictive model is characterized by a PPV of at least 0.17.

In various embodiments, the plurality of biomarkers comprises GFAP. In various embodiments, the plurality of biomarkers further comprises CDCP1. In various embodiments, the plurality of biomarkers further comprises APLP1. In various embodiments, the plurality of biomarkers further comprises CXCL13. In various embodiments, the plurality of biomarkers further comprises MOG. In various embodiments, the plurality of biomarkers further comprises OPG. In various embodiments, the plurality of biomarkers further comprises CDCP1 and APLP1. In various embodiments, the plurality of biomarkers further comprises MOG and CDCP1. In various embodiments, the plurality of biomarkers further comprises APLP1 and CXCL13. In various embodiments, the plurality of biomarkers further comprises CDCP1 and SERPINA9. In various embodiments, the plurality of biomarkers further comprises MOG and CXCL13. In various embodiments, the plurality of biomarkers further comprises CDCP1, CCL20, and APLP1. In various embodiments, the plurality of biomarkers further comprises CDCP1, APLP1 and CXCL13. In various embodiments, the plurality of biomarkers further comprises CDCP1, CCL20, and MOG. In various embodiments, the plurality of biomarkers further comprises CDCP1, APLP1, and SERPINA9. In various embodiments, the plurality of biomarkers further comprises CDCP1, MOG, and APLP1. In various embodiments, a performance of the predictive model is characterized by an AUROC of at least 0.70. In various embodiments, a performance of the predictive model is characterized by an AUROC between 0.72 and 0.78.

In various embodiments, the plurality of biomarkers further comprises MOG. In various embodiments, the plurality of biomarkers further comprises APLP1. In various embodiments, the plurality of biomarkers further comprises OPG. In various embodiments, the plurality of biomarkers further comprises TNFRSF10A. In various embodiments, the plurality of biomarkers further comprises CDCP1. In various embodiments, the plurality of biomarkers further comprises APLP1. In various embodiments, the plurality of biomarkers further comprises NEFL. In various embodiments, the plurality of biomarkers further comprises CNTN2. In various embodiments, the plurality of biomarkers further comprises GH. In various embodiments, the plurality of biomarkers further comprises CXCL9. In various embodiments, the plurality of biomarkers further comprises OPG and MOG. In various embodiments, the plurality of biomarkers further comprises OPG and APLP1. In various embodiments, the plurality of biomarkers further comprises TNFRSF10A and MOG. In various embodiments, the plurality of biomarkers further comprises CXCL9 and OPG. In various embodiments, the plurality of biomarkers further comprises TNFRSF10A and APLP1. In various embodiments, the plurality of biomarkers further comprises APLP1 and NEFL. In various embodiments, the plurality of biomarkers further comprises CXCL13 and APLP1. In various embodiments, the plurality of biomarkers further comprises FLRT2 and APLP1. In various embodiments, the plurality of biomarkers further comprises CXCL9 and APLP1. In various embodiments, the plurality of biomarkers further comprises GH and APLP1. In various embodiments, the plurality of biomarkers further comprises CXCL9, OPG, and MOG. In various embodiments, the plurality of biomarkers further comprises CNTN2, OPG, and MOG. In various embodiments, the plurality of biomarkers further comprises CXCL9, OPG, and APLP1. In various embodiments, the plurality of biomarkers further comprises OPG, PRTG, and MOG. In various embodiments, the plurality of biomarkers further comprises OPG, OPN, and MOG. In various embodiments, the plurality of biomarkers further comprises CXCL13, APLP1, and NEFL. In various embodiments, the plurality of biomarkers further comprises FLRT2, APLP1, and NEFL. In various embodiments, the plurality of biomarkers further comprises OPN, APLP1, and NEFL. In various embodiments, the plurality of biomarkers further comprises CXCL9, APLP1, and NEFL. In various embodiments, the plurality of biomarkers further comprises CXCL13, FLRT2, and APLP1. In various embodiments, a performance of the predictive model is characterized by a Pearson's R² coefficient of at least 0.10. In various embodiments, a performance of the predictive model is characterized by an Pearson's R² coefficient between 0.11 and 0.22.

In various embodiments, the plurality of biomarkers does not include GFAP. In various embodiments, the plurality of biomarkers comprises CDCP1 and OPG. In various embodiments, the plurality of biomarkers comprises CDCP1 and SERPINA9. In various embodiments, the plurality of biomarkers comprises OPG and TNFRSF10A. In various embodiments, the plurality of biomarkers comprises OPG and MOG. In various embodiments, the plurality of biomarkers comprises CDCP1 and MOG. In various embodiments, the plurality of biomarkers comprises CDCP1, MOG, and OPG. In various embodiments, the plurality of biomarkers comprises CDCP1, SERPIN A9, and OPG. In various embodiments, the plurality of biomarkers comprises CDCP1, OPG, and CXCL13. In various embodiments, the plurality of biomarkers comprises CDCP1, CXCL9, and OPG. In various embodiments, the plurality of biomarkers comprises CDCP1, FLRT2, and OPG. In various embodiments, the plurality of biomarkers comprises CDCP1, MOG, OPG, and CXCL13. In various embodiments, the plurality of biomarkers comprises CDCP1, MOG, TNFRSF10A, and OPG. In various embodiments, the plurality of biomarkers comprises CDCP1, CXCL9, SERPINA9, and OPG. In various embodiments, the plurality of biomarkers comprises CDCP1, CNTN2, SERPINA9, and OPG. In various embodiments, the plurality of biomarkers comprises CDCP1, SERPINA9, CD6, and OPG. In various embodiments, a performance of the predictive model is characterized by an AUROC of at least 0.65. In various embodiments, a performance of the predictive model is characterized by an AUROC between 0.66 and 0.70.

In various embodiments, the plurality of biomarkers comprises OPG and NEFL. In various embodiments, the plurality of biomarkers comprises OPG and OPN. In various embodiments, the plurality of biomarkers comprises OPG and FLRT2. In various embodiments, the plurality of biomarkers comprises OPG and MOG. In various embodiments, the plurality of biomarkers comprises CXCL9 and OPG. In various embodiments, the plurality of biomarkers comprises GH and NEFL. In various embodiments, the plurality of biomarkers comprises CXCL13 and NEFL. In various embodiments, the plurality of biomarkers comprises APLP1 and NEFL. In various embodiments, the plurality of biomarkers comprises CCL20 and NEFL. In various embodiments, the plurality of biomarkers comprises CXCL9 and NEFL. In various embodiments, the plurality of biomarkers comprises OPG, MOG, and NEFL. In various embodiments, the plurality of biomarkers comprises OPG, FLRT2, and NEFL. In various embodiments, the plurality of biomarkers comprises CXCL9, OPG, and NEFL. In various embodiments, the plurality of biomarkers comprises OPG, CDCP1, and NEFL. In various embodiments, the plurality of biomarkers comprises OPG, OPN, and NEFL. In various embodiments, the plurality of biomarkers comprises GH, APLP1, and NEFL. In various embodiments, the plurality of biomarkers comprises GH, CXCL13, and NEFL. In various embodiments, the plurality of biomarkers comprises GH, CDCP1, and NEFL. In various embodiments, the plurality of biomarkers comprises CXCL13, CCL20, and NEFL. In various embodiments, the plurality of biomarkers comprises GH, CCL20, and NEFL. In various embodiments, the plurality of biomarkers comprises CDCP1, CXCL13, MOG, and NEFL. In various embodiments, the plurality of biomarkers comprises CD6, CXCL9, CXCL13, and NEFL. In various embodiments, the plurality of biomarkers comprises CXCL9, CXCL13, MOG, and NEFL. In various embodiments, the plurality of biomarkers comprises CD6, CDCP1, CXCL13, and NEFL. In various embodiments, the plurality of biomarkers comprises CD6, CXCL9, CXCL13, and MOG. In various embodiments, the plurality of biomarkers comprises CDCP1, CXCL13, MOG, and NEFL. In various embodiments, the plurality of biomarkers comprises CD6, CDCP1, CXCL13, and NEFL. In various embodiments, the plurality of biomarkers comprises CXCL9, CXCL13, MOG, and NEFL. In various embodiments, the plurality of biomarkers comprises CD6, CXCL9, CXCL13, and NEFL. In various embodiments, the plurality of biomarkers comprises CD6, CDCP1, CXCL13, and MOG. In various embodiments, a performance of the predictive model is characterized by a Pearson's R² coefficient of at least 0.10. In various embodiments, a performance of the predictive model is characterized by an Pearson's R² coefficient between 0.015 and 0.090.

In various embodiments, the prediction of multiple sclerosis disease progression is a brain parenchymal fraction value. In various embodiments, the prediction of multiple sclerosis disease progression is an expanded disability status scale (EDSS) score. In various embodiments, an expanded disability status scale (EDSS) score less than or equal to 6 indicates a mild/moderate MS disease progression and a EDSS score greater than 6.5 indicates a severe MS disease progression. In various embodiments, the prediction of multiple sclerosis disease progression is a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression. In various embodiments, the prediction of multiple sclerosis disease progression is a PRO measurement information system (PROMIS) score. In various embodiments, the prediction of multiple sclerosis disease progression is a Multiple Sclerosis Rating Scale, Revised (MSRS-R) score. In various embodiments, generating the prediction of multiple sclerosis disease progression by applying the predictive model to the expression levels of the plurality of biomarkers further comprises applying the predictive model to one or more subject attributes of the subject, wherein subject attributes comprise any of age, sex, and disease duration. In various embodiments, generating the prediction of multiple sclerosis disease progression comprises comparing a score outputted by the predictive model to a reference score. In various embodiments, the reference score corresponds to any of: A) an EDSS score; B) a brain parenchymal fraction value; C) a PDDS score; D) a PROMIS score; or E) a MSRS-R score. In various embodiments, the reference score further corresponds to a mild/moderate MS disease progression or a severe MS disease progression. In various embodiments, the expression levels of the plurality of biomarkers is determined from a test sample obtained from the subject. In various embodiments, the test sample is a blood or serum sample. In various embodiments, the subject has multiple sclerosis, is suspected of having multiple sclerosis, or was previously diagnosed with multiple sclerosis. In various embodiments, obtaining or having obtained the dataset comprises performing an immunoassay to determine the expression levels of the plurality of biomarkers. In various embodiments, the immunoassay is a Proximity Extension Assay (PEA) or LUMINEX xMAP Multiplex Assay. In various embodiments, performing the immunoassay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies. In various embodiments, methods disclosed herein further comprise: selecting a therapy for administering to the subject based on the prediction of multiple sclerosis disease progression. In various embodiments, methods disclosed herein further comprise determining a therapeutic efficacy of a therapy previously administered to the subject based on the prediction of multiple sclerosis disease progression. In various embodiments, determining the therapeutic efficacy of the therapy comprises comparing the prediction to a prior prediction determined for the subject at a prior timepoint. In various embodiments, determining the therapeutic efficacy of the therapy comprises determining that the therapy exhibits efficacy responsive to a difference between the prediction and the prior prediction. In various embodiments, determining the therapeutic efficacy of the therapy comprises determining that the therapy lacks efficacy responsive to a lack of difference between the prediction and the prior prediction.

Additionally disclosed herein is a non-transitory computer readable medium for predicting multiple sclerosis disease progression in a subject, the non-transitory computer readable medium comprising instructions that, when executed by a processor, cause the processor to: obtain a dataset comprising expression levels of a plurality of biomarkers, wherein the plurality of biomarkers comprise one or more biomarkers in at least one group selected from group 1, group 2, and group 3, wherein group 1 comprises one or more of biomarker 1, biomarker 2, biomarker 3, biomarker 4, biomarker 5, biomarker 6, biomarker 7, and biomarker 8, wherein biomarker 1 is GFAP, NEFL, OPN, CXCL9, MOG, or CHI3L1, wherein biomarker 2 is CDCP1, IL-18BP, IL-18, GFAP, or MSR1, wherein biomarker 3 is MOG, CADM3, KLK6, BCAN, OMG, or GFAP, wherein biomarker 4 is CXCL13, NOS3, or MMP-2, wherein biomarker 5 is OPG, TFF3, or ENPP2, wherein biomarker 6 is APLP1, SEZ6L, BCAN, DPP6, NCAN, or KLK6, wherein biomarker 7 is VCAN, TINAGL1, CANT1, NECTIN2, MMP-9, or NPDC1, wherein biomarker 8 is NEFL, MOG, CADM3, or GFAP, and wherein group 2 comprises one or more of biomarker 9, biomarker 10, biomarker 11, biomarker 12, biomarker 13, biomarker 14, biomarker 15, biomarker 16, and biomarker 17, wherein biomarker 9 is CXCL9, CXCL10, IL-12B, CXCL11, or GFAP, wherein biomarker 10 is TNFRSF10A, TNFRSF11A, SPON2, CHI3L1, or IFI30, wherein biomarker 11 is CCL20, CCL3, or TWEAK, wherein biomarker 12 is TNFSF13B, CXCL16, ALCAM, or IL-18, wherein biomarker 13 is OPN, OMD, MEPE, or GFAP, wherein biomarker 14 is SERPINA9, TNFRSF9, or CNTN4, wherein biomarker 15 is CD6, CD5, CRTAM, CD244, or TNFRSF9, wherein biomarker 16 is FLRT2, DDR1, NTRK2, CDH6, MMP-2, wherein biomarker 17 is CNTN2, DPP6, GDNFR-alpha-3, or SCARF2, and wherein group 3 comprises one or more of biomarker 18, biomarker 19, biomarker 20, and biomarker 21, wherein biomarker 18 is COL4A1, IL-6, Notch 3, or PCDH17, wherein biomarker 19 is GH, GH2, or IGFBP-1, wherein biomarker 20 is IL-12B, IL12A, or CXCL9, and wherein biomarker 21 is PRTG, NTRK2, NTRK3, or CNTN4, and generate a prediction of multiple sclerosis disease progression by applying a predictive model to the expression levels of the plurality of biomarkers.

In various embodiments, the plurality of biomarkers comprise each biomarker in group 1, wherein biomarker 1 is GFAP, wherein biomarker 2 is CDCP1, wherein biomarker 3 is MOG, wherein biomarker 4 is CXCL13, wherein biomarker 5 is OPG, wherein biomarker 6 is APLP1, wherein biomarker 7 is VCAN, and wherein biomarker 8 is NEFL. In various embodiments, a performance of the predictive model is characterized by a correlation coefficient (R²) of at least 0.31. In various embodiments, a performance of the predictive model is characterized by an AUROC of at least 0.77. In various embodiments, a performance of the predictive model is characterized by a PPV of at least 0.19.

In various embodiments, the plurality of biomarkers further comprise each biomarker in group 2, wherein biomarker 9 is CXCL9, wherein biomarker 10 is TNFRSF10A, wherein biomarker 11 is CCL20, wherein biomarker 12 is TNFSF13B, wherein biomarker 13 is OPN, wherein biomarker 14 is SERPINA9, wherein biomarker 15 is CD6, wherein biomarker 16 is FLRTs, and wherein biomarker 17 is CNTN2. In various embodiments, a performance of the predictive model is characterized by a correlation coefficient (R²) of at least 0.35. In various embodiments, a performance of the predictive model is characterized by an AUROC of at least 0.76. In various embodiments, a performance of the predictive model is characterized by a PPV of at least 0.19.

In various embodiments, the plurality of biomarkers further comprise each biomarker in group 3, wherein biomarker 18 is COL4A1, wherein biomarker 19 is GH, wherein biomarker 20 is IL-12B, and wherein biomarker 21 is PRTG. In various embodiments, a performance of the predictive model is characterized by a correlation coefficient (R²) of at least 0.36. In various embodiments, a performance of the predictive model is characterized by an AUROC of at least 0.74. In various embodiments, a performance of the predictive model is characterized by a PPV of at least 0.19.

In various embodiments, the plurality of biomarkers comprises one or more biomarkers in group 1, wherein the one or more biomarkers in group 1 comprises GFAP. In various embodiments, a performance of the predictive model is characterized by an AUROC of at least 0.70. In various embodiments, a performance of the predictive model is characterized by an AUROC between 0.72 and 0.78. In various embodiments, a performance of the predictive model is characterized by a Pearson's R² coefficient of at least 0.10. In various embodiments, a performance of the predictive model is characterized by an Pearson's R² coefficient between 0.11 and 0.22.

In various embodiments, the plurality of biomarkers does not include GFAP. In various embodiments, a performance of the predictive model is characterized by an AUROC of at least 0.65. In various embodiments, a performance of the predictive model is characterized by an AUROC between 0.66 and 0.70. In various embodiments, a performance of the predictive model is characterized by a Pearson's R² coefficient of at least 0.10. In various embodiments, a performance of the predictive model is characterized by an Pearson's R² coefficient between 0.015 and 0.090. In various embodiments, the prediction of multiple sclerosis disease progression is a measure of brain parenchymal fraction value. In various embodiments, the prediction of multiple sclerosis disease progression is a measure of expanded disability status scale (EDSS) score. In various embodiments, an expanded disability status scale (EDSS) score less than 6 indicates a mild/moderate MS disease progression and a EDSS score greater than or equal to 6 indicates a severe MS disease progression. In various embodiments, the prediction of multiple sclerosis disease progression is a measure of a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression. In various embodiments, the prediction of multiple sclerosis disease progression is a PRO measurement information system (PROMIS) score. In various embodiments, the prediction of multiple sclerosis disease progression is a Multiple Sclerosis Rating Scale, Revised (MSRS-R) score.

Additionally disclosed herein is a non-transitory computer readable medium for predicting multiple sclerosis disease progression in a subject, the non-transitory computer readable medium comprising instructions that, when executed by a processor, cause the processor to: obtain a dataset comprising expression levels of a plurality of biomarkers comprising at least one of: one or more neuroaxonal integrity biomarkers selected from a group consisting of CNTN2, FLRT2, NEFL, PRTG, SERPINA9, OPG, GFAP, and TNFRSF10A; one or more neuroinflammation biomarkers selected from a group consisting of CCL20, GH, TNFRSF10A, CXCL9, CXCL13, IL-12B, CD6, and TNFSF13B; one or more immune modulation biomarkers selected from a group consisting of CDCP1, CD6, CXCL9, CXCL13, IL-12B, and TNFSF13B; or one or more myelination biomarkers selected from a group consisting of MOG, APLP1, and OPN; and generate a prediction of multiple sclerosis disease progression by applying a predictive model to the expression levels of the plurality of biomarkers. Additionally disclosed herein is a non-transitory computer readable medium for predicting multiple sclerosis disease progression in a subject, the non-transitory computer readable medium comprising instructions that, when executed by a processor, cause the processor to: obtain a dataset comprising expression levels of a plurality of biomarkers comprising at least one of: one or more neuroaxonal integrity biomarkers selected from a group consisting of CNTN2, FLRT2, NEFL, PRTG, SERPINA9, OPG, GFAP, and TNFRSF10A; one or more neuroinflammation biomarkers selected from a group consisting of CCL20, GH, TNFRSF10A, CXCL9, CXCL13, IL-12B, CD6, and TNFSF13B; one or more immune modulation biomarkers selected from a group consisting of CDCP1, CD6, CXCL9, CXCL13, IL-12B, and TNFSF13B; one or more myelination biomarkers selected from a group consisting of MOG, APLP1, and OPN; or one or more cerebrovascular function biomarkers selected from a group consisting of COL4A1, VCAN, GFAP, and CD6; and generate a prediction of multiple sclerosis disease progression by applying a predictive model to the expression levels of the plurality of biomarkers.

In various embodiments, the one or more neuroaxonal integrity biomarkers comprise NEFL, OPG, and GFAP, wherein the one or more neuroinflammation biomarkers comprise CXCL13, and CXCL9, wherein the one or more immune modulation biomarkers comprise CDCP1, and wherein the one or more myelination biomarkers comprise MOG and APLP1. In various embodiments, the one or more neuroaxonal integrity biomarkers further comprise SERPINA9, FLRT2, and CNTN2, wherein the one or more neuroinflammation biomarkers further comprise CCL20, CXCL9, TNFRSF10A, and CD6, wherein the one or more immune modulation biomarkers further comprise TNFSF13B, and wherein the one or more myelination biomarkers further comprise OPN. In various embodiments, the one or more neuroaxonal integrity biomarkers further comprise PRTG, and wherein the one or more immune modulation biomarkers further comprise IL-12B.

In various embodiments, a performance of the predictive model is characterized by a correlation coefficient (R²) of at least 0.35. In various embodiments, a performance of the predictive model is characterized by an AUROC of at least 0.74. In various embodiments, a performance of the predictive model is characterized by a PPV of at least 0.17. In various embodiments, the plurality of biomarkers comprises one or more neuroaxonal integrity biomarkers, wherein the one or more neuroaxonal integrity biomarkers comprises GFAP. In various embodiments, a performance of the predictive model is characterized by an AUROC of at least 0.70. In various embodiments, a performance of the predictive model is characterized by an AUROC between 0.72 and 0.78. In various embodiments, a performance of the predictive model is characterized by a Pearson's R² coefficient of at least 0.10. In various embodiments, a performance of the predictive model is characterized by an Pearson's R² coefficient between 0.11 and 0.22.

In various embodiments, the plurality of biomarkers does not include GFAP. In various embodiments, a performance of the predictive model is characterized by an AUROC of at least 0.65. In various embodiments, a performance of the predictive model is characterized by an AUROC between 0.66 and 0.70. In various embodiments, a performance of the predictive model is characterized by a Pearson's R² coefficient of at least 0.10. In various embodiments, a performance of the predictive model z is characterized by an Pearson's R² coefficient between 0.015 and 0.090. In various embodiments, the prediction of multiple sclerosis disease progression is a brain parenchymal fraction value. In various embodiments, the prediction of multiple sclerosis disease progression is an expanded disability status scale (EDSS) score. In various embodiments, an expanded disability status scale (EDSS) score less than 6 indicates a mild/moderate MS disease progression and a EDSS score greater than or equal to 6.0 indicates a severe MS disease progression. In various embodiments, the prediction of multiple sclerosis disease progression is a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression. In various embodiments, the prediction of multiple sclerosis disease progression is a PRO measurement information system (PROMIS) score. In various embodiments, the prediction of multiple sclerosis disease progression is a Multiple Sclerosis Rating Scale, Revised (MSRS-R) score.

Additionally disclosed herein is a non-transitory computer readable medium for predicting multiple sclerosis disease progression in a subject, the non-transitory computer readable medium comprising instructions that, when executed by a processor, cause the processor to: obtain a dataset comprising expression levels of a plurality of biomarkers, wherein the plurality of biomarkers comprises two or more of: GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG; and generate a prediction of multiple sclerosis disease progression by applying a predictive model to the expression levels of the plurality of biomarkers. In various embodiments, the plurality of biomarkers comprise each of GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG.

In various embodiments, a performance of the predictive model is characterized by a correlation coefficient (R²) of at least 0.35. In various embodiments, a performance of the predictive model is characterized by an AUROC of at least 0.74. In various embodiments, a performance of the predictive model is characterized by a PPV of at least 0.17.

In various embodiments, the plurality of biomarkers comprises GFAP. In various embodiments, the plurality of biomarkers further comprises CDCP1. In various embodiments, the plurality of biomarkers further comprises APLP1. In various embodiments, the plurality of biomarkers further comprises CXCL13. In various embodiments, the plurality of biomarkers further comprises MOG. In various embodiments, the plurality of biomarkers further comprises OPG. In various embodiments, the plurality of biomarkers further comprises CDCP1 and APLP1. In various embodiments, the plurality of biomarkers further comprises MOG and CDCP1. In various embodiments, the plurality of biomarkers further comprises APLP1 and CXCL13. In various embodiments, the plurality of biomarkers further comprises CDCP1 and SERPINA9. In various embodiments, the plurality of biomarkers further comprises MOG and CXCL13. In various embodiments, the plurality of biomarkers further comprises CDCP1, CCL20, and APLP1. In various embodiments, the plurality of biomarkers further comprises CDCP1, APLP1 and CXCL13. In various embodiments, the plurality of biomarkers further comprises CDCP1, CCL20, and MOG. In various embodiments, the plurality of biomarkers further comprises CDCP1, APLP1, and SERPINA9. In various embodiments, the plurality of biomarkers further comprises CDCP1, MOG, and APLP1. In various embodiments, a performance of the predictive model is characterized by an AUROC of at least 0.70. In various embodiments, a performance of the predictive model is characterized by an AUROC between 0.72 and 0.78.

In various embodiments, the plurality of biomarkers further comprises MOG. In various embodiments, the plurality of biomarkers further comprises APLP1. In various embodiments, the plurality of biomarkers further comprises OPG. In various embodiments, the plurality of biomarkers further comprises TNFRSF10A. In various embodiments, the plurality of biomarkers further comprises CDCP1. In various embodiments, the plurality of biomarkers further comprises APLP1. In various embodiments, the plurality of biomarkers further comprises NEFL. In various embodiments, the plurality of biomarkers further comprises CNTN2. In various embodiments, the plurality of biomarkers further comprises GH. In various embodiments, the plurality of biomarkers further comprises CXCL9. In various embodiments, the plurality of biomarkers further comprises OPG and MOG. In various embodiments, the plurality of biomarkers further comprises OPG and APLP1. In various embodiments, the plurality of biomarkers further comprises TNFRSF10A and MOG. In various embodiments, the plurality of biomarkers further comprises CXCL9 and OPG. In various embodiments, the plurality of biomarkers further comprises TNFRSF10A and APLP1. In various embodiments, the plurality of biomarkers further comprises APLP1 and NEFL. In various embodiments, the plurality of biomarkers further comprises CXCL13 and APLP1. In various embodiments, the plurality of biomarkers further comprises FLRT2 and APLP1. In various embodiments, the plurality of biomarkers further comprises CXCL9 and APLP1. In various embodiments, the plurality of biomarkers further comprises GH and APLP1. In various embodiments, the plurality of biomarkers further comprises CXCL9, OPG, and MOG. In various embodiments, the plurality of biomarkers further comprises CNTN2, OPG, and MOG. In various embodiments, the plurality of biomarkers further comprises CXCL9, OPG, and APLP1. In various embodiments, the plurality of biomarkers further comprises OPG, PRTG, and MOG. In various embodiments, the plurality of biomarkers further comprises OPG, OPN, and MOG. In various embodiments, the plurality of biomarkers further comprises CXCL13, APLP1, and NEFL. In various embodiments, the plurality of biomarkers further comprises FLRT2, APLP1, and NEFL. In various embodiments, the plurality of biomarkers further comprises OPN, APLP1, and NEFL. In various embodiments, the plurality of biomarkers further comprises CXCL9, APLP1, and NEFL. In various embodiments, the plurality of biomarkers further comprises CXCL13, FLRT2, and APLP1. In various embodiments, a performance of the predictive model is characterized by a Pearson's R² coefficient of at least 0.10. In various embodiments, a performance of the predictive model is characterized by an Pearson's R² coefficient between 0.11 and 0.22.

In various embodiments, the plurality of biomarkers does not include GFAP. In various embodiments, the plurality of biomarkers comprises CDCP1 and OPG. In various embodiments, the plurality of biomarkers comprises CDCP1 and SERPINA9. In various embodiments, the plurality of biomarkers comprises OPG and TNFRSF10A. In various embodiments, the plurality of biomarkers comprises OPG and MOG. In various embodiments, the plurality of biomarkers comprises CDCP1 and MOG. In various embodiments, the plurality of biomarkers comprises CDCP1, MOG, and OPG. In various embodiments, the plurality of biomarkers comprises CDCP1, SERPIN A9, and OPG. In various embodiments, the plurality of biomarkers comprises CDCP1, OPG, and CXCL13. In various embodiments, the plurality of biomarkers comprises CDCP1, CXCL9, and OPG. In various embodiments, the plurality of biomarkers comprises CDCP1, FLRT2, and OPG. In various embodiments, the plurality of biomarkers comprises CDCP1, MOG, OPG, and CXCL13. In various embodiments, the plurality of biomarkers comprises CDCP1, MOG, TNFRSF10A, and OPG. In various embodiments, the plurality of biomarkers comprises CDCP1, CXCL9, SERPINA9, and OPG. In various embodiments, the plurality of biomarkers comprises CDCP1, CNTN2, SERPINA9, and OPG. In various embodiments, the plurality of biomarkers comprises CDCP1, SERPINA9, CD6, and OPG. In various embodiments, a performance of the predictive model is characterized by an AUROC of at least 0.65. In various embodiments, a performance of the predictive model is characterized by an AUROC between 0.66 and 0.70.

In various embodiments, the plurality of biomarkers comprises OPG and NEFL. In various embodiments, the plurality of biomarkers comprises OPG and OPN. In various embodiments, the plurality of biomarkers comprises OPG and FLRT2. In various embodiments, the plurality of biomarkers comprises OPG and MOG. In various embodiments, the plurality of biomarkers comprises CXCL9 and OPG. In various embodiments, the plurality of biomarkers comprises GH and NEFL. In various embodiments, the plurality of biomarkers comprises CXCL13 and NEFL. In various embodiments, the plurality of biomarkers comprises APLP1 and NEFL. In various embodiments, the plurality of biomarkers comprises CCL20 and NEFL. In various embodiments, the plurality of biomarkers comprises CXCL9 and NEFL. In various embodiments, the plurality of biomarkers comprises OPG, MOG, and NEFL. In various embodiments, the plurality of biomarkers comprises OPG, FLRT2, and NEFL. In various embodiments, the plurality of biomarkers comprises CXCL9, OPG, and NEFL. In various embodiments, the plurality of biomarkers comprises OPG, CDCP1, and NEFL. In various embodiments, the plurality of biomarkers comprises OPG, OPN, and NEFL. In various embodiments, the plurality of biomarkers comprises GH, APLP1, and NEFL. In various embodiments, the plurality of biomarkers comprises GH, CXCL13, and NEFL. In various embodiments, the plurality of biomarkers comprises GH, CDCP1, and NEFL. In various embodiments, the plurality of biomarkers comprises CXCL13, CCL20, and NEFL. In various embodiments, the plurality of biomarkers comprises GH, CCL20, and NEFL. In various embodiments, the plurality of biomarkers comprises CDCP1, CXCL13, MOG, and NEFL. In various embodiments, the plurality of biomarkers comprises CD6, CXCL9, CXCL13, and NEFL. In various embodiments, the plurality of biomarkers comprises CXCL9, CXCL13, MOG, and NEFL. In various embodiments, the plurality of biomarkers comprises CD6, CDCP1, CXCL13, and NEFL. In various embodiments, the plurality of biomarkers comprises CD6, CXCL9, CXCL13, and MOG. In various embodiments, the plurality of biomarkers comprises CDCP1, CXCL13, MOG, and NEFL. In various embodiments, the plurality of biomarkers comprises CD6, CDCP1, CXCL13, and NEFL. In various embodiments, the plurality of biomarkers comprises CXCL9, CXCL13, MOG, and NEFL. In various embodiments, the plurality of biomarkers comprises CD6, CXCL9, CXCL13, and NEFL. In various embodiments, the plurality of biomarkers comprises CD6, CDCP1, CXCL13, and MOG. In various embodiments, a performance of the predictive model is characterized by a Pearson's R² coefficient of at least 0.10. In various embodiments, a performance of the predictive model is characterized by an Pearson's R² coefficient between 0.015 and 0.090. In various embodiments, the prediction of multiple sclerosis disease progression is a brain parenchymal fraction value. In various embodiments, the prediction of multiple sclerosis disease progression is an expanded disability status scale (EDSS) score. In various embodiments, an expanded disability status scale (EDSS) score less than or equal to 6 indicates a mild/moderate MS disease progression and a EDSS score greater than 6.5 indicates a severe MS disease progression. In various embodiments, the prediction of multiple sclerosis disease progression is a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression. In various embodiments, the prediction of multiple sclerosis disease progression is a PRO measurement information system (PROMIS) score. In various embodiments, the prediction of multiple sclerosis disease progression is a Multiple Sclerosis Rating Scale, Revised (MSRS-R) score.

In various embodiments, generating the prediction of multiple sclerosis disease progression by applying the predictive model to the expression levels of the plurality of biomarkers further comprises applying the predictive model to one or more subject attributes of the subject, wherein subject attributes comprise any of age, sex, and disease duration. In various embodiments, generating the prediction of multiple sclerosis disease progression comprises comparing a score outputted by the predictive model to a reference score. In various embodiments, the reference score corresponds to any of: A) an EDSS score; B) a brain parenchymal fraction value; C) a PDDS score; D) a PROMIS score; or E) a MSRS-R score. In various embodiments, the reference score further corresponds to a mild/moderate MS disease progression or a severe MS disease progression.

In various embodiments, the expression levels of the plurality of biomarkers is determined from a test sample obtained from the subject. In various embodiments, the test sample is a blood or serum sample. In various embodiments, the subject has multiple sclerosis, is suspected of having multiple sclerosis, or was previously diagnosed with multiple sclerosis. In various embodiments, obtaining or having obtained the dataset comprises performing an immunoassay to determine the expression levels of the plurality of biomarkers. In various embodiments, the immunoassay is a Proximity Extension Assay (PEA) or LUMINEX xMAP Multiplex Assay. In various embodiments, performing the immunoassay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies. In various embodiments, the non-transitory computer readable medium further comprises instructions that, when executed by a processor, cause the processor to: select a therapy for administering to the subject based on the prediction of multiple sclerosis disease progression. In various embodiments, the non-transitory computer readable medium further comprises instructions that, when executed by a processor, cause the processor to: determine a therapeutic efficacy of a therapy previously administered to the subject based on the prediction of multiple sclerosis disease progression. In various embodiments, the instructions that cause the processor to determine the therapeutic efficacy of the therapy further comprises instructions that, when executed by the processor, cause the processor to compare the prediction to a prior prediction determined for the subject at a prior timepoint. In various embodiments, the instructions that cause the processor to determine the therapeutic efficacy of the therapy further comprises instructions that, when executed by the processor, cause the processor to determine that the therapy exhibits efficacy responsive to a difference between the prediction and the prior prediction. In various embodiments, the instructions that cause the processor to determine the therapeutic efficacy of the therapy further comprises instructions that, when executed by the processor, cause the processor to determine that the therapy lacks efficacy responsive to a lack of difference between the prediction and the prior prediction.

### BRIEF DESCRIPTION OF THE DRAWINGS

These and other features, aspects, and advantages of the present invention will become better understood with regard to the following description and accompanying drawings.

Figure (FIG. 1A depicts an overview of an environment for assessing disease progression in a subject via a disease progression prediction system, in accordance with an embodiment.

FIG. 1B is an example block diagram of the disease progression system, in accordance with an embodiment.

FIG. 1C depicts an example set of training data, in accordance with an embodiment.

FIG. 1D depicts example biomarkers and their categorizations.

FIG. 2A depicts the sequential forward selection of features using samples from the F6 study.

FIG. 2B depicts the sequential forward selection of features using samples from the F4 study.

FIG. 3A depicts the ROC curve for a multivariate model (train and test) in comparison to a univariate model using neurofilament light as the single feature.

FIG. 3B depicts a confusion matrix for the multivariate model.

FIG. 4A depicts the sequential forward selection of biomarkers for the cross-sectional classification of the presence/absence of radiographically-defined disease activity.

FIG. 4B depicts the ROC curve of the trained model for predicting disease activity (subtle, general, and extreme disease activity).

FIG. 4C depicts the confusion matrix for each of the subtle disease model, general disease model, and extreme disease model.

FIG. 4D depicts the sequential forward selection of biomarkers for predicting the disease severity according to a predicted number of lesions.

FIG. 5A depicts the sequential forward selection of features for building a model for predicting annualized relapse rate (ARR).

FIG. 5B depicts the ROC curve of the trained model for predicting annualized relapse rate as HIGH (>=0.8) or LOW (<0.3).

FIG. 6A depicts the sequential forward selection of features for building a model for classifying exacerbation versus quiescent disease state on two separate patient cohorts.

FIG. 6B depicts the ROC curves for trained models for predicting clinically-defined disease as exacerbation v. quiescent.

FIG. 7 depicts the sequential forward selection of features on absolute quantitation data according to the expanded disability status scale (EDSS).

FIG. 8 illustrates an example computer for implementing the entities shown in FIGS. 1A, 1B, and 1C.

FIG. 9 depicts univariate analysis of individual biomarkers for predicting brain parenchymal fraction.

FIG. 10 depicts a multivariate analysis of a combination of biomarkers for predicting brain parenchymal fraction.

FIG. 11 depicts a multivariate analysis of a combination of biomarkers for predicting brain parenchymal fraction quartiles.

FIG. 12 depicts associations of sex, disease duration, and race/ethnicity with biomarkers (NfL and GFAP).

FIGS. 13A-13C depict characterization of patient-reported outcomes (patient determined disease steps (PDDS), Patient-reported outcomes measurement information system (PROMIS), and Multiple Sclerosis Rating Scale, Revised (MSRS-R).

FIG. 14A depicts a correlation matrix revealing associations between individual biomarkers and PDDS, PROMIS, and MSRS-R.

FIG. 14B depicts univariate analysis of individual biomarkers for predicting PDDS outcomes.

FIG. 14C depicts quantile-quantile plot of expected versus observed p-values of disability severity.

FIG. 14D depicts classification of PDDS-defined severity (e.g., mild/moderate v. severe) according to univariate protein analyses (NfL, CD6, and CXCL13).

FIG. 15A depicts classification of PDDS-defined severity (e.g., mild/moderate v. severe) according to a multivariate biomarker analysis (NfL, CD6, and CXCL13).

FIG. 15B depicts receiver operating characteristic (ROC) curve and precision-recall curve for classifying PDDS-defined severity (e.g., mild/moderate v. severe) through a multivariate biomarker analysis (NfL, CD6, and CXCL13).

FIG. 16 depicts univariate analysis of individual biomarkers for predicting PROMIS or MSRS-R outcomes.

### DETAILED DESCRIPTION

### I. Definitions

Terms used in the claims and specification are defined as set forth below unless otherwise specified.

The term “subject” encompasses a cell, tissue, or organism, human or non-human, whether in vivo, ex vivo, or in vitro, male or female.

The term “mammal” encompasses both humans and non-humans and includes but is not limited to humans, non-human primates, canines, felines, murines, bovines, equines, and porcines.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The term “disease activity” encompasses the disease activity of any neurodegenerative disease including multiple sclerosis, Parkinson's Disease, Lewy body disease, Alzheimer's Disease, Amyotrophic lateral sclerosis (ALS), motor neuron disease, Huntington's Disease, Spinal muscular atrophy, Friedreich's ataxia, Batten disease,

The term “multiple sclerosis” or “MS” encompasses all forms of multiple sclerosis including relapsing-remitting multiple sclerosis (RRMS), secondary progressive multiple sclerosis (SPMS), primary-progressive multiple sclerosis (PPMS), progressive relapsing multiple sclerosis (PRMS), and clinically isolated syndrome (CIS).

The term “multiple sclerosis disease activity” or “disease activity of multiple sclerosis” as used herein refers to any of a diagnosis of multiple sclerosis (MS), a presence or absence of MS (e.g., general disease, subtle disease), a shift (e.g., increase or decrease) in the disease activity, disease progression, a severity of MS, a relapse or flare event associated with MS, a future or impending relapse or flare event, a rate of relapse (e.g., an annualized rate of relapse), a MS state (e.g., exacerbation or quiescence), a confirmation of no evidence of disease status, a response of a subject diagnosed with multiple sclerosis to a therapy, a degree of multiple sclerosis disability, a risk (e.g., likelihood) of the subject developing multiple sclerosis at a subsequent time, a change in multiple sclerosis disease in comparison to a prior measurement (e.g., longitudinal change in a patient relative to a baseline measurement), a measurable that is informative of the disease activity, or a differential diagnosis of a type of multiple sclerosis, including relapsing-remitting multiple sclerosis (RRMS), secondary progressive multiple sclerosis (SPMS), primary-progressive multiple sclerosis (PPMS), progressive relapsing multiple sclerosis (PRMS), and clinically isolated syndrome (CIS).

In one embodiment, the term “multiple sclerosis disease activity” or “disease activity of multiple sclerosis” refers to a diagnosis of multiple sclerosis (MS). In one embodiment, the term “multiple sclerosis disease activity” or “disease activity of multiple sclerosis” refers to a presence or absence of MS (e.g., general disease, subtle disease). In one embodiment, the term “multiple sclerosis disease activity” or “disease activity of multiple sclerosis” refers to a shift (e.g., increase or decrease) in the disease activity. In one embodiment, the term “multiple sclerosis disease activity” or “disease activity of multiple sclerosis” refers to a severity of MS. In one embodiment, the term “multiple sclerosis disease activity” or “disease activity of multiple sclerosis” refers to a relapse or flare event associated with MS. In one embodiment, the term “multiple sclerosis disease activity” or “disease activity of multiple sclerosis” refers to a future or impending relapse or flare event. In one embodiment, the term “multiple sclerosis disease activity” or “disease activity of multiple sclerosis” refers to a rate of relapse (e.g., an annualized rate of relapse). In one embodiment, the term “multiple sclerosis disease activity” or “disease activity of multiple sclerosis” refers to a MS state (e.g., exacerbation or quiescence). In one embodiment, the term “multiple sclerosis disease activity” or “disease activity of multiple sclerosis” refers to a confirmation of no evidence of disease status. In one embodiment, the term “multiple sclerosis disease activity” or “disease activity of multiple sclerosis” refers to a response of a subject diagnosed with multiple sclerosis to a therapy. In one embodiment, the term “multiple sclerosis disease activity” or “disease activity of multiple sclerosis” refers to a degree of multiple sclerosis disability. In one embodiment, the term “multiple sclerosis disease activity” or “disease activity of multiple sclerosis” refers to a risk (e.g., likelihood) of the subject developing multiple sclerosis at a subsequent time. In one embodiment, the term “multiple sclerosis disease activity” or “disease activity of multiple sclerosis” refers to a change in multiple sclerosis disease in comparison to a prior measurement (e.g., longitudinal change in a patient relative to a baseline measurement). In one embodiment, the term “multiple sclerosis disease activity” or “disease activity of multiple sclerosis” refers to a measurable that is informative of the disease activity. In some embodiments, the term “multiple sclerosis disease activity” or “disease activity of multiple sclerosis” is not inclusive of the progression of MS (e.g., MS disease progression). Specifically, in such embodiments as disclosed herein, biomarker panels used for predicting “multiple sclerosis disease activity” are distinct from biomarker panels used for predicting “multiple sclerosis disease progression.”

In various embodiments, measurables that are informative of the MS disease activity include measures of subtle disease activity (e.g., presence or absence of a specific number of gadolinium enhancing lesions e.g., exactly one lesion), general disease activity (e.g., presence or absence of 1 or more gadolinium enhancing lesion), a shift in disease activity (e.g., an appearance or disappearance of active gadolinium enhancing lesions), a severity of disease activity (e.g., a number of gadolinium enhancing lesions, where more gadolinium enhancing lesions is indicative of increased disease severity). In one embodiment, a measure that is informative of MS disease activity includes a measure of subtle disease activity (e.g., presence or absence of a specific number of gadolinium enhancing lesions e.g., exactly one lesion). In one embodiment, a measure that is informative of MS disease activity includes a measure of general disease activity (e.g., presence or absence of 1 or more gadolinium enhancing lesion). In one embodiment, a measure that is informative of MS disease activity includes a measure of a shift in disease activity (e.g., an appearance or disappearance of active gadolinium enhancing lesions). In one embodiment, a measure that is informative of MS disease activity includes a measure of a severity of disease activity (e.g., a number of gadolinium enhancing lesions, where more gadolinium enhancing lesions is indicative of increased disease severity).

In particular embodiments, the term “multiple sclerosis disease activity” or “disease activity of multiple sclerosis” refers to progression of MS (e.g., MS disease progression). In one embodiment, a measure that is informative of MS disease activity includes a measure of disease progression. Examples of measures of disease progression include the expanded disability status scale (EDSS), brain parenchymal fraction (BPF), atrophy measured by brain volume loss, or volumetrics by particular anatomical brain region. Additional measures of disease progression can include patient-reported outcome measures, such as patient determined disease steps (PDDS), PRO measurement information system (PROMIS), Multiple Sclerosis Rating Scale, Revised (MSRS-R), timed 25-foot walk (T25-FW), or hand/arm function as measured by the 9-hole peg test (9-HPT).

In various embodiments, MS disease progression refers to advancing to milestones of MS disability, such as mild MS, moderate MS, or severe MS. Therefore, measures of MS disease progression can correspond to advancing to one or more of mild MS, moderate MS, or severe MS. For example, for a measure of MS disease progression that uses EDSS, an EDSS score less than 6 indicates mild/moderate MS disability and an EDSS score greater than or equal to 6 indicates severe MS disability. As another example, for a measure of MS disability that uses PDDS, a PDDS score less than equal to 4 indicates mild/moderate MS disability and a PDDS score greater than 4 indicates severe MS disability.

The terms “marker,” “markers,” “biomarker,” and “biomarkers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.).

The term “antibody” is used in the broadest sense and specifically covers monoclonal antibodies (including full length monoclonal antibodies), polyclonal antibodies, multispecific antibodies (e.g., bispecific antibodies), and antibody fragments that are antigen-binding so long as they exhibit the desired biological activity, e.g., an antibody or an antigen-binding fragment thereof.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

The term “biomarker panel” refers to a set biomarkers that are informative for predicting multiple sclerosis disease activity, and in particular embodiments, informative for predicting multiple sclerosis disease progression. For example, expression levels of the set of biomarkers in the biomarker panel can be informative for predicting multiple sclerosis disease progression. In various embodiments, a biomarker panel can include two, three, four, five, six, seven, eight, nine, ten eleven, twelve, thirteen, fourteen, fifteen, sixteen, seventeen, eighteen, nineteen, twenty, twenty one, twenty two, twenty three, twenty four, or twenty five biomarkers.

The term “obtaining a dataset associated with a sample” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

It must be noted that, as used in the specification, the singular forms “a,” “an” and “the” include plural referents unless the context clearly dictates otherwise.

### II. System Environment Overview

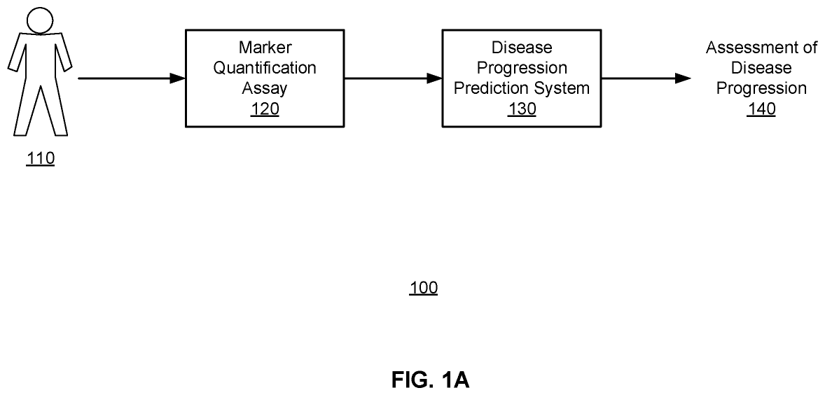

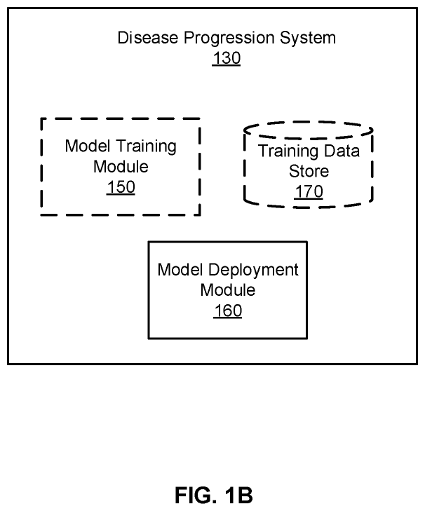

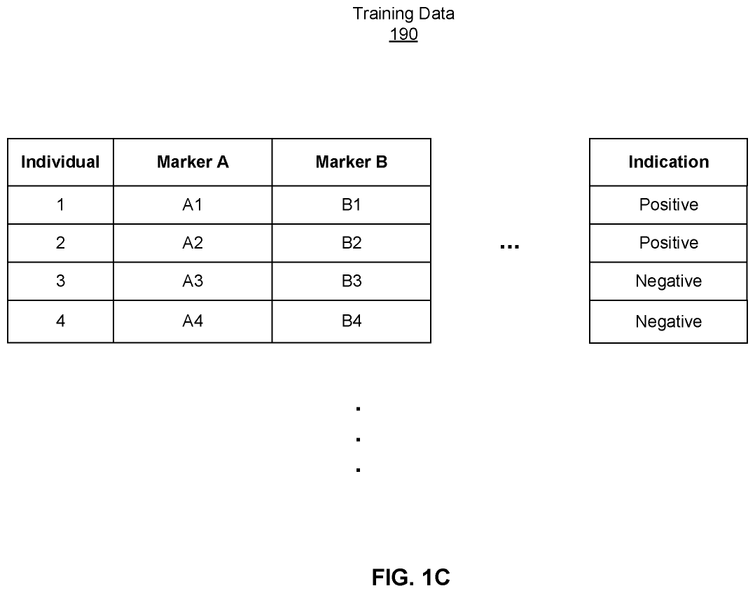

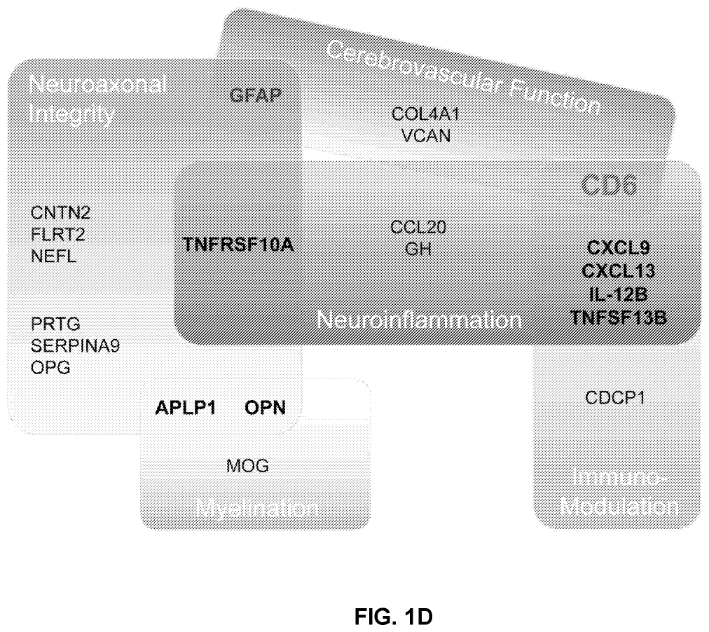

FIG. 1A depicts an overview of a system environment 100 for assessing disease progression in a subject, in accordance with an embodiment. The system environment 100 provides context in order to introduce a marker quantification assay 120 and an disease progression system 130.

In various embodiments, a test sample is obtained from the subject 110. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art.

The test sample is tested to determine values of one or more markers by performing the marker quantification assay 120. The marker quantification assay 120 determines quantitative expression values of one or more biomarkers from the test sample. The marker quantification assay 120 may be an immunoassay, and more specifically, a multi-plex immunoassay, examples of which are described in further detail below. The expression levels of various biomarkers can be obtained in a single run using a single test sample obtained from the subject 110. The quantified expression values of the biomarkers are provided to the disease progression system 130.

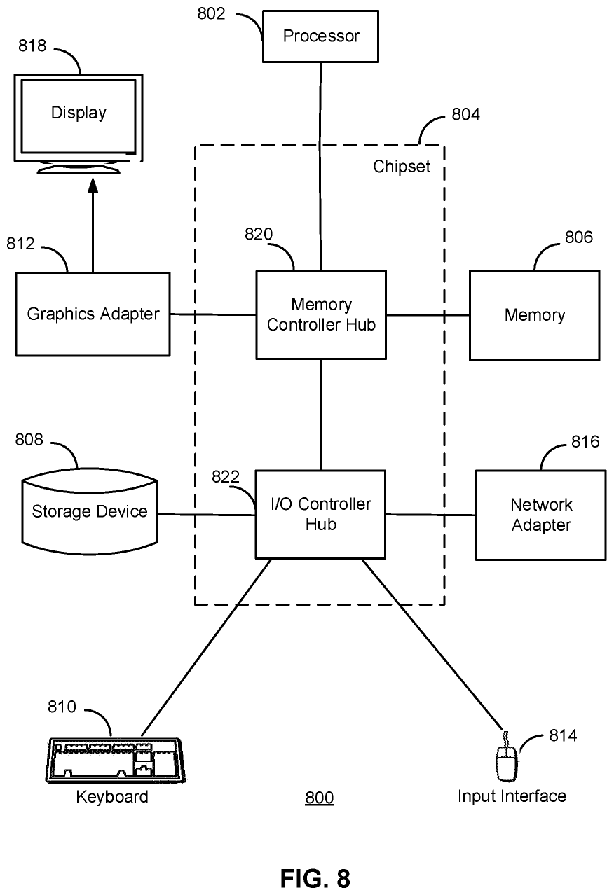

Generally, the disease progression system 130 includes one or more computers, embodied as a computer system 700 as discussed below with respect to FIG. 8. Therefore, in various embodiments, the steps described in reference to the disease progression system 130 are performed in silico. The disease progression system 130 analyzes the received biomarker expression values from the marker quantification assay 120 to generate an assessment of disease progression 140 in the subject 110.

In various embodiments, the marker quantification assay 120 and the disease progression system 130 can be employed by different parties. For example, a first party performs the marker quantification assay 120 which then provides the results to a second party which implements the disease progression system 130. For example, the first party may be a clinical laboratory that obtains test samples from subjects 110 and performs the assay 120 on the test samples. The second party receives the expression values of biomarkers resulting from the performed assay 120 analyzes the expression values using the disease progression system 130.

Reference is now made to FIG. 1B which depicts a block diagram illustrating the computer logic components of the disease progression system 130, in accordance with an embodiment. Specifically, the disease progression system 130 may include a model training module 150, a model deployment module 160, and a training data store 170.

Each of the components of the disease progression system 130 is hereafter described in reference to two phases: 1) a training phase and 2) a deployment phase. More specifically, the training phase refers to the building and training of one or more predictive models based on training data that includes quantitative expression values of biomarkers obtained from individuals that are known to be healthy, in a state of quiescence, in a state of remission, or in an earlier state of disease progression (e.g., mild/moderate MS as opposed to severe MS) or individuals that are known to have disease activity, in a state of exacerbation, in a state of relapse, or in a more advanced state of disease progression (e.g., severe MS as opposed to mild/moderate MS). Therefore, the predictive models are trained to predict disease activity in a subject based on quantitative biomarker expression values. During the deployment phase, a predictive model is applied to quantitative biomarker expression values from a test sample obtained from a subject of interest in order to generate a prediction of disease activity in the subject of interest.

In some embodiments, the components of the disease progression system 130 are applied during one of the training phase and the deployment phase. For example, the model training module 150 and training data store 170 (indicated by the dotted lines in FIG. 1B) are applied during the training phase whereas the model deployment module 160 is applied during the deployment phase. In various embodiments, the training phase and the deployment phase can be performed to enable continuously trained models. For example, the model training module 150 can train a model that the model deployment module 160 can subsequently deploy. The same model can undergo additional training by the model training module 150 (e.g., continuously trained using, for example, new training data that is obtained). Therefore, as the model is continuously trained, it can exhibit improved prediction capacity when analyzing samples during deployment.

In various embodiments, the components of the disease progression system 130 can be performed by different parties depending on whether the components are applied during the training phase or the deployment phase. In such scenarios, the training and deployment of the predictive model are performed by different parties. For example, the model training module 150 and training data store 170 applied during the training phase can be employed by a first party (e.g., to train a predictive model) and the model deployment module 160 applied during the deployment phase can be performed by a second party (e.g., to deploy the predictive model).

### III. Predictive Model

III.A. Training a Predictive Model

During the training phase, the model training module 150 trains one or more predictive models using training data comprising expression values of biomarkers. Referring to FIG. 1B, the training data may be stored in the training data store 170. In various embodiments, the disease progression system 130 generates the training data comprising expression values of biomarkers by analyzing biomarker expression values in test samples. In various embodiments, the disease progression system 130 obtains the training data comprising expression values of biomarkers from a third party. The third party may have analyzed test samples to determine the biomarker expression values.

In various embodiments, the training data comprising expression values of biomarkers are derived from clinical subjects. For example, the training data can be expression values of biomarkers that were measured from test samples obtained from clinical subjects. Examples of expression values of biomarkers derived from clinical subjects include biomarker expression values obtained through clinical studies such as the multiple sclerosis CLIMB study (e.g., Comprehensive Longitudinal Investigation of Multiple Sclerosis at Brigham and Women's Hospital), the Accelerated Cure Project (ACP) for Multiple Sclerosis, and the Expression, Proteomics, Imaging, Clinical (EPIC) study at UCSF, the University Hospital Basel Cohort (UHBC), and the Prospective Investigation of Multiple Sclerosis in the Three Rivers Region (PROMOTE) study at the University of Pittsburgh.

In various embodiments, the training data further includes reference ground truths that indicate a disease activity, such as a multiple sclerosis disease activity. As an example, the training data includes reference ground truths that identify a presence or absence of multiple sclerosis (MS), a relapse or flare event associated with MS, a rate of relapse (e.g., an annualized rate of relapse), a MS state (e.g., exacerbation or quiescence), a response of a subject diagnosed with multiple sclerosis to a therapy, a degree of multiple sclerosis disability (e.g., a measure of multiple sclerosis disease progression such as mild, moderate, or severe MS), a risk (e.g., likelihood) of the subject developing multiple sclerosis at a subsequent time, or a measure of subtle disease activity (e.g., presence or absence of a specific number of gadolinium enhancing lesions e.g., one, two, three, or four lesions), or a measure of general disease activity (e.g., presence or absence of 1 or more gadolinium enhancing lesion). In particular embodiments, training data includes reference ground truths that identify a degree of multiple sclerosis disability (e.g., a measure of multiple sclerosis disease progression such as mild, moderate, or severe MS). In various embodiments, reference ground truths are generated by analyzing images (e.g., brain MM images such as T1 or FLAIR images) captured from clinical subjects. Such images can be analyzed through computational means (e.g., image analysis algorithm) or can be manually analyzed. For example, images can be analyzed to determine a brain parenchymal fraction value, which is a known marker for MS disease progression. In various embodiments, the brain parenchymal fraction value of an image can serve as the reference ground truth. In various embodiments, the image analysis is performed by a third party and the reference ground truths can then be used for training the models described herein.

Reference is made to FIG. 1C, which depicts an example set of training data 190, in accordance with an embodiment. As shown in FIG. 1C, the training data 190 includes data corresponding to multiple individuals (e.g., column 1 depicting individual 1, 2, 3, 4 . . . ). For each individual, the training data 190 includes quantitative expression values (e.g., A1, B1, A2, B2, etc.) for different biomarkers obtained from the corresponding individual. In some embodiments, the quantitative expression values are determined by the marker quantification assay 120 shown in FIG. 1. Although FIG. 1C depicts 4 individuals and 2 different markers (marker A and marker B), the training data 190 may include tens, hundreds, or thousands of individuals as well as tens, hundreds, or thousands of markers.

As shown in FIG. 1C, a first training example (e.g., first row) of the training data refers to individual 1 and corresponding quantitative expression values of marker A (e.g., A1) and the quantitative expression value of marker B (e.g., B1). Similarly, the second training example (e.g., second row) of the training data refers to individual 2 and corresponding quantitative expression values of marker A (e.g., A2) and the quantitative expression value of marker B (e.g., B2). Individuals 3 and 4 have corresponding marker values as shown in FIG. 1C.

As shown in FIG. 1C, the training data 190 further includes a reference ground truth (“Indication” column) that identifies whether the corresponding individual has a positive or negative indication as to the disease activity. As an example, each indication may be an indication of multiple sclerosis disease progression in the patient. For example, referring to the first training example (e.g., first row), a “Positive” indication can reflect a presence of severe disease progression in individual 1. For example, a MRI scan of individual 1 may have revealed a presence of multiple gadolinium enhancing lesions. Similarly, an indication of a negative result (e.g., individual 3 or individual 4) reflects a presence of mild or moderate disease progression in the corresponding individual.

As another example, instead of the reference ground truth indicating a binary option (e.g., positive/negative), the reference ground truth may indicate one of multiple classes. For example, the reference ground truth may include a continuous range of values, wherein each value is indicative of one of the multiple classes. Specifically, the reference ground truth may include a value (e.g., value of “1”) which indicates that the corresponding individual has a presence of mild MS. A reference ground truth may include a value (e.g., value of “2”) which indicates that the corresponding individual has a presence of moderate MS. A reference ground truth may include a value (e.g., value of “3”) which indicates that the corresponding individual has a presence of severe MS. Additional values can be assigned that can further sub-divide the measures of disease progression into further classes.

In various embodiments, the reference ground truth may be a score, such as any of an EDSS score, a PDDS score, a PROMIS score, or a MSRS-R score. Therefore the reference ground truth score may itself be indicative of MS disease progression (e.g., PDDS score less or equal than 4 indicates mild/moderate MS whereas PDDS score greater than 4 indicates severe MS). Therefore, by training the predictive model using these reference ground truth scores, the predictive model is trained to predict a score (e.g., EDSS score, a PDDS score, a PROMIS score, or a MSRS-R score) for an individual that is indicative of MS disease progression.

In various embodiments, the reference ground truth may correspond to brain parenchymal fraction values that are derived from images captured from an individual, such as MM images (T1 or FLAIR images). Here, brain parenchymal fraction is a known correlate to MS patients' disease progression. Thus, by training the predictive model using these reference ground truth scores, the predictive model is trained to predict a value that corresponds to brain parenchymal fraction values.

In various embodiments, the reference ground truth may indicate a particular class according to brain parenchymal fraction values derived from MRI images. MRI images can be analyzed and separated into different subsets according to brain parenchymal fraction values of the MRI images. For example, MRI images can be separated into 4 subsets (e.g., brain parenchymal fraction quartiles), where the first subset includes MRI images with the lowest range of brain parenchymal fraction values, the second subset includes MRI images with the next lowest range of brain parenchymal fraction values, the third subset includes MM images with the third lowest range of brain parenchymal fraction values, and the fourth subset includes MRI images with the highest range of brain parenchymal fraction values. Thus, by training the predictive model using these reference ground truth scores, the predictive model can be trained to predict different classes according to predicted brain parenchymal fraction values.

In some embodiments, the model training module 150 retrieves the training data from the training data store 170 and randomly partitions the training data into a training set and a test set. As an example, 80% of the training data may be partitioned into the training set and the other 20% can be partitioned into the test set. Other proportions of training set and test set may be implemented. As such, the training set is used to train predictive models whereas the test set is used to validate the predictive models.

In various embodiments, the predictive model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), linear mixed effects (LME) model, or any combination thereof. For example, the predictive model can be a stacked classifier that includes both a linear regression and decision tree.

The predictive model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the cellular disease model is trained using supervised learning algorithms, unsupervised learning algorithms, semi-supervised learning algorithms (e.g., partial supervision), weak supervision, transfer, multi-task learning, or any combination thereof.

In various embodiments, the predictive model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the cellular disease model are trained (e.g., adjusted) using the training data to improve the predictive capacity of the cellular disease model.

The model training module 150 trains one or more predictive models, each predictive model receiving, as input, one or more biomarkers. In various embodiments, the model training module 150 constructs a predictive model that receives, as input, expression values of two biomarkers. In various embodiments, the model training module 150 constructs a predictive model that receives, as input, expression values of three biomarkers. In various embodiments, the model training module 150 constructs a predictive model that receives, as input, expression values of four biomarkers. In some embodiments, the model training module 150 constructs a predictive model for more than four biomarkers. For example, a predictive model receives, as input, expression values of 8 biomarkers (e.g., 8 biomarkers categorized as Tier 1 in Table 2, 8 biomarkers categorized as Tier A in Table 1, or 8 biomarkers categorized as Tier 1 in Table 3, or any of their corresponding substitute biomarkers in Table 4). As another example, the predictive model receives, as input, expression values of 17 biomarkers (e.g., 17 biomarkers categorized as Tier 1 or Tier 2 in Table 2, 17 biomarkers categorized as Tier A or Tier B in Table 1, 17 biomarkers categorized as Tier 1 or Tier 2 in Table 3, or any of their corresponding substitute biomarkers in Table 4). As another example, the predictive model receives, as input, expression values of 16 biomarkers (e.g., except for COL4A1 in Table 2, the 16 biomarkers categorized as Tier 1 or Tier 2 or any of their corresponding substitute biomarkers in Table 4). As another example, the predictive model receives, as input, expression values of 21 biomarkers (e.g., 21 biomarkers categorized as Tier 1, Tier 2, or Tier 3 in Table 2, 21 biomarkers categorized as Tier 1, Tier 2, or Tier 3 in Table 3, 21 biomarkers categorized as Tier A, Tier B, or Tier C in Table 1, or any of their corresponding substitute biomarkers in Table 4). As another example, the predictive model receives, as input, expression values of 20 biomarkers (e.g., except for COL4A1 in Table 2, the 20 biomarkers categorized as Tier 1, Tier 2, or Tier 3 in Table 2 or any of their corresponding substitute biomarkers in Table 4).

In various embodiments, the model training module 150 identifies a set of biomarkers that are to be used to train a predictive model. The model training module 150 may begin with a list of candidate biomarkers that are promising for predicting disease activity (e.g., MS disease progression). In one embodiment, candidate biomarkers may be biomarkers identified through a literature curation process. In some embodiments, candidate biomarkers may be biomarkers whose expression values in test samples obtained from individuals that are positive for a disease activity (e.g., presence of MS, in an exacerbated state, in a state of severe MS, and the like) are statistically significant in comparison to expression values of biomarkers in test samples obtained from individuals that are negative for the disease activity.

In one embodiment, the model training module 150 performs a feature selection process to identify the set of biomarkers to be included in the biomarker panel. For example, the model training module 150 performs a sequential forward feature selection based on the expression values of the biomarkers and their importance in predicting a particular endpoint. For example, candidate biomarkers that are determined to be highly correlated with a particular disease activity endpoint (e.g., disease progression endpoint) would be deemed highly important are therefore likely to be included in the biomarker panel in comparison to other biomarkers that are not highly correlated with the disease activity endpoint (e.g., disease progression endpoint).

In some embodiments, the importance of each biomarker for a disease activity endpoint (e.g., disease progression endpoint) is determined by using a method including one of random forest (RF), gradient boosting (GBM), extreme gradient boosting (XGB), or LASSO algorithms. For example, if using random forest algorithms, the model training module 150 may generate a variable importance plot that depicts the importance of each candidate biomarker. Specifically, the random forest algorithm may provide, for each candidate biomarker, 1) a mean decrease in model accuracy and 2) a mean decrease in a Gini coefficient which is a measure of how much each candidate biomarker contributes to the homogeneity of nodes and leaves in the random forest. In one scenario, the importance of each candidate biomarker is dependent on one or both of the mean decrease in model accuracy and mean decrease in Gini coefficient. Each of GBM, XGB, and LASSO, can also be used to rank the importance of each candidate biomarker based on an influence value. Therefore, the model training module 150 can generate a ranking of each of candidate biomarkers using one of the methods including RF, GBM, XGB, or LASSO.

Each predictive model is iteratively trained using, as input, the quantitative expression values of the markers for each individual. For example, referring again to FIG. 1C, one iteration involves providing a training example (e.g., a row of the training data) that includes the quantitative expression value of biomarkers (e.g., “A1” and “B 1”) for a particular individual (e.g., individual 1). Each predictive model is trained on reference ground truth data that includes the indication (e.g., the positive or negative result). In various embodiments, over training iterations, each predictive model is trained (e.g., the parameters are tuned) to minimize a prediction error between a prediction of MS activity (e.g., prediction of MS disease progression) outputted by the predictive model and the ground truth data. In various embodiments, the prediction error is calculated based on a loss function, examples of which include a L1 regularization (Lasso Regression) loss function, a L2 regularization (Ridge Regression) loss function, or a combination of L1 and L2 regularization (ElasticNet).

III.B. Deploying a Predictive Model

During the deployment phase, the model deployment module 160 (as shown in FIG. 1B) analyzes quantitative biomarker expression values from a test sample obtained from a subject of interest by applying a trained predictive model. In some embodiments, the subject has not previously been diagnosed with a disease and therefore, the deployment of the predictive model enables in silico diagnosis of the disease based on the quantitative biomarker expression values derived from the subject. In some embodiments, the subject has been previously diagnosed with a disease. Here, the deployment of the predictive model enables in silico prediction of disease activity (e.g., disease progression) based on the quantitative biomarker expression values derived from the subject.

In various embodiments, the quantitative biomarker expression values are provided as input to the predictive model. The predictive model analyzes the quantitative biomarker expression values and outputs an assessment of disease activity (e.g., disease progression). The predicted score can then be informative of the disease activity. For example, the predicted score can enable the classification of the subject into one of multiple disease progression categories (e.g., one of mild/moderate disease progression or severe progression). In various embodiments, the assessment of disease activity (e.g., disease progression) is a predicted score representing the learned combination of the quantitative biomarker expression values. Generally, the predicted score represents an aggregation of the quantitative expression values and therefore, is not directly dependent on solely one biomarker expression value.

In various embodiments, the assessment of disease activity is a predicted score that may be informative of the disease activity in the subject. In various embodiments, the predicted score outputted by the prediction model is compared to one or more reference scores to determine a measure of the disease activity. Reference scores refer to previously determined scores, further described below as “healthy scores” or “diseased scores,” that correspond to diseased patients or non-diseased patients. For example, the one or more scores may be “healthy scores” corresponding to healthy patients, a patient's own baseline at a prior timepoint when the patient did not exhibit disease activity (e.g., longitudinal analysis), patients clinically diagnosed with the disease but not exhibiting disease activity, or a threshold score (e.g., a cutoff). As another example, the one or more scores may be “diseased scores” corresponding to diseased patients, a patient's own score indicating disease activity at a prior timepoint, or a threshold score (e.g., a cutoff). As one example, the threshold score can correspond to healthy patients and can be generated by training a predictive model using expression values of biomarkers from healthy patients. As another example, the threshold score can correspond to diseased patients and can be generated by training a predictive model using expression values of biomarkers from the diseased patients.

In various embodiments, a threshold score corresponding to healthy patients can be lower than a threshold score corresponding to diseased patients. For example, the threshold score corresponding to healthy patients can be at least 5% lower than a threshold score corresponding to diseased patients. As another example, the threshold score corresponding to healthy patients can be at least 10% lower than a threshold score corresponding to diseased patients. As another example, the threshold score corresponding to healthy patients can be at least 15% lower than a threshold score corresponding to diseased patients. As another example, the threshold score corresponding to healthy patients can be at least 20% lower than a threshold score corresponding to diseased patients. As another example, the threshold score corresponding to healthy patients can be at least 25% lower than a threshold score corresponding to diseased patients. As another example, the threshold score corresponding to healthy patients can be at least 50% lower than a threshold score corresponding to diseased patients. As another example, the threshold score corresponding to healthy patients can be at least 75% lower than a threshold score corresponding to diseased patients.

In various embodiments, a threshold score corresponding to healthy patients can be higher than a threshold score corresponding to diseased patients. For example, the threshold score corresponding to healthy patients can be at least 5% higher than a threshold score corresponding to diseased patients. As another example, the threshold score corresponding to healthy patients can be at least 10% higher than a threshold score corresponding to diseased patients. As another example, the threshold score corresponding to healthy patients can be at least 15% higher than a threshold score corresponding to diseased patients. As another example, the threshold score corresponding to healthy patients can be at least 20% higher than a threshold score corresponding to diseased patients. As another example, the threshold score corresponding to healthy patients can be at least 25% higher than a threshold score corresponding to diseased patients. As another example, the threshold score corresponding to healthy patients can be at least 50% higher than a threshold score corresponding to diseased patients. As another example, the threshold score corresponding to healthy patients can be at least 75% higher than a threshold score corresponding to diseased patients. As another example, the threshold score corresponding to healthy patients can be at least 100% higher than a threshold score corresponding to diseased patients. Thus, in particular embodiments, the predicted score outputted by the prediction model is compared to one or both of the threshold score corresponding to healthy patients and threshold score corresponding to diseased patients, and based on the comparison, a measure of the disease activity is determined.

In various embodiments, the assessment of disease activity corresponds to the presence of absence of disease. In one embodiment, the predicted score outputted by the prediction model can be compared to a healthy score. The subject can be classified as having the disease if the predicted score of the subject is significantly different (e.g., p-value <0.05) in comparison to the healthy score. In one embodiment, the predicted score outputted by the prediction model can be compared to the diseased score. The subject can be classified as not having the disease if the predicted score of the subject is significantly different (e.g., p-value <0.05) in comparison to the diseased score. In some embodiments, the predicted score outputted by the prediction model is compared to both the healthy score and the diseased score. For example, the subject can be classified as having the disease if the predicted score of the subject is significantly different (e.g., p-value <0.05) in comparison to the healthy scores and not significantly different (e.g., p-value >0.05 in comparison to the diseased scores for patients that have been diagnosed with the disease. In various embodiments, depending on the classification of the subject, the subject can undergo treatment. In other words, the assessment can guide the treatment of the subject. For example, if the subject is classified as having the disease, the subject can be administered a therapeutic intervention to treat the disease.

In various embodiments, the assessment of disease activity corresponds to the presence of absence of subtle disease. In one embodiment, the predicted score outputted by the prediction model can be compared to a score corresponding to individuals previously determined to have a presence of subtle disease (e.g., a specific number of gadolinium enhancing lesion on a MRI scan e.g., exactly one lesion). The subject can be classified as having subtle disease if the predicted score of the subject is not significantly different (e.g., p-value >0.05) in comparison to the score corresponding to individuals previously determined to have a presence of subtle disease. The subject can be classified as having subtle disease if the predicted score of the subject is significantly different (e.g., p-value <0.05) in comparison to the score corresponding to individuals previously determined to not have a presence of subtle disease. In one embodiment, the predicted score outputted by the prediction score is compared to a score corresponding to individuals without subtle disease (e.g., zero gadolinium enhancing lesions on a MRI scan). The subject can be classified as not having subtle disease if the predicted score of the subject is not significantly different (e.g., p-value >0.05) from the score corresponding to individuals that do not have subtle disease (e.g., zero gadolinium enhancing lesion on a MM scan). Alternatively, the subject can be classified as having subtle disease if the predicted score of the subject is significantly different (e.g., p-value <0.05) from the score corresponding to individuals that do not have subtle disease (e.g., zero gadolinium enhancing lesion on a MRI scan).

In some embodiments, the predicted score outputted by the prediction model is compared to both a score corresponding to individuals previously determined to have a presence of subtle disease (e.g., a particular number of gadolinium enhancing lesions on a MRI scan) and a score corresponding to individuals without subtle disease (e.g., zero gadolinium enhancing lesions on a MRI scan). For example, the subject can be classified as having subtle disease if the predicted score of the subject is significantly different (e.g., p-value <0.05) in comparison to the score corresponding to individuals without subtle disease (e.g., zero gadolinium enhancing lesions on a MRI scan) and not significantly different (e.g., p-value >0.05) in comparison to the score corresponding to individuals previously determined to have a presence of subtle disease (e.g., a particular number of gadolinium enhancing lesions on a MM scan e.g., exactly one gadolinium enhancing lesion).

In various embodiments, the assessment of disease activity corresponds to the presence of absence of general disease. In one embodiment, the predicted score outputted by the prediction model can be compared to a score corresponding to individuals previously determined to have a presence of general disease (e.g., one or more gadolinium enhancing lesions on a MM scan). The subject can be classified as having general disease if the predicted score of the subject is significantly different (e.g., p-value <0.05) in comparison to the score corresponding to individuals previously determined to not have a presence of general disease. The subject can be classified as not having general disease if the predicted score of the subject is not significantly different (e.g., p-value >0.05) in comparison to the score corresponding to individuals previously determined to not have a presence of general disease. In one embodiment, the predicted score outputted by the prediction score is compared to a score corresponding to individuals without general disease (e.g., zero gadolinium enhancing lesions on a MRI scan). The subject can be classified as not having general disease if the predicted score of the subject is not significantly different (e.g., p-value >0.05) from the score corresponding to individuals that do not have general disease (e.g., zero gadolinium enhancing lesion on a MRI scan). The subject can be classified as having general disease if the predicted score of the subject is significantly different (e.g., p-value <0.05) from the score corresponding to individuals that do not have general disease (e.g., zero gadolinium enhancing lesion on a MM scan). In some embodiments, the predicted score outputted by the prediction model is compared to both a score corresponding to individuals previously determined to have a presence of general disease (e.g., one or more gadolinium enhancing lesions on a MM scan) and a score corresponding to individuals without general disease (e.g., zero gadolinium enhancing lesions on a MM scan). For example, the subject can be classified as having general disease if the predicted score of the subject is significantly different (e.g., p-value <0.05) in comparison to the score corresponding to individuals without general disease (e.g., zero gadolinium enhancing lesions on a MRI scan) and not significantly different (e.g., p-value >0.05) in comparison to the score corresponding to individuals previously determined to have a presence of general disease (e.g., one or more gadolinium enhancing lesions on a MRI scan).

In various embodiments, the assessment of disease activity corresponds to the directional shift in disease activity based on a predicted increase or decrease in the number of gadolinium enhancing lesions. In one embodiment, the predicted score outputted by the prediction model can be compared to a score corresponding to individuals previously determined to have undergone an increase in disease activity (e.g., increasing numbers of gadolinium enhancing lesions on a MRI scan). The subject can be classified as likely to encounter an increase in disease activity if the predicted score of the subject is significantly different (e.g., p-value <0.05) in comparison to the score corresponding to individuals previously determined to not have undergone an increase in disease activity. The subject can be classified as unlikely to encounter an increase in disease activity if the predicted score of the subject is not significantly different (e.g., p-value >0.05) in comparison to the score corresponding to individuals previously determined to not have undergone an increase in disease activity. In one embodiment, the predicted score outputted by the prediction score is compared to a score corresponding to individuals previously determined to have undergone a decrease in disease activity (e.g., decreasing numbers of gadolinium enhancing lesions on a MRI scan). The subject can be classified as likely to encounter a decrease in disease activity if the predicted score of the subject is not significantly different (e.g., p-value >0.05) from the score corresponding to individuals that have encountered a decrease in disease activity. The subject can be classified as likely to encounter a decrease in disease activity if the predicted score of the subject is significantly different (e.g., p-value <0.05) from the score corresponding to individuals that have not encountered a decrease in disease activity. In some embodiments, the predicted score outputted by the prediction model is compared to both a score corresponding to individuals previously determined to have undergone an increase in disease activity (e.g., increasing numbers of gadolinium enhancing lesions on a MRI scan) and a score corresponding to individuals who have undergone a decrease in disease activity (e.g., decreasing numbers of gadolinium enhancing lesions on a MM scan). For example, the subject can be classified as likely to undergo an increase in disease activity if the predicted score of the subject is significantly different (e.g., p-value <0.05) in comparison to the score corresponding to individuals who have undergone a decrease in disease activity (e.g., decreasing number of gadolinium enhancing lesions on a MRI scan) and not significantly different (e.g., p-value >0.05) in comparison to the score corresponding to individuals who have undergone an increase in disease activity (e.g., increasing numbers of gadolinium enhancing lesions on a MRI scan). In various embodiments, the subject can be classified as unlikely to encounter either an increase or decrease in disease activity (e.g., the disease activity in the subject is stable) if the predicted score of the subject is not significantly different (e.g., p-value >0.05) in comparison to both the score corresponding to individuals who have undergone an increase in disease activity and the score corresponding to individuals who have undergone a decrease in disease activity.

In various embodiments, the assessment of disease activity corresponds to a state of disease in a subject. For example, if the disease is MS, the state of disease in the subject is one of quiescent vs exacerbation. In one embodiment, the predicted score outputted by the prediction model can be compared to a score corresponding to individuals previously determined to be in a quiescent state (e.g., clinically determined to be in a quiescent state). The subject can be classified as being in a quiescent state if the predicted score of the subject is significantly different (e.g., p-value <0.05) in comparison to the score corresponding to individuals previously determined to not be in a quiescent state. The subject can be classified as not being in a quiescent state if the predicted score of the subject is not significantly different (e.g., p-value >0.05) in comparison to the score corresponding to individuals previously determined to be in a quiescent state. In one embodiment, the predicted score outputted by the prediction score is compared to a score corresponding to individuals previously determined to be in an exacerbated state. The subject can be classified as being in an exacerbated state if the predicted score of the subject is not significantly different (e.g., p-value >0.05) from the score corresponding to individuals previously determined to be in an exacerbated state. The subject can be classified as not being in an exacerbated state if the predicted score of the subject is significantly different (e.g., p-value <0.05) from the score corresponding to individuals previously determined to be in an exacerbated state. In some embodiments, the predicted score outputted by the prediction model is compared to both a score corresponding to individuals previously determined to be in a quiescent state and a score corresponding to individuals in an exacerbated state. For example, the subject can be classified as being in an exacerbated state if the predicted score of the subject is significantly different (e.g., p-value <0.05) in comparison to the score corresponding to individuals in a quiescent state and not significantly different (e.g., p-value >0.05) in comparison to the score corresponding to individuals previously determined to be in an exacerbated state.

In various embodiments, the assessment of disease activity corresponds to a likely response to a therapy of provided to the subject. In one embodiment, the predicted score outputted by the prediction model can be compared to a score corresponding to individuals previously determined to be responsive to the therapy (e.g., clinically determined to be responsive to the therapy). The subject can be classified as being a responder if the predicted score of the subject is significantly different (e.g., p-value <0.05) in comparison to the score corresponding to individuals previously determined to not be responsive to the therapy. The subject can be classified as being a responder if the predicted score of the subject is not significantly different (e.g., p-value >0.05) in comparison to the score corresponding to individuals previously determined to be responsive to the therapy. In one embodiment, the predicted score outputted by the prediction score is compared to a score corresponding to individuals previously determined to be non-responders. The subject can be classified as a non-responder if the predicted score of the subject is not significantly different (e.g., p-value >0.05) from the score corresponding to individuals previously determined to be non-responders. The subject can be classified as a non-responder if the predicted score of the subject is significantly different (e.g., p-value <0.05) from the score corresponding to individuals previously determined to be responders. In some embodiments, the predicted score outputted by the prediction model is compared to both a score corresponding to individuals previously determined to be responders and a score corresponding to individuals previously determined to be non-responders. For example, the subject can be classified as being a responder if the predicted score of the subject is significantly different (e.g., p-value <0.05) in comparison to the score corresponding to individuals previously determined to be non-responders and not significantly different (e.g., p-value >0.05) in comparison to the score corresponding to individuals previously determined to be responders.

In various embodiments, the assessment of disease activity is a classification of disease progression (e.g., mild/moderate disease versus severe disability). Thus, in such embodiments, the predicted score outputted by the prediction model can be compared to one or both scores corresponding to individuals previously identified as having mild/moderate disease and corresponding to individuals previously identified as having severe disability.

In various embodiments, a score corresponding to individuals previously identified as having mild/moderate disease can be lower than a score corresponding to individuals previously identified as having severe disability. For example, the score corresponding to individuals previously identified as having mild/moderate disease can be at least 5% lower than a score corresponding to individuals previously identified as having severe disability. As another example, the score corresponding to individuals previously identified as having mild/moderate disease can be at least 10% lower than a score corresponding to individuals previously identified as having severe disability. As another example, the score corresponding to individuals previously identified as having mild/moderate disease can be at least 15% lower than a score corresponding to individuals previously identified as having severe disability. As another example, the score corresponding to individuals previously identified as having mild/moderate disease can be at least 20% lower than a score corresponding to individuals previously identified as having severe disability. As another example, the score corresponding to individuals previously identified as having mild/moderate disease can be at least 25% lower than a score corresponding to individuals previously identified as having severe disability. As another example, the score corresponding to individuals previously identified as having mild/moderate disease can be at least 50% lower than a score corresponding to individuals previously identified as having severe disability. As another example, the score corresponding to individuals previously identified as having mild/moderate disease can be at least 75% lower than a score corresponding to individuals previously identified as having severe disability.

In various embodiments, the score corresponding to individuals previously identified as having mild/moderate disease can be higher than a score corresponding to individuals previously identified as having severe disability. For example, the score corresponding to individuals previously identified as having mild/moderate disease can be at least 5% higher than a score corresponding to individuals previously identified as having severe disability. As another example, the score corresponding to individuals previously identified as having mild/moderate disease can be at least 10% higher than a score corresponding to individuals previously identified as having severe disability. As another example, the score corresponding to individuals previously identified as having mild/moderate disease can be at least 15% higher than a score corresponding to individuals previously identified as having severe disability. As another example, the score corresponding to individuals previously identified as having mild/moderate disease can be at least 20% higher than a score corresponding to individuals previously identified as having severe disability. As another example, the score corresponding to individuals previously identified as having mild/moderate disease can be at least 25% higher than a score corresponding to individuals previously identified as having severe disability. As another example, the score corresponding to individuals previously identified as having mild/moderate disease can be at least 50% higher than a score corresponding to individuals previously identified as having severe disability. As another example, the score corresponding to individuals previously identified as having mild/moderate disease can be at least 75% higher than a score corresponding to individuals previously identified as having severe disability. As another example, the score corresponding to individuals previously identified as having mild/moderate disease can be at least 100% higher than a score corresponding to individuals previously identified as having severe disability. Thus, in particular embodiments, the predicted score outputted by the prediction model is compared to one or both of the score corresponding to individuals previously identified as having mild/moderate disease and score corresponding to individuals previously identified as having severe disability, and based on the comparison, a measure of the disease progression is determined.

In one embodiment, the assessment of disease activity is an assessment of disease progression and can correspond to a degree of MS disability in a subject diagnosed with multiple sclerosis. In one embodiment, the degree of MS disability corresponds to an EDSS score or to a range of EDSS scores. In various embodiments, the assessment (e.g., predicted score) corresponding to the subject is compared to multiple reference scores. Each reference score may correspond to a group of individuals that have been clinically categorized in a degree of disability. In various embodiments, a reference score is an EDSS score. In various embodiments, a reference score corresponds to an EDSS score. For example, a first reference score may correspond to individuals clinically categorized with a score of 1 on the EDSS. Additional reference scores may correspond to groups of individuals that have been clinically categorized with a score of 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0. As another example, a first reference score may correspond to individuals previously categorized with a score between 1 and 6 on the EDSS scale and a second reference score may correspond to individuals previously categorized with a score between 6.5 and 10 on the EDSS scale. In one scenario, the subject may be classified with one of the EDSS scores if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a PDDS score or a range of PDDS scores. In various embodiments, the assessment (e.g., predicted score) corresponding to the subject is compared to multiple reference scores. Each score may correspond to a group of individuals that have been clinically categorized in a degree of disability. In various embodiments, a reference score is a PDDS score. In various embodiments, a reference score corresponds to a PDDS score. For example, a first reference score may correspond to individuals previously categorized with a score of 1 on the PDDS scale. Additional reference scores may correspond to groups of individuals that have been previously categorized with a score of 2, 3, 4, 5, 6, 7, or 8. As another example, a first reference score may correspond to individuals previously categorized with a score between 1 and 4 on the PDDS scale and a second reference score may correspond to individuals previously categorized with a score between 5 and 8 on the PDDS scale. In one scenario, the subject may be classified with one of the PDDS scores if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a brain parenchymal fraction score. In various embodiments, the assessment (e.g., predicted score) corresponding to the subject is compared to one or more reference scores. For example, a first reference score may be a brain parenchymal fraction score corresponding to individuals previously categorized as having mild/moderate MS. A second reference score may be a brain parenchymal fraction score corresponding to individuals previously categorized as having severe MS. In one scenario, the subject may be classified with having mild/moderate or severe MS if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a PROMIS score. In various embodiments, the assessment (e.g., predicted score) corresponding to the subject is compared to one or more reference scores. For example, a first reference score may be a PROMIS score corresponding to individuals previously categorized as having mild/moderate MS. A second reference score may be a PROMIS score corresponding to individuals previously categorized as having severe MS. In one scenario, the subject may be classified with having mild/moderate or severe MS if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a MSRS-R score. In various embodiments, the assessment (e.g., predicted score) corresponding to the subject is compared to one or more reference scores. For example, a first reference score may be a MSRS-R score corresponding to individuals previously categorized as having mild/moderate MS. A second reference score may be a MSRS-R score corresponding to individuals previously categorized as having severe MS. In one scenario, the subject may be classified with having mild/moderate or severe MS if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In one embodiment, the assessment of disease activity corresponds to a risk (e.g., likelihood) of the subject developing a disease at a subsequent time. In various embodiments, the assessment (e.g., predicted score) corresponding to the subject is compared to multiple scores. Each score may correspond to a group of individuals in a risk group that have been clinically categorized with a particular risk of developing MS. As an example, the risk groups may be divided into a high risk group, medium risk group, and low risk group. In one scenario, the subject may be classified in a risk group if the subject's predicted score is not significantly different (e.g., p-value >0.05) from one group and is significantly different (e.g., p-value <0.05) in comparison to other groups. Therefore, the subject can undertake changes in lifestyle and/or treatments based on the prediction of a risk/likelihood of developing MS.

In various embodiments, a measure of the disease activity predicted by the predictive model provides additional utility for managing the disease activity in the patient. As one example, the measure of the disease activity predicted by the predictive model is useful for selecting a candidate therapeutic or for determining the effectiveness of a previously administered therapeutic.

In various embodiments, the measure of disease activity predicted by the predictive model for a patient can be compared to a prior measure of disease activity to determine whether a therapeutic administered to the patient is demonstrating efficacy. As one example, the prior measure of disease activity may be a prediction determined for the same patient (e.g., a baseline measure of disease activity). Thus in this example, the comparison of the measure of disease activity and the prior measure of disease activity is a longitudinal analysis of a patient that is undergoing treatment using the therapeutic. As such, a difference or lack of difference between the measure of disease activity and prior measure of disease activity can be an indication that the therapeutic is having an effect or lack of an effect. As another example, the prior measure of disease activity may be a measure determined for a population of patients (e.g., a reference set of patients). In this example, the comparison of the measure of disease activity and the prior measure of disease activity can reveal whether the patient is experiencing effects due to a therapeutic, as evidenced by the measure of disease activity, in comparison to the prior measure of disease activity for the population of patients.

In various embodiments, if the comparison between the measure of disease activity and prior measure of disease activity indicates that a currently administered therapeutic is not exhibiting an effect, or is not exhibiting an effect to a desired extent, a change in the patient's treatment can be undertaken. In one embodiment, the treatment dose of the currently administered therapeutic can be altered to effect a patient response. For example, the currently administered therapeutic can be increased in dosage. In one embodiment, a candidate therapeutic can be selected for administration to the patient. In various embodiments, a candidate therapeutic can be administered to the patient in place of the currently administered therapeutic or the candidate therapeutic can be administered to the patient in addition to the currently administered therapeutic.

As another example, a measure of the disease activity is useful for supporting symptom and medication tracking, nursing interventions, laboratory monitoring, and curated longitudinal MM reports. In such scenarios, the measure of disease activity can reduce unplanned healthcare utilization (e.g., unplanned visits to physician's office), thereby improving patient and physician satisfaction.

### IV. Biomarker Panel

In various embodiments, the assessment of disease activity (e.g., disease progression) involves implementing a univariate biomarker panel. Therefore, the univariate biomarker panel includes one biomarker. In other embodiments, the assessment of disease activity (e.g., disease progression) involves implementing a multivariate biomarker panel. In such embodiments, the multivariate biomarker panel includes more than one biomarker. In various embodiments, the multivariate biomarker panel includes two biomarkers. In various embodiments, the multivariate biomarker panel includes 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biomarkers. In particular embodiments, the multivariate biomarker panel includes 2 biomarkers. In particular embodiments, the multivariate biomarker panel includes 3 biomarkers. In particular embodiments, the multivariate biomarker panel includes 4 biomarkers. In particular embodiments, the multivariate biomarker panel includes 7 biomarkers. In particular embodiments, the multivariate biomarker panel includes 8 biomarkers. In particular embodiments, the multivariate biomarker panel includes 16 biomarkers. In particular embodiments, the multivariate biomarker panel includes 17 biomarkers. In particular embodiments, the multivariate biomarker panel includes 20 biomarkers. In particular embodiments, the multivariate biomarker panel includes 21 biomarkers.

In particular embodiments described herein, a biomarker panel is implemented for the assessment or prediction of disease progression, such as MS disease progression. In various embodiments, the assessment of disease progression involves implementing a univariate biomarker panel. Therefore, the univariate biomarker panel includes one biomarker. In other embodiments, the assessment of disease progression involves implementing a multivariate biomarker panel. In such embodiments, the multivariate biomarker panel for assessing disease progression includes more than one biomarker. In various embodiments, the multivariate biomarker panel for assessing disease progression includes two biomarkers. In various embodiments, the multivariate biomarker panel for assessing disease progression includes 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biomarkers. In particular embodiments, the multivariate biomarker panel includes 2 biomarkers. In particular embodiments, the multivariate biomarker panel includes 3 biomarkers. In particular embodiments, the multivariate biomarker panel includes 4 biomarkers. In particular embodiments, the multivariate biomarker panel includes 7 biomarkers. In particular embodiments, the multivariate biomarker panel includes 8 biomarkers. In particular embodiments, the multivariate biomarker panel includes 16 biomarkers. In particular embodiments, the multivariate biomarker panel includes 17 biomarkers. In particular embodiments, the multivariate biomarker panel includes 20 biomarkers. In particular embodiments, the multivariate biomarker panel includes 21 biomarkers.

In various embodiments, a multivariate biomarker panel further incorporates one or more subject attributes. For example, subject attributes can include an age of the subject, the gender of the subject, a disease duration experienced by the subject (e.g., disease duration of MS), racial/ethnic identity, weight, height, body mass index (BMI), and socioeconomic status.

In an embodiment, the biomarkers in the biomarker panel can include one or more of: 6Ckine, Adiponectin, Adrenomedullin (ADM), Alpha-1 Antitrypsin (AAT), Alpha-1-Microglobulin (A1Micro), Alpha-2-Macroglobulin (A2Macro), Alpha-Fetoprotein (AFP), Amphiregulin (AR), Angiogenin, Angiopoietin 1 (ANG-1), Angiopoietin 2 (ANG-2), Angiotensin Converting Enzyme (ACE), Antileukoproteinase (ALP), Antithrombin III (ATIII), Apolipoprotein A (Apo-A), Apolipoprotein D (Apo-D), Apolipoprotein E (Apo-E), AXL Receptor Tyrosine Kinase (AXL), B-cell activating factor (BAFF), B Lymphocyte Chemoattractant (BLC), Beta-Amyloid (1-40) (AB-40), Beta-Amyloid (1-42) (AB-42), Beta-2 Microglobulin (B2M), Betacellulin (BTC), Brain Derived Neurotrophic Factor (BDNF), C-Reactive Protein (CRP), Cadherin 1 (E-Cad), Calbindin, Cancer Antigen 125 (CA-125), Cancer Antigen 15-3 (CA 15-3), Cancer Antigen 19-9 (CA 19-9), Carbonic anhydrase 9 (CA-9), Carcinoembryonic Antigen (CEA), Carcinoembryonic antigen related cell adhesion molecule 1 (CEACAM1), Cathepsin D, CD40 Ligand (CD40-L), CD163, Ceruloplasmin, Chemokine CC-4 (HCC-4), Chromogranin A (CgA), Ciliary Neurotrophic Factor (CNTF), Clusterin (CLU), Complement C3 (C3), Complement Factor H (CFH), Complement Factor H Related Protein 1 (CFHR1), Cystatin B, CystatinC, Decorin, Dickkopf related protein 1 (DKK-1), Dopamine beta hydroxylase (DBH), E-Selectin, EN-RAGE, Eotaxin-1, Eotaxin-2, Eotaxin-3, Epidermal Growth Factor (EGF), Epidermal Growth Factor Receptor (EGFR), Epiregulin (EPR), Epithelial Derived Neutrophil Activating Protein 78 (ENA-78), Erythropoietin (EPO), Factor VII, Fas Ligand (FasL), FASLG Receptor (FAS), Ferritin (FRTN), Fibrinogen, Fibulin 1C (FibiC), Ficolin 3, Follicle Stimulating Hormone (FSH), Gastric inhibitory polypeptide (GIP), Gelsolin, Glucagon Like Peptide-1 (GLP-1), Glycogen phosphorylase isoenzyme BB (GPBB), Granulocyte Colony Stimulating Factor (GCSF), Granulocyte Macrophage Colony Stimulating Factor (GM-CSF), Growth differentiation factor 15 (GDF-15), Growth Hormone (GH), Growth Regulated alpha protein (GROalpha), Haptoglobin, Heat Shock protein 70 (HSP-70), Heparin Binding EGF Like Growth Factor (HB-EGF), Hepatocyte Growth Factor (HGF), Human Chorionic Gonadotropin beta (hCG), Immunoglobulin A (IgA), Immunoglobulin E (IgE), Immunoglobulin M (IgM), Insulin, Insulin like Growth Factor Binding Protein 2 (IGFBP2), Intercellular Adhesion Molecule 1 (ICAM-1), Interferon alpha (IFN-alpha), Interferon gamma (IFN-gamma), Interferon gamma Induced Protein 10 (IP-10), Interferon inducible T cell alpha chemoattractant (ITAC), Interleukin 1 alpha (IL-1alpha), Interleukin 1 beta (IL-1beta), Interleukin 1 receptor antagonist (IL1ra), Interleukin 2 (IL-2), Interleukin 2 receptor alpha (IL2receptoralpha), Interleukin 3 (IL-3), Interleukin 4 (IL-4), Interleukin 5 (IL-5), Interleukin 6 (IL-6), Interleukin 6 receptor (IL6r), Interleukin 6 receptor subunit beta (IL6Rbeta), Interleukin 7 (IL-7), Interleukin 8 (IL-8), Interleukin 10 (IL-10), Interleukin 12 Subunit p40 (IL12p40), Interleukin 12 Subunit p70 (IL12p70), Interleukin 13 (IL13), Interleukin 15 (IL15), Interleukin 16 (IL16), Interleukin 17 (IL17), Interleukin 18 (IL18), Interleukin 18 binding protein (IL18 bp), Interleukin 22 (IL22), Interleukin 23 (IL23), Interleukin 31 (IL31), Kidney Injury Molecule 1 (KIM-1), Lactoferrin (LTF), Latency Associated Peptide of Transforming Growth Factor beta 1 (LAP TGF b1), Leptin, Leptin Receptor (Leptin R), Leucine rich alpha 2 glycoprotein (LRG1), Luteinizing Hormone (LH), Macrophage Colony Stimulating Factor 1 (M-CSF), Macrophage Derived Chemokine (MDC), Macrophage Inflammatory Protein 1 alpha (MIP1-alpha), Macrophage Inflammatory Protein 1 beta (MIP1-beta), Macrophage Inflammatory Protein 3 alpha (MIP3-alpha), Macrophage Inflammatory Protein 3 beta (MIP3-beta), Macrophage Migration Inhibitory Factor (MIF), Macrophage Stimulating Protein (MSP), Mast stem cell growth factor receptor (SCFR), Matrix Metalloproteinase 1 (MMP-1), Matrix Metalloproteinase 2 (MMP-2), Matrix Metalloproteinase 3 (MMP-3), Matrix Metalloproteinase 7 (MMP-7), Matrix Metalloproteinase 9 (MMP-9), Matrix Metalloproteinase 9 total (MMP-9 total), Matrix Metalloproteinase 10 (MMP-10), Microalbumin, Monocyte Chemotactic Protein 1 (MCP-1), Monocyte Chemotactic Protein 2 (MCP-2), Monocyte Chemotactic Protein 3 (MCP-3), Monocyte Chemotactic Protein 4 (MCP-4), Monokine Induced by Gamma Interferon (MIG), Myeloid Progenitor Inhibitory Factor 1 (MPIF-1), Myeloperoxidase (MPO), Myoglobin, Nerve Growth Factor beta (NGF-beta), Neurofilament heavy polypeptide (NF-H), Neuron Specific Enolase (NSE), Neuronal Cell Adhesion Molecule (NrCAM), Neuropilin-1, Neutrophil Activating Peptide 2 (NAP-2), Omentin, Osteocalcin, Osteopontin, Osteoprotegerin (OPG), P-Selectin, Pancreatic Polypeptide (PPP), Pancreatic secretory trypsin inhibitor (TATI), Paraoxonase-1 (PON1), Pepsinogen-I (PGI), Periostin, Pigment Epithelium Derived Factor (PEDF), Placenta Growth Factor (PLGF), Plasminogen Activator Inhibitor 1 (PAI-1), Platelet endothelial cell adhesion molecule (PECAM-1), Platelet Derived Growth Factor BB (PDGF-BB), Prolactin (PRL), Prostate Specific Antigen Free (PSA-f), Protein DJ-1 (DJ-1), Pulmonary and Activation Regulated Chemokine (PARC), Pulmonary surfactant associated protein D (SP-D), Receptor for advanced glycosylation end products (RAGE), Resistin, S100 calcium binding protein B (S100B), Serum Amyloid A Protein (SAA), Serum Amyloid P Component (SAP), Sex Hormone Binding Globulin (SHBG), Sortilin, ST2, Stem Cell Factor (SCF), Stromal cell derived factor 1 (SDF-1), Superoxide Dismutase 1 soluble (SOD-1), T Cell Specific Protein RANTES (RANTES), T Lymphocyte Secreted Protein I 309 (1309), Tamm Horsfall Urinary Glycoprotein (THP), Tenascin C (TN-C), Tetranectin, Thrombin Activatable ibrinolysis (TAFI), Thrombospondin-1, Thymus and activation regulated chemokine (TARC), Thyroid Stimulating Hormone (TSH), Thyroxine Binding Globulin (TBG), Tissue Inhibitor of Metalloproteinases 1 (TIMP-1), Tissue Inhibitor of Metalloproteinases 2 (TIMP-2), TNF Related Apoptosis Inducing Ligand Receptor 3 (TRAIL-R3), Transferrin receptor protein 1 (TFR1), Transforming Growth Factor beta 3 (TGF-beta3), Tumor Necrosis Factor alpha (TNF-alpha), Tumor Necrosis Factor beta (TNF-beta), Tumor necrosis factor ligand superfamily member 12 (Tweak), Tumor necrosis factor ligand superfamily member 13 (APRIL), Tumor Necrosis Factor Receptor I (TNF-RI), Tumor necrosis factor receptor 2 (TNFR2), Vascular Cell Adhesion Molecule 1 (VCAM-1), Vascular Endothelial Growth Factor (VEGF), Visceral adipose tissue derived serpin A12 (Vaspin), Visfatin, Vitamin D Binding Protein (VDBP), Vitronectin, von Willebrand Factor (vWF), or YKL-40.

In some embodiments, the biomarkers in the biomarker panel include biomarkers shown in Tables 4-6. In some embodiments, the biomarkers can include one or more of: Neurofilament Light Polypeptide Chain (NEFL), Myelin Oligodendrocyte Glycoprotein (MOG), Cluster of Differentiation 6 (CD6), Chemokine (C-X-C motif) ligand 9 (CXCL9), Osteoprotegerin (OPG), Osteopontin (OPN), Matrix Metallopeptidase 9 (MMP-9), Glial Fibrillary Acidic Protein (GFAP), CUB domain-containing protein 1 (CDCP1), C-C Motif Chemokine Ligand 20 (CCL20/MIP 3-α), Interleukin-12 subunit beta (IL-12B), Amyloid Beta Precursor Like Protein 1 (APLP1), Tumor Necrosis Factor Receptor Superfamily Member 10A (TNFRSF10A), Collagen, type IV, alpha 1 (COL4A1), Serpin Family A Member 9 (SERPINA9), Fibronectin Leucine Rich Transmembrane Protein 2 (FLRT2), Chemokine (C-X-C motif) ligand 13 (CXCL13), Growth Hormone (GH), Versican core protein (VCAN), Protogenin (PRTG), Contactin-2 (CNTN2). In some embodiments, the biomarkers further include Growth Hormone (GH2), Interleukin-18 (IL18), Matrix Metalloproteinase-2 (MMP-2), Gamma-Interferon-Inducible Lysosomal Thiol Reductase (IFI30), and Chitinase-3-like protein 1 (CHI3L1/YkL40).

In some embodiments, the biomarkers can include one or more of: Cell Adhesion Molecule 3 (CADM3), Kallikrein Related Peptidase 6 (KLK6), Brevican (BCAN), Oligodendrocyte Myelin Glycoprotein (OMG), CD5 molecule (CD5), Cytotoxic and Regulatory T Cell Molecule (CRTAM), CD244 Molecule (CD244), Tumor Necrosis Factor Receptor Superfamily Member 9 (TNFRSF9), Proteinase 3 (PRTN3), Follistatin Like 3 (FSTL3), C-X-C Motif Chemokine Ligand 10 (CXCL10), C-X-C Motif Chemokine Ligand 11 (CXCL11), Interleukin 18 Binding Protein (IL-18BP), Macrophage Scavenger Receptor 1 (MSR1), C-C Motif Chemokine Ligand 3 (CCL3), Tumor Necrosis Factor Ligand Superfamily Member 12 (TWEAK), Trefoil Factor 3 (TFF3), Ectonucleotide Pyrophosphatase/Phosphodiesterase 2 (ENPP2), Insulin Like Growth Factor Binding Protein 1 (IGFBP-1), Interleukin 12A (IL12A), Seizure Related 6 Homolog Like (SEZ6L), Dipeptidyl Peptidase Like 6 (DPP6), Neurocan (NCAN), Tubulointerstitial Nephritis Antigen Like 1 (TINAGL1), Calcium Activated Nucleotidase 1 (CANT1), Nectin Cell Adhesion Molecule 2 (NECTIN2), Neural Proliferation, Differentiation and Control Protein 1 (NPDC1), Tumor Necrosis Factor Receptor Superfamily Member 11A (TNFRSF11A), Contactin 4 (CNTN4), Neutrophic Receptor Tyrosine Kinase 2 (NTRK2), Neutrophic Receptor Tyrosine Kinase 3 (NTRK3), Cadherin 6 (CDH6), Carcinoembryonic Antigen Related Cell Adhesion Molecule 8 (CEACAM8), Mitotic Arrest Deficient 1 Like 1 (MAD1L1), Fc Fragment of IgA Receptor (FCAR), Myeloperoxidase (MPO), Osteomodulin (OMD), Matrix Extracellular Phosphoglycoprotein (MEPE), GDNF Family Receptor Alpha 3 (GDNFR-alpha-3), Scavenger Receptor Class F Member 2 (SCARF2), CD40 Ligand (IgM), Tumor Necrosis Factor Receptor Superfamily Member 1B (TNF-R2), Programmed Cell Death 1 Ligand (PD-L1), Notch 3 (NOTCH3), Contactin 1 (CNTN1), Oncostatin M (OSM), Transforming Growth Factor Alpha (TGF-α), Peptidoglycan Recognition Protein 1 (PGLYRP1), Nitric Oxide Synthase 3 (NOS3).

In particular embodiments, the biomarker panel useful for generating a prediction (e.g., a prediction for MS disease activity or a prediction for MS disease progression) includes biomarkers identified as Tier A in Table 1, Tier 1 in Table 2, Tier 1 in Table 3, or any of their corresponding substitute biomarkers in Table 4. For example, the biomarker panel includes NEFL, MOG, CD6, CXCL9, OPG, OPN, MMP-9, and GFAP (Tier A in Table 1). As another example, the biomarker panel includes NEFL, MOG, CD6, CXCL9, OPG, OPN, CXCL13, and GFAP (Tier 1 in Table 2). As another example, the biomarker panel includes GFAP, CDCP1, MOG, CXCL13, OPG, APLP1, VCAN, and NEFL (Tier 1 in Table 3).

In particular embodiments, the biomarker panel useful for generating a prediction (e.g., a prediction for MS disease activity or a prediction for MS disease progression) includes biomarkers identified as Tier B in Table 1, Tier 2 in Table 2, Tier 2 in Table 3, or any of their corresponding substitute biomarkers in Table 4. For example, the biomarker panel includes CDCP1, CCL20/MIP 3-α, IL-12B, APLP1, TNFRSF10A, COL4A1, SERPINA9, FLRT2, and CXCL13 (Tier B in Table 1). As another example, the biomarker panel includes CDCP1, CCL20/MIP 3-α, IL-12B, APLP1, TNFRSF10A, COL4A1, SERPINA9, FLRT2, and TNFSF13B (Tier 2 in Table 2). As another example, the biomarker panel includes CDCP1, CCL20/MIP 3-α, IL-12B, APLP1, TNFRSF10A, SERPINA9, FLRT2, and TNFSF13B (all biomarkers listed as Tier 2 in Table 2 except for COL4A1). As another example, the biomarker panel includes CXCL9, TNFRSF10A, CCL20/MIP 3-α, TNFSF13B, CD6, SERPINA9, FLRT2, OPN, and CNTN2 (all biomarkers listed as Tier 2 in Table 3).

In particular embodiments, the biomarker panel useful for generating a prediction (e.g., a prediction for MS disease activity or a prediction for MS disease progression) includes biomarkers identified as Tier C in Table 1, Tier 3 in Table 2, Tier 3 in Table 3, or any of their corresponding substitute biomarkers in Table 4. For example, the biomarker panel includes GH, VCAN, PRTG, and CNTN2 (Tier C in Table 1 and Tier 3 in Table 2). For example, the biomarker panel includes COL4A1, GH, IL-12B, and PRTG (Tier 3 in Table 3).

In particular embodiments, the biomarker panel useful for generating a prediction (e.g., a prediction for MS disease activity or a prediction for MS disease progression) includes biomarkers identified as Tier A and Tier B in Table 1, Tier 1 and Tier 2 in Table 2, Tier 1 and Tier 2 in Table 3, or any of their corresponding substitute biomarkers in Table 4. For example, the biomarker panel includes NEFL, MOG, CD6, CXCL9, OPG, OPN, MMP-9, GFAP, CDCP1, CCL20/MIP 3-α, IL-12B, APLP1, TNFRSF10A, COL4A1, SERPINA9, FLRT2, and CXCL13 (Tiers A and B in Table 1). As another example, the biomarker panel includes NEFL, MOG, CD6, CXCL9, OPG, OPN, CXCL13, GFAP, CDCP1, CCL20/MIP 3-α, IL-12B, APLP1, TNFRSF10A, COL4A1, SERPINA9, FLRT2, and TNFSF13B (Tiers 1 and 2 in Table 2). As another example, the biomarker panel includes NEFL, MOG, CD6, CXCL9, OPG, OPN, CXCL13, GFAP, CDCP1, CCL20/MIP 3-α, IL-12B, APLP1, TNFRSF10A, SERPINA9, FLRT2, and TNF (all biomarkers listed as Tiers 1 and 2 in Table 2 except for COL4A1). As another example, the biomarker panel includes GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, and CNTN2 (all biomarkers listed as Tiers 1 and 2 in Table 3).

In particular embodiments, the biomarker panel useful for generating a prediction (e.g., a prediction for MS disease activity or a prediction for MS disease progression) includes biomarkers identified as Tier A, Tier B, or Tier C in Table 1, Tier 1, Tier 2, or Tier 3 in Table 2, Tier 1, Tier 2, and Tier 3 in Table 3, or any of their corresponding substitute biomarkers in Table 4. For example, the biomarker panel includes NEFL, MOG, CD6, CXCL9, OPG, OPN, MMP-9, GFAP, CDCP1, CCL20/MIP 3-α, IL-12B, APLP1, TNFRSF10A, COL4A1, SERPINA9, FLRT2, CXCL13, GH, VCAN, PRTG, and CNTN2 (Tiers A, B, and C in Table 1). As another example, the biomarker panel includes NEFL, MOG, CD6, CXCL9, OPG, OPN, CXCL13, GFAP, CDCP1, CCL20/MIP 3-α, IL-12B, APLP1, TNFRSF10A, COL4A1, SERPINA9, FLRT2, TNFSF13B, GH, VCAN, PRTG, CNTN2, GH2, IL18, MMP-2, IFI30, and CHI3L1/YkL40. (Tiers 1, 2, and 3 in Table 2). As another example, the biomarker panel includes NEFL, MOG, CD6, CXCL9, OPG, OPN, CXCL13, GFAP, CDCP1, CCL20/MIP 3-α, IL-12B, APLP1, TNFRSF10A, SERPINA9, FLRT2, TNFSF13B, GH, VCAN, PRTG, CNTN2, GH2, IL18, MMP-2, IFI30, and CHI3L1/YkL40 (all biomarkers listed in Tiers 1, 2, and 3 in Table 2 except for COL4A1). As another example, the biomarker panel includes GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG (Tiers 1, 2, and 3 in Table 3).

In various embodiments, the biomarker panel for generating a prediction (e.g., a prediction for disease activity or a prediction for disease progression) includes a minimal set of predictive biomarkers, such as a pair of biomarkers, a biomarker triplicate, or a biomarker quadruplicate. In various embodiments, at least one of the biomarkers in a biomarker pair, biomarker triplicate, or biomarker quadruplicate is NEFL. In various embodiments, at least one of the biomarkers in a biomarker pair, biomarker triplicate, or biomarker quadruplicate is MOG. In various embodiments, the biomarker pair, biomarker triplicate, or biomarker quadruplicate does not include NEFL. In such embodiments, the biomarker pair, biomarker triplicate, or biomarker quadruplicate not including NEFL does include MOG.

Examples of biomarker pairs useful for generating a prediction (e.g., a prediction for MS disease activity or a prediction for MS disease progression) include: 1) NEFL and MOG, 2) NEFL and CD6, 3) NEFL and CXCL9, 4) NEFL and TNFRSF10A, 5) MOG and IL-12B, 6) CXCL9 and CD6, 7) MOG and CXCL9, 8) MOG and CD6, 9) CXCL9 and COL4A1, and 10) CD6 and VCAN. Additional examples of biomarker pairs that are predictive of multiple sclerosis disease activity include: 1) NEFL and TNFSF13B, 2) NEFL and CNTN2, 3) NEFL and CXCL9, 4) MOG and CDCP1, 5) MOG and TNFSF13B, and 6) MOG and CXCL9. Additional examples of biomarker pairs that are predictive of multiple sclerosis disease activity include: 1) NEFL and TNFSF13B, 2) NEFL and SERPINA9, 3) NEFL and GH, 4) MOG and TNFSF13B, 5) MOG and CXCL9, and 6) MOG and IL-12B.

Examples of biomarker triplicates that are useful for generating a prediction (e.g., a prediction for MS disease activity or a prediction for MS disease progression) include: 1) MOG, IL-12B, and APLP1, 2) MOG, CD6, and CXCL9, 3) CXCL9, COL4A1, and VCAN, 4) NEFL, CD6, and CXCL9, 5) NEFL, TNFRSF10A, and COL4A1, 6) MOG, IL-12B, and CNTN2, and 7) CD6, CCL20, and VCAN. Additional examples of biomarker triplicates that are predictive of multiple sclerosis disease activity include: 1) NEFL, CNTN2, and TNFSF13B, 2) NEFL, APLP1, and TNFSF13B, 3) NEFL, TNFRSF10A, and TNFSF13B, 4) MOG, CXCL9, and TNFSF13B, 5) MOG, OPG, and TNFSF13B, and 6) MOG, CCL20, and TNFSF13B. Additional examples of biomarker triplicates that are predictive of multiple sclerosis disease activity include: 1) NEFL, SERPINA9, and TNFSF13B, 2) NEFL, CNTN2, and TNFSF13B, 3) NEFL, APLP1, and TNFSF13B, 4) MOB, CXCL9, and TNFSF13B, 5) MOG, SERPINA9, and TNFSF13B, and 6) MOG, OPG, and TNFSF13B.

Examples of biomarker quadruplicates that are useful for generating a prediction (e.g., a prediction for MS disease activity or a prediction for MS disease progression) include: 1) NEFL, MOG, CD6, and CXCL9, 2) NEFL, CXCL9, TNFRSF10A, and COL4A1, 3) MOG, CXCL9, IL-12B, and APLP1, 4) CXCL9, COL4A1, OPG, and VCAN, 5) CXCL9, OPG, APLP1, and OPN, 6) NEFL, CD6, CXCL9, and CXCL13, 7) NEFL, MOG, CD6, and CXCL9, 8) NEFL, TNFRSF10A, COL4A1, and CCL20, 9) MOG, IL-12B, OPN, and CNTN2, and 10) CD6, COL4A1, CCL20, and VCA. Additional examples of biomarker quadruplicates that are predictive of multiple sclerosis disease activity include: 1) NEFL, TNFRSF10A, CNTN2, and TNFSF13B, 2) NEFL, COL4A1, CNTN2, and TNFSF13B, 3) NEFL, TNFRSF10A, APLP1, and TNFSF13B, 4) MOG, CXCL9, APLP1, and TNFSF13B, 5) MOG, CXCL9, OPG, and TNFSF13B, and 6) MOG, CXCL9, OPG, and CNTN2. Additional examples of biomarker quadruplicates that are predictive of multiple sclerosis disease activity include: 1) NEFL, CCL20, SERPINA9, and TNFSF13B, 2) NEFL, APLP1, SERPINA9, and TNFSF13B, 3) NEFL, CCL20, APLP1, and TNFSF13B, 4) MOG, CXCL9, OPG, and TNFSF13B, 5) MOG, OPG, SERPINA9, AND TNFSF13B, 6) MOG, CXCL9, SERPINA9, and TNFSF13B, and 7) CDCP1 and IL-12B.

In various embodiments, the minimal set of predictive biomarkers is useful for generating a prediction for MS disease progression. Examples of biomarker pairs useful for generating a prediction for MS disease progression include: 1) MOG and GFAP, 2) NEFL and GFAP, 3) GFAP and APLP1, and 4) NEFL and MOG, Examples of biomarker triplicates useful for generating a prediction for MS disease progression include: 1) NEFL, MOG, and GFAP, 2) NEFL MOG, and GH, 3) NEFL, MOG, and SERPINA9, 4) OPN, CXCL13, and SERPINA9, 5) OPN, CXCL13, and TNFRSF10A, and 6) NEFL, CD6, and CXCL13. Examples of biomarker quadruplicates useful for generating a prediction for MS disease progression include: 1) NEFL, MOG, GFAP, and IL-12B, 2) NEFL, MOG, GFAP, and PRTG, 3) NEFL, MOG, GFAP, and APLP1, 4) NEFL, MOG, SERPINA9, and GH, 5) NEFL MOG, TNFRSF10A, and SERPINA9, 6) CD6, CCL20, IL-12B, and APLP1, 7) CD6, CDCP1, IL-12B, and VCAN.

In various embodiments, at least one of the biomarkers in the minimal set of predictive biomarkers is GFAP. For example, at least one biomarker in a pair, triplicate, or quadruplicate of biomarkers is GFAP. Examples of pairs of biomarkers that are predictive of MS disease progression in which one is GFAP include: 1) GFAP and MOG, 2) GFAP and APLP1, 3) OPG and GFAP, 4) TNFRSF10A and GFAP, 5) GFAP and CDCP1, 6) GFAP and NEFL, 7), CNTN2 and GFAP, 8) GH and GFAP, and 9) CXCL9 and GFAP. Examples of triplicates of biomarkers that are predictive of MS disease progression in which one is GFAP include: 1) OPG, GFAP, and MOG, 2) OPG, GFAP, and APLP1, 3), GFAP, TNFRSF10A, and MOG, 4) CXCL9, OPG, and GFAP, 5) GFAP, TNFRSF10A, and APLP1, 6) GFAP, APLP1, and NEFL, 7) GFAP, CXCL13, and APLP1, 8) GFAP, FLRT2, and APLP1, 9) CXCL9, GFAP, and APLP1, and 10) GH, GFAP, and APLP1. Examples of quadruplicates of biomarkers that are predictive of MS disease progression in which one is GFAP include: 1) CXCL9, OPG, GFAP, and MOG, 2) CNTN2, OPG, GFAP, and MOG, 3) CXCL9, OPG, GFAP, and APLP1, 4) OPG, GFAP, PRTG, and MOG, 5) OPG, GFAP, OPN, and MOG, 6) GFAP, CXCL13, APLP1, and NEFL, 7) GFAP, FLRT2, APLP1, and NEFL, 8) OPN, GFAP, APLP1, and NEFL, 9, CXCL9, GFAP, APLP1, and NEFL, and 10) GFAP, CXCL13, FLRT2, and APLP1. These example pairs, triplicates, or quadruplicates of biomarkers may be useful for predicting MS disease progression according to an ordinal scale (e.g., EDSS or PDDS scoring scale).

Additional examples of pairs of biomarkers that are predictive of MS disease progression in which one is GFAP include: 1) CDCP1 and GFAP, 2) APLP1 and GFAP, 3) GFAP and CXCL13, 4) MOG and GFAP, and 5) OPG and GFAP. Additional examples of triplicates of biomarkers that are predictive of MS disease progression in which one is GFAP include: 1) CDCP1, APLP1, and GFAP, 2) CDCP1, MOG, and GFAP, 3) APLP1, GFAP, and CXCL13, 4) CDCP1, GFAP, and SERPINA9, and 5) MOG, GFAP, and CXCL13.

Additional examples of quadruplicates of biomarkers that are predictive of MS disease progression in which one is GFAP include: 1) CDCP1, CCL20, APLP1, and GFAP, 2) CDCP1, APLP1, GFAP, and CXCL13, 3) CDCP1, CCL20, MOG, and GFAP, 4) CDCP1, GFAP, APLP1, and SERPINA9, and 5) CDCP1, MOG, GFAP, and APLP1. These example pairs, triplicates, or quadruplicates of biomarkers may be useful for differentiating between two classes of MS disease progression (e.g., mild/moderate vs. severe disability).

In various embodiments, the minimal set of predictive biomarkers need not include GFAP. Examples of pairs of biomarkers that are predictive of MS disease progression in which GFAP is excluded include: 1) OPG and NEFL, 2) OPG and OPN, 3) OPG and FLRT2, 4) OPG and MOG, 5) CXCL9 and OPG, 6) GH and NEFL, 7) CXCL13 and NEFL, 8) APLP1 and NEFL, 9) CCL20 and NEFL, and 10) CXCL9 and NEFL. Examples of triplicates of biomarkers that are predictive of MS disease progression in which one is GFAP include: 1) OPG, MOG, and NEFL, 2) OPG, FLRT2, and NEFL, 3) CXCL9, OPG, and NEFL, 4) OPG, CDCP1, and NEFL, 5) OPG, OPN, and NEFL, 6) GH, APLP1, NEFL, 7) GH, CXCL13, and NEFL, 8) GH, CDCP1, and NEFL, 9) CXCL13, CCL20, and NEFL, and 10) GH, CCL0, and NEFL. Examples of quadruplicates of biomarkers that are predictive of MS disease progression in which one is GFAP include: 1) CDCP1, CXCL13, MOG, and NEFL, 2) CD6, CXCL9, CXCL13, and NEFL, 3) CXCL9, CXCL13, MOG, and NEFL, 4) CD6, CDCP1, CXCl13, and NEFL, 5) CD6, CXCL9, CXCL13, and MOG, 6) CDCP1, CXCl13, MOG, and NEFL, 7) CD6, CDCP1, CXCL13, and NEFL, 8) CXCL9, CXCL13, MOG, and NEFL, 9) CD6, CXCL9, CXCL13, and NEFL, and 10) CD6, CDCP1, CXCL13, and MOG. These example pairs, triplicates, or quadruplicates of biomarkers may be useful for predicting MS disease progression according to an ordinal scale (e.g., EDSS or PDDS scoring scale).

### V. Biomarkers

The dysregulation of biomarkers disclosed herein may contribute to the development and/or progression of disease activity, such as disease activity and/or disease progression of a neurodegenerative disease including multiple sclerosis, Parkinson's Disease, Lewy body disease, Alzheimer's Disease, Amyotrophic lateral sclerosis (ALS), motor neuron disease, Huntington's Disease, Spinal muscular atrophy, Friedreich's ataxia, Batten disease, and the like. Biomarkers, and the corresponding categorization of the biomarkers, are shown below in Table 7. Example categories include: neurodegeneration, myelin integrity, neuroaxonal integrity, cerebrovascular function, neurite outgrowth and neurogenesis, inflammation, neuroinflammation, immune modulation, cell regulation, cell adhesion, gut-brain axis, metabolism, and neuroregulatory categories. Exemplary biomarker categorizations are shown in FIG. 1D. Additionally, biomarkers and their involvement in particular locations (e.g., brain, brain barrier, or blood) and cell types are shown in Tables 8, 9A, and 9B.

NEFL is a 68 kDa biomarker that reflects axonal damage in the microenvironment. In other words, NEFL often serves as a proxy for axonal degeneration. Additionally, NEFL interacts with other biomarkers such as MAP2, Protein Kinase N1, and Tuberous sclerosis (TSCl).

COL4A1 is a 26 kDa biomarker involved in cell proliferation, migration, extracellular matrix formation, as well as inhibition of endothelial cell proliferation, migration, and tube formation. COL4A1 is involved in the outgrowth of hippocampal embryonic neurons and is further involved in myelin integrity. Type IV collagen is a major structural component of glomerular basement membranes (GBM), forming a chicken-wire mesh work together with laminins, proteoglycans and entactin/nidogen. It comprises a C-terminal NC1 domain, which inhibits angiogenesis and tumor formation. The C-terminal half is found to possess the anti-angiogenic activity. Type IV collage also inhibits endothelial cell proliferation, migration and tube formation as well as also inhibiting expression of hypoxia-inducible factor 1alpha and ERK1/2 and p38 MAPK activation. COL4A1 mutations are associated with a wide range of phenotypes that include both ischemic and hemorrhagic strokes, migraines, leukomalacia, nephropathy, hematuria, chronic muscle cramps, and ocular anterior segment diseases including congenital cataracts, glaucoma, and Axenfeld-Rieger anomalies. Case Rep Neurol. 2015 May-August; 7(2): 142-147. Published online 2015 Jun. 2. doi:10.1159/000431309.

APLP1 is a 72 kDa biomarker involved in synaptic maturation during cortical development and regulation of neurite outgrowth. APLP1 is one of two homologs: amyloid-like proteins 1 and 2, or APLP1 and APLP2. The encoding gene of APLP1 is a member of the highly conserved amyloid precursor protein gene family. The encoded protein is a membrane-associated glycoprotein that is cleaved by secretases in a manner similar to amyloid beta A4 precursor protein cleavage. This cleavage liberates an intracellular cytoplasmic fragment that may act as a transcriptional activator. APLP1 may also play a role in synaptic maturation during cortical development. Can regulate neurite outgrowth through binding to components of the extracellular matrix such as heparin and collagen I. APLP1 is extensively expressed in humans. Functions attributed to APLP1 include neurite outgrowth and synaptogenesis, protein trafficking along axons, cell adhesion, calcium metabolism, neuronal damage, synaptic dysfunction, and signal transduction.

MMP2 (72 kDa) and MMP9 (78-92 kDa) are gelatinases, a type of proteolytic enzyme involved in the breakdown of extracellular matrices. MMP2 and MMP9 play role in physiological processes such as embryonic development, reproduction, and tissue remodeling. Serum MMP-2 and MMP-9 are elevated in different multiple sclerosis subtypes. Avolio, C., et al. Serum MMP-2 and MMP-9 are elevated in different multiple sclerosis subtypes, J. Neuroimmunol. March; 136(1-2):46-53. The integrity of the blood-brain barrier as the main structural interface between periphery and brain seems to play an important role in MS. Reducing the secretion of proteolytic matrix metalloproteinases (MMP), e.g., MMP2 and/or MMP9, as disruptors of blood-brain barrier integrity could have profound implications for MS. Proschinger et al. “Influence of combined functional resistance and endurance exercise over 12 weeks on matrix metalloproteinase-2 serum concentration in persons with relapsing-remitting multiple sclerosis—a community-based randomized controlled trial.” BMC Neurol 19, 314 (2019).

FLRT2 is a 74 kDa biomarker and is a member of the fibronectin leucine rich transmembrane protein family, which function in cell adhesion and/or receptor signaling. FLRT2 is expressed in brain as well as in the heart and several other organs, and is involved in fibroblast growth factor-mediated signaling cascades. In the heart, it is required for normal organization of the cardiac basement membrane during embryogenesis, and for normal embryonic epicardium and heart morphogenesis. In the neurology context, FLRT2 functions in cell-cell adhesion, cell migration and axon guidance. It may play a role in the migration of cortical neurons during brain development via its interaction with UNC5D. FLRT2 is also involved in glutamate excitotoxicity, neuronal cell death, and synaptic formation & plasticity.

VCAN (>200 kDa biomarker) is involved in cell motility, cell growth and differentiation, cell adhesion, cell proliferation, cell migration, and angiogenesis. VCAN is further involved in myelin protection, astrocytic excitotoxicity, and is a proinflammatory mediator secretion. VCAN is a key factor in inflammation through interactions with adhesion molecules on the surfaces of inflammatory leukocytes and interactions with chemokines that are involved in recruiting inflammatory cells. In the adult central nervous system, versican is found in perineuronal nets, where it may stabilize synaptic connections. Versican can also inhibit nervous system regeneration and axonal growth following an injury to the central nervous system.

TNFSF13B, also herein referred to as B-cell activating factor (BAFF), is a biomarker involved in T cell-independent B cell activation and ectopic lymphoid follicle formation.

CHI3L1 is a 40 kDa biomarker that plays a role in inflammation, innate immune system, tissue remodeling, and in the capacity of cells to respond to and cope with changes in their environment. CHIL3L1 further plays a role in T-helper cell type 2 (Th2) inflammatory response and IL-13-induced inflammation, regulating allergen sensitization, inflammatory cell apoptosis, dendritic cell accumulation and M2 macrophage differentiation. CHI3L1 facilitates invasion of pathogenic enteric bacteria into colonic mucosa and lymphoid organs, activation of AKT1 signaling pathway and subsequent IL8 production in colonic epithelial cells, antibacterial responses in lung by contributing to macrophage bacterial killing, controlling bacterial dissemination, and augmenting host tolerance. CHI3L1 also regulates hyperoxia-induced injury, inflammation, and epithelial apoptosis in lung.

IL-12B is a 40 kDa biomarker representing one subunit of the IL-12 heterodimer. IL-12A (35 kDa) represents the other subunit of the IL-12 heterodimer. IL-12B is involved in innate & adaptive immunity and in the regulation of memory/effector Th1 cells. IL-12B is a growth factor for activated T and NK cells. IL-12B associates with IL23A to form the IL-23 interleukin, a heterodimeric cytokine which functions in innate and adaptive immunity. Polymorphisms in the genes encoding interleukin 23 receptor (IL23R) and the p40 subunit of IL-12/23 (IL12B) have been implicated in multiple sclerosis (MS) risk. Huang et al., “Meta-analysis of the IL23R and IL12B polymorphisms in multiple sclerosis.” Int. J. of Neuroscience, 126:3, 205-212 (2016).

IFI30 is a 30-35 kDa biomarker that plays a role in antigen processing by facilitating complete unfolding of proteins destined for lysosomal degradation. IFI30 facilitates the generation of MHC class II-restricted epitopes from disulfide bond-containing antigen by the endocytic reduction of disulfide bonds. IFI30 also facilitates also MHC class I-restricted recognition of exogenous antigens containing disulfide bonds by CD8+ T-cells or cross presentation. IFI30 is expressed constitutively in antigen presenting cells and is induced by inflammatory cytokines.

SERPINA9 is a 42 kDa biomarker that is a member of the serpin family of serine protease inhibitors. SERPINA9 is involved in neuronal damage. The expression of SERPINA9 is likely restricted to germinal center B cells and lymphoid malignancies. SERPINA9 is likely to function in vivo in the germinal center as an efficient inhibitor of trypsin-like proteases.

IL18 is involved in immune response and inflammatory processes. IL18 is a proinflammatory cytokine primarily involved in polarized T-helper 1 (Th1) cell and natural killer (NK) cell immune responses. It serves as an inhibitor of the early Th1 cytokine response. It further plays a role in Th-1 response through its ability to induce IFN-gamma production in T cells and NK cells. IL-18 in CSF and serum were significantly higher in comparison with the levels found in patients without enhancing lesions. The results suggest involvement of IL-18 in immunopathogenesis of MS especially in the active stages of the disease. Losy, J., et al. IL-18 in patients with multiple sclerosis. Acta Neurologica Scandinavica, 104:171-173 (2001). Additionally, higher IL-18 serum levels and significant different frequencies of two polymorphisms of IL-18 were found in MS patients. Jahanbani-Ardakani, H. et al., Interleukin 18 polymorphisms and its serum level in patients with multiple sclerosis, Ann Indian Acad Neurol; 22:474-76 (2019).

CDCP1 is a 90-140 kDa biomarker involved in T-cell migration, cell adhesion, and cell matrix association. CDCP1 may play a role in the regulation of anchorage versus migration or proliferation versus differentiation via its phosphorylation. CDCP1 is expressed in cells with phenotypes reminiscent of mesenchymal stem cells and neural stem cells. Additionally, CDCP1 is a ligand for CD6, a receptor molecule expressed on certain T-cells and may play a role in their migration and chemotaxis.

CNTN2 is a 113 kDa biomarker involved in cell adhesion, proliferation, migration, axon guidance of neurons, neuronal damage, and axon-dendritic rearrangement. CNTN2 is a member of the contactin family of proteins, part of the immunoglobulin superfamily of cell adhesion molecules. CNTN2 is a glycosylphosphatidylinositol (GPI)-anchored neuronal membrane protein and plays a role in the proliferation, migration, and axon guidance of neurons of the developing cerebellum. A mutation in CNTN2 gene may be associated with adult myoclonic epilepsy. In conjunction with another transmembrane protein, CNTNAP2, which contributes to the organization of axonal domains at nodes of Ranvier by maintaining voltage-gated potassium channels at the juxtaparanodal region.

GFAP is a 50 kDa biomarker involved in demyelination, degeneration, and neuroaxonal injury. Astroglial activation is associated with activation of the immune cascade and is thought to play a role in the demyelination and neuroaxonal injury observed in MS. Glial fibrillar acidic protein (GFAP) is the major constituent of gliotic scarring. GFAP is used as a marker to distinguish astrocytes from other glial cells during development. Higher serum concentrations of both GFAP and NEFL were associated with higher EDSS, older age, longer disease duration, progressive disease course and MM pathology. Hogel, H., et al. Serum glial fibrillary acidic protein correlates with multiple sclerosis disease severity. Multiple Sclerosis Journal, 26(13) 2018.

MOG is a 28 kDa membrane protein expressed on the oligodendrocyte cell surface and the outermost surface of myelin sheaths. Due to this localization, it serves as a cell surface receptor or cell adhesion molecule and is a primary target antigen involved in immune-mediated demyelination. This protein may be involved in completion and maintenance of the myelin sheath and in cell-cell communication. Diseases associated with MOG include Narcolepsy and Rubella. Among its related pathways are Neural Stem Cell Differentiation Pathways and Lineage-specific Markers. A paralog of the MOG gene is BTN1A1.

CD6 is a 90-130 kDa biomarker involved in central nervous system development. CD6 is a cell-adhesion molecule involved in blood brain barrier breach and T-cell mediated acute inflammatory response. Recent studies have identified CD6 as a risk gene for multiple sclerosis (MS), a disease in which autoreactive T cells are integrally involved. De Jager, P L., et al., Meta-analysis of genome scans and replication identify CD6, IRF8 and TNFRSF1A as new multiple sclerosis susceptibility loci. Nat Genet. 2009; 41(7):776-782. CD6 is found on the outer membrane of T-lymphocytes and is involved in the transmigration of leukocytes across the blood-brain barrier.

CXCL9 is a 12 kDa biomarker involved in immune response and inflammatory processes. CXCL9 is a cytokine that affects the growth, movement, or activation state of cells that participate in immune and inflammatory response. CXCL9 (MIG) is a chemokine that upon binding to its receptor CXCR3 elicits chemotactic activity on T cells and is involved in inflammatory response. CXCL9 is not constitutively expressed but is inducible by IFN-gamma. CXCL9 has been described to be involved in several inflammation-related diseases such as hepatitis C, skin inflammation, rheumatoid arthritis, and pharyngitis. Consistent with this observation is the upregulation of ELR-CXC chemokines, CXCL9, CXCL10 and CXCL11, which are upregulated in the CNS of EAE-affected mice induced by transfer of Th1 cells. Lovett-Racke, A., et al. Th1 versus Th17: Are T cell cytokines relevant in multiple sclerosis? Biochimca et Biophysica Acta (BBA)—Molecular Basis of Disease. 1812(2): 246-251 (2011).

CXCL13 is a biomarker involved in cell growth, cell reproduction, regeneration and inflammatory responses. CXCL13 belongs to the CXC chemokine family and is selectively chemotactic for B cells. It interacts with chemokine receptor CXCR5 through which it regulates the organization of B cells. Serum levels of CXCL13 have been implicated in multiple sclerosis. Festa, E. et al. Serum levels of CXCL13 are elevated in active multiple sclerosis. Multiple Sclerosis Journal, 15(11): 1271-1279 (2009).

CCL20 is a 11 kDa biomarker involved in axonal guidance and chemotaxis of dendritic cells. CCL20 is a chemokine involved in immunoregulatory and inflammatory processes (e.g., acute inflammatory response) and is expressed in epithelial cells of choroid plexus in the human brain. It serves as a cognate ligand of CCR6.

OPG is a 55-60 kDa biomarker involved in inflammation, cell apoptosis, and T-cell activation processes. OPG is a decoy receptor of cytokines TNFSF11 (RANKL) and possibly TNFSF10 (TRAIL) and belongs to the TNF receptor superfamily. OPG is up-regulated by estrogens and increasing calcium concentrations, and it has a role in transcriptional regulation in inflammation, innate immunity, and cell survival and differentiation; for example, OPG binding to TNFSF11 inhibits the differentiation of osteoclast precursors into mature osteoclasts and OPG has been used experimentally for the treatment of osteoporosis. OPG has been described to be involved in several inflammation-related diseases such as rheumatoid arthritis, inflammatory bowel disease, and periodontitis.

OPN is a 33-44 kDa biomarker involved in inflammation and immune modulation. OPN is a pleiotropic integrin binding protein with functions in cell-mediated immunity, inflammation, tissue repair, and cell survival. OPN also plays a role in biomineralization.

PRTG is a 180 kDa biomarker involved in neurogenesis, neurotrophin binding, neuronal survival, and demyelination. It may play a role in anteroposterior axis elongation. PRTG is a membrane protein and member of the immunoglobulin superfamily. It is considered to be primarily a developmental protein that has some associations to neuralgia, demyelinating diseases and dyslexia.

TNFRSF10A is a 50 kDa biomarker that is a member of the TNF-receptor superfamily. TNFRSF10A is involved in inflammation and neurodegenerative processes. This receptor is activated by tumor necrosis factor-related apoptosis inducing ligand (TNFSF10/TRAIL), and thus transduces cell death signal and induces cell apoptosis.

GH, also known as somatotropin or somatropin, is a neuroendocrine marker that stimulates growth, cell reproduction and regeneration in humans and other animals. It regulates energy homeostasis and metabolism. It is a type of mitogen which is specific only to certain kinds of cells. Prior studies have shown that it is decreased in the serum of severe MS patients. Gironi, M., et al. Growth hormone and Disease Severity in Early Stage of Multiple Sclerosis. Multiple Sclerosis International 2013: (2013).

GH2, also known as growth hormone 2, placenta-specific growth hormone, and growth hormone variant, is a biomarker involved in growth control, differentiation, and proliferation of myoblasts. It regulates energy homeostasis and metabolism. It is produced and secreted by the placenta during pregnancy and is the dominant form of growth hormone during the pregnancy phase.

VCAM-1 is a 80 kDa transmembrane biomarker typically expressed in blood vessels that mediates the adhesion of cells to vascular endothelium. VCAM-1 is characterized by its multiple immunoglobulin domains. VCAM-1 has been implicated in multiple sclerosis. Peterson, J. et al., VCAM-1-Positive Microglia Target Oligodendrocytes at the Border of Multiple Sclerosis Lesions, Journal of Neuropathology & Experimental Neurology, Volume 61, Issue 6, June 2002, Pages 539-546. Matsuda, M. et al. Increased levels of soluble vascular cell adhesion molecule-1 (VCAM-1) in the cerebrospinal fluid and sera of patients with multiple sclerosis and human T lymphotropic virus type-1-associated myelopathy. J. Neuroimmunology, 59(1-2): 35-40 (1995).

In various embodiments, the biomarker panel may include yet further additional biomarkers described herein. In various embodiments, these further additional biomarkers serve as substitutable biomarkers for the biomarkers described above. In various embodiments, further additional biomarkers include: Cell Adhesion Molecule 3 (CADM3), Kallikrein Related Peptidase 6 (KLK6), Brevican (BCAN), Oligodendrocyte Myelin Glycoprotein (OMG), CD5 molecule (CD5), Cytotoxic and Regulatory T Cell Molecule (CRTAM), CD244 Molecule (CD244), Tumor Necrosis Factor Receptor Superfamily Member 9 (TNFRSF9), Proteinase 3 (PRTN3), Follistatin Like 3 (FSTL3), C-X-C Motif Chemokine Ligand 10 (CXCL10), C-X-C Motif Chemokine Ligand 11 (CXCL11), Interleukin 18 Binding Protein (IL-18BP), Macrophage Scavenger Receptor 1 (MSR1), C-C Motif Chemokine Ligand 3 (CCL3), Tumor Necrosis Factor Ligand Superfamily Member 12 (TWEAK), Trefoil Factor 3 (TFF3), Ectonucleotide Pyrophosphatase/Phosphodiesterase 2 (ENPP2), Insulin Like Growth Factor Binding Protein 1 (IGFBP-1), Interleukin 12A (IL12A), Seizure Related 6 Homolog Like (SEZ6L), Dipeptidyl Peptidase Like 6 (DPP6), Neurocan (NCAN), Tubulointerstitial Nephritis Antigen Like 1 (TINAGL1), Calcium Activated Nucleotidase 1 (CANT1), Nectin Cell Adhesion Molecule 2 (NECTIN2), Neural Proliferation, Differentiation and Control Protein 1 (NPDC1), Tumor Necrosis Factor Receptor Superfamily Member 11A (TNFRSF11A), Contactin 4 (CNTN4), Neutrophic Receptor Tyrosine Kinase 2 (NTRK2), Neutrophic Receptor Tyrosine Kinase 3 (NTRK3), Cadherin 6 (CDH6), Carcinoembryonic Antigen Related Cell Adhesion Molecule 8 (CEACAM8), Mitotic Arrest Deficient 1 Like 1 (MAD1L1), Fc Fragment of IgA Receptor (FCAR), Myeloperoxidase (MPO), Osteomodulin (OMD), Matrix Extracellular Phosphoglycoprotein (MEPE), GDNF Family Receptor Alpha 3 (GDNFR-alpha-3), Scavenger Receptor Class F Member 2 (SCARF2), CD40 Ligand (IgM), Tumor Necrosis Factor Receptor Superfamily Member 1B (TNF-R2), Programmed Cell Death 1 Ligand (PD-L1), Notch 3 (NOTCH3), Contactin 1 (CNTN1), Oncostatin M (OSM), Transforming Growth Factor Alpha (TGF-α), Peptidoglycan Recognition Protein 1 (PGLYRP1), Nitric Oxide Synthase 3 (NOS3), Discoidin Domain Receptor Tyrosine Kinase 1 (DDR1), C-X-C Motif Chemokine Ligand 16 (CXCL16), CD166 antigen (ALCAM), Spondin-2 (SPON2), and Protocadherin-17 (PCDH17).

CADM3 is involved in cell-cell adhesion and interacts with any of IGSF4, NECTIN1, NECTIN 3, and EPB41L1. CADM3 is involved in the biological processes of adherens junction organization, heterophilic cell-cell adhesion, homophilic cell adhesion, and protein localization.

KLK6 is a serine protease that demonstrates activity against proteins such as alpha-synuclein, amyloid precursor protein, myelin basic protein, gelatin, casein and extracellular matrix proteins such as fibronectin, laminin, vitronectin and collagen. KLK6 is involved in the biological processes of amyloid precursor protein metabolic process, CNS development, myelination, protein autoprocessing, regulation of cell differentiation and neuron development, response to wounding, and tissue regeneration.

BCAN is a proteoglycan and is a member of the lectican protein family. BCAN is involved in the biological processes of cell adhesion, CNS development, chondroitin sulfate biosynthesis and catabolic processes, extracellular matrix organization, and axonal synapse maturation.

OMG is a cell adhesion molecules involved in the myelination of the central nervous system. OMG is involved in the biological processes of cell adhesion, regulation of axonogenesis, and neuron projection regeneration.

CD5 is a signal transducing molecule that can be expressed on the surface of cells, such as T-lymphocytes. CD5 is involved in the biological processes of apoptotic signaling pathways, cell recognition, T cell proliferation, and T cell costimulation.

CRTAM is an immunoglobulin-superfamily transmembrane protein and is involved in the biological processes of the adaptive immune response, cell recognition, detection of stimulus or cells, and regulation of immune response. CRTAM is further involved in heterophilic cell-cell adhesion which regulates the activation, differentiation and tissue retention of various T-cell subsets.

CD244 is a member of the signaling lymphocyte activation molecule expressed on natural killer cells. CD244 is involved in the biological processes of immune response (adaptive and innate), leukocyte migration, regulation of cytokine secretion, and signal transduction. CD244 modulates the activation and differentiation of a wide variety of immune cells and thus are involved in the regulation and interconnection of both innate and adaptive immune response

TNFRSF9 is a member of the tumor necrosis factor receptor family and is involved in the biological processes of the TNFR signaling pathway, cell proliferation, and apoptotic processes. TNFRSF9 is expressed by activated T cells.

PRTN3 is a serine protease expressed by neutrophil granulocytes. PRTN3 is involved in the biological processes of antimicrobial humoral response, blood coagulation, neutrophil activity, proteolysis, and cytokine-mediated signaling pathways.

FSTL3 is a secreted glycoprotein of the follistatin-module-protein family. FSTL3 is involved in the biological processes of activin/fibronectin binding, organ development, osteogenesis, ossification, and regulation of cell-cell adhesion.

CXCL10 and CXCL11 are each cytokines in the CXC chemokine family. CXCL10 and CXCL11 are involved in the biological processes of immune response, inflammatory response, cell signaling, chemotaxis, T-cell recruitment, and cell proliferation.

IL-18BP is a protein that serves as an inhibitor of the proinflammatory cytokine IL18. IL-18BP is involved in the biological processes of cytokine stimulus, IL-18 mediated signaling pathway, and immune response.

MSR1 is a membrane glycoprotein expressed by macrophages. MSR1 is involved in the biological processes of endocytosis and cholesterol transport and storage and can be implicated in the pathologic deposition of cholesterol in arterial walls during atherogenesis.

CCL3 is a monokine with inflammatory and chemokinetic properties that binds to CCR1, CCR4, and CCR5. CCL3 is involved in the biological processes of cell migration (e.g., lymphocyte and macrophage chemotaxis), calcium-mediated signaling, cell-cell signaling, cytokine secretion, and inflammatory response.

TWEAK is a cytokine in the tumor necrosis factor ligand family. TWEAK is involved in the biological processes of angiogenesis, cell differentiation, immune response, signal transduction, apoptosis, and the TNF mediated signaling pathway. TWEAK further promotes proliferation and migration of endothelial cells.

TFF3 is a 6 kDa glycoprotein generally produced by goblet cells and involved in the gastrointestinal tract. TFF3 is involved in the biological processes of the maintenance and healing of gastrointestinal epithelium and regulation of glucose metabolic processes.

ENPP2 is a phosphodiesterase involved in the generation of the lipid signaling molecule lysophosphatidic acid. ENPP2 hydrolyzes lysophospholipids and is involved in the biological processes of cell motility, chemotaxis, immune response, and angiogenesis.

IGFBP-1 is a member of the insulin-like growth factor binding protein family. It binds insulin-like growth factors (IGF) I and II. IGFBP-1 is involved in the biological processes of aging, cell metabolic processes, cell growth, signal transduction, and tissue regeneration.

IL-12A is a subunit that, along with the other IL-12B subunit, together form the IL-12 heterodimer. IL-12A is involved in the biological processes of cell migration, cell proliferation, cell adhesion, cell differentiation, regulation of immune cell (e.g., T-cell, dendritic cell, natural killer cell) activation.

SEZ6L is a protein primarily located in the endoplasmic reticulum membrane and it regulates endoplasmic reticulum functions in neurons. SEZ6L is involved in the biological processes of synapse maturation, adult locomotory behavior, and regulation of protein kinase C signaling.

DPP6 is a membrane protein that is a member of the peptidase S9B family of serine proteases. DPP6 is involved in the biological processes of regulation of potassium channels and protein localization to the plasma membrane, and can influence susceptibility to amyotrophic lateral sclerosis.

NCAN is a protein that is a member of the lectican/chondroitin sulfate proteoglycan families. NCAN is involved in the biological processes of cell adhesion, CNS development, ECM organization, and synthesis of chondroitin sulfate and dermatan. NCAN is involved in neuronal adhesion and neurite growth during development by binding to neural cell adhesion molecules.

TINAGL1 is an extracellular matrix protein involved in the biological processes of cell adhesion, proliferation, migration, and differentiation. TINAGL1 further plays a role in endocytosis and endosomal transport.

CANT1 is a calcium-dependent nucleotidase with a preference of uridine diphosphate. CANT1 is involved in the biological processes of regulation of calcium ion binding, neutrophil degranulation, regulation of NF-kappa B signaling, and proteoglycan biosynthetic process. CANT1 regulates metabolism processes, such as metabolism of nucleotides.

NECTIN2 is a membrane glycoprotein serving as a component of adherens junction. NECTIN2 is involved in the biological processes of cell-cell adhesion (through adherens junction organization), cytoskeletal organization, virus receptor activity, and regulation of NK cell and T cell activities.

NPDC1 is a protein expressed primarily in brain. NPDC1 is involved in the biological processes of the regulation of immune response and neural cell development and proliferation. NPDC1 suppresses oncogenic transformation in neural and non-neural cells and down-regulates neural cell proliferation.

TNFRSF11A is a member of the TNF-receptor superfamily. TNFRSF11A is involved in the biological processes of cell-cell signaling, immune response, monocyte chemotaxis, and TNF-mediated signaling pathway. Furthermore, TNFSF11A plays a role in osteoclastogenesis.

CNTN4 is a member of the contactin family of immunoglobulins. CNTN4 is involved in the biological processes of axonal guidance and development, synaptogenesis, cell surface interactions during nervous system development, brain development, neuron cell-cell adhesion, neuron projection, neuron differentiation, and regulation of synaptic plasticity.

NTKR2 is a receptor tyrosine kinase (part of the neurotrophic tyrosine receptor kinase family) that binds to brain-derived neurotropic factor. NTKR2 is involved in the biological processes of neuron survival, proliferation, migration, differentiation, synapse formation, and synapse plasticity.

NTKR3 is a receptor tyrosine kinase (part of the neurotrophic tyrosine receptor kinase family) that binds to neurotrophin-3. NTKR3 is involved in the biological processes of regulation of GTPase and MAPK activity, regulation of astrocyte differentiation, nervous system development, and neuron migration.

CDH6 is a member of the cadherin superfamily which mediates cell-cell adhesion. CDH6 is involved in the biological processes of cell-cell adhesion (adherens junction organization), cell morphogenesis, and Notch signaling pathway. CDH6 mediates both heterotypic cell-cell contacts via its interaction with CD6, as well as homotypic cell-cell contacts. CDH6 is further involved in axon extension and axon guidance.

CEACAM8 is a cell surface glycoprotein belonging to the carcinoembryonic antigen (CEA) superfamily. CEACAM8 is involved in the biological processes of regulation of the immune response, leukocyte migration, neutrophil degranulation, and cell-cell adhesion.

MAD1L1 is a mitotic spindle assembly checkpoint protein. MAD1L1 is involved in the biological processes of cell division and mitotic cell cycle checkpoint.

FCAR is a transmembrane glycoprotein on the surface of immune cells such as neutrophils, monocytes, and macrophages. FCAR is involved in the biological processes of regulation of the immune response, neutrophil activation/degranulation, and response to cytokines (e.g., interferons, interleukins, TNF).

MPO is a heme protein (enzyme) expressed in neutrophil granulocytes. MPO is involved in the biological processes of the immune response, neutrophil degranulation, and chromatin/heme/heparin binding.

OMD is suggested to be involved in biomineralization processes and in binding osteoblasts. OMD is involved in biological processes of cell adhesion and regulation of bone mineralization.

MEPE is a calcium-binding phosphoprotein in the small integrin-binding ligand, N-linked glycoprotein (SIBLING) family. MEPE is involved in biological processes of extracellular matrix binding/regulation, biomineral tissue development, skeletal system development, and bone and cartilage mineralization. MEPE is involved in renal phosphate excretion and inhibits intestinal phosphate absorption. MEPE is further involved in dental pulp stem cell proliferation and differentiation.

GDNFR-alpha-3 is a glial cell line-derived neurotrophic factor and is a member of the GDNF receptor family and binds to artemin (ARTN). GDNFR-alpha-3 is involved in the biological processes of axon guidance, nervous system development, neuron migration, GDNF receptor activity, and signaling receptor activity and binding.

SCARF2 is a member of the scavenger receptor type F family. SCARF 2 is an adhesion protein and is involved in the biological processes of scavenger receptor activity and cell-cell adhesion.

CD40 ligand is primary expressed on activated T cells and a member of the TNF superfamily of molecules. CD40 ligand is involved in the biological processes of B cell differentiation and proliferation, inflammatory response, leukocyte cell-cell adhesion, platelet activation, T cell costimulation, and TNF-mediated signaling pathway.

TNF-R2 is a membrane receptor that binds tumor necrosis factor-alpha. TNF-R2 is involved in the biological processes of TNF-mediated signaling pathway, neutrophil degranulation, regulation of neuroinflammatory response, and cell signaling. TNF-R2 protects neurons from apoptosis by stimulating antioxidative pathways and protects neurons from apoptosis by stimulating antioxidative pathways

PD-L1 is a ligand that binds to PD-1 and is an important target in cancer immunotherapy checkpoint inhibitor research. PD-L1 is involved in biological processes of immune response, interferon regulation, T cell proliferation, T cell costimulation, cell migration, and cytokine production.

NOTCH3 is a protein in the NOTCH receptor family involved in the Notch signaling pathway. NOTCH3 is involved in the biological processes of gene activation, calcium ion binding, signaling receptor activity, neuron fate commitment, and brain development. NOTCH3 further regulates cell-fate determination.

CNTN1 is a neuronal membrane protein that plays a role in cell adhesion. CNTN1 is involved in the biological processes of neuron projection development, brain development, cell adhesion, and Notch signaling.

OSM is a cytokine in the interleukin 6 cytokine family. OSM is involved in the biological processes of regulation of immune response, cell proliferation/division, inflammatory response, and cytokine activity (e.g., MAPK and STAT pathways).

TGFA is a mitogenic polypeptide and is part of the epidermal growth factor family. TGFA is involved in the biological processes of growth factor activity, signaling pathways (e.g., MAPK, EGF), cell division/proliferation, and signal transduction.

PGLYRP1 is a peptidtoglycan binding protein and is involved in the biological processes of the innate immune response, inflammatory response, neutrophil degranulation, and peptidoglycan immune receptor activity.

NOS3 regulates production of nitric oxide. NOS3 is involved in the biological processes of angiogenesis, blood vessel remodeling, endothelial cell migration, vasodilation, vascular smooth muscle relaxation, and promotion of blood clotting through the activation of platelets.

DDR1 regulates cell attachment to the extracellular matrix, remodeling of the extracellular matrix, cell migration, differentiation, and survival and cell proliferation. DDR1 further promotes smooth muscle cell migration.

CXCL166 plays a role in immune modulation and serves as a scavenger receptor on macrophages.

IL6 is a cytokine involved in differentiation of B-cells, lymphocytes, and monocytes.

ALCAM is involved in axon guidance, embryonic and induced pluripotent stem cell differentiation pathways and lineage-specific markers, as well as L1CAM interactions.

NTRK2 is involved in the development and the maturation of the central and the peripheral nervous systems through regulation of neuron survival, proliferation, migration, differentiation, and synapse formation and plasticity.

SPON2 is involved in the outgrowth of hippocampal embryonic neurons.

NTRK3 is involved in regulating the nervous system and development of the heart.

PCDH17 is involved in establishment and function of specific cell-cell connections in the brain.

### VI. Assays

As shown in FIG. 1A, the system environment 100 involves implementing a marker quantification assay 120 for evaluating expression levels of one or more biomarkers. Examples of an assay (e.g., marker quantification assay 120) for one or more markers include DNA assays, microarrays, polymerase chain reaction (PCR), RT-PCR, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, MA, immunofluorescence, immunochemiluminescence, immunoelectrochemiluminescence, or competitive immunoassays, immunoprecipitation, and the assays described in the Examples section below. The information from the assay can be quantitative and sent to a computer system of the invention. The information can also be qualitative, such as observing patterns or fluorescence, which can be translated into a quantitative measure by a user or automatically by a reader or computer system.

Various immunoassays designed to quantitate markers can be used in screening including multiplex assays. Measuring the concentration of a target marker in a sample or fraction thereof can be accomplished by a variety of specific assays. For example, a conventional sandwich type assay can be used in an array, ELISA, MA, etc. format. Other immunoassays include Ouchterlony plates that provide a simple determination of antibody binding. Additionally, Western blots can be performed on protein gels or protein spots on filters, using a detection system specific for the markers as desired, conveniently using a labeling method.

Protein based analysis, using an antibody that specifically binds to a polypeptide (e.g. marker), can be used to quantify the marker level in a test sample obtained from a subject. In various embodiments, an antibody that binds to a marker can be a monoclonal antibody. In various embodiments, an antibody that binds to a marker can be a polyclonal antibody. For multiplex analysis of markers, arrays containing one or more marker affinity reagents, e.g. antibodies can be generated. Such an array can be constructed comprising antibodies against markers. Detection can utilize one or a panel of marker affinity reagents, e.g. a panel or cocktail of affinity reagents specific for one, two, three, four, five, six, seven, eight, nine, ten, eleven, twelve, thirteen, fourteen, fifteen, sixteen, seventeen, eighteen, nineteen, twenty, twenty one, or more markers.

In various embodiments, the multiplex assay involves the use of oligonucleotide labeled antibody probes that bind to target biomarkers and allow for subsequent quantification of biomarkers. One example of a multiplex assay that involves oligonucleotide labeled antibody probes is the Proximity Extension Assay (PEA) technology (Olink Proteomics). Briefly, a pair of oligonucleotide labeled antibodies bind to a biomarker, wherein the two oligonucleotide sequences are complementary to one another. Thus, only when both antibodies bind to the target biomarker will the oligonucleotide sequences hybridize with one another. Mismatched oligonucleotide sequences (which occurs due to non-specific binding of antibodies or cross-reactivity of antibodies) will not hybridize and therefore, will not result in a readout. Hybridized oligonucleotide sequences undergo nucleic acid extension and amplification, followed by quantification using microfluidic qPCR. The quantified levels correlate to the quantitative expression values of the respective biomarkers.

In various embodiments, the multiplex assay involves the use of bead conjugated antibodies (e.g., capture antibodies) that enable the binding and detection of biomarkers. One example of a multiplex assay involving bead conjugated antibodies is Luminex's xMAP® Technology. Here, bead conjugated antibodies are added to the sample along with biotinylated detection antibodies. Both antibodies are specific to the biomarkers of interest and therefore, form an antibody-antigen sandwich. Streptavidin is further added, which binds to the biotinylated detection antibodies and enables detection of the complex. The Luminex 200™ or FlexMap® analyzer are employed to identify and quantify the amount of the biomarker in the sample. In various embodiments, the multiplex assay represents an improvement over Luminex's xMAP® technology, such as the Multi-Analyte Profile (MAP) technology by Myriad Rules Based Medicine (RBM), Inc.

In various embodiments, prior to implementation of a marker quantification assay 120 (e.g., an immunoassay), a sample obtained from a subject can be processed. In various embodiments, processing the sample enables the implementation of the marker quantification assay 120 to more accurately evaluate expression levels of one or more biomarkers in the sample.

In various embodiments, the sample from a subject can be processed to extract biomarkers from the sample. In one embodiment, the sample can undergo phase separation to separate the biomarkers from other portions of the sample. For example, the sample can undergo centrifugation (e.g., pelleting or density gradient centrifugation) to separate larger and/or more dense entities in the sample (e.g., cells and other macromolecules) from the biomarkers. Other examples include filtration (e.g., ultrafiltration) to phase separate the biomarkers from other portions of the sample.

In various embodiments, the sample from a subject can be processed to produce a sub-sample with a fraction of biomarkers that were in the sample. In various embodiments, producing a fraction of biomarkers can involve performing a protein fractionation procedure. One example of protein fractionation procedures include chromatography (e.g., gel filtration, ion exchange, hydrophobic chromatography, or affinity chromatography). In particular embodiments, the protein fractionation procedure involves affinity purification or immunoprecipitation where biomarkers are bound by specific antibodies. Such antibodies can be immobilized on a support, such as a magnetic particle or nanoparticle or a plate.

In various embodiments, the sample from the subject is processed to extract biomarkers from the sample and further processed to produce a sub-sample with a fraction of extracted biomarkers. Altogether, this enables a purified sub-sample of biomarkers that are of particular interest. Thus, implementing an assay (e.g., an immunoassay) for evaluating expression levels of the biomarkers of particular interest can be more accurate and of higher quality. In various embodiments, the biomarkers of particular can be biomarkers of a biomarker panel, embodiments of which are described herein. As an example, biomarkers of a biomarker panel can include two or more of: NEFL, MOG, CD6, CXCL9, OPG, OPN, CXCL13, GFAP, CDCP1, CCL20, IL-12B, APLP1, TNFRSF10A, COL4A1, SERPINA9, FLRT2, TNFSF13B, GH, VCAN, PRTG, and CNTN2.

### VII. Therapeutic Agents and Compositions for Therapeutic Agents

In various embodiments, a therapeutic agent is provided to an individual prior to and/or subsequent to obtaining the sample from the individual and determining quantitative expression values of one or more markers in the obtained sample. As one example, a predictive model that receives the quantitative expression values predicts that an individual is to be diagnosed with multiple sclerosis and a therapeutic agent is to be provided. In another example, the predictive model predicts that a provided therapeutic agent is demonstrating therapeutic efficacy against a multiple in a previously diagnosed individual.

In various embodiments the therapeutic agent is a biologic, e.g. a cytokine, antibody, soluble cytokine receptor, anti-sense oligonucleotide, siRNA, etc. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists, e.g. IL-1Ra, IL-1 Trap, sIL-4Ra, etc. Also included are biosimilar or bioequivalent drugs to the active agents set forth herein.

Therapeutic agents for multiple sclerosis include corticosteroids, plasma exchange, ocrelizumab (Ocrevus®), IFN-β (Avonex®, Betaseron®, Rebif®, Extavia®, Plegridy®), Glatiramer acetate (Copaxone®, Glatopa®), anti-VLA4 (Tysabri, natalizumab), dimethyl fumarate (Tecfidera®, Vumerity®), teriflunomide (Aubagio®), monomethyl fumarate (Bafiertam™), ozanimod (Zeposia®), siponimod (Mayzent®), fingolimod (Gilenya®), anti-CD52 antibody (e.g., alemtuzumab (Lemtrada®), mitoxantrone (Novantrone®), methotrexate, cladribine (Mavenclad®, simvastatin, and cyclophosphamide. In addition or alternative to therapeutic agents, other treatments for multiple sclerosis include lifestyle changes such as physical therapy or a change in diet. The method also provide for combination therapy of one or more therapeutic agents and/or additional treatments, where the combination can provide for additive or synergistic benefits.

A pharmaceutical composition administered to an individual includes an active agent such as the therapeutic agent described above. The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients. Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

Such a pharmaceutical composition may be administered for prophylactic (e.g., before diagnosis of a patient with multiple sclerosis) or for treatment (e.g., after diagnosis of a patient with multiple sclerosis) purposes. Preventing, prophylaxis or prevention of a disease or disorder as used in the context of this invention refers to the administration of a composition to prevent the occurrence or onset of multiple sclerosis or some or all of the symptoms of multiple sclerosis or to lessen the likelihood of the onset of a disease or disorder. Treating, treatment, or therapy of multiple sclerosis shall mean slowing, stopping or reversing the disease's progression by administration of treatment according to the present invention. In the preferred embodiment, treating multiple sclerosis means reversing the disease's progression, ideally to the point of eliminating the disease itself.

### VIII. Disease Activity in a Subject

Methods described herein focus on assessing disease activity in a subject by applying quantitative expression levels of biomarkers as input to a predictive model. In various embodiments, the subject is classified in a category based on the predicted assessment of the disease activity. To classify the subject, the prediction for the subject may be compared to results of individuals that have been previously classified in a clinically diagnosed category. For example, individuals may be clinically categorized in one of a diagnosis of MS (e.g., presence of MS), a categorization of a subtype of MS (e.g., radiologically isolated syndrome (RIS), clinically isolated syndrome (CIS), relapsing-remitting MS (RRMS), primary progressive MS (PPMS), and secondary-progressive MS (SPMS)), a categorization in a quiescent or exacerbated state, a categorization in a level of disability according to the expanded disability status scale (EDSS), an identified clinical response to a therapy, and a clinical identification of a risk of developing MS. Clinical categories can also be determined using any of a MS functional composite (MSFC), timed 25-foot walk (T25Fw), 9-hole peg test (9HPT), or patient-reported outcomes (e.g., patient determined disease steps (PDDS)/MSSS (patient-derived disability status scale), PRO measurement information system (PROMIS), or Multiple Sclerosis Rating Scale, Revised (MSRS-R)). Individuals may be clinically categorized based on a measurable for MS disease activity, such as a particular number of gadolinium enhancing lesions (e.g., subtle disease activity) or the presence of at least one gadolinium enhancing lesion (e.g., general disease activity). Clinical categorization can also occur based on other radiographic measures including T2 lesions (new or enlarging), slowly expanding lesions, rim-expanding lesions, Brain Parenchymal Fraction (BPF) & percentage change, Gray matter fraction, White matter fraction, Thalamic volume, Cortical gray matter volume, Deep gray matter volume, or Radiologist notes of auxiliary features (e.g. Dawson's Fingers). Categorization of previously individuals may occur based on clinical standards.

Clinical diagnosis of MS can occur through various methods. As an example, a clinical diagnosis of MS can be made through magnetic resonance imaging (MM) of the brain and spinal cord to identify lesions or plaques that form as a result of MS. The McDonald criteria can be employed in making the diagnosis. Clinical diagnosis of MS can also occur through a lumbar puncture (spinal tap) that observes abnormalities in antibody concentrations in the spinal fluid due to the presence of MS. Clinical diagnosis of MS can also occur through evoked potential tests, where electrical signals produced by neurons of the nervous system are recorded in response to a stimulus. An impaired transmission is indicative of the presence of MS.

Clinical categorization of a patient previously diagnosed with MS in a quiescent state versus an exacerbated state can depend on a variety of factors. Namely, a patient can be clinically categorized in an exacerbated state after presenting with a new disease that is related to MS (e.g., a comorbidity or symptom such as clinical depression or optic neuritis). As another example, a patient is clinically categorized in an exacerbated state if the patient presents with significant worsening of symptoms. Examples may include a worsening of balance and/or mobility, vision, pain in the eye, fatigue, and/or heart-related problems. Patients previously diagnosed with MS can be clinically categorized in a quiescent state if the patient does not present with a new disease or a change or worsening of symptoms.

Determination that a patient previously diagnosed with MS is responding to a therapy can be dependent on a variety of clinical variables. For example, a response to therapy can be determined based on the occurrence or lack of a relapse. A patient can be deemed responsive to a therapy if relapses do not occur. A response to therapy can also be determined based on a total number of relapses, a time to a first relapse, the patient's EDSS score, a change in the patient's EDSS score (e.g., an increase in the score corresponds to a lack of response to therapy), a change in MRI status (e.g., the development of additional lesions or plaques corresponds to a lack of response to therapy).

Patients can be clinically categorized in a level of disability, which can be a measure of disease progression. For example, the EDSS can be used to determine a severity of MS in a patient. Therefore, patients are categorized in categories that correspond to an EDSS score between 1.0 and 10.0 in 0.5 point intervals. Generally, EDSS scores of 1.0 to 4.5 refer to patients with MS who are able to walk without any aid. EDSS scores of 5.0 to 9.5 refer to patients with MS whose ability to walk is impaired, with a higher score corresponding to a higher degree of impairment. In various embodiments, an EDSS score less than 6 indicates mild/moderate MS disease progression. In various embodiments an EDSS score greater than or equal to 6 indicates severe MS disease progression. In particular embodiments, an EDSS score between 0-3.0 represents mild MS, an EDSS score between 3.5-5.5 represents moderate MS, an EDSS score between 6.0-9.5 represents severe MS.

As another example, patents can be clinically categorized in a level of disability according to PDDS, which is a validated scale as a self-reported proxy for EDSS and therefore, can be used to determine a severity of MS in a patient. A PDDS score of 0 indicates a normal disability level with mild, sensory symptoms with no limit on activity. A PDDS score of 1 indicates a mild disability with minor, noticeable symptoms that have only a small effect on lifestyle. A PDDS score of 2 indicates moderate disability with no limitation in walking ability but significant problems that limit daily activities in other ways. A PDDS score of 3 indicates gait disability with interferences with activities such as walking. A PDDS score of 4 indicates early cane disability which is characterized by use of a cane or single crutch for walking all or part of the time (e.g., can walk 25 feet in 20 seconds without a cane or crutch). A PDDS score of 5 indicates late cane disability which is character the use of a cane or crutch to walk 25 feet. A PDDS score of 6 indicates bilateral support disability which is characterize by the need to use 2 canes, crutches, or a walker to walk 25 feet. A PDDS score of 7 indicates wheelchair/scooter disability in which the individual's main form of mobility is a wheelchair/scooter. A PDDS score of 8 indicates bedridden disability in which the individual is unable to sit in a wheelchair for more than 1 hour. In various embodiments, a PDDS score less than or equal to 4 indicates disease progression to mild/moderate MS disability. In various embodiments a PDDS score greater than 4 indicates disease progression to severe MS disability. In particular embodiments, a PDDS score between 0 and 1 represents mild MS, a PDDS score between 2-4 represents moderate MS, and a PDDS score between 5-8 represents severe MS.

As another example, patents can be clinically categorized in a level of disability according to PROMIS measure. Generally, PROMIS scores are based on the T-score metric in which a score of 50 represents a mean score of a corresponding reference population with a standard deviation of 10. Therefore, a score of 40 for an individual indicates that the individual is one standard deviation lower than the mean of the corresponding reference population (e.g., score of 40 indicates that individual's MS disability is a standard deviation lower than the MS disability of the mean of the population). A score of 60 for an individual indicates that the individual is one standard deviation higher than the mean of the corresponding reference population (e.g., score of 60 indicates that individual's MS disability is a standard deviation higher than the MS disability of the mean of the population).

As another example, patents can be clinically categorized in a level of disability according to a MSRS-R measure. Generally, MSRS-R scores can be measured according to the following items: 1) Walking, 2) Using your arms and hands, 3) Vision (with glasses or contacts if you use them), 4) Speaking clearly, 5) Swallowing, 6) Thinking, Memory, or Cognition, 7) Numbness, Tingling, Burning Sensation or Pain, and 8) Bowel or bladder. Each of the items is rated according to a level of disability: “0—Normal status”, “1—Symptoms causing no disability”, “2—Mild disability not requiring help from others”, “3—Moderate disability requiring help from others”, and 4—“Total loss of function, maximal help required”). Total MSRS-R score represents the sum of the scores across the 8 items.

### IX. Computer Implementation

The methods of the invention, including the methods of assessing multiple sclerosis activity (e.g., multiple sclerosis disease progression) in an individual, are, in some embodiments, performed on one or more computers.

For example, the building and deployment of a predictive model and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and execution and results of a predictive model of this invention. Such data can be used for a variety of purposes, such as patient monitoring, treatment considerations, and the like. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods of assessing multiple sclerosis disease activity (e.g., multiple sclerosis disease progression) in an individual, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

VIII.A Example Computer

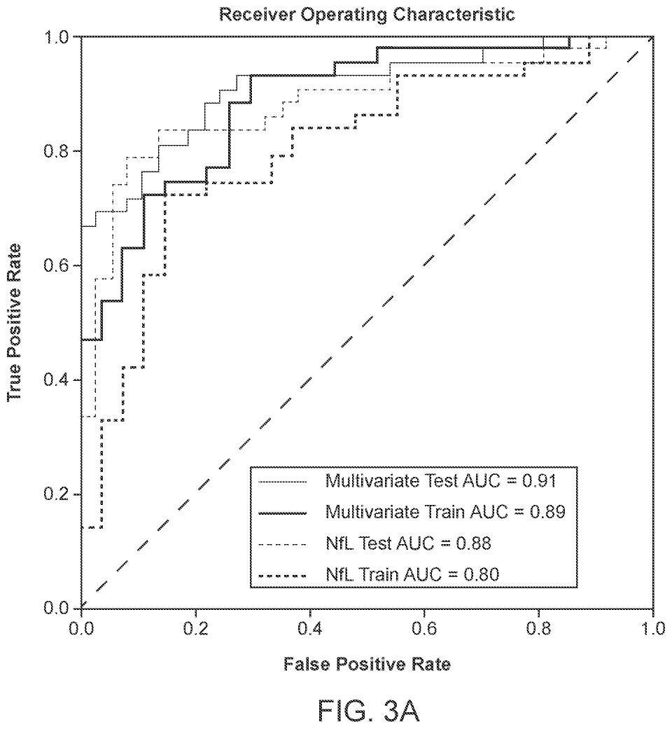

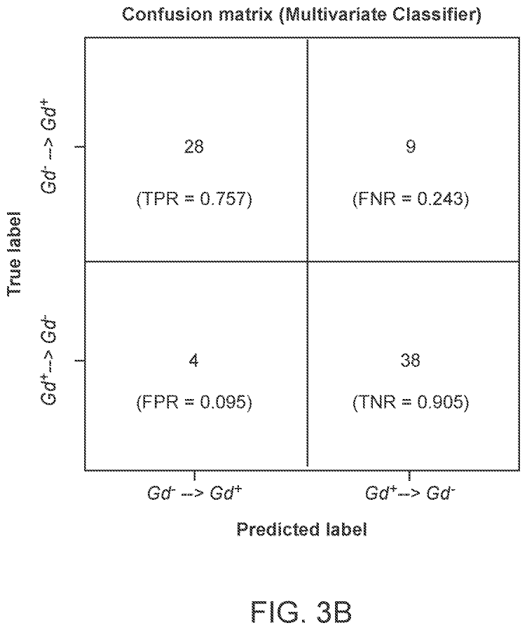

FIG. 8 illustrates an example computer 800 for implementing the entities shown in FIGS. 1 and 3. The computer 800 includes at least one processor 802 coupled to a chipset 804. The chipset 804 includes a memory controller hub 820 and an input/output (I/O) controller hub 822. A memory 806 and a graphics adapter 812 are coupled to the memory controller hub 820, and a display 818 is coupled to the graphics adapter 812. A storage device 808, an input device 814, and network adapter 816 are coupled to the I/O controller hub 822. Other embodiments of the computer 800 have different architectures.

The storage device 808 is a non-transitory computer-readable storage medium such as a hard drive, compact disk read-only memory (CD-ROM), DVD, or a solid-state memory device. The memory 806 holds instructions and data used by the processor 802. The input interface 814 is a touch-screen interface, a mouse, track ball, or other type of pointing device, a keyboard, or some combination thereof, and is used to input data into the computer 800. In some embodiments, the computer 800 may be configured to receive input (e.g., commands) from the input interface 814 via gestures from the user. The graphics adapter 812 displays images and other information on the display 818. The network adapter 816 couples the computer 800 to one or more computer networks.

The computer 800 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 808, loaded into the memory 806, and executed by the processor 802.

The types of computers 800 used by the entities of FIG. 1 can vary depending upon the embodiment and the processing power required by the entity. For example, the disease progression system 130 can run in a single computer 800 or multiple computers 800 communicating with each other through a network such as in a server farm. The computers 800 can lack some of the components described above, such as graphics adapters 812, and displays 818.

### X. Kit Implementation

Also disclosed herein are kits for assessing multiple sclerosis disease activity (e.g., multiple sclerosis disease progression) in an individual. Such kits can include reagents for detecting expression levels of one or biomarkers and instructions for assessing disease activity (e.g., multiple sclerosis disease progression) based on the detected expression levels.

The detection reagents can be provided as part of a kit. Thus, the invention further provides kits for detecting the presence of a panel of biomarkers of interest in a biological test sample. A kit can comprise a set of reagents for generating a dataset via at least one protein detection assay (e.g., immunoassay) that analyzes the test sample from the subject. In various embodiments, the set of reagents enable detection of quantitative expression levels of biomarkers from any one of Tables 4-6. In particular embodiments, the set of reagents enable detection of quantitative expression levels of biomarkers categorized as Tier A, Tier B, or Tier C biomarkers in Table 1. In particular embodiments, the set of reagents enable detection of quantitative expression levels of biomarkers categorized as Tier 1, Tier 2, or Tier 3 biomarkers in Table 2. In particular embodiments, the set of reagents enable detection of quantitative expression levels of biomarkers categorized as Tier 1, Tier 2, or Tier 3 biomarkers in Table 3. In certain aspects, the reagents include one or more antibodies that bind to one or more of the markers. The antibodies may be monoclonal antibodies or polyclonal antibodies. In some aspects, the reagents can include reagents for performing ELISA including buffers and detection agents.

A kit can include instructions for use of a set of reagents. For example, a kit can include instructions for performing at least one biomarker detection assay such as an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, proximity extension assay, and an immunoassay selected from RIA, immunofluorescence, immunochemiluminescence, immunoelectrochemiluminescence, immunoelectrophoretic, a competitive immunoassay, and immunoprecipitation.

In various embodiments, the kits include instructions for practicing the methods disclosed herein (e.g., methods for training or deploying a predictive model to predict an assessment of disease activity, such as multiple sclerosis disease progression). These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### XI. Systems

Further disclosed herein are system for analyzing quantitative expression levels of biomarkers for assessing disease activity (e.g., multiple sclerosis disease progression). In various embodiments, such a system can include a set of reagents for detecting expression levels of biomarkers in the biomarker panel, an apparatus configured to receive a mixture of the set of reagents and a test sample obtained from a subject to measure the expression levels of the soluble mediators, and a computer system communicatively coupled to the apparatus to obtain the measured expression levels and to implement the predictive model to assess the disease activity (e.g., multiple sclerosis disease progression).

The set of reagents enable the detection of quantitative expression levels of the biomarkers in the biomarker panel. In various embodiments, the set of reagents involve reagents used to perform an assay, such as an assay or immunoassay as described above. For example, the reagents include one or more antibodies that bind to one or more of the biomarkers. The antibodies may be monoclonal antibodies or polyclonal antibodies. As another example, the reagents can include reagents for performing ELISA including buffers and detection agents.

The apparatus is configured to detect expression levels of biomarkers in a mixture of a reagent and test sample. For example, the apparatus can determine quantitative expression levels of biomarkers through an immunologic assay or assay for nucleic acid detection. The mixture of the reagent and test sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading to generate quantitative expression values of biomarkers. Examples of an apparatus include a plate reader (e.g., a luminescent plate reader, absorbance plate reader, fluorescence plate reader), a spectrometer, and a spectrophotometer.

The computer system, such as example computer 800 described in FIG. 8, communicates with the apparatus to receive the quantitative expression values of biomarkers. The computer system implements, in silico, a predictive model to analyze the quantitative expression values of the biomarkers to predict an assessment of the disease activity (e.g., multiple sclerosis disease progression).

### XII. Additional Embodiments

Additionally disclosed herein are methods for predicting multiple sclerosis progression in a subject, the method comprising: obtaining or having obtained a dataset comprising expression levels of a plurality of biomarkers, wherein the plurality of biomarkers comprise each biomarker in at least one group selected from group 1, group 2, and group 3, wherein group 1 comprises one or more of biomarker 1, biomarker 2, biomarker 3, biomarker 4, biomarker 5, biomarker 6, biomarker 7, and biomarker 8, wherein biomarker 1 is NEFL, MOG, CADM3, or GFAP, wherein biomarker 2 is MOG, CADM3, KLK6, BCAN, OMG, or GFAP, wherein biomarker 3 is CD6, CD5, CRTAM, CD244, or TNFRSF9, wherein biomarker 4 is CXCL9, CXCL10, IL-12B, CXCL11, or GFAP, wherein biomarker 5 is OPG, TFF3, or ENPP2, wherein biomarker 6 is OPN, OMD, MEPE, or GFAP, wherein biomarker 7 is CXCL13, NOS3, or MMP-2, and wherein biomarker 8 is GFAP, NEFL, OPN, CXCL9, MOG, or CHI3L1, and wherein group 2 comprises one or more of biomarker 9, biomarker 10, biomarker 11, biomarker 12, biomarker 13, biomarker 14, biomarker 15, and biomarker 16, wherein biomarker 9 is CDCP1, IL-18BP, IL-18, GFAP, or MSR1, wherein biomarker 10 is CCL20, CCL3, or TWEAK, wherein biomarker 11 is IL-12B, IL12A, or CXCL9, wherein biomarker 12 is APLP1, SEZ6L, BCAN, DPP6, NCAN, or KLK6, wherein biomarker 13 is TNFRSF10A, TNFRSF11A, SPON2, CHI3L1, or IFI30, wherein biomarker 14 is SERPINA9, TNFRSF9, or CNTN4, wherein biomarker 15 is FLRT2, DDR1, NTRK2, CDH6, MMP-2, and wherein biomarker 16 is TNFSF13B, CXCL16, ALCAM, or IL-18, wherein group 3 comprises one or more of biomarker 17, biomarker 18, biomarker 19, and biomarker 20, wherein biomarker 17 is GH, GH2, or IGFBP-1, wherein biomarker 18 is VCAN, TINAGL1, CANT1, NECTIN2, MMP-9, or NPDC1, wherein biomarker 19 is PRTG, NTRK2, NTRK3, or CNTN4, and wherein biomarker 20 is CNTN2, DPP6, GDNFR-alpha-3, or SCARF2, and generating a prediction of multiple sclerosis disease progression by applying a predictive model to the expression levels of the plurality of biomarkers.

In various embodiments, the plurality of biomarkers comprise each biomarker in group 1, wherein biomarker 1 is NEFL, wherein biomarker 2 is MOG, wherein biomarker 3 is CD6, wherein biomarker 4 is CXCL9, wherein biomarker 5 is OPG, wherein biomarker 6 is OPN, wherein biomarker 7 is CXCL13, and wherein biomarker 8 is GFAP. In various embodiments, the plurality of biomarkers further comprise each biomarker in group 2, wherein biomarker 9 is CDCP1, wherein biomarker 10 is CCL20, wherein biomarker 11 is IL-12B, wherein biomarker 12 is APLP1, wherein biomarker 13 is TNFRSF10A, wherein biomarker 14 is SERPINA9, wherein biomarker 15 is FLRT2, and wherein biomarker 16 is TNFSF13B. In various embodiments, the plurality of biomarkers further comprise each biomarker in group 3, wherein biomarker 17 is GH, wherein biomarker 18 is VCAN, wherein biomarker 19 is PRTG, and wherein biomarker 20 is CNTN2.

In various embodiments, a performance of the predictive model is characterized by a correlation coefficient (R²) of at least 0.517. In various embodiments, the prediction of multiple sclerosis disease progression is a brain parenchymal fraction value.

In various embodiments, the plurality of biomarkers comprise biomarkers in group 1 comprising biomarker 2 and biomarker 8, wherein biomarker 2 is MOG and biomarker 8 is GFAP. In various embodiments, the plurality of biomarkers comprise biomarkers in group 1 comprising biomarker 1 and biomarker 8, wherein biomarker 1 is NEFL and biomarker 8 is GFAP. In various embodiments, the plurality of biomarkers comprise biomarkers in group 1 comprising biomarker 8 and group 2 comprising biomarker 12, wherein biomarker 8 is GFAP, and biomarker 12 is APLP1. In various embodiments, the plurality of biomarkers comprise biomarkers in group 1 comprising biomarker 1 and biomarker 2, wherein biomarker 1 is NEFL and biomarker 2 is MOG. In various embodiments, the plurality of biomarkers comprise biomarkers in group 1 comprising biomarker 1, biomarker 2, and biomarker 8, wherein biomarker 1 is NEFL, biomarker 2 is MOG, and biomarker 8 is GFAP. In various embodiments, the plurality of biomarkers comprise biomarkers in group 1 comprising biomarker 1 and biomarker 2 and group 3 comprising biomarker 18, wherein biomarker 1 is NEFL, biomarker 2 is MOG, and biomarker 18 is GH. In various embodiments, the plurality of biomarkers comprise biomarkers in group 1 comprising biomarker 1 and biomarker 2 and group 2 comprising biomarker 15, wherein biomarker 1 is NEFL, biomarker 2 is MOG, and biomarker 15 is SERPINA9. In various embodiments, the plurality of biomarkers comprise biomarkers in group 1 comprising biomarker 6 and biomarker 7 and group 2 comprising biomarker 15, wherein biomarker 6 is OPN, biomarker 7 is CXCL13, and biomarker 15 is SERPINA9. In various embodiments, the plurality of biomarkers comprise biomarkers in group 1 comprising biomarker 6 and biomarker 7 and group 2 comprising biomarker 13, wherein biomarker 6 is OPN, biomarker 7 is CXCL13, and biomarker 13 is TNFRSF10A. In various embodiments, the plurality of biomarkers comprise biomarkers in group 1 comprising biomarker 1, biomarker 2, and biomarker 8 and group 2 comprising biomarker 11, wherein biomarker 1 is NEFL, biomarker 2 is MOG, biomarker 8 is GFAP, and biomarker 11 is IL-12B. In various embodiments, the plurality of biomarkers comprise biomarkers in group 1 comprising biomarker 1, biomarker 2, and biomarker 8 and group 3 comprising biomarker 20, wherein biomarker 1 is NEFL, biomarker 2 is MOG, biomarker 8 is GFAP, and biomarker 20 is PRTG. In various embodiments, the plurality of biomarkers comprise biomarkers in group 1 comprising biomarker 1, biomarker 2, and biomarker 8 and group 2 comprising biomarker 12, wherein biomarker 1 is NEFL, biomarker 2 is MOG, biomarker 8 is GFAP, and biomarker 12 is APLP1. In various embodiments, the plurality of biomarkers comprise biomarkers in group 1 comprising biomarker 1 and biomarker 2, group 2 comprising biomarker 15, and group 3 comprising biomarker 18, wherein biomarker 1 is NEFL, biomarker 2 is MOG, biomarker 15 is SERPINA9, and biomarker 18 is GH. In various embodiments, the plurality of biomarkers comprise biomarkers in group 1 comprising biomarker 1 and biomarker 2, group 2 comprising biomarker 13 and biomarker 15, wherein biomarker 1 is NEFL, biomarker 2 is MOG, biomarker 13 is TNFRSF10A, and biomarker 15 is SERPINA9. In various embodiments, the plurality of biomarkers comprise biomarkers in group 1 comprising biomarker 3 and group 2 comprising biomarker 10, biomarker 11, and biomarker 12, wherein biomarker 3 is CD6, biomarker 10 is CCL20, biomarker 11 is IL-12B, and biomarker 12 is APLP1. In various embodiments, the prediction of multiple sclerosis disease progression is an expanded disability status scale (EDSS) score. In various embodiments, an expanded disability status scale (EDSS) score less than 6 indicates a mild/moderate MS disease progression and a EDSS score greater than or equal to 6 indicates a severe MS disease progression.

In various embodiments, the plurality of biomarkers comprise biomarkers in group 1 comprising biomarker 1, biomarker 3, and biomarker 7, wherein biomarker 1 is NEFL, biomarker 3 is CD6, and biomarker 7 is CXCL13. In various embodiments, a performance of the predictive model is characterized by an area under the ROC curve (AUROC) value of at least 0.8. In various embodiments, a performance of the predictive model is characterized by an area under the ROC curve (AUROC) value of at least 0.9. In various embodiments, the prediction of multiple sclerosis disease progression is a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression.

In various embodiments, the plurality of biomarkers comprise biomarkers in group 2 comprising biomarker 9 and biomarker 11, wherein biomarker 9 is CDCP1 and biomarker 11 is IL-12B. In various embodiments, the prediction of multiple sclerosis disease progression is a PRO measurement information system (PROMIS) score.

In various embodiments, the plurality of biomarkers comprise biomarkers in group 1 comprising biomarker 3, biomarkers in group 2 comprising biomarker 9 and biomarker 11, and biomarkers in group 3 comprising biomarker 19, wherein biomarker 3 is CD6, biomarker 9 is CDCP1, biomarker 11 is IL-12B, and biomarker 19 is VCAN. In various embodiments, the prediction of multiple sclerosis disease progression is a Multiple Sclerosis Rating Scale, Revised (MSRS-R) score.

Additionally disclosed herein is a method for predicting multiple sclerosis progression in a subject, the method comprising: obtaining or having obtained a dataset comprising expression levels of a plurality of biomarkers comprising: one or more neurodegeneration biomarkers selected from a group consisting of NEFL, APLP1, OPG, SERPINA9, PRTG, GFAP, CNTN2, and FLRT2; one or more inflammation biomarkers selected from a group consisting of CCL20, GH, CXCL13, IL-12B, VCAN, TNFRSF10A, TNFSF13B, CD6, and CXCL9; one or more immune modulation biomarkers selected from a group consisting of CDCP1, and OPN; or one or more myelin integrity biomarkers selected from a group consisting of MOG; and generating a prediction of multiple sclerosis progression by applying a predictive model to the expression levels of the plurality of biomarkers.

In various embodiments, the one or more neurodegeneration biomarkers comprise NEFL, OPG, and GFAP, wherein the one or more inflammation biomarkers comprise CXCL13, CD6, and CXCL9, wherein the one or more immune modulation biomarkers comprise OPN, and wherein the one or more myelin integrity biomarkers comprise MOG. In various embodiments, the one or more neurodegeneration biomarkers further comprise APLP1, SERPINA9, and FLRT2, wherein the one or more inflammation biomarkers further comprise CCL20, IL-12B, TNFRSF10A, and TNFSF13B, wherein the one or more immune modulation biomarkers further comprise CDCP1. In various embodiments, the one or more neurodegeneration biomarkers further comprise PRTG and CNTN2, and wherein the one or more inflammation biomarkers comprise GH and VCAN. In various embodiments, a performance of the predictive model is characterized by a correlation coefficient (R²) of at least 0.517. In various embodiments, the prediction of multiple sclerosis disease progression is a brain parenchymal fraction value.

In various embodiments, the one or more myelin integrity biomarkers comprise MOG and wherein the one or more neurodegeneration biomarkers comprise GFAP. In various embodiments, the one or more neurodegeneration biomarkers comprise NEFL and GFAP. In various embodiments, the one or more neurodegeneration biomarkers comprise GFAP and APLP1. In various embodiments, the one or more neurodegeneration biomarkers comprise NEFL and wherein the one or more myelin integrity biomarkers comprise MOG. In various embodiments, the one or more neurodegeneration biomarkers comprise NEFL and GFAP and wherein the one or more myelin integrity biomarkers comprise MOG. In various embodiments, the one or more neurodegeneration biomarkers comprise NEFL, wherein the one or more myelin integrity biomarkers comprise MOG, and wherein the one or more inflammation biomarkers comprise GH. In various embodiments, the one or more neurodegeneration biomarkers comprise NEFL and SERPINA9 and wherein the one or more myelin integrity biomarkers comprise MOG, In various embodiments, the one or more immune modulation biomarkers comprise OPN, wherein one or more inflammation biomarkers comprise CXCL13, and wherein one or more neurodegeneration biomarkers comprise SERPINA9. In various embodiments, the one or more immune modulation biomarkers comprise OPN, wherein one or more inflammation biomarkers comprise CXCL13 and TNFRSF10A. In various embodiments, the one or more neurodegeneration biomarkers comprise NEFL, wherein the one or more myelin integrity biomarkers comprise MOG, and wherein one or more inflammation biomarkers comprise IL-12B. In various embodiments, the one or more neurodegeneration biomarkers comprise NEFL, PRTG, and GFAP and wherein the one or more myelin integrity biomarkers comprise MOG. In various embodiments, the one or more neurodegeneration biomarkers comprise NEFL, APLP1, and GFAP and wherein the one or more myelin integrity biomarkers comprise MOG. In various embodiments, the one or more neurodegeneration biomarkers comprise NEFL and SERPINA9, wherein one or more inflammation biomarkers comprise GH, and wherein the one or more myelin integrity biomarkers comprise MOG. In various embodiments, the one or more neurodegeneration biomarkers comprise NEFL and SERPINA9, wherein one or more inflammation biomarkers comprise TNFRSF10A, and wherein the one or more myelin integrity biomarkers comprise MOG. In various embodiments, the one or more neurodegeneration biomarkers comprise APLP1 and wherein one or more inflammation biomarkers comprise CCL20, IL-12B, and CD6. In various embodiments, the prediction of multiple sclerosis disease progression is an expanded disability status scale (EDSS) score. In various embodiments, an expanded disability status scale (EDSS) score less than 6 indicates a mild/moderate MS disease progression and a EDSS score greater than or equal to 6.0 indicates a severe MS disease progression.

In various embodiments, the one or more neurodegeneration biomarkers comprise NEFL and wherein the one or more inflammation biomarkers comprise CD6 and CXCL13. In various embodiments, a performance of the predictive model is characterized by an area under the ROC curve (AUROC) value of at least 0.8. In various embodiments, a performance of the predictive model is characterized by an area under the ROC curve (AUROC) value of at least 0.9. In various embodiments, the prediction of multiple sclerosis disease progression is a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression.

In various embodiments, one or more inflammation biomarkers comprises IL-12B and wherein one or more immune modulation biomarkers comprise CDCP1. In various embodiments, the prediction of multiple sclerosis disease progression is a PRO measurement information system (PROMIS) score.

In various embodiments, one or more inflammation biomarkers comprise CD6, IL-12B, and VCAN, and wherein one or more immune modulation biomarkers comprise CDCP1. In various embodiments, the prediction of multiple sclerosis disease progression is a Multiple Sclerosis Rating Scale, Revised (MSRS-R) score.

Additionally disclosed herein is a method for predicting multiple sclerosis progression in a subject, the method comprising: obtaining or having obtained a dataset comprising expression levels of a plurality of biomarkers, wherein the plurality of biomarkers comprises two or more of: NEFL, MOG, CD6, CXCL9, OPG, OPN, CXCL13, GFAP, CDCP1, CCL20, IL-12B, APLP1, TNFRSF10A, SERPINA9, FLRT2, TNFSF13B, GH, VCAN, PRTG, and CNTN2; and generating a prediction of multiple sclerosis disease progression by applying a predictive model to the expression levels of the plurality of biomarkers. In various embodiments, the plurality of biomarkers comprise each of NEFL, MOG, CD6, CXCL9, OPG, OPN, CXCL13, GFAP, CDCP1, CCL20, IL-12B, APLP1, TNFRSF10A, SERPINA9, FLRT2, TNFSF13B, GH, VCAN, PRTG, and CNTN2. In various embodiments, a performance of the predictive model is characterized by a correlation coefficient (R²) of at least 0.517. In various embodiments, the prediction of multiple sclerosis disease progression is a brain parenchymal fraction value.

In various embodiments, the plurality of biomarkers comprise MOG and GFAP. In various embodiments, the plurality of biomarkers comprise NEFL and GFAP. In various embodiments, the plurality of biomarkers comprise GFAP and APLP1. In various embodiments, the plurality of biomarkers comprise NEFL and MOG. In various embodiments, the plurality of biomarkers comprise NEFL, MOG, and GFAP. In various embodiments, the plurality of biomarkers comprise NEFL MOG, and GH. In various embodiments, the plurality of biomarkers comprise NEFL, MOG, and SERPINA9. In various embodiments, the plurality of biomarkers comprise OPN, CXCL13, and SERPINA9. In various embodiments, the plurality of biomarkers comprise OPN, CXCL13, and TNFRSF10A. In various embodiments, the plurality of biomarkers comprise NEFL, MOG, GFAP, and IL-12B. In various embodiments, the plurality of biomarkers comprise NEFL, MOG, GFAP, and PRTG. In various embodiments, the plurality of biomarkers comprise NEFL, MOG, GFAP, and APLP1. In various embodiments, the plurality of biomarkers comprise NEFL, MOG, SERPINA9, and GH. In various embodiments, the plurality of biomarkers comprise NEFL MOG, TNFRSF10A, and SERPINA9. In various embodiments, the plurality of biomarkers comprise CD6, CCL20, IL-12B, and APLP1. In various embodiments, the prediction of multiple sclerosis disease progression is an expanded disability status scale (EDSS) score. In various embodiments, an expanded disability status scale (EDSS) score less than or equal to 6 indicates a mild/moderate MS disease progression and a EDSS score greater than 6.5 indicates a severe MS disease progression.

In various embodiments, the plurality of biomarkers comprise NEFL, CD6, and CXCL13. In various embodiments, a performance of the predictive model is characterized by an area under the ROC curve (AUROC) value of at least 0.8. In various embodiments, a performance of the predictive model is characterized by an area under the ROC curve (AUROC) value of at least 0.9. In various embodiments, the prediction of multiple sclerosis disease progression is a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression.

In various embodiments, the plurality of biomarkers comprise CDCP1 and IL-12B. In various embodiments, the prediction of multiple sclerosis disease progression is a PRO measurement information system (PROMIS) score.

In various embodiments, the plurality of biomarkers comprise CD6, CDCP1, IL-12B, and VCAN. In various embodiments, the prediction of multiple sclerosis disease progression is a Multiple Sclerosis Rating Scale, Revised (MSRS-R) score.

In various embodiments, generating the prediction of multiple sclerosis disease progression by applying the predictive model to the expression levels of the plurality of biomarkers further comprises applying the predictive model to one or more subject attributes of the subject, wherein subject attributes comprise any of age, sex, and disease duration. In various embodiments, generating the prediction of multiple sclerosis disease progression comprises comparing a score outputted by the predictive model to a reference score. In various embodiments, the reference score corresponds to any of: A) an EDSS score; B) a brain parenchymal fraction value; C) a PDDS score; D) a PROMIS score; or E) a MSRS-R score. In various embodiments, the reference score further corresponds to a mild/moderate MS disease progression or a severe MS disease progression.

In various embodiments, the expression levels of the plurality of biomarkers is determined from a test sample obtained from the subject. In various embodiments, the test sample is a blood or serum sample. In various embodiments, the subject has multiple sclerosis, is suspected of having multiple sclerosis, or was previously diagnosed with multiple sclerosis.

In various embodiments, obtaining or having obtained the dataset comprises performing an immunoassay to determine the expression levels of the plurality of biomarkers. In various embodiments, the immunoassay is a Proximity Extension Assay (PEA) or LUMINEX xMAP Multiplex Assay. In various embodiments, performing the immunoassay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies. In various embodiments, the method further comprises: selecting a therapy for administering to the subject based on the prediction of multiple sclerosis disease progression. In various embodiments, the method further comprises: determining a therapeutic efficacy of a therapy previously administered to the subject based on the prediction of multiple sclerosis disease progression. In various embodiments, determining the therapeutic efficacy of the therapy comprises comparing the prediction to a prior prediction determined for the subject at a prior timepoint In various embodiments, determining the therapeutic efficacy of the therapy comprises determining that the therapy exhibits efficacy responsive to a difference between the prediction and the prior prediction. In various embodiments, determining the therapeutic efficacy of the therapy comprises determining that the therapy lacks efficacy responsive to a lack of difference between the prediction and the prior prediction.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should be allowed for.

### Example 1: Human Clinical Studies

Multiple human clinical studies were used for the development of this biomarker panel including: ACP=Accelerated Cure Project (study code: F2), CLIMB=Comprehensive Longitudinal Investigation of Multiple Sclerosis at Brigham and Women's Hospital (study code: F3A, F3B, F3C, and F4), EPIC=Expression, Proteomics, Imaging, Clinical at UCSF (study code: F5), and UHBC=University Hospital Basel Cohort (study code: F6). ACP (n=124) focuses on clinically-defined exacerbation events and AIM1 (n=60) and F4 Unpaired (n=326) focus on annualized relapse rate (ARR). AIM3 Unpaired (n=58), F4 Unpaired (n=326) and F5 EPIC (n=180) focus on the cross-sectional perspective of the Gadolinium (Gd) enhanced MRI lesion endpoint, while AIM3 Paired (n=58), F4 Paired (n=196), and F6 (n=205) do the same through a longitudinal analysis (e.g., consider patient pairs to establish a baseline normal). Additionally, in some scenarios, samples from different studies with the same end point were combined to conduct biomarker analyses. For example, study code F4 and study code F5, with a common endpoint of presence or absence of Gadolinium enhancing lesion, were combined to conduct biomarker analyses. For each of the three broad categories, each sample was given equal weighting across the 7 study paradigms (i.e. studies with more samples had more weight proportionally). The study codes and other information for the analyses are documented below in Tables 5A and 5B. Altogether, over 1300 proteins across more than 1000 individual samples were screened using 2 immunoassay platforms (Rules Based Medicine (RBM) and Olink biomarker panel). Multiple endpoints were investigated including presence/absence of gadolinium enhancing lesions, clinical relapse status, EDSS, annualized relapse rate, and T2 volume.

### Example 2: Univariate Analysis

Three different statistical measures were calculated for univariate analysis of individual biomarkers.
1) Univariate population—p-values from standard statistical tests
   P-values were converted back to their t-statistic using the conventional inverse norm function, and then Stauffer's method was used to combine the statistics based on their respective sample sizes. The final p-value/test-statistic reflecting the cumulative power was then normalized to be on a scale of [0,1].
2) Univariate separation—AUC values from integration across the true positive rate and false positive rate on the ROC curve
   AUC's were computed for each individual marker on the selected dataset. They were then normalized to a scale of [0,1] and then the weighted sum of them (based on the sample sizes below) was used to convert the cumulative separation power into a single value between 0 and 1.
3) Univariate regression—adjusted r-squared values from OLS between lesion burden—clipped at an upper lesion count (e.g., 5 lesions) to exclude outliers—and the distribution of normalized protein expression values
   This was conducted on each cohort independently, in addition to the blended cohorts in a train/test split.

### Example 3: Multivariate Analysis

The following was conducted for multivariate biomarker analysis:
1) Multivariate biomarker ranking—the accuracy-weighted aggregated importance across millions of simulated support vector machines, logistic regression, random forest, linear discriminant analysis, and stochastic gradient descent models of various feature sizes were combined). Hyperparameter-tuning (e.g. regularization or choice of numerical solver technique) was exhausted in a grid search. Forward selection multivariate model building was simulated through many thousands of times to then add up in aggregate the biomarkers that were most commonly selected on different cross-validated slicings of the dataset. Forward selection iteratively incorporated features according to an optimization metric (e.g. AUC, F1 in classification and Adj-R 2, RMSE in regression). Since multiple data cohorts are involved, the importance of each study was weighted by sample size and rank Gd as the primary endpoint to then finalize the spatially constrained 21-plex. Next, certain features were sequentially removed, wherein after removing the features, the remaining features still have predictive power for each endpoint/study. Cross-validation and bootstrapping was used to reduce overfitting. A test holdout set was used when possible.
2) Bivariate (2-feature) models to investigate improvement of orthogonal signal
3) Interacting terms (product, ratio, quadratic terms)

To ensure replicability of results:
The models were trained on the CLIMB dataset and tested on the EPIC dataset (before/after batch normalization and demographic adjustment)
Paired Samples (Baseline-Normalized Signal) between AIM3 and F4

### Example 4: Univariate APLP1 Biomarker Analysis for Predicting Multiple Sclerosis Disease Activity

Univariate analysis of APLP1 was conducted across the different human clinical studies according to the methods described in Example 2. The statistical measures of the univariate analysis (p-value, area under the curve (AUC) and correlation values (R-squared)) are shown below in Table 10A.

### Example 5: Univariate CCL20 Biomarker Analysis for Predicting Multiple Sclerosis Disease Activity

Univariate analysis of CCL20 was conducted across the different human clinical studies according to the methods described in Example 2. The statistical measures of the univariate analysis (p-value, area under the curve (AUC) and correlation values (R-squared)) are shown below in Table 10B.

### Example 6: Univariate CD6 Biomarker Analysis for Predicting Multiple Sclerosis Disease Activity

Univariate analysis of CD6 was conducted across the different human clinical studies according to the methods described in Example 2. The statistical measures of the univariate analysis (p-value, area under the curve (AUC) and correlation values (R-squared)) are shown below in Table 10C.

### Example 7: Univariate CDCP1 Biomarker Analysis for Predicting Multiple Sclerosis Disease Activity

Univariate analysis of CDCP1 was conducted across the different human clinical studies according to the methods described in Example 2. The statistical measures of the univariate analysis (p-value, area under the curve (AUC) and correlation values (R-squared)) are shown below in Table 10D.

### Example 8: Univariate CNTN2 Biomarker Analysis for Predicting Multiple Sclerosis Disease Activity

Univariate analysis of CNTN2 was conducted across the different human clinical studies according to the methods described in Example 2. The statistical measures of the univariate analysis (p-value, area under the curve (AUC) and correlation values (R-squared)) are shown below in Table 10E.

### Example 9: Univariate COL4A1 Biomarker Analysis for Predicting Multiple Sclerosis Disease Activity

Univariate analysis of COL4A1 was conducted across the different human clinical studies according to the methods described in Example 2. The statistical measures of the univariate analysis (p-value, area under the curve (AUC) and correlation values (R-squared)) are shown below in Table 10F.

### Example 10: Univariate CXCL9 Biomarker Analysis for Predicting Multiple Sclerosis Disease Activity

Univariate analysis of CXCL9 was conducted across the different human clinical studies according to the methods described in Example 2. The statistical measures of the univariate analysis (p-value, area under the curve (AUC) and correlation values (R-squared)) are shown below in Table 10G.

### Example 11: Univariate FLRT2 Biomarker Analysis for Predicting Multiple Sclerosis Disease Activity

Univariate analysis of CDCP1 was conducted across the different human clinical studies according to the methods described in Example 2. The statistical measures of the univariate analysis (p-value, area under the curve (AUC) and correlation values (R-squared)) are shown below Table 10H.

### Example 12: Univariate Growth Hormone Biomarker Analysis for Predicting Multiple Sclerosis Disease Activity

Univariate analysis of Growth Hormone (GH) was conducted across the different human clinical studies according to the methods described in Example 2. The statistical measures of the univariate analysis (p-value, area under the curve (AUC) and correlation values (R-squared)) are shown below in Table 101.

### Example 13: Univariate IFI30 Biomarker Analysis for Predicting Multiple Sclerosis Disease Activity

Univariate analysis of IFI30 was conducted across the different human clinical studies according to the methods described in Example 2. The statistical measures of the univariate analysis (p-value, area under the curve (AUC) and correlation values (R-squared)) are shown below in Table 10J.

### Example 14: Univariate MOG Biomarker Analysis for Predicting Multiple Sclerosis Disease Activity

Univariate analysis of MOG was conducted across the different human clinical studies according to the methods described in Example 2. The statistical measures of the univariate analysis (p-value, area under the curve (AUC) and correlation values (R-squared)) are shown below in Table 10K.

### Example 15: Univariate NEFL Biomarker Analysis for Predicting Multiple Sclerosis Disease Activity

Univariate analysis of NEFL was conducted across the different human clinical studies according to the methods described in Example 2. The statistical measures of the univariate analysis (p-value, area under the curve (AUC) and correlation values (R-squared)) are shown below in Table 10L.

### Example 16: Univariate OPG Biomarker Analysis for Predicting Multiple Sclerosis Disease Activity

Univariate analysis of OPG was conducted across the different human clinical studies according to the methods described in Example 2. The statistical measures of the univariate analysis (p-value, area under the curve (AUC) and correlation values (R-squared)) are shown below in Table 10M.

### Example 17: Univariate OPN Biomarker Analysis for Predicting Multiple Sclerosis Disease Activity

Univariate analysis of OPN was conducted across the different human clinical studies according to the methods described in Example 2. The statistical measures of the univariate analysis (p-value, area under the curve (AUC) and correlation values (R-squared)) are shown below in Table 10N.

### Example 18: Univariate PRTG Biomarker Analysis for Predicting Multiple Sclerosis Disease Activity

Univariate analysis of PRTG was conducted across the different human clinical studies according to the methods described in Example 2. The statistical measures of the univariate analysis (p-value, area under the curve (AUC) and correlation values (R-squared)) are shown below in Table 100.

### Example 19: Univariate SERPINA9 Biomarker Analysis for Predicting Multiple Sclerosis Disease Activity

Univariate analysis of SERPINA9 was conducted across the different human clinical studies according to the methods described in Example 2. The statistical measures of the univariate analysis (p-value, area under the curve (AUC) and correlation values (R-squared)) are shown below in Table 10P.

### Example 20: Univariate TNFRSF10A Biomarker Analysis for Predicting Multiple Sclerosis Disease Activity

Univariate analysis of TNFRSF10A was conducted across the different human clinical studies according to the methods described in Example 2. The statistical measures of the univariate analysis (p-value, area under the curve (AUC) and correlation values (R-squared)) are shown below in Table 10Q.

### Example 21: Univariate VCAN Biomarker Analysis for Predicting Multiple Sclerosis Disease Activity

Univariate analysis of VCAN was conducted across the different human clinical studies according to the methods described in Example 2. The statistical measures of the univariate analysis (p-value, area under the curve (AUC) and correlation values (R-squared)) are shown below in Table 10R.

### Example 22: Univariate CHI3L1 Biomarker Analysis for Predicting Multiple Sclerosis Disease Activity

Univariate analysis of CHI3L1 was conducted across the different human clinical studies according to the methods described in Example 2. The statistical measures of the univariate analysis (p-value, area under the curve (AUC) and correlation values (R-squared)) are shown below in Table 10S.

### Example 23: Univariate CXCL13 Biomarker Analysis for Predicting Multiple Sclerosis Disease Activity

Univariate analysis of CXCL13 was conducted across the different human clinical studies according to the methods described in Example 2. The statistical measures of the univariate analysis (p-value, area under the curve (AUC) and correlation values (R-squared)) are shown below in Table 10T.

### Example 24: Univariate Growth Hormone 2 Biomarker Analysis for Predicting Multiple Sclerosis Disease Activity

Univariate analysis of Growth Hormone 2 (GH2) was conducted across the different human clinical studies according to the methods described in Example 2. The statistical measures of the univariate analysis (p-value, area under the curve (AUC) and correlation values (R-squared)) are shown below in Table 10U.

### Example 25: Univariate IL-12B Biomarker Analysis for Predicting Multiple Sclerosis Disease Activity

Univariate analysis of IL-12B was conducted across the different human clinical studies according to the methods described in Example 2. The statistical measures of the univariate analysis (p-value, area under the curve (AUC) and correlation values (R-squared)) are shown below in Table 10V.

### Example 26: Univariate IL18 Biomarker Analysis for Predicting Multiple Sclerosis Disease Activity

Univariate analysis of IL18 was conducted across the different human clinical studies according to the methods described in Example 2. The statistical measures of the univariate analysis (p-value, area under the curve (AUC) and correlation values (R-squared)) are shown below in Table 10W.

### Example 27: Univariate MMP-2 Biomarker Analysis for Predicting Multiple Sclerosis Disease Activity

Univariate analysis of MMP-2 was conducted across the different human clinical studies according to the methods described in Example 2. The statistical measures of the univariate analysis (p-value, area under the curve (AUC) and correlation values (R-squared)) are shown below in Table 10X.

### Example 28: Univariate MMP-9 Biomarker Analysis for Predicting Multiple Sclerosis Disease Activity

Univariate analysis of MMP-9 was conducted across the different human clinical studies according to the methods described in Example 2. The statistical measures of the univariate analysis (p-value, area under the curve (AUC) and correlation values (R-squared)) are shown below in Table 10Y.

### Example 29: Univariate VCAM-1 Biomarker Analysis for Predicting Multiple Sclerosis Disease Activity

Univariate analysis of VCAM-1 was conducted across the different human clinical studies according to the methods described in Example 2. The statistical measures of the univariate analysis (p-value, area under the curve (AUC) and correlation values (R-squared)) are shown below in Table 10Z.

### Example 30: Univariate TNFSF13B Biomarker Analysis for Predicting Multiple Sclerosis Disease Activity

Univariate analysis of TNFSF13B was conducted across the different human clinical studies according to the methods described in Example 2. The statistical measures of the univariate analysis (p-value, area under the curve (AUC) and correlation values (R-squared)) are shown below in Table 10AA.

### Example 31: Univariate GFAP Biomarker Analysis for Predicting Multiple Sclerosis Disease Activity

Univariate analysis of GFAP was conducted across the different human clinical studies according to the methods described in Example 2. The statistical measures of the univariate analysis (p-value, area under the curve (AUC) and correlation values (R-squared)) are shown below in Table 10BB.

### Example 32: Univariate Biomarker Analysis for Predicting Different Types of Multiple Sclerosis Disease Activity

Univariate analysis of various biomarkers was conducted across the different human clinical studies according to the methods described in Example 2. Statistical p-values and R² values are shown below in Table 11 for each of the individual biomarkers in relation to the different MS disease activity endpoints.

### Example 33: Model Training and Validation—Baseline Normalization Shifts

A linear regression model (L2 Ridge Regularization) was trained to predict baseline-normalization shifts of Gd Analysis on two different populations (F4 and F6). Here, the model was trained to classify sample pairs with and without a Gd-enhancing MRI lesion that manifests relative to a baseline (both serum drawn in proximity to MRI).

A logistic regression classification algorithm was applied to positive versus negative shifts in Gd count. Since the predictors are shifts in biomarker protein levels (from a baseline sample without Gd activity in corresponding MM), no demographic correction is necessary since intra-patient variation is accounted for. The dataset was split into training and 5-fold cross validation sets and the parameters of the model have been hypertuned to optimize the model; all biomarkers from the custom assay panel (Tiers 1, 2, and 3 in Table 2 below) were included as features in the model. The Area under the Receiver-Operating Characteristic Curve (AUROC) resulting from the 5-fold cross validation for classifying Gd shifts is 0.958+/−0.034.

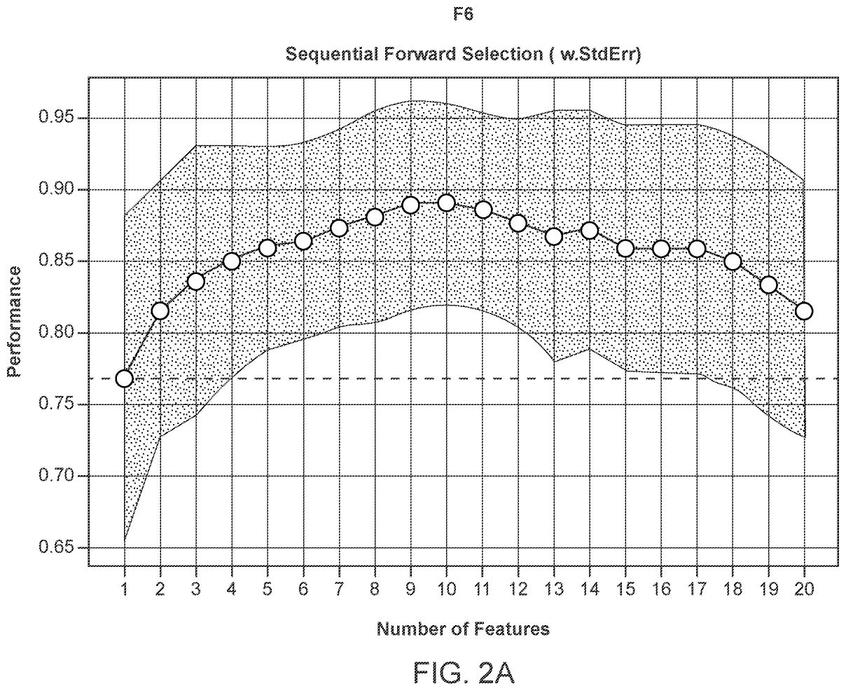

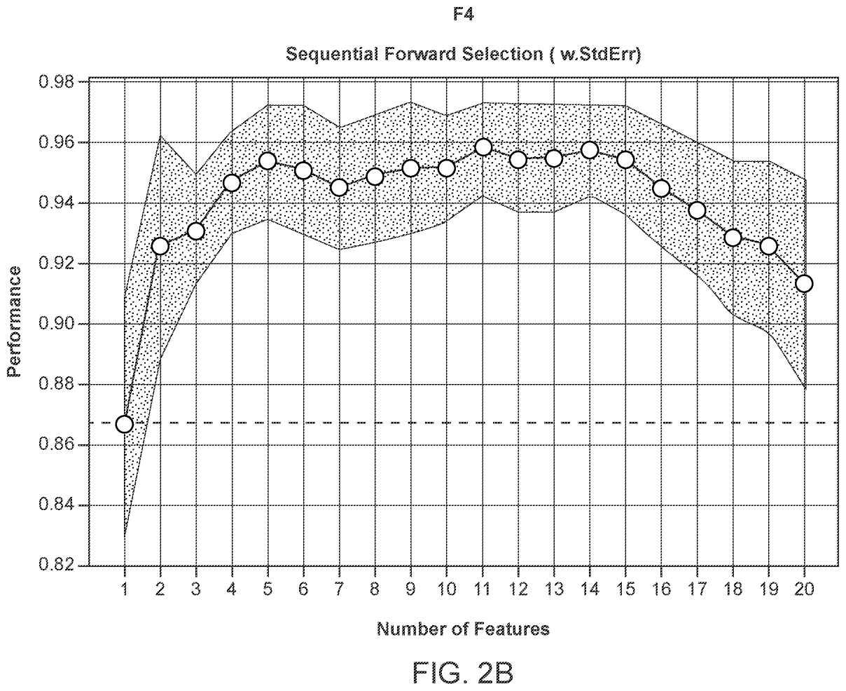

FIG. 2A and FIG. 2B depict sequential forward selection performance profiles for the F6 and F4 study, respectively.

Next, an independent Train/Test Holdout of a 4-feature Model was trained and tested. Here, a logistic regression (L1 regularization) model was trained on F6 data and tested on F4 data. The model feature space is restricted to the top 4 features from the analysis. A logistic regression classification algorithm was applied to positive versus negative shifts in Gd count. Since the predictors are shifts in biomarker protein levels (from a baseline without Gd activity), no demographic correction is necessary since intra-patient variation is accounted for. Bridge normalization was applied to the dataset, using an overlapping set of samples that were re-run, to utilize the same model coefficients for both the training and test sets. The parameters of the model were hypertuned to optimize the model; all biomarkers from the custom assay panel have been included as features in the model. This analysis was done to assess the multivariate predictive performance (relative to the univariate performance of serum Neurofilament Light Chain, or sNfL), which is shown in FIG. 3A. The model exhibited a performance of AUROC: 0.91 (sNfL—0.88), Accuracy: 0.84 (sNfL—0.77), Sensitivity: 0.76 (sNfL—0.70), Specificity: 0.91 (sNfL—0.83), and Youden's Statistic=0.67 (sNfL—0.53). FIG. 3B depicts the corresponding confusion matrix of the multivariate classifier, which establishes the high true positive rate (TPR)=0.757 and high true negative rate (TNR)=0.905.

### Example 34: Model Training and Validation—Cross-Sectional Classification of Presence/Absence of Disease

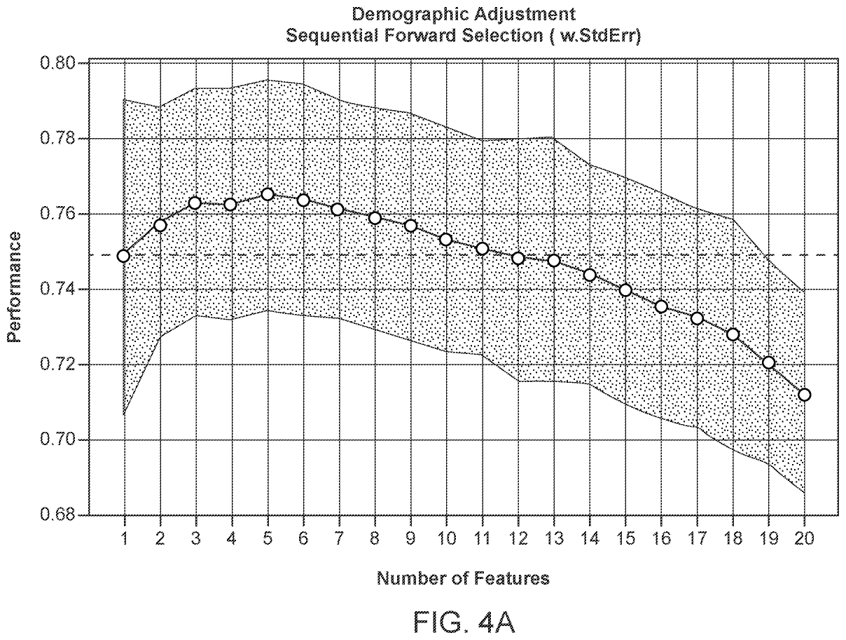

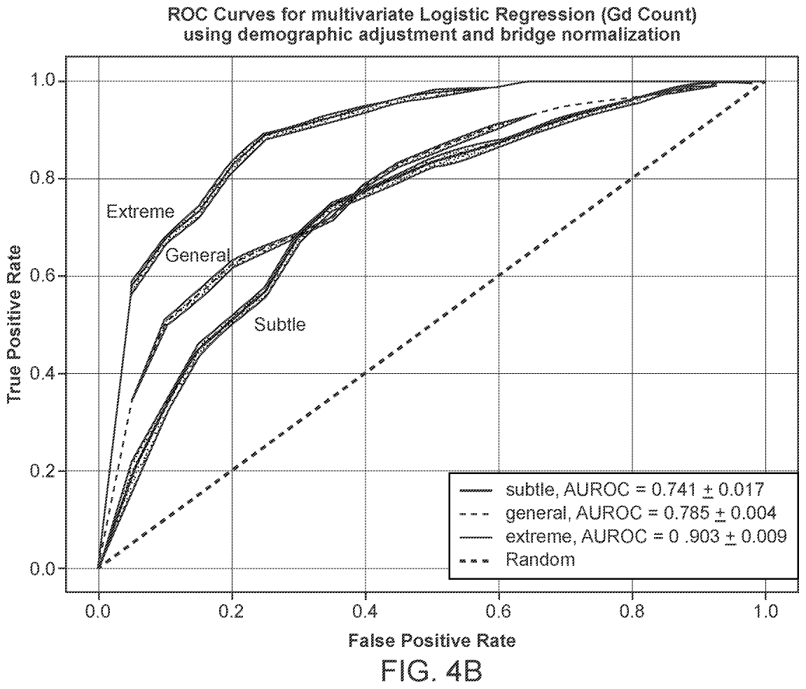

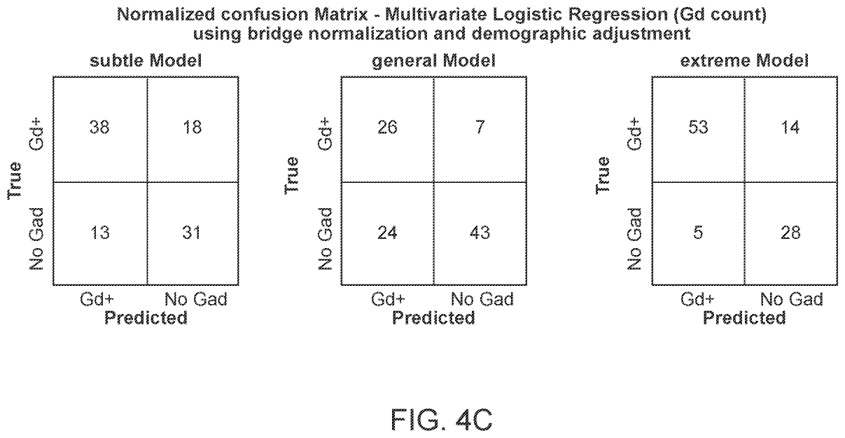

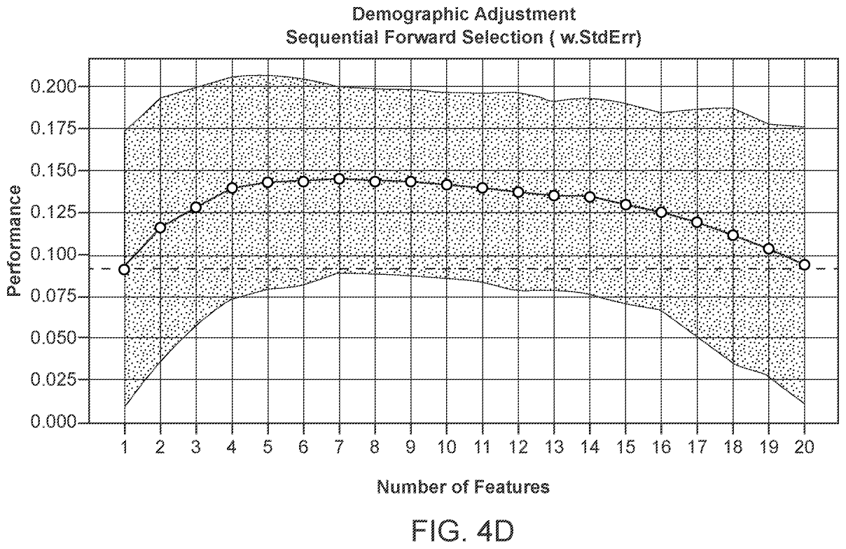

A logistic regression model (L1 regularization) was trained to predict presence/absence of disease based on Gd count. Specifically, the model was trained to predict either: subtle disease (corresponds to having or not 1 lesion per MRI), general disease (corresponds to having or not having a lesion), and extreme disease (corresponds to having or not having more than three lesions. The model was built using 321 samples from F4, 180 samples from F5, and 155 samples from F6. FIG. 4A depicts the sequential forward selection performance profile.

To build the models, bridge normalization and demographic correction were applied to the dataset resulting from merging the three studies. The dataset has been split into training and 5-fold cross validation sets and the parameters of the model were hypertuned to optimize the model; all biomarkers from the custom assay panel have been included as features in the model. FIG. 4B depicts the ROC curves for the three models. Specifically, the AUROC resulting from the 5-fold cross validation is 0.741±0.017 for the subtle model; 0.785±0.004 for the general model and 0.903±0.009 for the extreme model. Further gadolinium based classification predictions are shown in Table 12.

Furthermore, FIG. 4C depicts the normalized confusion matrix for each model. Gd+ classification comparison is shown below using 1) univariate NFL model, 2) multivariate model including NFL, and 3) multivariate model excluding NFL.

### Example 35: Model Training and Validation—Predicting Severity of Disease by Predicting the Number of Lesions

A linear regression model (L2 regularization) was trained and tested using 321 samples from F4, 180 samples from F5, and 155 samples from F6. A Ridge regression algorithm was applied to the Gd count. Bridge normalization and demographic correction was applied to the dataset resulting from merging the three studies. The dataset was split into training and 5-fold cross validation sets; all biomarkers from the custom assay panel have been included as features in the model and a forward fitting procedure has been applied to estimate which combination of features results in a higher model performance. FIG. 4D depicts the sequential forward selection of features. A corresponding best regression plot of predicted vs. actual Gd lesion count (i.e. proxy for MS disease activity burden in this analysis) was generated. The best performance, based on R² is obtained using 7 features (NEFL, MOG, CDCP1, OPG, APLP1 and COL4A1): 0.219±0.050. Looking into the Spearman r score, the best model is obtained from 4 features (NEFL, CDCP1, APLP1 and TNFSF13B), obtaining a Spearman r score of 0.496±0.038.

### Example 36: Model Training and Validation—Predicting Annualized Relapse Rate

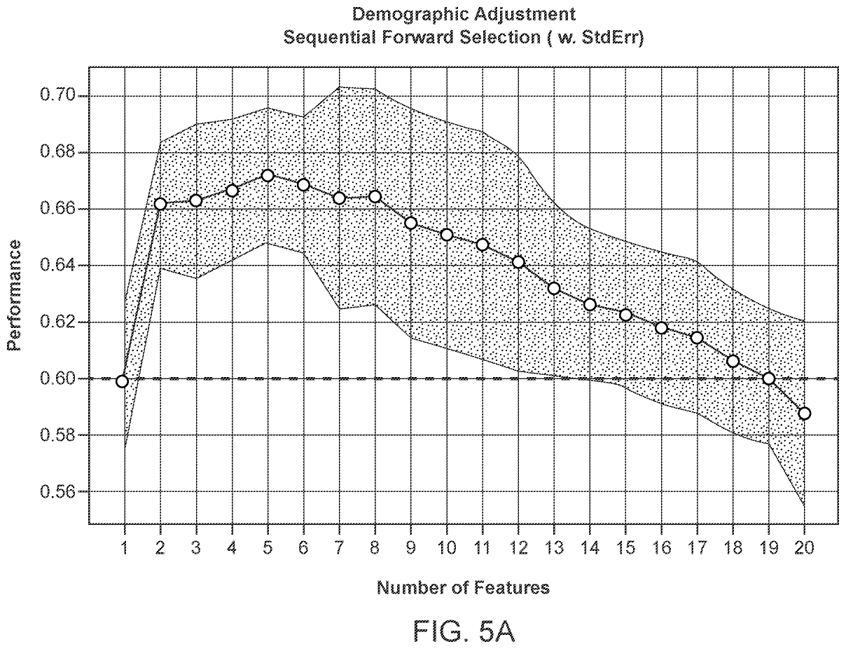

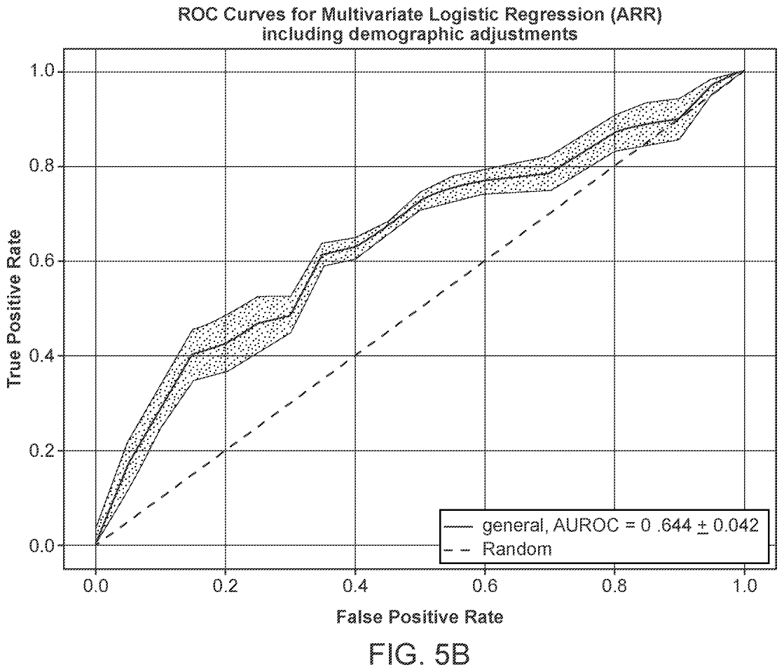

A logistic regression model (Elasticnet 0.2) was trained and tested on 282 samples with 161 LOW samples <0.3 ARR and 121 HIGH samples >0.8 ARR. FIG. 5A depicts the sequential forward selection of features. Applying a logistic regression classification algorithm, after forward fitting the features and hypertuning the parameters, the best model exhibits an AUROC of 0.672+/−0.053 with the following 5 features: NEFL, MOG, CDCP1, IL-12B, and TNFRSF10A. FIG. 5B depicts a ROC curve for predicting ARR.

### Example 37: Model Training and Validation—Predicting Clinically-Defined Relapse

A logistic regression model was built using LBFGS solver with L2 regularization (C=1.0) and balanced class weight. Relapse status is set by a clinically defined criterion in the ACP study (“Exacerbated” vs. “Quiescent”) and in F6 by whether the patient has had a relapse within 90 days of the blood draw. Because there are no bridging samples between ACP and F6, two independent analyses of the two studies were performed. The NPX values from both studies were demographically corrected and the studies in F6 that were taken more than 30 days from the MRI were dropped (for consistency with the Gd studies).

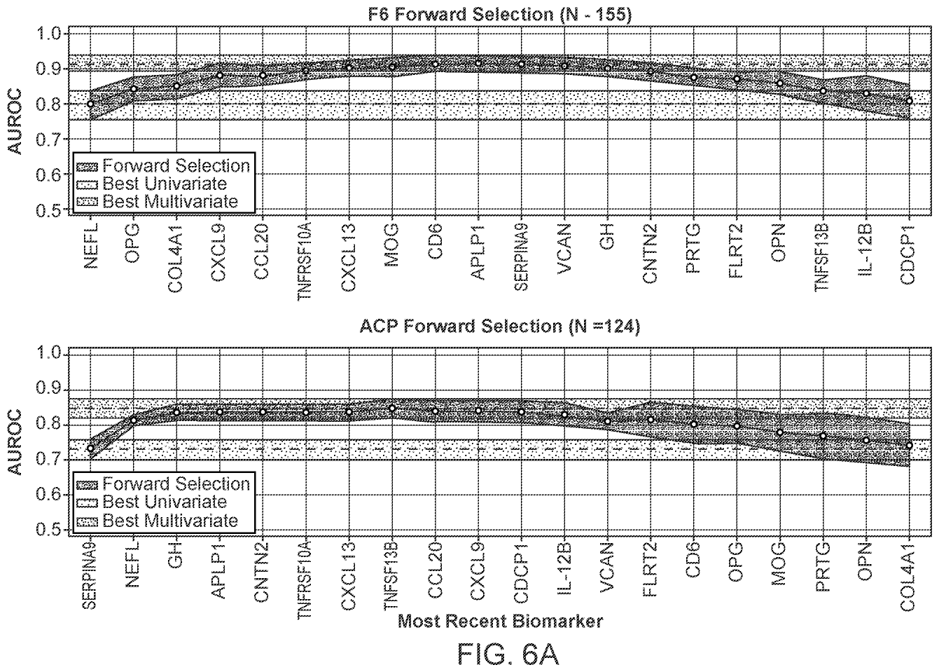

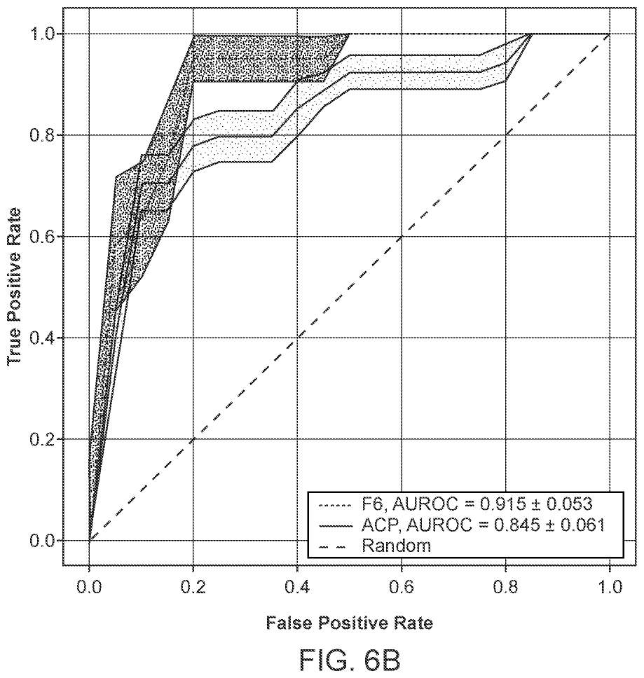

FIG. 6A depicts the sequential forward selection of features using the F6 study (n=155, 136 patients with no relapses, 19 with relapses in the previous 90 days) and ACP study (n=124, 64 Quiescent MS samples, 60 Exacerbation MS samples). New features are added cumulatively to the total as the plot moves from left to right. FIG. 6B depicts the performance of the respective models. Specifically, the model trained on the F6 study exhibited a AUROC of 0.915±0.053 and the model trained on the ACP study exhibited a AUROC of 0.845±0.061.

### Example 38: Model Training and Validation—Predicting Disease Progression

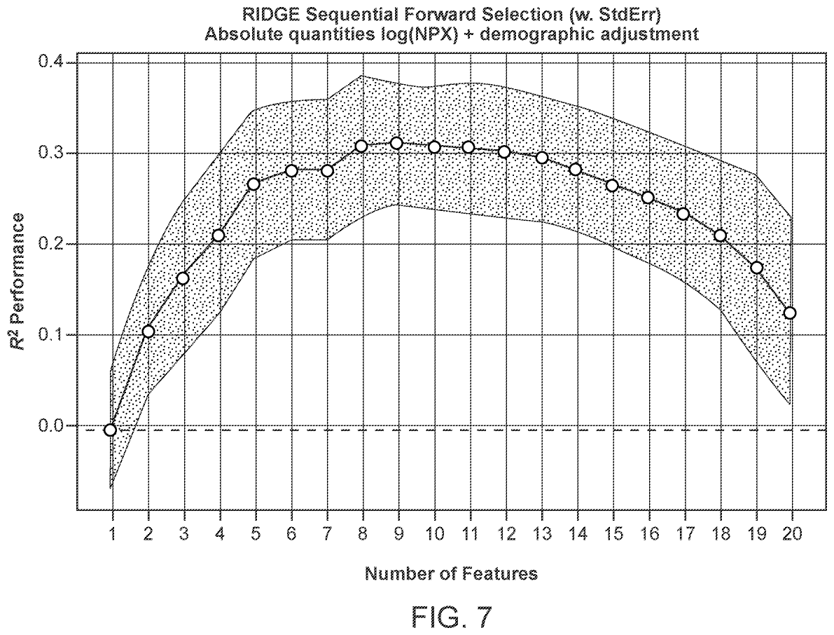

FIG. 7 depicts the sequential forward selection of features on absolute quantitation data according to the expanded disability status scale (EDSS). Here, biomarkers and their absolute quantitation measurements for 163 samples where all data is available were considered. Samples were from the F6 (University of Basel) study cohort. For each protein, the serum measurements were directly correlated to the respective endpoint (EDSS, T2-weighted lesion volume). Only serum draws which fell within 30 days of MM were considered for the radiographic endpoint (i.e. T2-weighted lesion volume). A 5-fold cross-validation was used to train and test a logistic model (L2 regularization) and evaluate the performance. The optimization metric used was R² (square of the Pearson's R correlation) and standardized coefficients are shown. Values in Table 13 show the EDSS R² for different biomarkers. Of note, raw serum GFAP correlates to EDSS with a R² value of 0.201 and raw serum OPG correlates to EDSS with a R² value of 0.204.

The values shown in Table 14 represent absolute quantitation (log-transform of pg/mL) vs. the relative quantitation (normalized protein expression, or NPX) data for the n=205 samples in the F6 Basel cohort. AvN1 represents the Pearson's R² value corresponding to the correlation between the absolute quantitation and the NPX values measured on the exploratory panels to guide initial research and development. N2vN1 represents the same metric between the relative NPX underlying the absolute quantitation (before a standard curve is fit to map the values to concentrations) and the exploratory panel NPX measured before. *GFAP does not have a corresponding relative quantitation measurement since no exploratory assay existed at the time. This example serves to show that quantitative information from the markers on the panel, regardless of the source format, may be used to predict MS disease activity

### Example 39: Multivariate Biomarker Panel for Predicting Multiple Sclerosis Disease Activity (Tier a, Tier B, Tier C Biomarkers)

Biomarkers were selected for a multivariate custom panel according to their correlation with the various endpoints (e.g., subtle disease (e.g., between 0 and 1 Gd enhancing lesion), general disease (e.g., between 0 and at least 1 Gd enhancing lesion), annualized relapse rate, and exacerbation v. quiescent).

Multivariate analyses of biomarker panels were conducted across the different human clinical studies according to the methods described in Example 3. In particular biomarkers were categorized into different tiers (e.g., Tier A, Tier B, and Tier C). Biomarker panels were constructed from one or more tiers (e.g., Tier A alone, tier B alone, tier C alone, tiers A+B, or tiers A+B+C). The 21 total biomarkers evaluated through this multivariate example are:

Linear regression models (L1 regularization) were trained and cross-validated on each dataset (AIM1-ARR, ACP-E vs. Q, independently (with the exception of F4+F5 which was blended through normalization with bridging samples for the primary endpoint Gd). Disease activity was split into subtle (0 vs. 1 Gd lesions), General (0 vs. any Gd lesions), and Extreme (0 vs. 3+ lesions) when possible for the classification problem. The same model-building strategy was re-deployed on progressively larger subsets/tiers of markers on the panel to report AUC/PPV. Statistical measures of the multivariate analysis (area under the curve (AUC) and positive predictive value (PPV)) are shown below.

Generally, biomarker panels that employed biomarkers from each of tier A, tier B, and tier C (21 total biomarkers) corresponded to predictive models that exhibited improved predictive capacity across the different disease activity endpoints (e.g., subtle disease activity, general disease activity, extreme disease activity, annualized relapse rate, or disease state). Tables 15A-15E show the performance metrics of the different biomarker panels. Specifically, the AUC across these different disease activity endpoints ranged from 0.771 up to 0.961, whereas the PPV ranged from 0.687 up to 0.895. Biomarker panels employing biomarkers from tiers A and B (17 total biomarkers) achieved AUC values across the different disease endpoints that ranged from 0.737 up to 0.968, whereas the PPV ranged from 0.620 up to 0.896. Biomarker panels employing biomarkers from only tier A (8 total biomarkers) achieved AUC values across the different disease endpoints that ranged from 0.763 up to 0.880, whereas the PPV ranged from 0.716 up to 0.871. Biomarker panels employing biomarkers solely from tier B or biomarkers solely from tier C remained predictive, but noticeably were less predictive than the biomarker panels employing tier A biomarkers or combinations of tier A+A or tier A+B+C. Specifically, biomarker panels employing biomarkers solely from tier B achieved AUC values across the different disease endpoints that ranged from 0.562 up to 0.841, whereas the PPV ranged from 0.462 up to 0.999. Biomarker panels employing biomarkers solely from tier C achieved AUC values across the different disease endpoints that ranged from 0.589 up to 0.779, whereas the PPV ranged from 0.410 up to 0.781.

### Example 40: Multivariate Biomarker Panel for Predicting Multiple Sclerosis Disease Activity

Multivariate analyses of biomarker panels were conducted across the different human clinical studies according to the methods described in Example 3. In particular biomarkers were categorized into different tiers (e.g., Tier 1, Tier 2, and Tier 3). Biomarker panels were constructed from one or more tiers (e.g., Tier 1 alone, tier 2 alone, tier 3 alone, tiers 1+2, or tiers 1+2+3). The 21 total biomarkers evaluated through this multivariate example are the 21 biomarkers shown in Table 2. Additional backup biomarkers that can be used to substitute in for any of the 21 biomarkers are identified as tier 4 biomarkers in Table 2.

Linear regression models (L1 regularization) were trained and cross-validated on each dataset (AIM1-ARR, ACP-E vs. Q, independently (with the exception of F4+F5 which was blended through normalization with bridging samples for the primary endpoint Gd). Disease activity was split into subtle (0 vs. 1 Gd lesions), General (0 vs. any Gd lesions), and Extreme (0 vs. 3+ lesions) when possible for the classification problem. The same model-building strategy was re-deployed on progressively larger subsets/tiers of markers on the panel to report AUC/PPV. Statistical measures of the multivariate analysis (area under the curve (AUC) and positive predictive value (PPV)) are shown below.

Generally, biomarker panels that employed biomarkers from each of tier 1, tier 2, and tier 3 (21 total biomarkers) corresponded to predictive models that exhibited improved predictive capacity across the different disease activity endpoints (e.g., subtle disease activity, general disease activity, extreme disease activity, annualized relapse rate, or disease state). Tables 16A-16E show the performance metrics of the different biomarker panels.

Specifically, the AUC across these different disease activity endpoints ranged from 0.686 up to 0.889, whereas the PPV ranged from 0.648 up to 0.835. Biomarker panels employing biomarkers from tiers 1 and 2 (17 total biomarkers) achieved AUC values across the different disease endpoints that ranged from 0.693 up to 0.892, whereas the PPV ranged from 0.613 up to 0.843. Biomarker panels employing biomarkers from only tier 1 (8 total biomarkers) achieved AUC values across the different disease endpoints that ranged from 0.667 up to 0.869, whereas the PPV ranged from 0.617 up to 0.861. Biomarker panels employing biomarkers solely from tier 2 or biomarkers solely from tier 3 remained predictive, but noticeably were less predictive than the biomarker panels employing tier 1 biomarkers or combinations of tier 1+2 or tier 1+2+3. Specifically, biomarker panels employing biomarkers solely from tier 2 achieved AUC values across the different disease endpoints that ranged from 0.595 up to 0.761, whereas the PPV ranged from 0.462 up to 0.769. Biomarker panels employing biomarkers solely from tier 3 achieved AUC values across the different disease endpoints that ranged from 0.566 up to 0.626, whereas the PPV ranged from 0.370 up to 0.634.

### Example 41: Additional Multivariate (Pairs, Triplets, and Quadruplets) Biomarker Panel for Predicting Directional Shift of Multiple Sclerosis Disease Activity

Multivariate analyses of minimal sets of biomarkers (e.g., pairs, triplicates, and quadruplets) were conducted across the different human clinical studies. Specifically, minimal sets of biomarkers were analyzed for their ability to predict the directional shift of MS disease activity (e.g., increasing or decreasing disease activity, as measured by number of gadolinium enhancing lesions).

To analyze the minimal predictive set of biomarkers for longitudinal patient samples, combinations of biomarker pairs, triplicates, and quadruplets were analyzed in a shifts (baseline-normalized, pairwise differences) analysis to predict the increase or decrease of Gd-enhancing lesions. The overarching procedure for the analysis is:
1. Read in the list of biomarkers.
2. Read in the data for these biomarkers and their attendant demographic and clinical features for each study from the data lake.
3. Drop all samples that were collected more than a threshold time (e.g., more than 30 days) from their associated MRI scan.
4. Compute the difference in NPX values between all pairs of samples that have increasing or decreasing lesion activity.
   a. Filter out only the sample pairs where one sample has an associated MRI with 0 Gd lesions for the true baseline-normalization approach.
5. For each study:
   a. using the increasing/decreasing or Non-Gad+→Gad+/Gad+→Non-Gad+ as an endpoint,
   b. perform a five-fold cross validation split,
   c. select a logistic regression model configuration that worked well in past multivariate analyses,
   d. create feature matrices from every combination of two, three, and four proteins (no demographic/clinical features are necessary as covariates since baseline-normalization bakes it into the process),
   e. train a copy of the logistic regression model on four of the five splits, and evaluate its performance on the fifth,
6. Average the AUROC of each model across the five splits and the three studies. Take standard deviation of performances to compute a metric of uncertainty (repeat for PPV).
7. Sort the models by their AUROC, and create ROC curves for each study for the model corresponding to the highest performing feature set.

We ran the above analysis in two ways:

The above analysis was conducted in two ways:
A. including full list of biomarkers, and
B. a follow-up analysis excluding the highest performing biomarker (NEFL).

Tables 17 and 18 show that biomarker panels including two, three, or four biomarkers are predictive of the directional shift of MS disease activity.

In particular, for pairs of biomarkers, a panel including NEFL and CD6 biomarkers achieved an area under the receiver operating characteristic curve (AUROC) of 0.860 and a mean PPV of 0.77. Additionally, a biomarker panel including NEFL and CXCL9 biomarkers achieved an AUROC of 0.85 and a mean PPV of 0.74. Additional pairs of biomarkers (not including NEFL) were also predictive. For example, a biomarker panel including MOG and CXCL9 achieved an AUROC of 0.761 and a PPV of 0.686. A biomarker panel including CD6 and CXCL9 achieved an AUROC of 0.766 and a PPV of 0.699. A biomarker panel including MOG and CD6 achieved an AUROC of 0.745 and a PPV of 0.705.

In particular, for biomarker triplicates, a panel including NEFL, CXCL9, and CD6 achieved an AUROC of 0.885 and a mean PPV of 0.79. Additionally, a biomarker panel including MOG, CD6, and CXCL9 achieved an AUROC of 0.798 and a PPV of 0.71.

In particular, for biomarker quadruplets, a biomarker panel including NEFL, MOG, CXCL9, and CD6 achieved an AUROC of 0.884 and a PPV of 0.763. A biomarker panel including NEFL, CXCL9, CD6, and CXCL13 achieved an AUROC of 0.889 and a PPV of 0.764. A biomarker panel including NEFL, TNFRSF10A, COL4A1, and CCL20 achieved an AUROC of 0.836 and a PPV of 0.725. Additional biomarker quadruplets (not including NEFL) also demonstrated predictiveness. For example, a combination of MOG, CXCL9, IL-12B and APLP1 achieved an AUROC of 0.795 and PPV of 0.67. A combination of CXCL9, OPG, APLP1, and OPN achieved an AUROC of 0.741 and a PPV of 0.65. A combination of MOG, IL-12B, OPN, and CNTN2 achieved an AUROC of 0.765 and a PPV of 0.69.

### Example 42: Additional Multivariate (Pairs, Triplets, and Quadruplets) Biomarker Panel for Predicting Presence or Absence of Multiple Sclerosis

Multivariate analyses of minimal sets of biomarkers (e.g., pairs, triplicates, and quadruplets) were conducted across the different human clinical studies. Specifically, minimal sets of biomarkers were analyzed for their ability to predict a classification of presence or absence of general MS disease (e.g., presence indicated by at least 1 Gd-enhancing lesion and absence indicated by 0 Gd-enhancing lesions).

To analyze the minimal predictive set of biomarkers, combinations of biomarker pairs, triplicates, and quadruplets were analyzed in a cross-sectional analysis to predict the presence or absence of Gd-enhancing lesions (“General Disease Activity,” or GDA). The overarching procedure for the analysis is:
1. Read in the list of biomarkers.
2. Read in the data for these biomarkers and their attendant demographic and clinical features for each study from the data lake.
3. Drop all samples that were collected beyond a threshold time (e.g., more than 30 days) from their associated MRI scan.
4. Perform optimized demographic and clinical adjustment.
   A. Run an OLS regression between the optimal subset of demographic/clinical features and the NPX level of each individual biomarker value, using only the Gd-samples (with no disease activity) for a given study, filtering out outliers.
      a. DiseaseDuration: the number of years since MS diagnosis.
      b. Age: the age of a patient in number of years.
      c. Sample Age: the length of storage of a sample before it was formally analyzed.
      d. Delta BloodMinusDiagnosis: the number of days between an MRI and respective blood sample (must be between 30).
   B. Extract residuals from this procedure and use that as input into model-building procedures.
5. For each study:
   a. construct the GDA endpoint,
   b. perform a five-fold cross validation split,
   c. select a logistic regression model configuration that worked well in past multivariate analyses,
   d. create feature matrices from every combination of two, three, and four proteins (and no demographic/clinical features as covariates),
   e. train a copy of the logistic regression model on four of the five splits, and evaluate its performance on the fifth,
6. Average the AUROC of each model across the five splits and the three studies.
7. Sort the models by their AUROC, and create ROC curves for each study for the model corresponding to the highest performing feature set.

The above analysis was conducted in two ways:
A. including full list of biomarkers, and
B. a follow-up analysis excluding the highest performing biomarker (NEFL).

Table 19 below shows that biomarker panels including two, three, or four biomarkers are predictive for presence or absence of MS. In particular, for pairs of biomarkers, a panel including NEFL and TNFSF13B achieved an AUROC of 0.788 and a PPV of 0.708, a panel including NEFL and CNTN2 achieved an AUROC of 0.777 and a PPV of 0.732, and a panel of NEFL and CXCL9 achieved an AUROC of 0.777 and a PPV of 0.713. Additional pairs of biomarkers (without NEFL) were also predictive for presence or absence of multiple sclerosis. A panel including MOG and CDCP1 achieved an AUROC of 0.672 and a PPV of 0.597, a panel including MOG and TNFSF13B achieved an AUROC of 0.672 and a PPV of 0.599, and a panel including MOG and CXCL9 achieved an AUROC of 0.670 and a PPV of 0.606.

In particular, for biomarker triplicates, a panel including NEFL, CNTN2, and TNFSF13B achieved an AUROC of 0.794 and a PPV of 0.745. Here, substitution of CNTN2 in the biomarker triplicate with either APLP1 or TNFRSF10A achieved similar AUROC values of 0.794 and 0.792, respectively. For a biomarker triplicate without NEFL, a panel including MOG, CXCL9, and TNFSF13B achieved an AUROC of 0.690 and a PPV of 0.623, a panel including MOG, OPG, and TNFSF13B achieved an AUROC of 0.685 and a PPV of 0.637, and a panel including MOG, CCL20, and TNFSF13B achieved an AUROC of 0.685 and a PPV of 0.664. APLP1, CCL20, and CNTN2 are the next proteins that help improve signal beyond biomarker triplicates. For biomarker quadruplets, a panel including NEFL, TNFRSF10A, CNTN2, and TNFSF13B achieved an AUROC of 0.798 and a PPV of 0.742.

### Example 43: Additional Multivariate (Pairs, Triplets, and Quadruplets) Biomarker Panel for Predicting Severity of Multiple Sclerosis

Multivariate analyses of minimal sets of biomarkers (e.g., pairs, triplicates, and quadruplets) were conducted across the different human clinical studies according to the methods described in Example 3. Specifically, minimal sets of biomarkers were analyzed for their ability to predict a number of Gd-enhancing lesions (e.g., a measure of subtle MS disease).

A regression analysis was performed analogously to the cross-sectional and shift analysis, with the following procedure:
1. Read in the list of biomarkers.
2. Read in the data for these biomarkers and their attendant demographic and clinical features for each study from the data lake.
3. Drop all samples that were collected more than a threshold time (e.g., more than 30 days) from their associated MRI scan.
4. Perform optimized demographic and clinical adjustment.
   A. Run an OLS regression between the optimal subset of demographic/clinical features and the NPX level of each individual biomarker value, using only the Gd-samples (with no disease activity) for a given study, filtering out outliers.
      a. DiseaseDuration: the number of years since MS diagnosis.
      b. Age: the age of a patient in number of years.
      c. Sample Age: the length of storage of a sample before it was formally analyzed.
      d. Delta BloodMinusDiagnosis: the number of days between an MM and respective blood sample (must be between 30).
   B. Extract residuals from this procedure and use that as input into model-building procedures.
5. For each study:
   a. use the Gd lesion count (clipped at a maximum of 5) as an endpoint for the regression,
   b. perform a five-fold cross validation split,
   c. select a ridge regression model configuration that worked well in past multivariate analyses,
   d. create feature matrices from every combination of two, three, and four proteins (and no demographic/clinical features as covariates),
   e. train a copy of the ridge regression model on four of the five splits, and evaluate its performance on the fifth,
6. Average the adjusted R² of each model across the five splits and the three studies.
7. Sort the models by their adjusted R² and create correlation plots between the predictions and the endpoints for each study for the model corresponding to the highest performing feature set.

The above analysis was conducted in two ways:
A. including full list of biomarkers, and
B. a follow-up analysis excluding the highest performing biomarker (NEFL).

Table 20 below shows that biomarker panels including two, three, or four biomarkers are generally predictive for determining subtle MS disease activity.

In particular, for pairs of biomarkers, a panel including NEFL and TNF achieved a Spearman's R coefficient value of 0.524, a panel including NEFL and SERPINA9 achieved a Spearman's R coefficient value of 0.501, and a panel including NEFL and GH achieved a Spearman's R coefficient value of 0.505. Additional pairs of biomarkers (without NEFL) were also predictive for determining subtle MS disease activity. For example, a panel including MOG and TNFSF13B achieved a Spearman's R coefficient value of 0.286 and a panel including MOG and CXCL9 achieved a Spearman's R coefficient value of 0.290.

For biomarker triplicates, a panel including NEFL, SERPINA9, and TNFSF13B achieved a achieved a Spearman's R coefficient value of 0.533, a panel including NEFL, CNTN2, and TNFSF13B achieved a Spearman's R coefficient value of 0.525, and a panel including NEFL, APLP1, and TNFSF13B achieved a Spearman's R coefficient value of 0.537. Additionally, a panel of MOG, CXCL9, and TNFSF13B achieved a Spearman's R coefficient value of 0.306, a panel of MOG, CCL20, and COL4A1 achieved a Spearman's R coefficient value of 0.210, and a panel of CXCL13, APLP1, and FLRT2 achieved a Spearman's R coefficient value of 0.143.

For biomarker quadruplicates, a panel including NEFL, CXCL13, CCL20, and TNFSF13B achieved a Spearman's R coefficient value of 0.520. Additionally, a panel including NEFL, CXCL13, CXCL9, and TNFSF13B achieved a Spearman's R coefficient value of 0.513 and a panel including NEFL, CXCL13, SERPINA9, and TNFSF13B achieved a Spearman's R coefficient value of 0.513. A biomarker quadruplicate (not including NEFL) of MOG, CXCL9, OPG, SERPINA9, and TNFSF13B achieved a Spearman's R coefficient value of 0.302.

### Example 44: Multivariate (Pairs, Triplets, and Quadruplets) Biomarker Panel for Predicting Multiple Sclerosis Disease Progression

Multivariate analyses of minimal sets of biomarkers (e.g., pairs, triplicates, and quadruplets) were conducted n=205 samples from the University of Basel Hospital according to the methods described in Example 3. Specifically, minimal sets of biomarkers were analyzed for their ability to predict a primary disease endpoint of disease progression (e.g., association of serum protein measurement with Expanded Disability Status Score (EDSS)). All sets use mathematical combinations (such as a logistic regression model or decision tree) to combine individual biomarker levels into a multivariate score.

A ridge (L2) regularization linear model was evaluated with 5-fold cross-validation (to estimate mean and uncertainty) across all exhaustive combinations of pairs, triplets, and quadruplets of protein subsets. Performance was then sorted by Spearman R 2 (per the rationale above), and this procedure was repeated for:
1. Models built with just the logarithmic transform of the protein concentrations
2. Models built with the logarithmic transform of the protein concentrations plus age, sex, and disease duration covariates incorporated as eligible features
3. Models built with the logarithmic transform of the protein concentrations after the residuals have been extracted from an OLS regression procedure to predict each respective biomarker concentration from just the demographic information (age, sex, and disease duration) contained within the samples that present no disease activity (i.e. contain 0 Gd lesions in their corresponding MM).

Table 21 documents best performing minimally predictive biomarker sets (e.g., pairs, triplets, and quadruplets) according to regression on EDSS predicting the number of lesions that an associated MM had based on protein signatures in blood serum for a single blood draw within 30 days of the MM. Of note, the biomarker triplicate of GFAP, NEFL, and MOG exhibited the highest overall adjusted R² (with a measurable improvement above the best univariate feature in field—GFAP). Additionally, biomarker pairs of A) GFAP and MOG, B) GFAP and NEFL, and C) APLP1 and GFAP as well as biomarker quadruplets GFAP, NEFL, MOG, and IL-12B/PRTG/APLP1 also exhibited predictiveness.

The best biomarker sets with Covariate (age, sex, disease duration) adjusted Log(pg/mL)) include: the biomarker pair of NEFL and MOG which exhibited the highest overall adjusted R². Biomarker triplicates of NEFL, MOG, and GH as well as NEFL, MOG, and SERPINA9 also predict EDSS in an improved fashion. Biomarker quadruplets NEFL, MOG, GH, and SERPINA9 and NEFL, MOG, GH, and TNFRSF10A also improve further. Additionally, biomarker triplicate CXCL9, OPG, and SERPINA9 is predictive of disease progression and further improves when MOG is added as a biomarker quadruplet.

The best biomarker sets with Demographic (age, sex, disease duration) adjusted include biomarker pair CXCL9 and OPG and biomarker triplicate CXCL9, OPG, and TNFRSF10A. Biomarker quadruplet CD6, IL-12B, APLP1, and CCL20 forms the highest Pearson R² correlation of any of the demographic-adjusted models.

### Example 45: Biomarker Panel for Predicting Disease Progression (Brain Parenchymal Fraction)

Univariate and multivariate biomarker panels were constructed for predicting brain parenchyma fraction (BPF), which is a known correlate with MS disease progression. The F6 study (University Hospital Basel Cohort (UHBC)) was used to construct and analyze the biomarker panels. Here, the cohort included 205 samples from 88 patients (67 female, 21 male) at University Hospital Basel. Samples banked between July 2012 to August 2019 with paired MRI scans. Patients have between 2-6 longitudinal samples (all patients have at least one sample with Gd enhancing lesions and one without). The cohort was selected to be balanced across age, sex, disease duration, EDSS for a study whose primary endpoint was Gd lesion count. Characteristics of the UHBC cohort is shown in Table 22.

Patient images were analyzed and labeled. Specifically, 3D T1 and FLAIR images were uploaded in Octave's cloud environment and quality control of raw data was performed by two experienced raters. Data was rated for quality on 1-5 scale (1=poor, 3=average, 5=excellent). Image segmentation of T1 and FLAIR images was performed through Cortechs' FDA-approved LesionQuant software. Second quality control of the segmentation was performed by raters.

Univariate biomarker panels were constructed using individual biomarkers selected from NfL, MOG, CD6, CXCL14, CXCL9, CDCP1, CCL20, OPG, IL-12B, APLP1, GH, VCAN, TNFRSF10A, SERPINA9, PRTG, FLRT2, TNFSF13B (BAFF), OPN, CNTN2, and GFAP. Additionally, a multivariate panel was constructed using each of the 20 biomarkers of NfL, MOG, CD6, CXCL14, CXCL9, CDCP1, CCL20, OPG, IL-12B, APLP1, GH, VCAN, TNFRSF10A, SERPINA9, PRTG, FLRT2, TNFSF13B (BAFF), OPN, CNTN2, and GFAP.

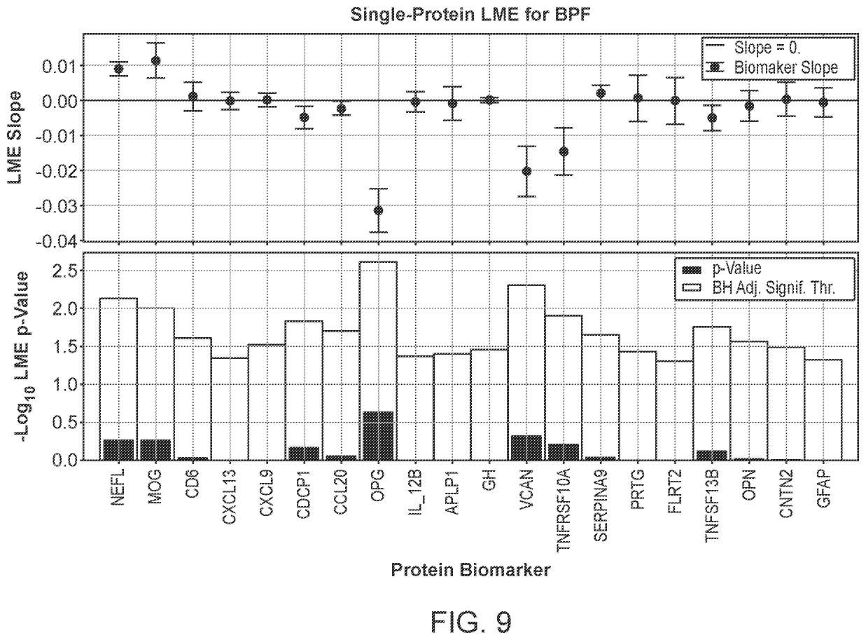

FIG. 9 depicts univariate analysis of individual biomarkers for predicting brain parenchymal fraction. Linear mixed effects (LME) models were constructed to account for variation within and across patients. The dependent variable was brain parenchymal fraction and independent variables included age, sex, disease duration, and log₁₀(serum biomarker concentration). The slope for each protein (top panel of FIG. 9) and associated p-value (bottom panel of FIG. 9) was determined using the model estimating BPF based on the quantitative levels of the protein and demographic information. Notably, no single protein biomarker passes the multiple hypothesis significance threshold (Benjamin-Hochberg).

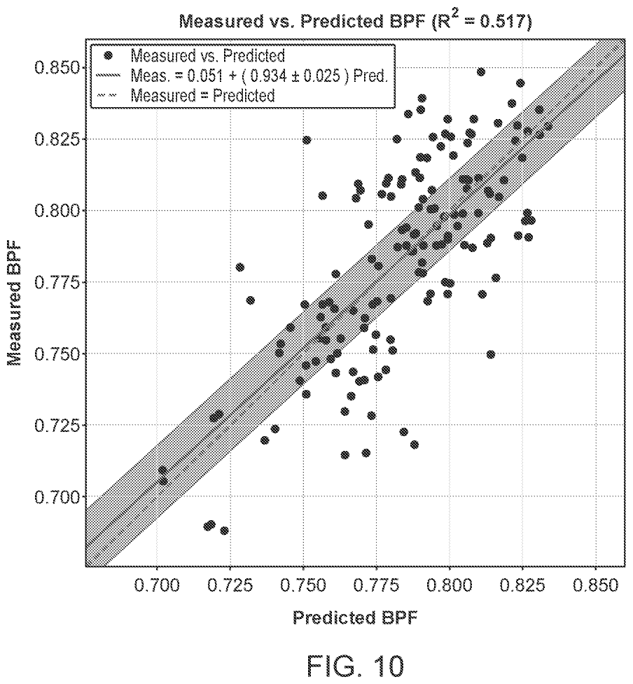

FIG. 10 depicts a multivariate analysis of a combination of biomarkers for predicting brain parenchymal fraction. Here, an ensemble of LME models were constructed, the LME models predicting BPF using the 20 protein biomarkers, age, disease duration, and sex using Leave One Out Cross Validation (LOOCV) to minimize overfitting. The importance of each feature from each model was extracted across LOOCV splits. FIG. 10 depicts the performance of the multivariate model performance which analyzes quantitative protein concentrations and demographic information. As shown in FIG. 10, the R² is 0.517. The linear regression of measured vs. predicted linear regression is consistent with the unity function (y=x). Altogether, multivariate modeling incorporating the 20 biomarkers outperforms any single protein biomarker.

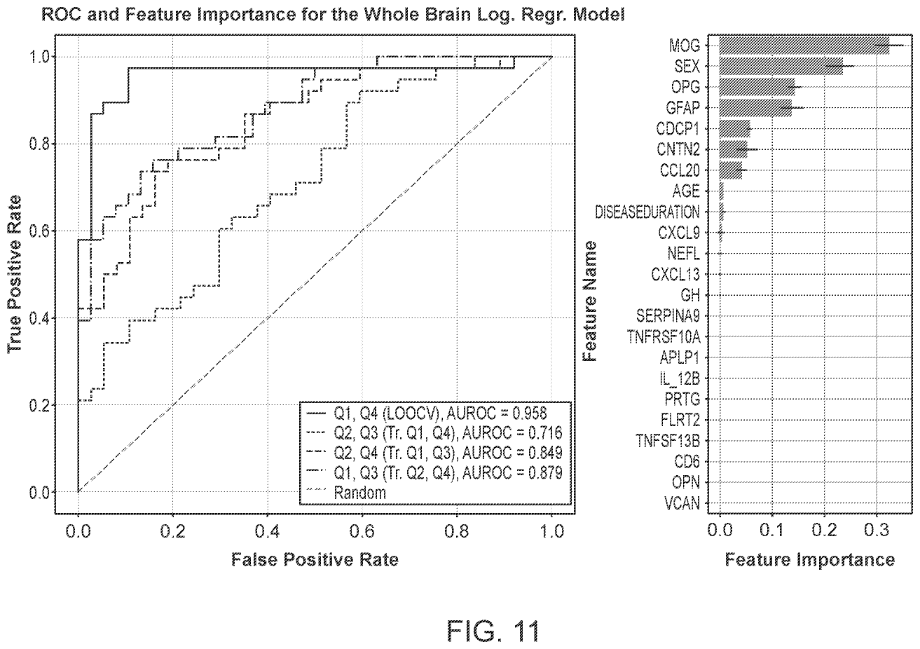

FIG. 11 depicts a multivariate analysis of a combination of biomarkers for predicting brain parenchymal fraction quartiles. Here, 3D T1 and FLAIR images from patients were separated into BPF quartiles. Biomarker levels were then used (along with demographic features) to predict into which quartile a scanned brain falls.

Four training/testing methods were used to split the data: 1) Separate quartile 1 from quartile 4 and LOOCV to check overfitting (Q1, Q4 (LOOCV), AUROC 0.958), 2) train on quartile 1 and quartile 4 and test on separating quartile 2 and quartile 3 (Q2, Q3 (Tr. Q1, Q4)), AUROC=0.716), 3) train on quartile 1 and quartile 4 and test on separating quartile 2 and quartile 4 (Q2, Q4 (Tr. Q1, Q3), AUROC=0.849), 4) train on quartile 2 and quartile 4 and test on separating quartile 1 and quartile 3 (Q1, Q3 (Tr. Q2, Q4), AUROC=0.879).

Altogether, the results described in this example demonstrate that predicting imaging biomarkers using serum protein concentration is a viable endeavor as evidenced by the prediction and classification results described herein. For BPF estimation, no single protein shows statistically significant performance after multiple hypothesis correction. However, the multivariate biomarker model shows promising performance (R²=0.517), with ten proteins showing significant importance averaged across all patients in the study. Multivariate separation of high from low BPF brains shows strong performance for the multiple framings of “low” and “high” that were investigated.

### Example 46: Biomarker Panel for Predicting Disease Progression (PDDS, PROMIS, and MSRS-R)

Univariate and multivariate biomarker panels were constructed for predicting patient-reported outcomes that correlate with MS disease progression. Patient-reported outcomes include patient determined disease steps (PDDS), which is a validated scale as a self-reported proxy for EDSS. The PDDS scale is rated on a scale of 0-8, where a score of 0 refers to a normal disability level, a score of 1 refers to a mild disability level, a score of 2 refers to a moderate disability level, a score of 3 refers to a gait disability level, a score of 4 refers to an early cane level, a score of 5 refers to a late cane level, a score of 6 refers to a bilateral support level, a score of 7 refers to a wheelchair/scooter level, and a score of 8 refers to a bedridden level. Patient-reported outcomes further include PRO measurement information system (PROMIS), and Multiple Sclerosis Rating Scale, Revised (MSRS-R), which is a composite score including domains of walking, arms, cognition, vision, speech, swallowing, sensation, and bladder/bowel control.

The Prospective Investigation of Multiple Sclerosis in the Three Rivers Region (PROMOTE) study (IRB: STUDY19080007B) was used for building the biomarker panels. The study utilized the following inclusion criteria: Individuals must be 7 years or older, diagnosed with multiple sclerosis or related disorders, including a first central nervous system demyelinating episode with a positive MRI scan or abnormal MM scans characteristic of MS but no clinical symptoms of the disease. Table 23 identifies characteristics of patients in the PROMOTE study.

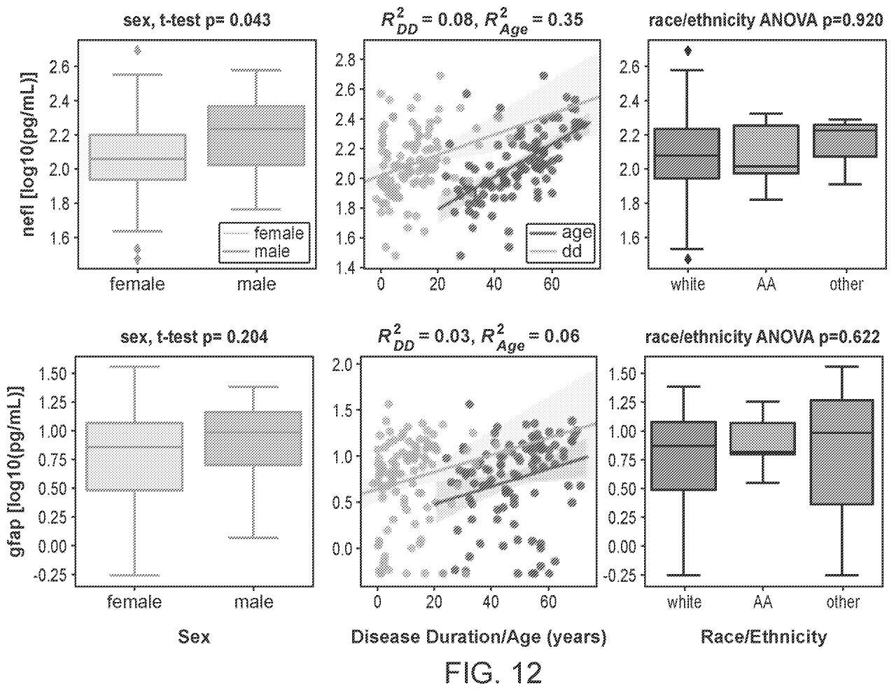

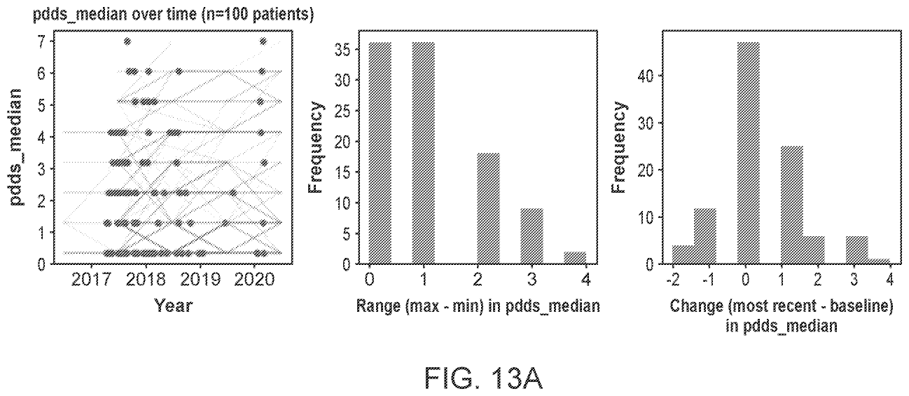

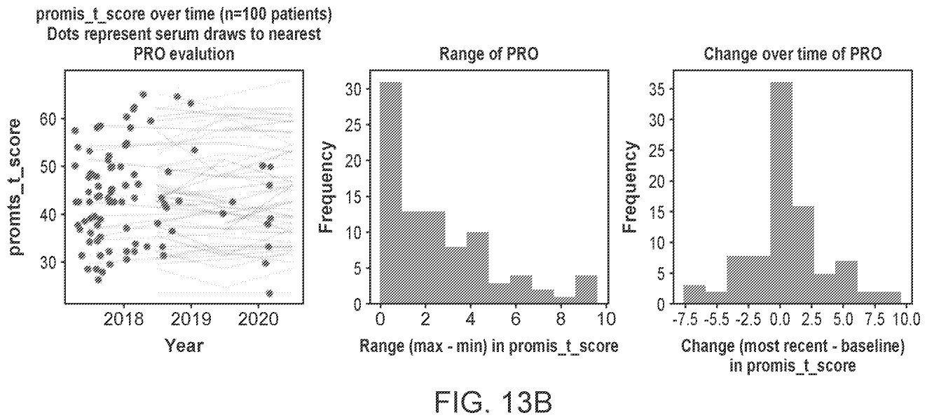

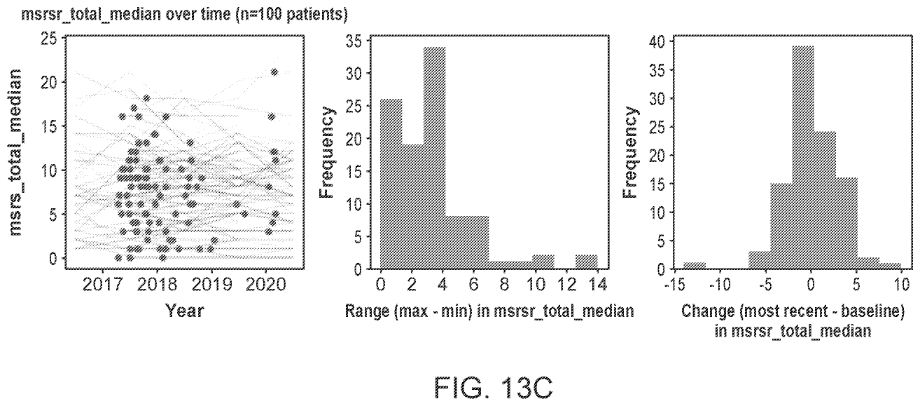

FIG. 12 depicts associations of sex, disease duration, and race/ethnicity with biomarkers (NfL and GFAP). FIGS. 13A-13C depict characterization of patient-reported outcomes (patient determined disease steps (PDDS), PRO measurement information system (PROMIS), and Multiple Sclerosis Rating Scale, Revised (MSRS-R). To account for variance in PRO assessments, only the median from each year was considered. The time lag between PRO assessments and serum draws was taken into account to avoid bias. Associations with age, sex, disease duration, and race/ethnicity were examined for each protein (NfL, GFAP shown in FIG. 12 across 100 samples, pre-adjustment).

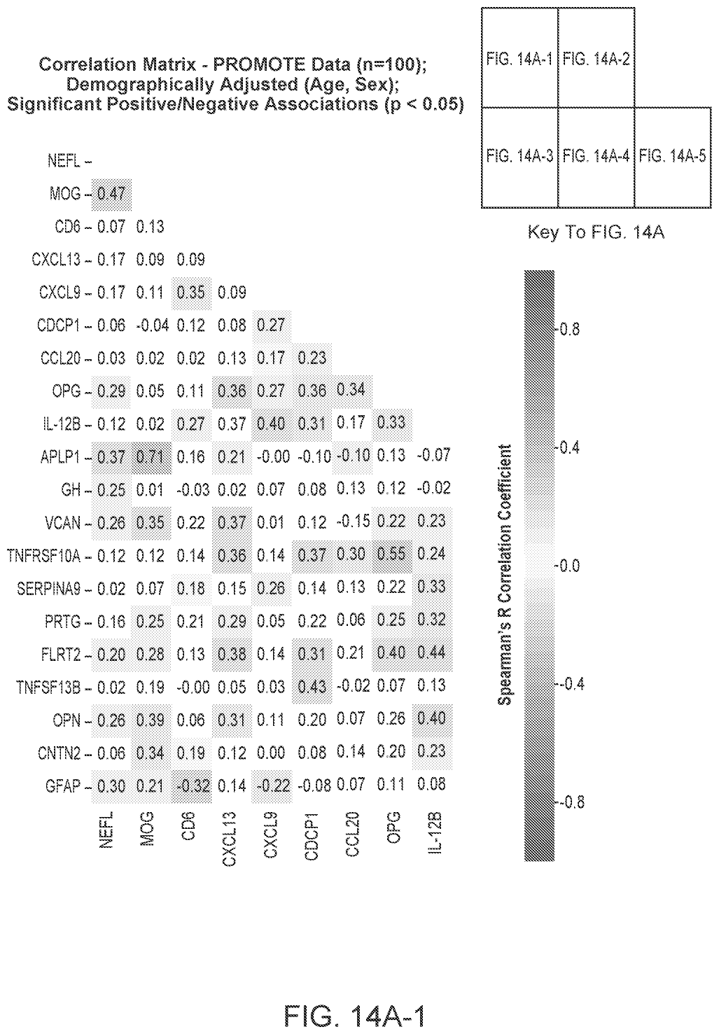

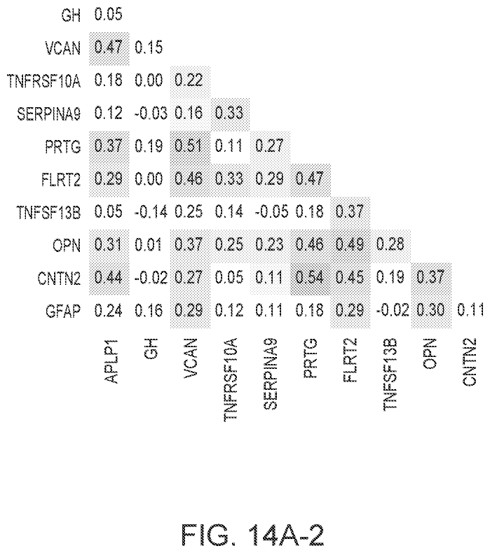

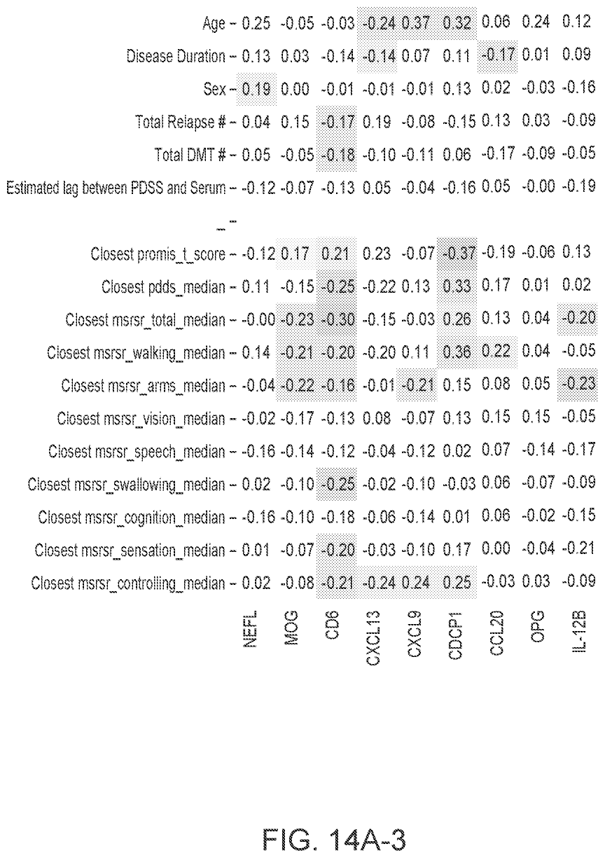

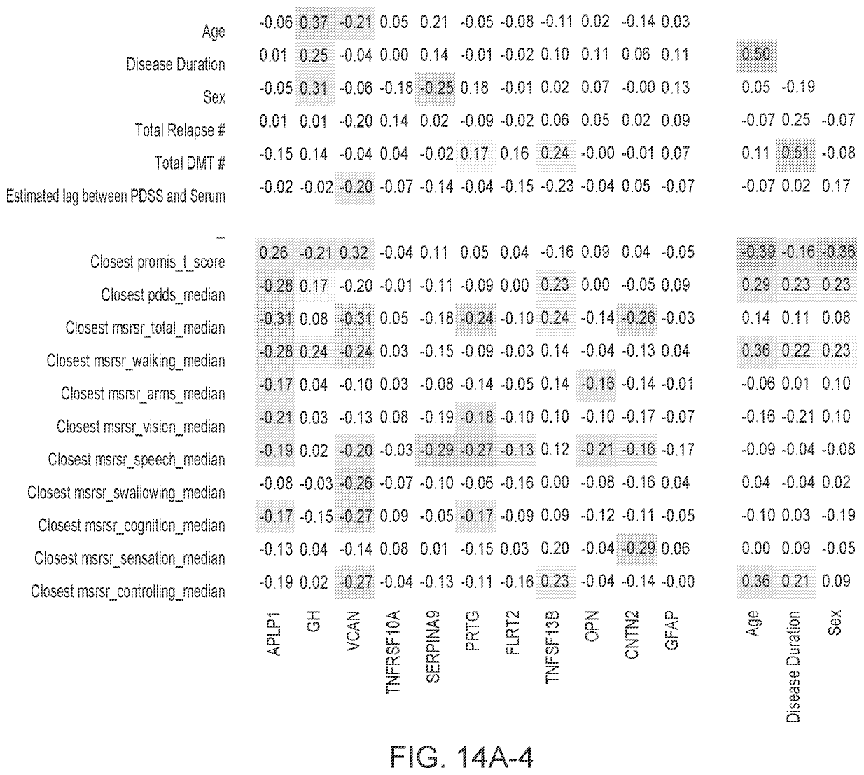

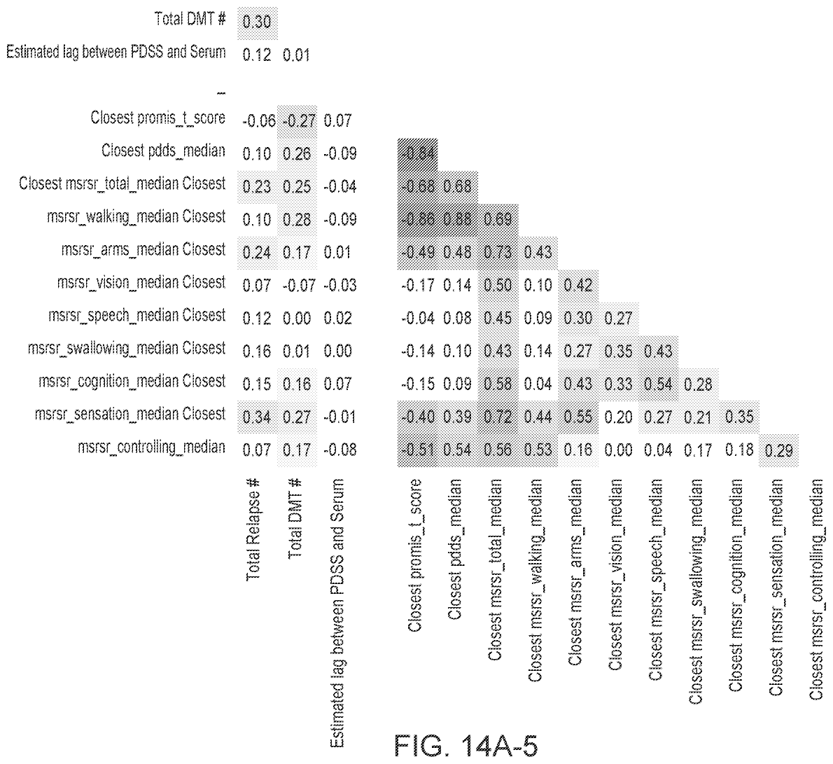

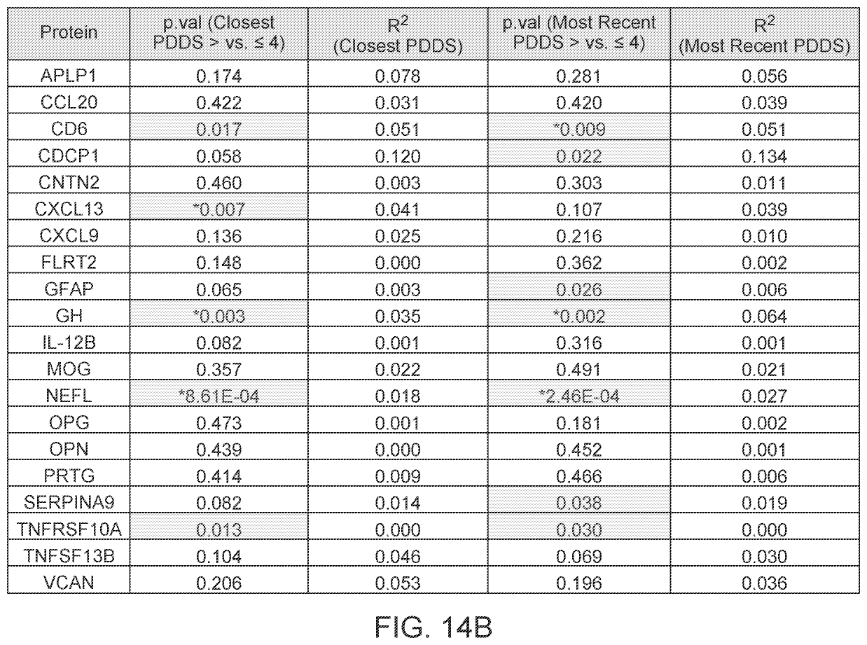

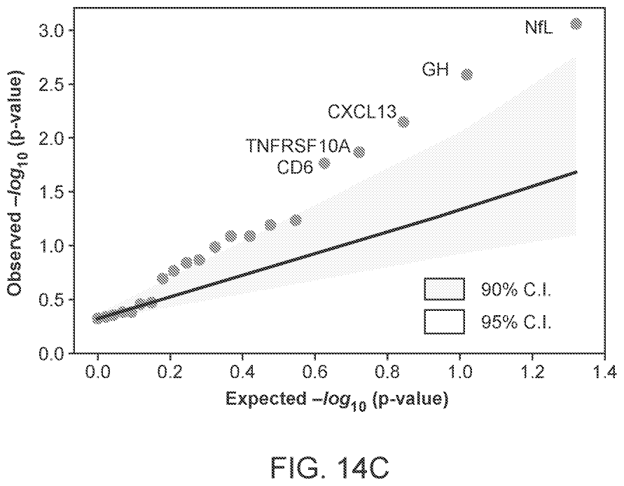

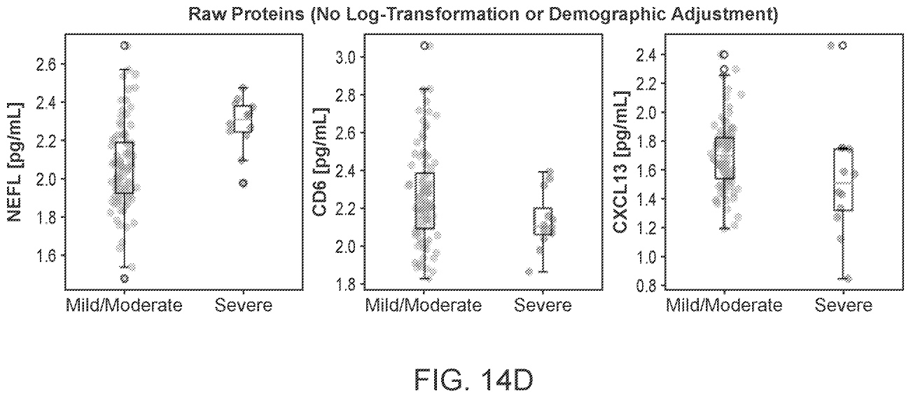

FIG. 14A depicts a correlation matrix revealing associations between individual biomarkers and PDDS, PROMIS, and MSRS-R. Significant positive/negative associations (p<0.05) are shown in green and red, respectively. For PDDS, the milestone of 4 is a common separator for classifying disability. For PROMIS and MSRS-R (and PDDS), an ordinary least squares general linear model was constructed to look at which markers were the most significantly associated with risk of advancing to a PRO milestone. As indicated in FIG. 14A, the strongest direct correlations include: NEFL/MOG/APLP1, TNFRSF10A/OPG, FLRT2/VCAN, PRTG/CNTN2, MSRS-R/PROMIS, Strongest inverse correlations include: PROMIS t-score/MSRS-R walking, PROMIS t-score/PDDS median, and GFAP/CD6.

FIG. 14B depicts univariate analysis of individual biomarkers for predicting PDDS outcomes. Statistically significant P-values <0.05 are highlighted in green (non-parametric Mann-Whitney U test). *indicates that marker was significant at Benjamini-Hochberg multiple hypothesis correction (FDR=0.05). R² reflects square of Spearman's R correlation. PDDS milestone of 4 (equivalent to EDSS of 6) was used to separate patients into groups of severe vs. mild/moderate disability. For improved technical validation, PwMS samples were divided based on both 1) closest PDDS assessment to serum draw and 2) end-of-study PDDS assessment (i.e. farthest measure of disability into the future, “most recent”).

Notably, protein biomarkers of CD6, CXCL13, GH, NEFL, and TNFRSF10A exhibited statistically significant p-values less than 0.05 in differentiating mild/moderate disability (e.g., PDDS <4) and severe disability (e.g., PDDS >4) based on closest PDDS. Additionally protein biomarkers of CD6, CDCP1, GFAP, GH, NEFL, SERPINA9, and TNFRSF10A exhibited statistically significant p-values less than 0.05 in differentiating mild/moderate disability (e.g., PDDS <4) and severe disability (e.g., PDDS >4) based on most recent PDDS.

FIG. 14C depicts quantile-quantile plot of expected versus observed p-values of disability severity. The dark gray and light gray areas reflect the confidence interval as generated by 10,000 bootstrapped permutations at a threshold of p=0.10 and p=0.05 respectively. The observed p-values for disability severity are outside of the gray areas, suggesting that serum biomarkers (namely NfL, GH, CXCL13, TNFRSF10A, CD6) are associated with the PDDS severity score beyond chance after accounting for multiple testing burden.

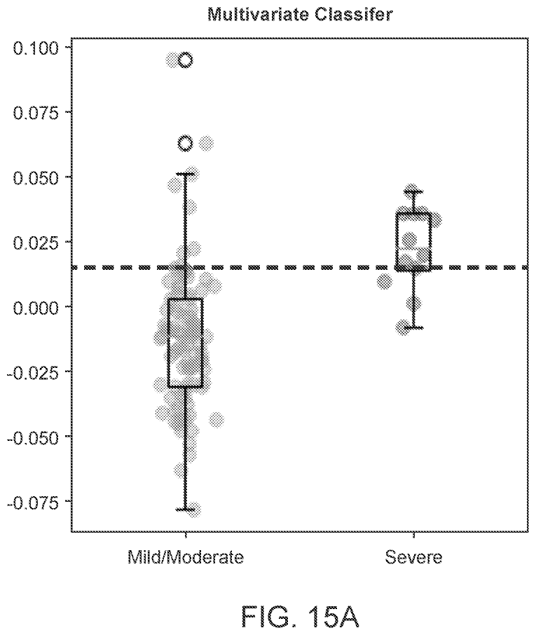

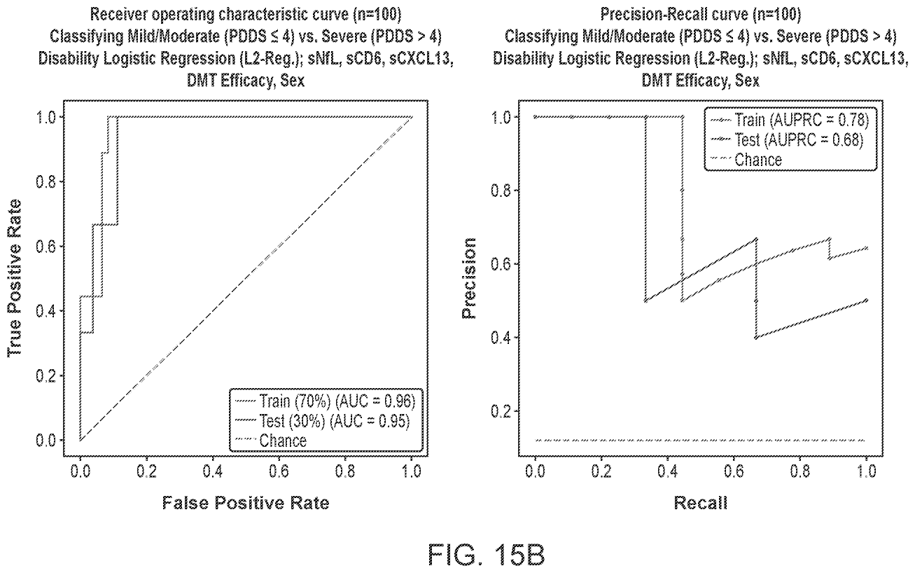

FIG. 14D depicts classification of PDDS-defined severity (e.g., mild/moderate v. severe) according to univariate protein analyses (NfL, CD6, and CXCL13). FIG. 14D shows that different protein biomarker concentrations of NfL, CD6, and CXCL13 are informative for differentiating between mild/moderate (PDDS <4) v. severe disability (PDDS >4). FIG. 15A depicts classification of PDDS-defined severity (e.g., mild/moderate v. severe) according to a multivariate biomarker analysis (NfL, CD6, and CXCL13). Here, the objective was to optimize the classifier for PDDS-defined severity (closest assessment >vs. ≤4). The dataset was divided 70/30 into a train/validation and holdout test set (stratification was used due to class imbalance). 5-fold cross-validation was used to reduce overfitting and determine the best model.

FIG. 15B depicts receiver operating characteristic (ROC) curve and precision-recall curve for classifying PDDS-defined severity (e.g., mild/moderate v. severe) through a multivariate biomarker analysis (NfL, CD6, and CXCL13). The cross-validated multivariate logistic regression classifier based on NfL, CD6, and CXCL13 was able to distinguish disease severity (e.g., mild/moderate v. severe). Specifically, the classifier exhibited performance metrics of: AUC: 0.96 (Train)/0.95 (Test), Accuracy: 0.87/0.90, Sensitivity (Recall): 0.87/0.90, PPV (Precision): 0.76/0.81, and F1-score: 0.81/0.85. These results demonstrate that serum biomarkers can predict the risk of future MS disability based on progression to PDDS milestone.

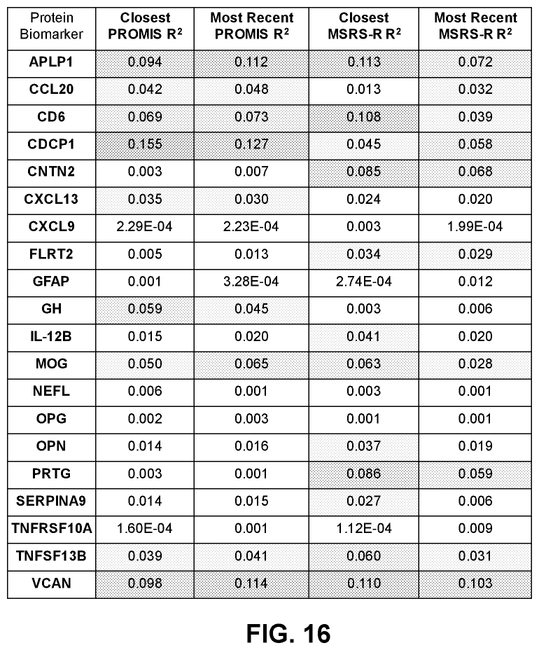

FIG. 16 depicts univariate analysis of individual biomarkers for predicting PROMIS or MSRS-R outcomes. Darker shading in FIG. 16 indicates higher correlation between the protein biomarker and either the PROMIS or MSRS-R self-reported outcome. Based on sequential feature selection with 5-fold cross-validation, the best multivariate predictor of PROMIS annualized t-score (R²=0.378) includes: CDCP1, IL-12B, Age, Sex, and Disease Duration. Additionally, the best Multivariate Predictor of MSRS-R (R²=0.454) includes: CD6, CDCP1, IL-12B, VCAN, Age, Sex, and Disease Duration.

### Example 47: Multivariate Panels for Predicting Multiple Sclerosis Disease Progression

Biomarkers were selected for a multivariate custom panel. Multivariate biomarker panels were constructed according to the biomarker tiers shown in Table 24A. Specifically, Tier 1 biomarkers include GFAP, CDCP1, MOG, CXCL13, OPG, APLP1, VCAN, and NEFL. Tier 2 biomarkers include CXCL9, TNFRSF10A, CCL20, TNFSF13B, CD6, SERPINa9, FLRT2, OPN, and CNTN2. Tier 3 biomarkers include COL4A1, GH, IL-12B, and PRTG.

429 samples were included in the clinical validation training set and were sourced from University of Massachusetts (UMass), Brigham Women's Hospital, Rocky Mountain MS Center (RMMSC), and American University of Beirut (AUB). Characteristics of these patient cohorts are shown in Table 25. An additional 1028 samples were included and derived from BWH (CLIMB), UCSF (EPIC), Basel, and Pitt (PROMOTE), the studies of which are described in Table 5A.

Different combinations of the tier 1, tier 2, and tier 3 biomarkers were implemented across the different human clinical studies. In particular, biomarker panels were constructed from one or more tiers (e.g., Tier 1 alone, tier 2 alone, tier 3 alone, tiers 1+2, or tiers 1+2+3 biomarkers of Table 24A). A first set of biomarker panels were constructed for predicting classification of patients (e.g., mild/moderate v. severe disability). For this first set of biomarker panels, stratified 5-fold cross-validation was used to assess the performance (with AUROC and PPV as the reported metrics). A regularized logistic regression model was used. Statistical measures of the performance of these classifiers are shown in Table 24B and include area under the curve (AUROC) and positive predictive value (PPV). Additionally, a second set of biomarker panels were constructed for predicting in a linear regression model for disability. Stratified 5-fold cross-validation was used to assess the performance (with R² and Adjusted R²). A ridge regularized linear regression model was used to find the best associations between combinations of serum biomarkers and the endpoint. Statistical measures of the performance of these regression models are shown in Table 24C and include measures such as Pearson's correlation coefficient (e.g., Pearson's R²—square of the Pearson's R correlation).

Generally, biomarker panels that employed biomarkers from each of tier 1, tier 2, and tier 3 (21 total biomarkers) achieved improved classifier performance (e.g., Table 24B) and improved regression performance (e.g., Table 24C) in comparison to smaller biomarker panels (e.g., Tier 1 biomarkers only, tier 2 biomarkers only, tier 1+2 biomarkers only). Notably, a significant amount of predictive power arises from the biomarkers included in Tier 1. Specifically, referring to the classifier performance in Table 24B, Tier 1 only biomarkers achieved an AUROC of 0.77 and a PPV of 0.19. Similarly, referring to the regression performance in Table 24C, Tier 1 only biomarkers achieved a Pearson's correlation coefficient (e.g., Pearson's R²) of 0.36 which is further matched when Tier 2 and Tier 3 biomarkers were added in addition to the Tier 1 biomarkers (Pearson's R²=0.36).

Referring specifically to the classifier performance in Table 24B, using raw biomarker values, the biomarker panels achieved an AUROC of at least 0.74 and a PPV of at least 0.17. Specifically, Tier 1+2+3 achieved an AUROC of 0.74 and PPV of 0.17, Tiers 1+2 achieved an AUROC of 0.76 and PPV of 0.19, and Tier 1 biomarkers alone achieved an AUROC of 0.77 and PPV of 0.19. Using adjusted biomarker values (e.g., demographically adjusted), biomarker panels achieved an AUROC of at least 0.79 and PPV of 0.19. Specifically, Tier 1 biomarkers alone achieved an AUROC of 0.79 and PPV of 0.19, Tier 1+2 biomarkers achieved an AUROC of 0.81 and PPV of 0.20, and Tier 1+2+3 biomarkers achieved an AUROC of 0.81 and PPV of 0.21.

Referring specifically to the regression performance in Table 24C, using raw biomarker values, the biomarker panels achieved a Pearson's R-sq value of at least 0.35. Specifically, Tier 1 biomarkers alone achieved a Pearson's R-sq value of 0.36, Tiers 1+2 biomarkers achieved a Pearson's R-sq value of 0.35, and Tiers 1+2+3 biomarkers achieved a Pearson's R-sq value of 0.36. Using adjusted biomarker values (e.g., demographically adjusted), biomarker panels achieved a Pearson's R-sq value of at least 0.35. Specifically, Tier 1 biomarkers alone achieved a Pearson's R-sq value of 0.40, Tiers 1+2 biomarkers achieved a Pearson's R-sq value of 0.35, and Tiers 1+2+3 biomarkers achieved a Pearson's R-sq value of 0.36.

### Example 48: Additional Multivariate (Pairs, Triplets, and Quadruplets) Biomarker Panel for Predicting Multiple Sclerosis Disease Progression Using Classifier that Differentiates Mild/Moderate Vs. Severe Disability

Regularized logistic regression models were trained and tested for their ability to distinguish between two disease progression classifications: 1) mild/moderate multiple sclerosis and 2) severe disability multiple sclerosis. The models were trained and tested using samples in which the patient characteristics are shown in Table 25. Specifically, 429 samples (70%) were used for the training set and 188 samples (30%) were used for testing the trained model.

Logistic regression models were built using pairs, triplets, or quadruplets of biomarkers that can predict in a classifier of mild/moderate vs. severe disability (i.e. disease progression status at the time of blood draw). The endpoint is based on the Expanded Disability Status Scale (EDSS) and Patient Determined Disease Steps (PDDS) scales. EDSS >=6 and PDDS >=4 are the thresholds used to define severe (instead of mild/moderate) disability because those are the minimum clinician-defined (in the case of EDSS) and patient-reported (in the case of PDDS) outcomes for requiring ambulatory assistance (i.e. a cane). Stratified 5-fold cross-validation was used to assess the performance and these minimally predictive sets (with AUROC and PPV as the reported metrics). AUROC was used to rank. Performance of the respective logistic regression models is shown in Table 26.

In particular, logistic regression models incorporating GFAP (e.g., GFAP as one biomarker of the pairs, triplets, or quadruplets) demonstrated AUROC values of at least 0.70. In particular, logistic regression models incorporating GFAP (e.g., GFAP as one biomarker of the pairs, triplets, or quadruplets) demonstrated AUROC values between 0.72 and 0.78. Specifically, logistic regression models including two biomarkers of which one is GFAP demonstrated AUROC values ranging from 0.725 to 0.732. Logistic regression models including three biomarkers of which one is GFAP demonstrated AUROC values ranging from 0.748 to 0.764. Logistic regression models including four biomarkers of which one is GFAP demonstrated AUROC values ranging from 0.765 to 0.779.

In particular, logistic regression models that do not incorporate GFAP (e.g., GFAP is not included as one biomarker of the pairs, triplets, or quadruplets) demonstrated AUROC values of at least 0.65. For example, logistic regression models that do not incorporate GFAP (e.g., GFAP is not included as one biomarker of the pairs, triplets, or quadruplets) demonstrated AUROC values between 0.66 and 0.70. Specifically, logistic regression models including two biomarkers of which GFAP is not included demonstrated AUROC values ranging from 0.661 to 0.689. Logistic regression models including three biomarkers of which GFAP is not included demonstrated AUROC values ranging from 0.687 to 0.689. Logistic regression models including four biomarkers of which GFAP is not included demonstrated AUROC values ranging from 0.689 to 0.696.

These results demonstrate minimal sets of predictive biomarkers that are capable of predicting mild/moderate vs. severe disability.

### Example 49: Additional Multivariate (Pairs, Triplets, and Quadruplets) Biomarker Panel for Predicting Multiple Sclerosis Disease Progression Using Linear Regression Model that Predicts Disease Progression on an Ordinal Scale

Regularized linear regression models were trained and tested for their ability to predict disease progression status (e.g., at the time of blood draw). The models were trained and tested using samples in which the patient characteristics are shown in Table 25. Specifically, 429 samples (70%) were used for the training set and 188 samples (30%) were used for testing the trained model.

Linear regression models were built using pairs, triplets, or quadruplets of biomarkers that can predict in a linear regression model for disability (i.e. disease progression status at the time of blood draw). EDSS and PDDS scales were used according to a documented interpolation equation (EDSS=0.63*PDDS+2.9) in accordance with Learmonth, Y. C., et al. Validation of patient determined disease steps (PDDS) scale scores in persons with multiple sclerosis. BMC Neurol 13, 37 (2013), which is hereby incorporated by reference in its entirety. EDSS was the ground truth label used. Stratified 5-fold cross-validation was used to assess the performance and these minimally predictive sets (with R² and Adjusted R²).

Pearson's correlation coefficient (e.g., R²—square of the Pearson's R correlation) was used to rank the models deriving from log-transformed and demographically adjusted data while Spearman's nonparametric correlation coefficient was used to rank the models deriving from the raw data. A ridge regularized linear regression model was used to find the best associations between combinations of serum biomarkers and the endpoint. Performance of the respective linear regression models is shown below in Table 27.

In particular, regression models incorporating GFAP (e.g., GFAP as one biomarker of the pairs, triplets, or quadruplets) demonstrated R² values of at least 0.10, and in most cases, of at least 0.12. Generally, regression models incorporating GFAP (e.g., GFAP as one biomarker of the pairs, triplets, or quadruplets) demonstrated R² values between 0.11 and 0.22. Specifically, regression models including two biomarkers of which one is GFAP demonstrated R² values ranging from 0.115 to 0.216. Regression models including three biomarkers of which one is GFAP demonstrated R² values ranging from 0.148 to 0.217. Regression models including four biomarkers of which one is GFAP demonstrated AUROC values ranging from 0.171 to 0.214.

In particular, regression models that do not incorporate GFAP (e.g., GFAP is not included as one biomarker of the pairs, triplets, or quadruplets) demonstrated R² values of at least 0.01. Generally, regression models incorporating GFAP (e.g., GFAP as one biomarker of the pairs, triplets, or quadruplets) demonstrated R² values between 0.015 and 0.090. Specifically, regression models including two biomarkers of which GFAP is not included demonstrated R² values ranging from 0.056 to 0.083. Regression models including three biomarkers of which GFAP is not included demonstrated R² values ranging from 0.063 to 0.089. Logistic regression models including four biomarkers of which GFAP is not included demonstrated R² values ranging from 0.015 to 0.083.

These results demonstrate minimal sets of predictive biomarkers that are capable of predicting in a linear regression model for disability (i.e. disease progression status at the time of blood draw)

TABLE 1
21 biomarker panel grouped in tiers A, B, and C.

| | | | Accession |
|---|---|---|---|
| | | | Number |
| | | | (Uniprot |
| Tier | Biomarker Name | Biomarker Symbol | Database |
| A | Neurofilament Light Polypeptide | NEFL | P07196 |
| | Chain | | |
| A | Myelin Oligodendrocyte | MOG | Q16653 |
| | Glycoprotein | | |
| A | Cluster of Differentiation 6 | CD6 | P30203 |
| A | Chemokine (C-X-C motif) ligand 9 | CXCL9 | Q07325 |
| A | Osteoprotegerin | OPG | O00300 |
| A | Osteopontin | OPN | P10451 |
| A | Matrix Metallopeptidase 9 | MMP-9 | P14780 |
| A | Glial Fibrillary Acidic Protein | GFAP | 043155 |
| B | CUB domain-containing protein 1 | CDCP1 | Q9H5V8 |
| B | C-C Motif Chemokine Ligand 20 | CCL20/MIP 3-α | P78556 |
| B | Interleukin-12 subunit beta | IL-12B | P29460 |
| B | Amyloid Beta Precursor Like | APLP1 | P51693 |
| | Protein 1 | | |
| B | Tumor Necrosis Factor Receptor | TNFRSF10A | 000220 |
| | Superfamily Member 10A | | |
| B | Collagen, type IV, alpha 1 | COL4A1 | P02462 |
| B | Serpin Family A Member 9 | SERPINA9 | Q86WD7 |
| B | Fibronectin Leucine Rich | FLRT2 | O43155 |
| | Transmembrane Protein 2 | | |
| B | Chemokine (C-X-C motif) ligand 13 | CXCL13 | O43927 |
| C | Growth Hormone | GH | P01241 |
| C | Versican core protein | VCAN | P13611 |
| C | Protogenin | PRTG | Q2VWP7 |
| C | Contactin-2 | CNTN2 | Q02246 |

TABLE 2
Tiered biomarkers for use in predicting multiple sclerosis disease activity.

| | | | Accession |
|---|---|---|---|
| | | | Number |
| | | | (Uniprot |
| Tier | Biomarker Name | Biomarker Symbol | Database) |
| 1 | Neurofilament Light Polypeptide | NEFL | P07196 |
| | Chain | | |
| 1 | Myelin Oligodendrocyte | MOG | Q16653 |
| | Glycoprotein | | |
| 1 | Cluster of Differentiation 6 | CD6 | P30203 |
| 1 | Chemokine (C-X-C motif) ligand 9 | CXCL9 | Q07325 |
| 1 | Osteoprotegerin | OPG | O00300 |
| 1 | Osteopontin | OPN | P10451 |
| 1 | Chemokine (C-X-C motif) ligand 13 | CXCL13 | O43927 |
| 1 | Glial Fibrillary Acidic Protein | GFAP | O43155 |
| 2 | CUB domain-containing protein 1 | CDCP1 | Q9H5V8 |
| 2 | C-C Motif Chemokine Ligand 20 | CCL20/MIP 3-α | P78556 |
| 2 | Interleukin-12 subunit beta | IL-12B | P29460 |
| 2 | Amyloid Beta Precursor Like | APLP1 | P51693 |
| | Protein 1 | | |
| 2 | Tumor Necrosis Factor Receptor | TNFRSF10A | O00220 |
| | Superfamily Member 10A | | |
| 2 | Collagen, type IV, alpha 1 | COL4A1 | P02462 |
| 2 | Serpin Family A Member 9 | SERPINA9 | Q86WD7 |
| 2 | Fibronectin Leucine Rich | FLRT2 | O43155 |
| | Transmembrane Protein 2 | | |
| 2 | Tumor necrosis factor ligand | TNFSF13B | Q9Y275 |
| | superfamily member 13B | | |
| 3 | Growth Hormone | GH | P01241 |
| 3 | Versican core protein | VCAN | P13611 |
| 3 | Protogenin | PRTG | Q2VWP7 |
| 3 | Contactin-2 | CNTN2 | Q02246 |
| 4 | Growth Hormone 2 | GH2 | P01242 |
| 4 | Interleukin-18 | IL18 | Q14116 |
| 4 | Matrix Metalloproteinase-2 | MMP-2 | P08253 |
| 4 | Gamma-Interferon-Inducible | IFI30 | P13284 |
| | Lysosomal Thiol Reductase | | |
| 4 | Chitinase-3-like protein 1 | CHI3L1/YkL40 | P36222 |

TABLE 3
Tiered biomarkers for use in predicting multiple sclerosis
disease progression

| | | | Accession |
|---|---|---|---|
| | | | Number |
| | | Biomarker | (Uniprot |
| Tier | Biomarker Name | Symbol | Database) |
| 1 | Glial Fibrillary Acidic Protein | GFAP | 043155 |
| 1 | CUB domain-containing protein 1 | CDCP1 | Q9H5V8 |
| 1 | Myelin Oligodendrocyte | MOG | Q16653 |
| | Glycoprotein | | |
| 1 | Chemokine (C-X-C motif) ligand 13 | CXCL13 | O43927 |
| 1 | Osteoprotegerin | OPG | O00300 |
| 1 | Amyloid Beta Precursor Like | APLP1 | P51693 |
| | Protein 1 | | |
| 1 | Versican core protein | VCAN | P13611 |
| 1 | Neurofilament Light Polypeptide | NEFL | P07196 |
| | Chain | | |
| 2 | Chemokine (C-X-C motif) ligand 9 | CXCL9 | Q07325 |
| 2 | Tumor Necrosis Factor Receptor | TNFRSF10A | O00220 |
| | Superfamily Member 10A | | |
| 2 | C-C Motif Chemokine Ligand 20 | CCL20/MIP 3-α | P78556 |
| 2 | Tumor necrosis factor ligand | TNFSF13B | Q9Y275 |
| | superfamily member 13B | | |
| 2 | Cluster of Differentiation 6 | CD6 | P30203 |
| 2 | Serpin Family A Member 9 | SERPINA9 | Q86WD7 |
| 2 | Fibronectin Leucine Rich | FLRT2 | O43155 |
| | Transmembrane Protein 2 | | |
| 2 | Osteopontin | OPN | P10451 |
| 2 | Contactin-2 | CNTN2 | Q02246 |
| 3 | Collagen, type IV, alpha 1 | COL4A1 | P02462 |
| 3 | Growth Hormone | GH | P01241 |
| 3 | Interleukin-12 subunit beta | IL-12B | P29460 |
| 3 | Protogenin | PRTG | Q2VWP7 |

TABLE 4
Substitutable biomarkers for use in predicting multiple sclerosis disease activity.

| | | Substitute | Substitute | Substitute | |
|---|---|---|---|---|---|
| Biomarker | Substitute | Biomarker | Biomarker | Biomarker | Substitute |
| Symbol | Biomarker 1 | 2 | 3 | 4 | Biomarker 5 |
| NEFL | MOG (0.200- | CADM3 | GFAP (0.330 | | |
| | 0.470) | (0.142-0.184) | in CSF, | | |
| | | | 0.509 in | | |
| | | | Serum) | | |
| MOG | CADM3 | KLK6 | BCAN | OMG | GFAP (0.164 in |
| | (0.217-0.515) | (0.226-0.483) | (0.424-648) | (0.285- | Serum) |
| | | | | 0.371) | |
| CD6 | CD5 (0.206- | CRTAM | CD244 | TNFRSF9 | |
| | 0.448) | (0.172-0.364) | (0.159- | (0.154- | |
| | | | 0.419) | 0.344) | |
| CXCL9 | CXCL10 | CXCL11 | IL-12B | GFAP | |
| | (0.181-0.621) | (0.152-0.176) | (0.206- | (0.198 in | |
| | | | 0.272) | Serum) | |
| OPG | TFF3 (0.159- | ENPP2 | | | |
| | 0.513) | (0.281-0.330) | | | |
| OPN | OMD (0.459- | MEPE | GFAP (0.250 | | |
| | 0.553) | (0.347-0.515) | in Serum) | | |
| CXCL13 | NOS3 (0.371- | MMP-2 | | | |
| | 0.404) | (0.222 in | | | |
| | | Serum) | | | |
| GFAP | NEFL (0.509 | OPN (0.250 | CXCL9 | MOG | CHI3L1/YkL40 |
| | in Serum) | in Serum) | (0.198 in | (0.164 in | (0.593 in CSF) |
| | | | Serum) | Serum) | |
| CDCP1 | MSR1 (0.365- | IL-18BP | IL-18 (0.321- | GFAP | |
| | 0.410) | (0.270-0.481) | 0.330) | (0.136 in | |
| | | | | Serum) | |
| CCL20/MIP 3- | CCL3 (0.145- | TWEAK | | | |
| α | 0.150) | (0.164-0.199) | | | |
| IL-12B | IL-12 (0.877- | CXCL9 | | | |
| | 0.949) | (0.206-0.272) | | | |
| APLP1 | SEZ6L (0.299- | BCAN | DPP6 | NCAN | KLK6 (0.217- |
| | 0.369) | (0.435-0.561) | (0.201- | (0.213- | 0.423) |
| | | | 0.314 | 0.294) | |
| TNFRSF10A | TNFRSF11A | SPON2 | CHI3L1 | IFI30 | |
| | (0.244-0.355) | (0.204-0.549) | (0.208- | (0.224- | |
| | | | 0.261) | 0.247) | |
| COL4A1 | IL-6 (0.186- | Notch 3 | PCDH17 | | |
| | 0.212) | (0.155-0.246) | (0.107- | | |
| | | | 0.320) | | |
| SERPINA9 | TNFRSF9 | CNTN4 | | | |
| | (0.169-0.202) | (0.089-0.181) | | | |
| FLRT2 | DDR1 (0.344- | NTRK2 | CDH6 | MMP-2 | |
| | 0.633) | (0.293-0.698) | (0.495- | (0.134- | |
| | | | 0.522 | 0.305) | |
| TNFSF13B | CXCL16 | ALCAM | IL-18 (0.329 | | |
| | (0.514) | (0.180-0.501) | in Serum) | | |
| IFI30 | CEACAM8 | MAD1L1 | FCAR | MPO | PRTN3 (0.474) |
| | (0.547) | (0.403) | (0.450) | (0.354) | |
| GH | IGFBP-1 | GH2 | | | |
| | (0.162-0.250) | | | | |
| VCAN | TINAGLI | CANT1 | NECTIN2 | NPDC1 | MMP-9 (0.301 |
| | (0.244-0.460) | (0.182-0.517) | (0.278- | (0.173- | in Serum) |
| | | | 0.457) | 0.426) | |
| PRTG | NTRK2 | NTRK3 | CNTN4 | | |
| | (0.219-0.539) | (0.249-0.485) | (0.187- | | |
| | | | 0.446) | | |
| CNTN2 | DPP6 (0.238- | GDNFR- | SCARF2 | | |
| | 0.384) | alpha-3 | (0.113- | | |
| | | (0.168-0.325) | 0.290) | | |
| GH2 | IgM (0.158) | | | | |
| IL 18 | IL-18BP | TNF-R2 | PD-L1 | | |
| | (0.338) | (0.390) | (0.334) | | |
| MMP-2 | Notch 3 | CNTN1 | | | |
| | (0.616) | (0.582) | | | |

TABLE 5A
Study codes for analyses of biomarkers

| Study | Study | | | Sample | |
|---|---|---|---|---|---|
| Code | Name | Endpoint | Type | Size | Additional Description |
| F1 | Serum | MS v. Normal and | Serum Pools | 4 | 47 individual samples were pooled to 4 serum pools representing MS |
| | Pools | other | | | subjects with shorter disease duration, MS subjects with longer |
| | | Inflammatory | | | disease duration, healthy controls, and rheumatoid arthritis subjects |
| | | Disease | | | (representing non-neurological inflammatory disease). |
| F2 | ACP | Exacerbation vs. | Cross | 125 | Protein profiles of 124 patient serum samples measured cross- |
| | | Quiescence | Sectional | | sectionally to classify whether patient was in the state of |
| | | | | | exacerbation versus quiescence, as confirmed by a clinician. |
| | | | | | Whereas the rest of the data in this analysis was accumulated from |
| | | | | | the CLIMB and EPIC cohorts, this data was assessed based on a |
| | | | | | cohort of patients from the Accelerated Cure Project. |
| F3A | Brigham | Relapse/Remission | Longitudinal | 60 | Longitudinal assessment of 30 paired sample sets in Clinical |
| | AIM2, | (clinically defined) | | | Relapse/Remission. Clinically defined Relapse/Remission status |
| | CLIMB | | | | was assessed by Physician's examination at the time of the blood |
| | cohort | | | | draw. |
| F3B | Brigham | GAD+ v. Non- | Longitudinal | 60 | Longitudinal assessment of 30 paired sample sets in Radiographic |
| | AIM3, | GAD+ | | | Relapse/Remission. Radiographic defined Relapse/Remission status |
| | CLIMB | | | | was assessed by presence of gadolinium-enhancing lesions on an |
| | cohort | | | | MRI administered within 30 days of blood draw. |
| F3C | Brigham | High annualized | Cross | 60 | Protein profiles of 60 patient serum samples measured cross- |
| | AIM1, | relapse rate (ARR) | Sectional | | sectionally to classify whether patient had a low (<0.2) ARR versus a |
| | CLIMB | V. Low ARR | | | high (>1.0) ARR. |
| | cohort | | | | |
| Normals | ACP | MS v. Normal: | MS | 78 | |
| | Normal and | Age, Sex, BMI | (Longitudinal) | | |
| | MS | and MS Remission | Normals | | |
| | longitudinal | → Remission | (Cross | | |
| | | | sectional) | | |
| F4 | Brigham | GAD+ v. Non- | Longitudinal | 268 | Longitudinal: Protein profiles of 146 MS serum samples (73 pairs) |
| | AIM3 | GAD+ (enhance | and Cross | | where 1 time point corresponded to a patient with 0 Gd lesions in |
| | expansion | high disease | Sectional | | time-matched MRI, and the other time point corresponded to the |
| | | activity) | | | same patient with at least 1 Gd lesion in time-matched MRI. The |
| | | | | | objective was to classify if a patient had changed from 0 to at least 1 |
| | | | | | lesion or vice versa. |
| | | | | | Cross-Sectional: Protein profiles of 326 MS serum samples measured |
| | | | | | cross-sectionally to classify whether patient had 0 Gd lesions on MRI |
| | | | | | versus at least 1 Gd lesion on MRI. Analysis of the F4 samples |
| | | | | | included reanalysis of the AIM3 samples (same endpoint and study). |
| | | | | | After exclusions this resulted in n=326. |
| F5 | UCSF | GAD+ v. Non- | Cross | 180 | Protein profiles of subset of 180 patient serum samples across the F5 |
| | EPIC | GAD+ | Sectional | | EPIC study measured cross-sectionally to classify whether patient |
| | Cohort | | | | had 0 Gd lesions on MRI versus at least 1 Gd lesion on MRI. |
| F6 | Univ. of | Primary: GAD+ v. | Longitudinal | 205 | Study investigating MS-specific blood biomarker signatures relevant |
| | Basel - | Non-GAD+ | | | to clinical & neuroimaging outcomes in samples from the University |
| | AIM A | Secondary: EDSS, | | | Hospital Basel Cohort (UHBC). |
| | SMSC | T2 volume, ARR | | | |
| | Cohort | and clinically | | | |
| | | defined relapse | | | |
| | | status | | | |

TABLE 5B
Additional Descriptors of Studies (e.g., combined studies)

| Study Code | Description for classification |
|---|---|
| F4 Paired - | Protein profiles of 146 MS serum samples (73 pairs) where 1 time point corresponded to a patient with 0 Gd lesions in time- |
| Subtle DA | matched MRI, and the other time point corresponded to the same patient with exactly 1 Gd lesion in time-matched MRI. The |
| | objective was to classify if a patient had changed from 0 to 1 lesion or vice versa. |
| F4 Paired - | Protein profiles of 196 MS serum samples (98 pairs) where 1 time point corresponded to a patient with 0 Gd lesions in time- |
| General DA | matched MRI, and the other time point corresponded to the same patient with at least 1 Gd lesion in time-matched MRI. The |
| | objective was to classify if a patient had changed from 0 to positive lesions or vice versa. |
| F4 + F5 (Gd | Protein profiles of subset of 506 patient serum samples measured cross-sectionally to classify whether patient had 0 Gd lesions |
| presence) - | on MRI versus exactly 1 Gd lesion on MRI. This is a proxy for subtle disease activity, as measured radiographically. |
| Subtle DA | |
| F4 + F5 (Gd | Protein profiles of 506 patient serum samples (across the EPIC and CLIMB cohorts) measured cross-sectionally to classify |
| presence) - | whether patient had 0 Gd lesions on MRI versus any Gd lesions on MRI. This is a proxy for general disease activity, as measured |
| General DA | radiographically. |
| F4 + F5 (Gd | Protein profiles of subset of 506 patient serum samples measured cross-sectionally to classify whether patient had 0 Gd lesions |
| presence) - | on MRI versus at least 3 Gd lesion on MRI. This is a proxy for extreme radiographic disease activity. |
| Extreme DA | |
| AIM1 | Protein profiles of 60 patient serum samples measured cross-sectionally to classify whether patient had a low (<0.2) ARR versus |
| (annualized | a high (>1.0) ARR. |
| relapse rate) | |
| AIM3 -Paired | Protein profiles of 58 MS serum samples (98 pairs) where 1 time point corresponded to a patient with 0 Gd lesions in time- |
| | matched MRI, and the other time point corresponded to the same patient with at least 1 Gd lesion in time-matched MRI. The |
| | objective was to classify if a patient had changed from 0 to positive lesions or vice versa. |
| ACP | Protein profiles of 124 patient serum samples measured cross-sectionally to classify whether patient was in the state of |
| (exacerbation | exacerbation versus quiescence, as confirmed by a clinician. Whereas the rest of the data in this analysis was accumulated from |
| vs. quiescence) | the CLIMB and EPIC cohorts, this data was assessed based on a cohort of patients from the Accelerated Cure Project. |

TABLE 6
Additional biomarkers for use in predicting multiple
sclerosis disease activity.

| | Biomarker | Accession Number |
|---|---|---|
| Biomarker Name | Symbol | (Uniprot Database) |
| Cell Adhesion Molecule 3 | CADM3 | Q8N126 |
| Kallikrein Related Peptidase 6 | KLK6 | Q92876 |
| Brevican | BCAN | Q96GW7 |
| Oligodendrocyte Myelin | OMG | P23515 |
| Glycoprotein | | |
| CD5 molecule | CD5 | P06127 |
| Cytotoxic and Regulatory T Cell | CRTAM | O95727 |
| Molecule | | |
| CD244 Molecule | CD244 | Q9BZW8 |
| Tumor Necrosis Factor Receptor | TNFRSF9 | Q07011 |
| Superfamily Member 9 | | |
| Proteinase 3 | PRTN3 | P24158 |
| Follistatin Like 3 | FSTL3 | O95633 |
| C—X—C Motif Chemokine Ligand 10 | CXCL10 | P02778 |
| C—X—C Motif Chemokine Ligand 11 | CXCL11 | O14625 |
| Interleukin 18 Binding Protein | IL-18BP | O95998 |
| Macrophage Scavenger Receptor 1 | MSR1 | P21757 |
| C-C Motif Chemokine Ligand 3 | CCL3 | P10147 |
| Tumor Necrosis Factor Ligand | TWEAK | 043508 |
| Superfamily Member 12 | | |
| Trefoil Factor 3 | TFF3 | Q07654 |
| Matrix Metallopeptidase 9 | MMP-9 | P14780 |
| Insulin Like Growth Factor Binding | IGFBP-1 | P08833 |
| Protein 1 | | |
| Interleukin 12A | IL12A | P29459 |
| Seizure Related 6 Homolog Like | SEZ6L | Q9BYH1 |
| Dipeptidy1 Peptidase Like 6 | DPP6 | P42658 |
| Neurocan | NCAN | O14594 |
| Tubulointerstitial Nephritis Antigen | TINAGLI | Q9GZM7 |
| Like 1 | | |
| Calcium Activated Nucleotidase 1 | CANT1 | Q8WVQ1 |
| Nectin Cell Adhesion Molecule 2 | NECTIN2 | Q92692 |
| Neural Proliferation, | NPDC1 | Q9NQX5 |
| Differentiation and Control Protein 1 | | |
| Tumor Necrosis Factor Receptor | TNFRSF11A | Q9Y6Q6 |
| Superfamily Member 11A | | |
| Contactin 4 | CNTN4 | Q8IWV2 |
| Neutrophic Receptor Tyrosine | NTRK2 | Q16620 |
| Kinase 2 | | |
| Neutrophic Receptor Tyrosine | NTRK3 | Q16288 |
| Kinase 3 | | |
| Cadherin 6 | CDH6 | P55285 |
| Carcinoembryonic Antigen Related | CEACAM8 | P31997 |
| Cell Adhesion Molecule 8 | | |
| Mitotic Arrest Deficient 1 Like 1 | MAD1L1 | Q9Y6D9 |
| Fc Fragment of IgA Receptor | FCAR | P24071 |
| Myeloperoxidase | MPO | P05164 |
| Osteomodulin | OMD | Q99983 |
| Matrix Extracellular | MEPE | Q9NQ76 |
| Phosphoglycoprotein | | |
| GDNF Family Receptor Alpha 3 | GDNFR- | Q60609 |
| | alpha-3 | |
| Scavenger Receptor Class F | SCARF2 | Q96GP6 |
| Member 2 | | |
| CD40 Ligand | IgM | P29965 |
| Tumor Necrosis Factor Receptor | TNF-R2 | P20333 |
| Superfamily Member 1B | | |
| Programmed Cell Death 1 Ligand | PD-L1 | Q9NZQ7 |
| Notch 3 | NOTCH3 | Q9UM47 |
| Contactin 1 | CNTN1 | Q12860 |
| Oncostatin M | OSM | P13725 |
| Transforming Growth Factor Alpha | TGF-a | P01135 |
| Peptidoglycan Recognition Protein 1 | PGLYRP1 | O75594 |
| Nitric Oxide Synthase 3 | NOS3 | P29474 |
| Discoidin Domain Receptor | DDR1 | Q08345 |
| Tyrosine Kinase 1 | | |
| C—X—C Motif Chemokine Ligand 16 | CXCL16 | Q9H2A7 |
| Interleukin 18 | IL-18 | Q14116 |
| Interleukin 6 | IL-6 | PO5231 |
| CD166 antigen | ALCAM | Q13740 |
| Spondin-2 | SPON2 | Q9BUD6 |
| Protocadherin-17 | PCDH17 | O14917 |

TABLE 7
Biomarker categorizations

| | | Pathways, Cell Types, |
|---|---|---|
| Biomarkers | Category | Summary |
| NEFL | Neurodegeneration, Neuroaxonal | Neurodegeneration |
| | Integrity | |
| APLP1 | Neurodegeneration, Neuroaxonal | Neuronal damage, synaptic |
| | Integrity, Myelination | dysfunction |
| OPG | Neurodegeneration, Neuroaxonal | Cell apoptosis |
| | Integrity | |
| SERPINA9 | Neurodegeneration, Neuroaxonal | Neuronal damage |
| | Integrity | |
| PRTG | Neurodegeneration, Neuroaxonal | Neurotrophin binding |
| | Integrity | |
| GFAP | Neurodegeneration, Myelin | Distinguishes astrocytes from |
| | Integrity, Neuroaxonal Integrity, | other glial cells |
| | Cerebrovascular Function | |
| CNTN2 | Neurodegeneration, Neuroaxonal | Axon-dendritic rearrangement |
| | Integrity | |
| FLRT2 | Neurodegeneration, Neurite | Glutamate excitotoxicity, |
| | Outgrowth & Neurogenesis, | neuronal cell death, synaptic |
| | Neuroaxonal Integrity | formation & plasticity |
| CCL20 | Inflammation, Neuroinflammation | Acute inflammatory response, |
| | | cytokine |
| GH | Inflammation, Neuroinflammation | Neuroendocrine marker, energy |
| | | homeostasis, metabolism |
| CXCL13 | Inflammation, Neuroinflammation, | Inflammatory response |
| | Immune modulation | |
| IL-12B | Inflammation, Neuroinflammation, | Growth factor for activated T |
| | Immune modulation | and NK cells |
| TNFRSF10A | Inflammation, Neuroinflammation, | Neurodegeneration |
| | Neurodegeneration, Neuroaxonal | |
| | Integrity | |
| TNFSF13B | Inflammation, Neuroinflammation, | T cell-independent B cell |
| (BAFF) | Immune Modulation | activation |
| CD6 | Inflammation, Neuroinflammation, | Blood brain barrier breach, cell |
| | Immune Modulation, | adhesion molecule, t-cell |
| | Cerebrovascular Function | mediated acute inflammatory |
| | | response |
| CXCL9 | Inflammation, Neuroinflammation, | Cytokine that affects the growth, |
| | Immune Modulation | movement, or activation state of |
| | | cells that participate in immune |
| | | and inflammatory response |
| VCAN | Inflammation, Myelin Integrity, | Myelin protection |
| | Myelination, Cerebrovascular | |
| | Function | |
| COL4A1 | Myelin Integrity, Myelination, | Blood brain barrier breach, |
| | Neurodegeneration, Neurite | collagen network, focal adhesion |
| | Outgrowth & Neurogenesis, | |
| | Cerebrovascular Function | |
| MOG | Myelin Integrity, Myelination, | Homophilic cell-cell adhesion |
| | Immune modulation | |
| CDCP1 | Immune modulation | Cell adhesion and cell matrix |
| | | association |
| OPN | Immune Modulation, Neuroaxonal | Biomineralization |
| | Integrity, Myelination | |
| CDH6 | Cell Regulation, Cell Adhesion | Calcium-dependent cell adhesion |
| | | proteins |
| CADM3 | Cell Regulation | Cell junction organization |
| DDR1 | Cell Regulation | Regulates cell attachment to the |
| | | extracellular matrix, remodeling |
| | | of the extracellular matrix, cell |
| | | migration, differentiation, |
| | | survival and cell proliferation |
| DPP6 | Cell Regulation | Potassium channel regulator |
| | | activity |
| IGFBP-1 | Cell Regulation | Inhibit or stimulate the growth |
| | | promoting effects of the IGFs |
| MEPE | Cell Regulation | Renal phosphate excretion and |
| | | inhibits intestinal phosphate |
| | | absorption |
| NOS3 | Cell Regulation | Vascular smooth muscle |
| | | relaxation |
| Notch 3 | Cell Regulation | Regulate cell-fate determination |
| OMD | Cell Regulation | Biomineralization |
| SCARF2 | Cell Regulation | Adhesion protein |
| TINAGLI | Cell Regulation | Endocytosis |
| TNFRSF11A | Cell Regulation | Osteoclastogenesis |
| TFF3 | Gut-Brain Axis | Expressed in gastrointestinal |
| | | mucosa |
| CD244 | Immune Modulation | Modulating the activation and |
| | | differentiation of a wide variety |
| | | of immune cells and thus are |
| | | involved in the regulation and |
| | | interconnection of both innate |
| | | and adaptive immune response |
| CD5 | Immune Modulation | Receptor in regulating T-cell |
| | | proliferation |
| CRTAM | Immune Modulation | Heterophilic cell-cell adhesion |
| | | which regulates the activation, |
| | | differentiation and tissue |
| | | retention of various T-cell |
| | | subsets |
| CXCL11 | Immune Modulation | Important role in CNS diseases |
| | | which involve T-cell recruitment |
| CXCL16 | Immune Modulation | Scavenger receptor on |
| | | macrophages |
| NECTIN2 | Immune Modulation | Modulator of T-cell signaling |
| PD-L1 | Immune Modulation | Implicated in autoimmunity |
| TNFRSF9 | Immune Modulation | Expressed by activated T cells |
| CCL3 | Inflammation | Inflammatory and chemokinetic |
| | | properties |
| GH2 | Inflammation | Neuroendocrine marker, energy |
| | | homeostasis, metabolism |
| IFI30 (GILT) | Inflammation | Antigen processing through |
| | | MHC class II and up-regulated |
| | | expression on macrophages and |
| | | microglia in active |
| | | demyelinating lesions in multiple |
| | | sclerosis. GILT is expressed |
| | | constitutively in antigen |
| | | presenting cells and is induced |
| | | by inflammatory cytokines, |
| IL-18 | Inflammation | Inhibitor of the early TH1 |
| | | cytokine response |
| IL-18BP | Inflammation | Inhibitor of the early TH1 |
| | | cytokine response |
| IL12A | Inflammation | Cytokine that can act as a growth |
| | | factor for activated T and NK |
| | | cells |
| MMP-2 | Inflammation | Remodeling of the vasculature, |
| | | angiogenesis, tissue repair, tumor |
| | | invasion, inflammation, and |
| | | atherosclerotic plaque rupture |
| MMP-9 | Inflammation | Local proteolysis of the |
| | | extracellular matrix and in |
| | | leukocyte migration |
| TWEAK | Inflammation, Cell Regulation | Cytokine |
| CXCL10 | Inflammation, Immune Modulation | Pro-inflammatory cytokine |
| IL6 | Inflammation, Immune Modulation | Cytokine |
| YkL40 | Inflammation, Immune Modulation | Inflammation |
| (CHI3L1) | | |
| ENPP2 | Metabolism | Hydrolyzes lysophospholipids |
| MSR1 | Metabolism | Pathologic deposition of |
| | | cholesterol in arterial walls |
| | | during atherogenesis |
| CANT1 | Metabolism | Metabolism of nucleotides |
| OMG | Immune Modulation, Myelin | Myelin |
| | Integrity | |
| ALCAM | Neurite Outgrowth & Neurogenesis | Axon guidance |
| BCAN | Neurite Outgrowth & Neurogenesis | Terminally differentiating and |
| | | the adult nervous system during |
| | | postnatal development |
| CNTN4 | Neurite Outgrowth & Neurogenesis | Cell surface interactions during |
| | | nervous system development |
| KLK6 | Neurite Outgrowth & Neurogenesis | Activity against amyloid |
| | | precursor protein, myelin basic |
| | | protein, gelatin, casein and |
| | | extracellular matrix proteins such |
| | | as fibronectin, laminin, |
| | | vitronectin and collagen |
| NCAN | Neurite Outgrowth & Neurogenesis | Neuronal adhesion and neurite |
| | | growth during development by |
| | | binding to neural cell adhesion |
| | | molecules |
| NTRK2 | Neurite Outgrowth & Neurogenesis | Development and the maturation |
| | | of the central and the peripheral |
| | | nervous systems through |
| | | regulation of neuron survival, |
| | | proliferation, migration, |
| | | differentiation, and synapse |
| | | formation and plasticity |
| SPON2 | Neurite Outgrowth & Neurogenesis | Outgrowth of hippocampal |
| | | embryonic neurons |
| GDNFR-alpha- | Neuroregulatory | Glial cell line-derived |
| 3 | | neurotrophic factor |
| NPDC1 | Neuroregulatory | Suppresses oncogenic |
| | | transformation in neural and non- |
| | | neural cells and down-regulates |
| | | neural cell proliferation |
| NTRK3 | Neuroregulatory | Involved in nervous system and |
| | | probably heart development |
| PCDH17 | Neuroregulatory | Establishment and function of |
| | | specific cell-cell connections in |
| | | the brain |
| SEZ6L | Neuroregulatory | Endoplasmic reticulum functions |
| | | in neurons |
| TNF-R2 | Neuroregulatory | Potentiate TNF-induced |
| | | apoptosis |

TABLE 8
Biomarker involvement in particular locations
(brain, brain barrier, blood).
Numerical indicators are on a scale of 1-5, with 1 indicating
that corresponding biomarker is minimally found in
the location whereas 5 indicating that corresponding
biomarker is heavily found in the location.

| | Location | | |
|---|---|---|---|
| Biomarker | Brain | Brain Barrier | Blood |
| TNFSF13B | 1 | | 5 |
| CXCL13 | | | 1 |
| GFAP | 5 | | 1 |
| IL12B | | | 2 |
| MOG | 4 | | |
| NFL | 4 | | 2 |
| OPG | | | 2 |
| OPN | 4 | | 1 |
| TNFRSF10A | | | 2 |
| APLP1 | 4 | | |
| CCL20 | | | 2 |
| CD6 | | | 3 |
| CDCP1 | | | 2 |
| CNTN2 | 4 | | |
| COL4A1 | 1 | 4 | |
| CXCL9 | | 4 | 1 |
| FLRT2 | 2 | 3 | |
| GH | | | 1 |
| PRTG | 1 | 1 | |
| SERPINA9 | | | 3 |
| VCAN | 2 | | 4 |

TABLE 9A
Biomarker involvement in particular cell types. Numerical indicators are on a scale of 1-4, with 1 indicating that corresponding
biomarker is minimally found in the cell type whereas 4 indicating that corresponding biomarker is heavily found in the cell type.

| | Cell Type | | | | | | | |
|---|---|---|---|---|---|---|---|---|
| | Neuronal | | | | | | Endothelial | |
| Biomarker | Cells | OPCs | Oligodendrocytes | Astrocytes | Microglia | Pericytes | Cells | VLMCs |
| TNFSF13B | 1 | | | | | | | |
| CXCL13 | | | | | | | | |
| GFAP | | | | 4 | | | | |
| IL12B | | | | | | | | |
| MOG | | | 4 | | | | | |
| NFL | 4 | | | | | | | |
| OPG | | | | | | | | |
| OPN | 3 | | 4 | | 3 | | | |
| TNFRSF10A | | | | | | | | |
| APLP1 | 3 | | 4 | | | | | |
| CCL20 | | | | | | | | |
| CD6 | | | | | | | | |
| CDCP1 | | | | | | | | |
| CNTN2 | 3 | | 4 | | | | | |
| COL4A1 | 2 | | | | | 3 | | 3 |
| CXCL9 | | | | | | | 3 | |
| FLRT2 | 3 | 2 | | 2 | | | | 4 |
| GH | | | | | | | | |
| PRTG | 2 | | | 2 | | | 2 | |
| SERPINA9 | | | | | | | | |
| VCAN | 2 | 4 | | 3 | | | | 3 |

TABLE 9B
Biomarker involvement in particular cell types. Numerical indicators are on a scale
of 1-4, with 1 indicating that corresponding biomarker is minimally found in the cell
type whereas 4 indicating that corresponding biomarker is heavily found in the cell type.

| | Cell Type | | | | | |
|---|---|---|---|---|---|---|
| | T | B | NK | | Dendritic | |
| Biomarker | Cells | Cells | Cells | Granulocytes | Cells | Monocytes |
| TNFSF13B | 1 | | | 4 | 2 | 2 |
| CXCL13 | 1 | | | | | |
| GFAP | 1 | 1 | 1 | 1 | | |
| IL12B | | | | 2 | | |
| MOG | | | | | | |
| NFL | 2 | | | | | |
| OPG | | | | | 2 | |
| OPN | | | | 1 | | |
| TNFRSF10A | 2 | 2 | 1 | 1 | 1 | 1 |
| APLP1 | | | | | | |
| CCL20 | 2 | 2 | | | | |
| CD6 | 3 | | | | | |
| CDCP1 | | | 1 | | 1 | |
| CNTN2 | | | | | | |
| COL4A1 | | | | | | |
| CXCL9 | | | | | | 1 |
| FLRT2 | | | | | | |
| GH | 1 | 1 | | | | |
| PRTG | | | | | | |
| SERPINA9 | | | | | | 2 |
| VCAN | 2 | | | | 3 | 4 |

TABLE 10A
Statistical measures of univariate APLP1 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.509 | 0.530 | 0.668 | 0.549 | 0.543 | 0.659 | 0.567 |
| P-value | 0.426 | 0.479 | 0.041 | 0.067 | 0.243 | 4.10E−4 | 0.006 |
| R-squared | NA | NA | 0.069 | 0.005 | 0.001 | 0.002 | 2.80E−04 |

TABLE 10B
Statistical measures of univariate CCL20 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.612 | 0.526 | 0.613 | 0.620 | 0.660 | 0.653 | 0.522 |
| P-value | 0.028 | 0.212 | 0.053 | 2.60E−4 | 0.004 | 0.002 | 0.131 |
| R-squared | NA | NA | 0.061 | 0.044 | 0.094 | 0.003 | 0.003 |

TABLE 10C
Statistical measures of univariate CD6 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.502 | 0.760 | 0.581 | 0.559 | 0.675 | 0.663 | 0.507 |
| P-value | 0.459 | 2.00E−4 | 0.218 | 0.061 | 0.001 | 2.91E−4 | 0.394 |
| R-squared | NA | NA | 0.057 | 0.005 | 0.140 | 4.45E−4 | 0.003 |

TABLE 10D
Statistical measures of univariate CDPCP1 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.634 | 0.563 | 0.513 | 0.622 | 0.607 | 0.545 | 0.528 |
| P-value | 0.008 | 0.130 | 0.281 | 1.40E−4 | 0.012 | 0.253 | 0.086 |
| R-squared | NA | NA | 0.036 | 4.13E−2 | 8.82E−2 | 0.002 | 0.004 |

TABLE 10E
Statistical measures of univariate CNTN2 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.505 | 0.645 | 0.622 | 0.533 | 0.590 | 0.537 | 0.501 |
| P-value | 0.416 | 0.020 | 0.247 | 0.131 | 0.061 | 0.326 | 0.266 |
| R-squared | NA | NA | 0.035 | 5.40E−3 | 4.39E−2 | 0.002 | 0.001 |

TABLE 10F
Statistical measures of univariate COL4A1 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.565 | 0.679 | 0.544 | 0.523 | 0.628 | 0.505 | 0.516 |
| P-value | 0.137 | 0.002 | 0.196 | 0.187 | 0.008 | 0.259 | 0.269 |
| R-squared | NA | NA | 0.001 | 2.84E−3 | 6.95E−3 | 2.61E−4 | 0.002 |

TABLE 10G
Statistical measures of univariate CXCL9 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.508 | 0.591 | 0.763 | 0.627 | 0.651 | 0.595 | 0.526 |
| P-value | 0.186 | 0.123 | 0.018 | 2.50E−4 | 0.003 | 0.019 | 0.182 |
| R-squared | NA | NA | 0.349 | 0.003 | 0.005 | 4.56E−5 | 0.001 |

TABLE 10H
Statistical measures of univariate FLRT2 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.633 | 0.668 | 0.528 | 0.506 | 0.550 | 0.744 | 0.569 |
| P-value | 0.003 | 0.004 | 0.356 | 0.495 | 0.269 | 4.00E−7 | 0.047 |
| R-squared | NA | NA | 0.051 | 0.001 | 8.24E−3 | 0.008 | 2.22E−4 |

TABLE 10I
Statistical measures of univariate GH biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.649 | 0.602 | 0.594 | 0.535 | 0.532 | 0.578 | 0.507 |
| P-value | 0.002 | 0.052 | 0.335 | 0.152 | 0.294 | 0.076 | 0.342 |
| R-squared | NA | NA | 0.001 | 0.002 | 2.46E−3 | 0.014 | 0.004 |

TABLE 10J
Statistical measures of univariate IFI30 biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.520 | 0.507 | 0.600 | 0.618 | 0.550 | 0.658 | 0.519 |
| P-value | 0.141 | 0.493 | 0.206 | 0.001 | 0.197 | 0.002 | 0.247 |
| R-squared | NA | NA | 0.028 | 0.017 | 0.000 | 3.56E−04 | 0.002 |

TABLE 10K
Statistical measures of univariate MOG biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.530 | 0.636 | 0.572 | 0.589 | 0.604 | 0.738 | 0.620 |
| P-value | 0.300 | 0.036 | 0.090 | 0.005 | 0.080 | 4.61E−07 | 1.45E−04 |
| R-squared | NA | NA | 0.056 | 0.031 | 0.062 | 0.048 | 0.022 |

TABLE 10L
Statistical measures of univariate NEFL biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.717 | 0.611 | 0.876 | 0.746 | 0.859 | 0.834 | 0.759 |
| P-value | 2.51E−05 | 0.0316 | 1.80E−04 | 6.06E−15 | 4.84E−09 | 7.46E−12 | 1.47E−16 |
| R-squared | NA | NA | 0.415 | 0.189 | 0.321 | 0.213 | 0.147 |

TABLE 10M
Statistical measures of univariate OPG biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.509 | 0.517 | 0.531 | 0.604 | 0.638 | 0.632 | 0.514 |
| P-value | 0.494 | 0.373 | 0.432 | 0.005 | 0.012 | 0.004 | 0.269 |
| R-squared | NA | NA | 0.001 | 0.012 | 0.072 | 0.001 | 0.001 |

TABLE 10N
Statistical measures of univariate OPN biomarker analysis.

| | | | AIM3 | F4 | F4 | | F4 + F5 (Gd. |
|---|---|---|---|---|---|---|---|
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.509 | 0.500 | 0.512 | 0.549 | 0.598 | 0.603 | 0.557 |
| P-value | 0.446 | 0.454 | 0.149 | 0.082 | 0.060 | 0.018 | 0.012 |
| R-squared | NA | NA | 2.97E−05 | 3.84E−03 | 0.003 | 0.001 | 0.003 |

TABLE 10O
Statistical measures of univariate PRTG biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.619 | 0.664 | 0.617 | 0.542 | 0.628 | 0.630 | 0.512 |
| P-value | 0.021 | 0.031 | 0.102 | 0.079 | 0.013 | 0.006 | 0.487 |
| R-squared | NA | NA | 0.099 | 0.002 | 0.032 | 0.005 | 0.001 |

TABLE 10P
Statistical measures of univariate SERPINA9 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.603 | 0.689 | 0.667 | 0.558 | 0.608 | 0.535 | 0.525 |
| P-value | 0.002 | 0.031 | 0.059 | 0.013 | 0.115 | 0.180 | 0.079 |
| R-squared | NA | NA | 0.082 | 0.006 | 0.075 | 0.002 | 0.002 |

TABLE 10Q
Statistical measures of univariate TNFRSF10A biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.587 | 0.542 | 0.688 | 0.582 | 0.667 | 0.545 | 0.545 |
| P-value | 0.036 | 0.305 | 0.058 | 0.006 | 0.002 | 0.296 | 0.024 |
| R-squared | NA | NA | 0.040 | 0.001 | 0.033 | 0.002 | 4.42E−04 |

TABLE 10R
Statistical measures of univariate VCAN biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.511 | 0.562 | 0.637 | 0.549 | 0.623 | 0.658 | 0.567 |
| P-value | 0.421 | 0.085 | 0.027 | 0.061 | 0.034 | 0.002 | 0.014 |
| R-squared | NA | NA | 0.055 | 0.001 | 0.006 | 0.003 | 3.63E−04 |

TABLE 10S
Statistical measures of univariate CHI3L1 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.501 | 0.641 | 0.606 | 0.575 | 0.564 | 0.528 | 0.533 |
| P-value | 0.409 | 0.029 | 0.091 | 0.027 | 0.131 | 0.324 | 0.117 |
| R-squared | NA | NA | 0.083 | 0.005 | 0.008 | 0.001 | 0.001 |

TABLE 10T
Statistical measures of univariate CXCL13 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.506 | 0.502 | 0.539 | 0.527 | 0.511 | 0.656 | 0.538 |
| P-value | 0.311 | 0.496 | 0.486 | 0.245 | 0.234 | 0.002 | 0.215 |
| R-squared | NA | NA | 0.002 | 1.82E−04 | 2.24E−03 | 0.035 | 0.004 |

TABLE 10U
Statistical measures of univariate GH2 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.582 | 0.502 | 0.694 | 0.560 | 0.703 | 0.521 | 0.530 |
| P-value | 0.018 | 0.469 | 0.087 | 0.093 | 0.003 | 0.239 | 0.165 |
| R-squared | NA | NA | 0.166 | 0.011 | 0.061 | 0.023 | 0.011 |

TABLE 10V
Statistical measures of univariate IL-12B biomarker analysis.

| | | | | | | | F4 + F5 |
|---|---|---|---|---|---|---|---|
| | | | AIM3 | F4 | | | (Gd. |
| | ACP | AIM1 | Paired | Unpaired | F4 Paired | F5 EPIC | Endpoint) |
| Study | (n = 124) | (n = 60) | (n = 58) | (n = 326) | (n = 196) | (n = 180) | (n = 506) |
| AUC | 0.506 | 0.548 | 0.519 | 0.526 | 0.607 | 0.562 | 0.506 |
| P-value | 0.495 | 0.281 | 0.389 | 0.047 | 0.021 | 0.113 | 0.208 |
| R-squared | NA | NA | 0.024 | 0.048 | 0.168 | 1.75E−04 | 0.008 |

TABLE 10W
Statistical measures of univariate IL18 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.643 | 0.592 | 0.519 | 0.569 | 0.616 | 0.548 | 0.520 |
| P-value | 0.004 | 0.077 | 0.219 | 0.014 | 0.038 | 0.168 | 0.237 |
| R-squared | NA | NA | 0.041 | 0.020 | 0.068 | 2.70E−06 | 0.001 |

TABLE 10X
Statistical measures of univariate MMP-2 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.561 | 0.617 | 0.547 | 0.504 | 0.513 | 0.702 | 0.564 |
| P-value | 0.087 | 0.035 | 0.412 | 0.319 | 0.399 | 3.21E−05 | 0.025 |
| R-squared | NA | NA | 0.036 | 1.07E−04 | 4.16E−04 | 0.004 | 0.002 |

TABLE 10Y
Statistical measures of univariate MMP-9 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.503 | 0.623 | 0.618 | 0.508 | 0.561 | 0.721 | 0.550 |
| P-value | 0.461 | 0.027 | 0.043 | 0.434 | 0.083 | 9.30E−07 | 0.034 |
| R-squared | NA | NA | 0.087 | 0.004 | 0.037 | 0.007 | 0.001 |

TABLE 10Z
Statistical measures of univariate VCAM-1 biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.637 | 0.556 | 0.600 | 0.524 | 0.589 | 0.544 | 0.546 |
| P-value | 0.001 | 0.208 | 0.261 | 0.257 | 0.106 | 1.23E−01 | 0.064 |
| R- | NA | NA | 0.063 | 9.04E−05 | 0.001 | 1.20E−05 | 0.001 |
| squared | | | | | | | |

TABLE 10AA
Statistical measures of univariate TNFSF13B biomarker analysis.

| | | | | | | | F4 + F5 |
|---|---|---|---|---|---|---|---|
| | | | AIM3 | F4 | F4 | | (Gd. |
| | ACP | AIM1 | Paired | Unpaired | Paired | F5 EPIC | Endpoint) |
| Study | (n = 124) | (n = 60) | (n = 58) | (n = 326) | (n = 196) | (n = 180) | (n = 506) |
| AUC | 0.587 | 0.504 | 0.618 | 0.524 | 0.582 | 0.599 | 0.516 |
| P-value | 0.131 | 0.426 | 0.126 | 0.106 | 0.086 | 0.051 | 0.429 |
| R- | NA | NA | 0.149 | 0.05 | 0.075 | 0.006 | 7.98E−03 |
| squared | | | | | | | |

TABLE 10BB
Statistical measures of univariate GFAP biomarker analysis.

| | | | | | | | F4 + F5 |
|---|---|---|---|---|---|---|---|
| | | | AIM3 | F4 | | | (Gd. |
| | ACP | AIM1 | Paired | Unpaired | F4 Paired | F5 EPIC | Endpoint) |
| Study | (n = 124) | (n = 60) | (n = 58) | (n = 326) | (n = 196) | (n = 180) | (n = 506) |
| AUC | NA | NA | NA | 0.558 | 0.541 | 0.646 | 0.633 |
| P-value | NA | NA | NA | 0.069 | 0.073 | 0.112 | 0.044 |
| R-squared | NA | NA | NA | 0.025 | 0.003 | 0.039 | 0.008 |

TABLE 11
Univariate biomarker analysis results for various MS disease activity endpoints

| | Gad | | | | T2- |
|---|---|---|---|---|---|
| | presence p- | | Relapse | ARR | weighted |
| Protein | value | EDSS | p-value | p-value | Volume |
| Biomarker | (n = 155) | R2 (n = 205) | (n = 205) | (n = 144) | R2 (n = 128) |
| APLP1 | 0.228 | 3.40E−04 | 0.084 | 0.937 | 0.012 |
| CCL20 | 0.326 | 0.038 | 0.107 | 0.609 | 0.038 |
| CD6 | 0.012 | 0.001 | 0.463 | 0.511 | 0.001 |
| CDCP1 | 0.010 | 0.087 | 0.306 | 0.046 | 2.22E−03 |
| CNTN2 | 0.050 | 0.006 | 0.514 | 0.141 | 0.022 |
| COL4A1 | 0.248 | 0.006 | 0.250 | 0.208 | 0.007 |
| CXCL13 | 0.699 | 0.008 | 0.093 | 0.750 | 4.36E−04 |
| CXCL9 | 0.010 | 0.017 | 0.028 | 0.001 | 0.026 |
| FLRT2 | 0.292 | 0.003 | 0.435 | 0.825 | 0.007 |
| GFAP | 0.582 | 0.161 | 0.221 | 0.453 | 0.122 |
| GH | 0.197 | 1.30E−03 | 0.665 | 0.381 | 0.019 |
| IL-12B | 0.007 | 0.007 | 0.068 | 0.238 | 0.014 |
| MOG | 0.646 | 4.03E−04 | 0.062 | 0.599 | 0.025 |
| NEFL | 0.001 | 0.090 | 2.50E−05 | 0.042 | 0.125 |
| OPG | 0.652 | 0.152 | 0.612 | 0.634 | 0.044 |
| OPN | 0.178 | 0.046 | 0.086 | 0.436 | 0.044 |
| PRTG | 0.236 | 0.004 | 0.400 | 0.272 | 2.22E−03 |
| SERPINA9 | 0.081 | 0.010 | 0.936 | 0.076 | 8.75E−04 |
| TNFRSF10A | 0.348 | 0.033 | 0.984 | 0.012 | 0.016 |
| TNFSF13B | 7.63E−05 | 0.038 | 0.335 | 0.816 | 0.018 |
| VCAN | 0.198 | 0.043 | 0.045 | 0.911 | 0.018 |

TABLE 12
Gadolinium-based classification prediction
Gd+ Classification Comparison

| | Subtle | General | Extreme | |
|---|---|---|---|---|
| | (AUROC) | (AUROC) | (AUROC) | Regression (R²) |
| Univariate NFL | 0.697 ± 0.085 | 0.791 ± 0.046 | 0.890 ± 0.037 | 0.251 ± 0.020 |
| Multivariate Model | 0.732 ± 0.079 | 0.821 ± 0.037 | 0.914 ± 0.052 | 0.279 ± 0.022 |
| (best features) | | | | |
| Multivariate Model | 0.701 ± 0.055 | 0.645 ± 0.075 | 0.734 ± 0.130 | 0.071 ± 0.018 |
| (without NFL) | | | | |

TABLE 13
Optimization metrics for individual biomarkers

| Biomarker | EDSS R² | T2-weighted Volume R² |
|---|---|---|
| APLP1 | 0.000227 | 0.037332 |
| CCL20 | 0.041031 | 0.091351 |
| CD6 | 0.004626 | 0.07696 |
| CDCP1 | 0.03931 | 0.001367 |
| CNTN2 | 0.001594 | 0.023916 |
| COL4A1 | 0.00246 | 0.011448 |
| CXCL13 | 0.02188 | 0.053104 |
| CXCL9 | 0.019516 | 0.113253 |
| FLRT2 | 0.00237 | 0.042253 |
| GFAP | 0.201107 | 0.176669 |
| GH | 0.001453 | 0.005613 |
| IL12B | 0.004874 | 0.000005 |
| MOG | 0.001258 | 0.016593 |
| NEFL | 0.054577 | 0.077404 |
| OPG | 0.204099 | 0.081336 |
| OPN | 0.052497 | 0.055554 |
| PRTG | 0.00736 | 0.001347 |
| SERPINA9 | 0.015369 | 0.000716 |
| TNFRSF10A | 0.038961 | 0.026022 |
| TNFSF13B | 0.046195 | 0.063893 |
| VCAN | 0.043875 | 0.030339 |

TABLE 14
Pearson coefficients for absolute and relative quantitation
for individual biomarkers

| Biomarker | AvN1 R² | N2VN1 R² |
|---|---|---|
| APLP1 | 0.616 | 0.618 |
| CCL20 | 0.950 | 0.951 |
| CD6 | 0.792 | 0.793 |
| CDCP1 | 0.847 | 0.525 |
| CNTN2 | 0.751 | 0.751 |
| COL4A1 | 0.537 | 0.538 |
| CXCL13 | 0.811 | 0.811 |
| CXCL9 | 0.915 | 0.916 |
| FLRT2 | 0.349 | 0.346 |
| GFAP | 0.999 | N/A |
| GH | 0.909 | 0.915 |
| IL12B | 0.786 | 0.788 |
| MOG | 0.802 | 0.802 |
| NEFL | 0.808 | 0.813 |
| OPG | 0.718 | 0.718 |
| OPN | 0.713 | 0.710 |
| PRTG | 0.479 | 0.478 |
| SERPINA9 | 0.885 | 0.892 |
| TNFRSF10A | 0.695 | 0.695 |
| TNFSF13B | 0.733 | 0.732 |
| VCAN | 0.398 | 0.398 |

TABLE 15A
Performance of biomarker panel incorporating Tiers A, B, and C biomarkers

| | | | | | | F4 + F5 (Gd | | ACP |
|---|---|---|---|---|---|---|---|---|
| Tiers | | | F4 Paired - | F4 + F5 (Gd | F4 + F5 (Gd | presence) - | AIM1 | (exacerbation |
| A + | | F4 Paired - | General | presence) - | presence) - | Extreme | (annualized | vs. |
| B + C | Study | Subtle DA | DA | Subtle DA | General DA | DA | relapse rate) | quiescence) |
| | AUC | 0.890 | 0.961 | 0.771 | 0.834 | 0.890 | 0.925 | 0.802 |
| | PPV | 0.810 | 0.890 | 0.687 | 0.867 | 0.765 | 0.895 | 0.846 |

TABLE 15B
Performance of biomarker panel incorporating Tiers A and B biomarkers

| | | | F4 | | | | | |
|---|---|---|---|---|---|---|---|---|
| | | | Baseline- | | | F4 + | | |
| | | F4 Baseline- | Normalized | F4 + | F4 + | F5 (Gd | | ACP |
| | | Normalized | (Gd pairs) - | F5 (Gd | F5 (Gd | presence) - | AIM1 | (exacerbation |
| Tiers | | (Gd pairs) - | General | presence) - | presence) - | Extreme | (annualized | VS. |
| A + B | Study | Subtle DA | DA | Subtle DA | General DA | DA | relapse rate) | quiescence) |
| | AUC | 0.890 | 0.968 | 0.737 | 0.785 | 0.888 | 0.923 | 0.783 |
| | PPV | 0.810 | 0.896 | 0.620 | 0.854 | 0.775 | 0.821 | 0.828 |

TABLE 15C
Performance of biomarker panel incorporating Tier A biomarkers

| | | | F4 | | | | | |
|---|---|---|---|---|---|---|---|---|
| | | | Baseline- | | | F4 + | | |
| | | F4 Baseline- | Normalized | F4 + | F4 + | F5 (Gd | | ACP |
| | | Normalized | (Gd pairs) - | F5 (Gd | F5 (Gd | presence) - | AIM1 | (exacerbation |
| Tier | | (Gd pairs) - | General | presence) - | presence) - | Extreme | (annualized | VS. |
| A | Study | Subtle DA | DA | Subtle DA | General DA | DA | relapse rate) | quiescence) |
| | AUC | 0.869 | 0.876 | 0.763 | 0.790 | 0.880 | 0.869 | 0.778 |
| | PPV | 0.792 | 0.781 | 0.716 | 0.868 | 0.820 | 0.871 | 0.802 |

TABLE 15D
Performance of biomarker panel incorporating Tier B biomarkers

| | | | F4 | | | | | |
|---|---|---|---|---|---|---|---|---|
| | | | Baseline- | | | F4 + | | |
| | | F4 Baseline- | Normalized | F4 + | F4 + | F5 (Gd | | ACP |
| | | Normalized | (Gd pairs) - | F5 (Gd | F5 (Gd | presence) - | AIM1 | (exacerbation |
| | | (Gd pairs) - | General | presence) - | presence) - | Extreme | (annualized | VS. |
| Tier B | Study | Subtle DA | DA | Subtle DA | General DA | DA | relapse rate) | quiescence) |
| | AUC | 0.745 | 0.750 | 0.633 | 0.674 | 0.562 | 0.841 | 0.732 |
| | PPV | 0.578 | 0.735 | 0.609 | 0.817 | 0.462 | 0.770 | 0.999 |

TABLE 15E
Performance of biomarker panel incorporating Tier C biomarkers

| | | | F4 | | | | | |
|---|---|---|---|---|---|---|---|---|
| | | | Baseline- | | | F4 + | | |
| | | F4 Baseline- | Normalized | F4 + | F4 + | F5 (Gd | | ACP |
| | | Normalized | (Gd pairs) - | F5 (Gd | F5 (Gd | presence) - | AIM1 | (exacerbation |
| | | (Gd pairs) - | General | presence) - | presence) - | Extreme | (annualized | VS. |
| Tier C | Study | Subtle DA | DA | Subtle DA | General DA | DA | relapse rate) | quiescence) |
| | AUC | 0.710 | 0.605 | 0.660 | 0.615 | 0.589 | 0.779 | 0.631 |
| | PPV | 0.410 | 0.577 | 0.610 | 0.781 | 0.439 | 0.774 | 0.553 |

TABLE 16A
Performance of biomarker panel incorporating Tiers 1, 2, and 3 biomarkers

| | | | | F4 + | F4 | F4 + | | ACP |
|---|---|---|---|---|---|---|---|---|
| Tiers | | | | F5 (Gd | F5 (Gd | F5 (Gd | AIM1 | (exacerbation |
| 1 + | | F4 Paired - | F4 Paired - | presence) - | presence) - | presence) - | (annualized | VS. |
| 2 + 3 | Study | Subtle DA | General DA | Subtle DA | General DA | Extreme DA | relapse rate) | quiescence) |
| | AUC | 0.825 +/− | 0.889 +/− | 0.718 +/− | 0.754 +/− | 0.882 +/− | 0.816 +/− | 0.686 +/− |
| | | 0.086 | 0.055 | 0.052 | 0.043 | 0.024 | 0.091 | 0.101 |
| | PPV | 0.670 +/− | 0.748 +/− | 0.648 +/− | 0.835 +/− | 0.725 +/− | 0.760 +/− | 0.675 +/− |
| | | 0.140 | 0.142 | 0.047 | 0.043 | 0.057 | 0.077 | 0.131 |

TABLE 16B
Performance of biomarker panel incorporating Tiers 1 and 2 biomarkers

| | | F4 Baseline- | F4 Baseline- | F4 + | F4 + | F4 + | | ACP |
|---|---|---|---|---|---|---|---|---|
| | | Normalized | Normalized | F5 (Gd | F5 (Gd | F5 (Gd | AIM1 | (exacerbation |
| Tiers | | (Gd pairs) - | (Gd pairs) - | presence) - | presence) - | presence) - | (annualized | VS. |
| 1 + 2 | Study | Subtle DA | General DA | Subtle DA | General DA | Extreme DA | relapse rate) | quiescence) |
| | AUC | 0.825 +/− | 0.892 +/− | 0.693 +/− | 0.756 +/− | 0.880 +/− | 0.860 +/− | 0.726 +/− |
| | | 0.086 | 0.051 | 0.024 | 0.033 | 0.031 | 0.073 | 0.081 |
| | PPV | 0.670 +/− | 0.748 +/− | 0.613 +/− | 0.843 +/− | 0.696 +/− | 0.720 +/− | 0.733 +/− |
| | | 0.140 | 0.142 | 0.044 | 0.031 | 0.045 | 0.142 | 0.111 |

TABLE 16C
Performance of biomarker panel incorporating Tier 1 biomarkers

| | | F4 Baseline- | F4 Baseline- | F4 + | F4 + | F4 + | | ACP |
|---|---|---|---|---|---|---|---|---|
| | | Normalized | Normalized | F5 (Gd | F5 (Gd | F5 (Gd | AIM1 | (exacerbation |
| Tier | | (Gd pairs) - | (Gd pairs) - | presence) - | presence) - | presence) - | (annualized | VS. |
| 1 | Study | Subtle DA | General DA | Subtle DA | General DA | Extreme DA | relapse rate) | quiescence) |
| | AUC | 0.767 +/− | 0.825 +/− | 0.667 +/− | 0.761 +/− | 0.869 +/− | 0.756 +/− | 0.693 +/− |
| | | 0.113 | 0.064 | 0.079 | 0.035 | 0.022 | 0.107 | 0.121 |
| | PPV | 0.632 +/− | 0.661 +/− | 0.617 +/− | 0.861 +/− | 0.746 +/− | 0.620 +/− | 0.663 +/− |
| | | 0.160 | 0.056 | 0.112 | 0.035 | 0.048 | 0.065 | 0.095 |

TABLE 16D
Performance of biomarker panel incorporating Tier 2 biomarkers

| | | F4 Baseline- | F4 Baseline- | F4 + | F4 + | F4 + | | ACP |
|---|---|---|---|---|---|---|---|---|
| | | Normalized | Normalized | F5 (Gd | F5 (Gd | F5 (Gd | AIM1 | (exacerbation |
| Tier | | (Gd pairs) - | (Gd pairs) - | presence) - | presence) - | presence) - | (annualized | VS. |
| 2 | Study | Subtle DA | General DA | Subtle DA | General DA | Extreme DA | relapse rate) | quiescence) |
| | AUC | 0.655 +/− | 0.657 +/− | 0.627 +/− | 0.595 +/− | 0.604 +/− | 0.761 +/− | 0.658 +/− |
| | | 0.100 | 0.052 | 0.027 | 0.081 | 0.063 | 0.052 | 0.096 |
| | PPV | 0.559 +/− | 0.523 +/− | 0.543 +/− | 0.769 +/− | 0.462 +/− | 0.667 +/− | 0.669 +/− |
| | | 0.081 | 0.141 | 0.025 | 0.051 | 0.093 | 0.052 | 0.108 |

TABLE 16E
Performance of biomarker panel incorporating Tier 3 biomarkers

| | | F4 Baseline- | F4 Baseline- | F4 + | F4 + | F4 + | | ACP |
|---|---|---|---|---|---|---|---|---|
| | | Normalized | Normalized | F5 (Gd | F5 (Gd | F5 (Gd | AIM1 | (exacerbation |
| | | (Gd pairs) - | (Gd pairs) - | presence) - | presence) - | presence) - | (annualized | VS. |
| Tier 3 | Study | Subtle DA | General DA | Subtle DA | General DA | Extreme DA | relapse rate) | quiescence) |
| | AUC | 0.644 +/− | 0.582 +/− | 0.626 +/− | 0.566 +/− | 0.578 +/− | 0.624 +/− | 0.612 +/− |
| | | 0.102 | 0.041 | 0.048 | 0.049 | 0.024 | 0.121 | 0.061 |
| | PPV | 0.370 +/− | 0.530 +/− | 0.557 +/− | 0.742 +/− | 0.382 +/− | 0.634 +/− | 0.060 +/− |
| | | 0.074 | 0.140 | 0.040 | 0.039 | 0.050 | 0.112 | 0.120 |

TABLE 17
Baseline-normalization shifts prediction: Biomarker panels for anticipating a
shift (e.g., increase or decrease) in MS disease activity.

| | | | AUROC | | | PPV | |
|---|---|---|---|---|---|---|---|
| | | AUROC | (weighted | AUROC | PPV | (weighted | PPV |
| Biomarkers | Study | (mean) | mean) | (stdev) | (mean) | mean) | (stdev) |
| NEFL, MOG, | F6 | 0.884 | 0.8854 | 0.0859 | 0.763 | 0.7635 | 0.1382 |
| CD6, CXCL9 | | | | | | | |
| NEFL, CXCL9, | F4 | 0.846 | 0.8516 | 0.1064 | 0.7485 | 0.7541 | 0.0945 |
| TNFRSF10A, COL4A1 | | | | | | | |
| NEFL, CD6, CXCL9 | F6 | 0.885 | 0.8862 | 0.0911 | 0.7865 | 0.7876 | 0.1255 |
| NEFL, TNFRSF10A, | F4 | 0.819 | 0.8258 | 0.1755 | 0.756 | 0.7636 | 0.0752 |
| COL4A1 | | | | | | | |
| NEFL, MOG | Blended | 0.802 | 0.8038 | 0.1764 | 0.698 | 0.7002 | 0.0520 |
| NEFL, CD6 | F6 | 0.860 | 0.8621 | 0.1082 | 0.7745 | 0.7774 | 0.0873 |
| NEFL, CXCL9 | Blended | 0.854 | 0.8564 | 0.1093 | 0.7435 | 0.7472 | 0.1211 |
| NEFL, TNFRSF10A | F4 | 0.824 | 0.8297 | 0.1679 | 0.734 | 0.7402 | 0.0766 |
| MOG, CXCL9, IL-12B, | F6 | 0.795 | 0.7917 | 0.1350 | 0.6665 | 0.6646 | 0.0844 |
| APLP1 | | | | | | | |
| CXCL9, COL4A1, OPG, | F4 | 0.736 | 0.7410 | 0.0947 | 0.674 | 0.6761 | 0.0613 |
| VCAN | | | | | | | |
| CXCL9, OPG, APLP1, | F4 | 0.741 | 0.7449 | 0.1303 | 0.6515 | 0.6513 | 0.0664 |
| OPN | | | | | | | |
| MOG, IL-12B, APLP1 | F6 | 0.737 | 0.7310 | 0.1699 | 0.6495 | 0.6454 | 0.0757 |
| MOG, CD6, CXCL9 | Blended | 0.798 | 0.7947 | 0.0832 | 0.7125 | 0.7105 | 0.0915 |
| CXCL9, COL4A1, VCAN | F4 | 0.711 | 0.7148 | 0.0578 | 0.6575 | 0.6596 | 0.0525 |
| MOG, IL-12B | F6 | 0.735 | 0.7296 | 0.1707 | 0.628 | 0.6246 | 0.0618 |
| CXCL9, CD6 | Blended | 0.766 | 0.7636 | 0.0919 | 0.699 | 0.6962 | 0.0854 |
| MOG, CXCL9 | Blended | 0.761 | 0.7588 | 0.1383 | 0.686 | 0.6861 | 0.0937 |
| MOG, CD6 | Blended | 0.745 | 0.7419 | 0.1083 | 0.705 | 0.7023 | 0.0596 |
| CXCL9, COL4A1 | F4 | 0.716 | 0.7177 | 0.0869 | 0.6445 | 0.6454 | 0.0395 |

TABLE 18
Biomarker panels for predicting a decrease in MS disease activity.

| | | | AUROC | | | PPV | |
|---|---|---|---|---|---|---|---|
| | | AUROC | (weighted | AUROC | PPV | (weighted | PPV |
| Biomarkers | Cohort | (mean) | mean) | (stdev) | (mean) | mean) | (stdev) |
| NEFL, CD6, CXCL9, CXCL13 | F6 | 0.889 | 0.8882 | 0.0654 | 0.764 | 0.7645 | 0.0804 |
| NEFL, MOG, CD6, CXCL9 | F6 | 0.859 | 0.8585 | 0.0847 | 0.7845 | 0.7848 | 0.1088 |
| NEFL, TNFRSF10A, COL4A1, | F4 | 0.836 | 0.8414 | 0.1604 | 0.725 | 0.7310 | 0.1247 |
| CCL20 | | | | | | | |
| NEFL, CD6, CXCL9 | F6 | 0.876 | 0.8758 | 0.0770 | 0.763 | 0.7638 | 0.1050 |
| NEFL, TNFRSF10A, COL4A1 | F4 | 0.834 | 0.8387 | 0.1628 | 0.7055 | 0.7110 | 0.1316 |
| NEFL, CD6 | F6 | 0.857 | 0.8579 | 0.0991 | 0.7585 | 0.7590 | 0.1140 |
| NEFL, TNFRSF10A | F4 | 0.840 | 0.8445 | 0.1573 | 0.697 | 0.7012 | 0.1229 |
| MOG, IL-12B, OPN, CNTN2 | F6 | 0.765 | 0.7587 | 0.1181 | 0.6935 | 0.6891 | 0.0959 |
| CD6, COL4A1, CCL20, VCA | F4 | 0.693 | 0.6960 | 0.0794 | 0.6545 | 0.6565 | 0.0759 |
| MOG, IL-12B, CNTN2 | F6 | 0.760 | 0.7538 | 0.1238 | 0.6975 | 0.6925 | 0.1085 |
| CD6, CCL20, VCAN | F4 | 0.691 | 0.6926 | 0.1037 | 0.657 | 0.6581 | 0.0771 |
| MOG, IL-12B | F6 | 0.732 | 0.7261 | 0.1275 | 0.691 | 0.6864 | 0.0549 |
| CD6, VCAN | F4 | 0.693 | 0.6936 | 0.1228 | 0.6125 | 0.6117 | 0.0544 |

TABLE 19
Biomarker panels for determining whether an associated MRI
had the presence or absence of Gadolinium-enhancing
lesions based on protein signatures in
blood serum for a single blood draw within
30 days of the MRI.

| | Avg. Performance | | | |
|---|---|---|---|---|
| Biomarker Set (List of | | Delta | | |
| proteins/features) | AUROC | AUROC | PPV | PPV Unc. |
| NEFL, TNFSF13B | 0.788 | 0.002 | 0.708 | 0.002 |
| NEFL, CNTN2 | 0.777 | 0.003 | 0.732 | 0.002 |
| NEFL, CXCL9 | 0.777 | 0.004 | 0.713 | 0.002 |
| MOG, CDCP1 | 0.672 | 0.008 | 0.597 | 0.002 |
| MOG, TNFSF13B | 0.672 | 0.007 | 0.599 | 0.002 |
| MOG, CXCL9 | 0.670 | 0.005 | 0.606 | 0.002 |
| NEFL, CNTN2, | 0.794 | 0.002 | 0.745 | 0.002 |
| TNFSF13B | | | | |
| NEFL, APLP1, | 0.794 | 0.002 | 0.715 | 0.002 |
| TNFSF13B | | | | |
| NEFL, TNFRSF10A, | 0.792 | 0.003 | 0.718 | 0.002 |
| TNFSF13B | | | | |
| MOG, CXCL9, | 0.690 | 0.006 | 0.623 | 0.002 |
| TNFSF13B | | | | |
| MOG, OPG, | 0.685 | 0.006 | 0.637 | 0.002 |
| TNFSF13B | | | | |
| MOG, CCL20, | 0.685 | 0.008 | 0.664 | 0.002 |
| TNFSF13B | | | | |
| NEFL, TNFRSF10A, | 0.798 | 0.002 | 0.742 | 0.002 |
| CNTN2, TNFSF13B | | | | |
| NEFL, COL4A1, | 0.798 | 0.002 | 0.749 | 0.002 |
| CNTN2, TNFSF13B | | | | |
| NEFL, TNFRSF10A, | 0.797 | 0.003 | 0.705 | 0.002 |
| APLP1, TNFSF13B | | | | |
| MOG, CXCL9, APLP1, | 0.697 | 0.005 | 0.634 | 0.002 |
| TNFSF13B | | | | |
| MOG, CXCL9, OPG, | 0.696 | 0.005 | 0.643 | 0.002 |
| TNFSF13B | | | | |
| MOG, CXCL9, OPG, | 0.693 | 0.006 | 0.631 | 0.002 |
| CNTN2 | | | | |

TABLE 20
Biomarker panels for predicting the number of lesions that an
associated MRI had based on protein signatures in blood
serum for a single blood draw within 30 days of the MRI.

| | Avg. Performance | | | | | | | |
|---|---|---|---|---|---|---|---|---|
| Biomarker Set | Spear- | Delta | | Delta | | | | |
| (List of | man's | Spearman's | Adj. | Adj. | | Delta | | Delta |
| proteins/features) | R | R | R² | R₂ | MSE | MSE | MAE | MAE |
| NEFL, TNFSF13B | 0.524 | 0.020 | 0.266 | 0.003 | 1.362 | 0.033 | 0.886 | 0.006 |
| NEFL, SERPINA9 | 0.501 | 0.017 | 0.243 | 0.007 | 1.394 | 0.027 | 0.894 | 0.005 |
| NEFL, GH | 0.505 | 0.010 | 0.242 | 0.003 | 1.386 | 0.027 | 0.891 | 0.007 |
| MOG, TNFSF13B | 0.286 | 0.009 | 0.053 | 0.006 | 1.698 | 0.043 | 1.011 | 0.015 |
| MOG CXCL9 | 0.290 | 0.016 | 0.047 | 0.008 | 1.695 | 0.038 | 1.010 | 0.013 |
| MOG, IL-12B | 0.272 | 0.006 | 0.035 | 0.005 | 1.700 | 0.029 | 1.018 | 0.012 |
| NEFL, SERPINA9, TNFSF13B | 0.533 | 0.024 | 0.258 | 0.009 | 1.363 | 0.033 | 0.883 | 0.003 |
| NEFL, CNTN2, TNFSF13B | 0.525 | 0.022 | 0.251 | 0.008 | 1.338 | 0.035 | 0.876 | 0.004 |
| NEFL, APLP1, TNFSF13B | 0.537 | 0.013 | 0.250 | 0.008 | 1.345 | 0.022 | 0.883 | 0.004 |
| MOG, CXCL9, TNFSF13B | 0.306 | 0.011 | 0.041 | 0.007 | 1.680 | 0.045 | 1.007 | 0.014 |
| MOG, SERPINA9, TNFSF13B | 0.297 | 0.013 | 0.037 | 0.012 | 1.702 | 0.043 | 1.009 | 0.014 |
| MOG, OPG, TNFSF13B | 0.300 | 0.009 | 0.035 | 0.006 | 1.692 | 0.046 | 1.003 | 0.015 |
| NEFL, CCL20, SERPINA9, TNFSF13B | 0.535 | 0.026 | 0.238 | 0.012 | 1.367 | 0.029 | 0.888 | 0.002 |
| NEFL, APLP1, SERPINA9, TNFSF13B | 0.542 | 0.018 | 0.237 | 0.012 | 1.348 | 0.022 | 0.883 | 0.003 |
| NEFL, CCL20, APLP1, TNFSF13B | 0.544 | 0.016 | 0.236 | 0.013 | 1.345 | 0.021 | 0.886 | 0.003 |
| MOG, CXCL9, OPG, TNFSF13B | 0.318 | 0.012 | 0.023 | 0.009 | 1.674 | 0.048 | 1.000 | 0.014 |
| MOG, OPG, SERPINA9, TNFSF13B | 0.312 | 0.013 | 0.020 | 0.011 | 1.696 | 0.046 | 1.002 | 0.014 |
| MOG, CXCL9, SERPINA9, TNFSF13B | 0.317 | 0.010 | 0.018 | 0.006 | 1.687 | 0.045 | 1.007 | 0.014 |

TABLE 21
Biomarker panels for predicting disease progression
(e.g., predicting the number of lesions that an associated
MRI had based on protein signatures in blood serum
for a single blood draw within 30 days of the MRI.

| | F6 Avg. Performance | | | | | | | | |
|---|---|---|---|---|---|---|---|---|---|
| Biomarker Set | Spear- | Delta | | Delta | | Delta | | | |
| (List of | man's | Spearman's | Pearson's | Pearson's | R² | R² | Progression | | |
| proteins/features) | R² | R | R² | R² | Adj | Adj | Direction | MSE | MAE |
| APLP1, GFAP | 0.267 | 0.084 | 0.306 | 0.094 | 0.269 | 0.098 | Positive | 2.175 | 1.131 |
| MOG, GFAP | 0.261 | 0.081 | 0.334 | 0.103 | 0.299 | 0.108 | Positive | 2.233 | 1.154 |
| NEFL, GFAP | 0.233 | 0.076 | 0.297 | 0.105 | 0.260 | 0.110 | Positive | 2.370 | 1.161 |
| NEFL, MOG | 0.288 | 0.090 | 0.442 | 0.058 | 0.413 | 0.061 | Positive | 1.596 | 0.954 |
| NEFL, SERPINA9 | 0.287 | 0.085 | 0.439 | 0.059 | 0.410 | 0.063 | Positive | 1.608 | 0.966 |
| SERPINA9, | 0.286 | 0.085 | 0.434 | 0.061 | 0.405 | 0.065 | Positive | 1.724 | 1.029 |
| FLRT2 | | | | | | | | | |
| CXCL9, OPG | 0.141 | 0.046 | 0.169 | 0.058 | 0.125 | 0.062 | Negative | 2.743 | 1.207 |
| CD6, IL12B | 0.140 | 0.036 | 0.126 | 0.035 | 0.080 | 0.037 | Negative | 2.870 | 1.235 |
| CD6, APLP1 | 0.133 | 0.054 | 0.152 | 0.053 | 0.107 | 0.056 | Negative | 2.777 | 1.215 |
| NEFL, APLP1, | 0.279 | 0.089 | 0.309 | 0.103 | 0.253 | 0.111 | Positive | 2.183 | 1.119 |
| GFAP | | | | | | | | | |
| NEFL, MOG, | 0.279 | 0.085 | 0.351 | 0.115 | 0.299 | 0.124 | Positive | 2.216 | 1.135 |
| GFAP | | | | | | | | | |
| MOG, APLP1, | 0.278 | 0.086 | 0.330 | 0.099 | 0.276 | 0.107 | Positive | 2.154 | 1.132 |
| GFAP | | | | | | | | | |
| NEFL, MOG, GH | 0.290 | 0.089 | 0.441 | 0.057 | 0.396 | 0.062 | Positive | 1.595 | 0.950 |
| CXCL9, OPG, | 0.290 | 0.086 | 0.439 | 0.061 | 0.394 | 0.066 | Positive | 1.502 | 0.951 |
| SERPINA9 | | | | | | | | | |
| NEFL, MOG, | 0.290 | 0.087 | 0.445 | 0.061 | 0.400 | 0.065 | Positive | 1.555 | 0.945 |
| SERPINA9 | | | | | | | | | |
| CXCL9, OPG, | 0.152 | 0.050 | 0.169 | 0.063 | 0.101 | 0.069 | Negative | 2.755 | 1.215 |
| TNFRSF10A | | | | | | | | | |
| CD6, IL12B, | 0.151 | 0.039 | 0.138 | 0.039 | 0.068 | 0.042 | Negative | 2.938 | 1.255 |
| APLP1 | | | | | | | | | |
| CD6, IL12B, | 0.146 | 0.030 | 0.116 | 0.037 | 0.044 | 0.040 | Negative | 2.884 | 1.236 |
| TNFSF13B | | | | | | | | | |
| NEFL, MOG, | 0.309 | 0.092 | 0.345 | 0.112 | 0.272 | 0.124 | Positive | 2.144 | 1.108 |
| APLP1, GFAP | | | | | | | | | |
| NEFL, MOG, | 0.298 | 0.090 | 0.355 | 0.120 | 0.283 | 0.133 | Positive | 2.162 | 1.104 |
| PRTG, GFAP | | | | | | | | | |
| NEFL, MOG, | 0.297 | 0.100 | 0.333 | 0.123 | 0.259 | 0.137 | Positive | 2.130 | 1.090 |
| IL12B, GFAP | | | | | | | | | |
| MOG, CXCL9, | 0.293 | 0.087 | 0.441 | 0.061 | 0.378 | 0.068 | Positive | 1.491 | 0.946 |
| OPG, SERPINA9 | | | | | | | | | |
| NEFL, MOG, GH, | 0.292 | 0.089 | 0.445 | 0.059 | 0.383 | 0.066 | Positive | 1.553 | 0.943 |
| SERPINA9 | | | | | | | | | |
| NEFL, MOG, GH, | 0.292 | 0.089 | 0.442 | 0.057 | 0.380 | 0.064 | Positive | 1.592 | 0.948 |
| TNFRSF10A | | | | | | | | | |
| CD6, CCL20, | 0.158 | 0.050 | 0.142 | 0.056 | 0.047 | 0.062 | Negative | 2.919 | 1.243 |
| IL12B, FLRT2 | | | | | | | | | |
| CD6, IL12B, | 0.156 | 0.034 | 0.137 | 0.040 | 0.042 | 0.044 | Negative | 2.967 | 1.261 |
| APLP1, PRTG | | | | | | | | | |
| CD6, CCL20, | 0.156 | 0.057 | 0.171 | 0.061 | 0.079 | 0.068 | Negative | 2.973 | 1.257 |
| IL12B, APLP1 | | | | | | | | | |

TABLE 22
Characteristics of the UHBC cohort

| | Gd⁺ | Gd⁻ | Total |
|---|---|---|---|
| Age [y] | 39.8 ± 11.4 | 41.9 ± 11.7 | 40.8 ± 11.6 |
| Disease Duration [y] | 11.4 ± 10.0 | 12.4 ± 10.9 | 11.9 ± 10.5 |
| % Female | 78% | 76% | 77% |
| EDSS | 2.4 ± 1.6 | 2.5 ± 1.6 | 2.4 ± 1.6 |
| Blood draw within 30 days of MRI | 69.8% | 81.9% | 75.6% |
| Sample Count | 106 | 99 | 205 |

TABLE 23
Characteristics of patients in PROMOTE study.

| | PwMS Closest | PwMS Closest | |
|---|---|---|---|
| | PDDS to | PDDS | |
| | Serum ≤ 4 | to Serum > 4 | All |
| % Women | 85.2% (75) | 50% (6) | 81% (81) |
| Age (mean ± SD) | 46.5 ± 11.8 | 57.0 ± 11.7 | 47.8 ± 12.3 |
| Disease Duration | 9.6 ± 8.3 | 21.8 ± 10.9 | 11.1 ± 9.5 |
| DMT Efficacy¹ | 44 High/29 | 12 High | 56 High/29 |
| (at time of serum) | Standard/15 | | Standard/15 |
| | None | | None |
| # of Relapses | 2.8 ± 2.1 | 4.4 ± 4.3 | 3.0 ± 2.5 |
| Total | 88 | 12 | 100 |

TABLE 24A
Tiered biomarkers for use in predicting multiple sclerosis
disease progression

| | | | Accession |
|---|---|---|---|
| | | | Number |
| | | Biomarker | (Uniprot |
| Tier | Biomarker Name | Symbol | Database) |
| 1 | Glial Fibrillary Acidic Protein | GFAP | O43155 |
| 1 | CUB domain-containing protein 1 | CDCP1 | Q9H5V8 |
| 1 | Myelin Oligodendrocyte | MOG | Q16653 |
| | Glycoprotein | | |
| 1 | Chemokine (C—X—C motif) ligand 13 | CXCL13 | O43927 |
| 1 | Osteoprotegerin | OPG | O00300 |
| 1 | Amyloid Beta Precursor Like | APLP1 | P51693 |
| | Protein 1 | | |
| 1 | Versican core protein | VCAN | P13611 |
| 1 | Neurofilament Light Polypeptide | NEFL | P07196 |
| | Chain | | |
| 2 | Chemokine (C—X—C motif) ligand 9 | CXCL9 | Q07325 |
| 2 | Tumor Necrosis Factor Receptor | TNFRSF10A | O00220 |
| | Superfamily Member 10A | | |
| 2 | C-C Motif Chemokine Ligand 20 | CCL20/MIP | P78556 |
| | | 3-α | |
| 2 | Tumor necrosis factor ligand | TNFSF13B | Q9Y275 |
| | superfamily member 13B | | |
| 2 | Cluster of Differentiation 6 | CD6 | P30203 |
| 2 | Serpin Family A Member 9 | SERPINA9 | Q86WD7 |
| 2 | Fibronectin Leucine Rich | FLRT2 | O43155 |
| | Transmembrane Protein 2 | | |
| 2 | Osteopontin | OPN | P10451 |
| 2 | Contactin-2 | CNTN2 | Q02246 |
| 3 | Collagen, type IV, alpha 1 | COL4A1 | P02462 |
| 3 | Growth Hormone | GH | P01241 |
| 3 | Interleukin-12 subunit beta | IL-12B | P29460 |
| 3 | Protogenin | PRTG | Q2VWP7 |

TABLE 24B
Classifier Performance of Tier 1/Tier2/Tier 3 biomarkers

| Raw or Log- | | | | | |
|---|---|---|---|---|---|
| Demographically | | | | | |
| Adjusted? | Biomarker | Classifier Performance | | | |
| Transformed, | Subset | AUROC | +/−SD | PPV | +/−SD |
| Raw | Tier 1 | 0.77 | 0.10 | 0.19 | 0.05 |
| Raw | Tier 2 | 0.58 | 0.09 | 0.10 | 0.02 |
| Raw | Tier 3 | 0.44 | 0.06 | 0.09 | 0.02 |
| Raw | Tier 1 + 2 | 0.76 | 0.08 | 0.19 | 0.05 |
| Raw | Tier 1 + 2 + 3 | 0.74 | 0.09 | 0.17 | 0.03 |
| Adjusted | Tier 1 | 0.79 | 0.05 | 0.19 | 0.01 |
| Adjusted | Tier 2 | 0.60 | 0.12 | 0.13 | 0.04 |
| Adjusted | Tier 3 | 0.46 | 0.07 | 0.08 | 0.02 |
| Adjusted | Tier 1 + 2 | 0.81 | 0.06 | 0.20 | 0.03 |
| Adjusted | Tier 1 + 2 + 3 | 0.81 | 0.06 | 0.21 | 0.03 |

TABLE 24C
Regression Performance of Tier 1/Tier2/Tier 3 biomarkers

| Raw or Log- | | | | | | | | | | | |
|---|---|---|---|---|---|---|---|---|---|---|---|
| Transformed, | | Regression Performance | | | | | | | | | |
| Demographically | Biomarker | Pearson's | | Pearson's | | Spearman's | | Spearman's | | Adj. | |
| Adjusted? | Subset | R | +/−SD | R-sq | +/−SD | R | +/−SD | R-sq | +/−SD | R-sq | +/−SD |
| Raw | Tier 1 | 0.31 | 0.17 | 0.36 | 0.14 | 0.15 | 0.10 | 0.12 | 0.11 | 0.08 | 0.11 |
| Raw | Tier 2 | 0.14 | 0.07 | 0.12 | 0.08 | 0.02 | 0.02 | 0.02 | 0.02 | −0.06 | 0.02 |
| Raw | Tier 3 | −0.06 | 0.07 | −0.05 | 0.06 | 0.01 | 0.01 | 0.01 | 0.01 | −0.03 | 0.01 |
| Raw | Tier 1 + 2 | 0.33 | 0.09 | 0.35 | 0.08 | 0.13 | 0.06 | 0.11 | 0.06 | −0.02 | 0.07 |
| Raw | Tier 1 + | 0.34 | 0.06 | 0.36 | 0.07 | 0.13 | 0.05 | 0.12 | 0.04 | −0.06 | 0.06 |
| | 2 + 3 | | | | | | | | | | |
| Adjusted | Tier 1 | 0.36 | 0.12 | 0.40 | 0.11 | 0.17 | 0.09 | 0.14 | 0.10 | 0.11 | 0.09 |
| Adjusted | Tier 2 | 0.05 | 0.11 | 0.09 | 0.13 | 0.03 | 0.03 | 0.02 | 0.03 | −0.06 | 0.04 |
| Adjusted | Tier 3 | −0.05 | 0.08 | −0.02 | 0.05 | 0.00 | 0.01 | 0.01 | 0.01 | −0.03 | 0.01 |
| Adjusted | Tier 1 + 2 | 0.32 | 0.08 | 0.35 | 0.07 | 0.13 | 0.05 | 0.11 | 0.05 | −0.02 | 0.06 |
| Adjusted | Tier 1 + | 0.32 | 0.07 | 0.36 | 0.06 | 0.13 | 0.04 | 0.10 | 0.04 | −0.06 | 0.05 |
| | 2 + 3 | | | | | | | | | | |

TABLE 25
Patient characteristics from whom samples were obtained and used to train and test machine learning models.

| Metric (test of significance) | Train (70%) | Test (30%) |
|---|---|---|
| Sample Size (N) | 429.00 (0.695) | 188.00 (0.305) |
| Active (Gd or T2) vs. Stable | Stable 261 (0.608392) | Stable 110 (0.585106) |
| z-test p-value = 0.470 > 0.05 | Active 168 (0.391608) | Active 78 (0.414894) |
| Gd Status | Non-Gd+ (0) 270 (0.629371) | Non-Gd+ 112 (0.595745) |
| Chi-Squared p-value = 0.973 > 0.05 | Subtle (1) 108 (0.251748) | Subtle 55 (0.292553) |
| | Moderate (2-3) 32 (0.074592) | Moderate 14 (0.074468) |
| | Extreme (4+) 19 (0.044289) | Extreme 7 (0.037234) |
| Patient Set Sizes | 1 250 → 250 (0.582751) | 1 108 → 108 (0.574468) |
| Chi-Squared p-value = 0.812 > 0.05 | 2 88 → 44 (0.205128) | 2 40 → 20 (0.212766) |
| | 3 66 → 22 (0.153846) | 3 30 → 10 (0.159574) |
| | 4 20 → 5(0.046620) | 4 4 → 1 (0.021277) |
| | 5 5 → 1 (0.011655) | 6 6 → 1 (0.031915) |
| | No groups of 6 | No groups of 5 |
| Site | AUB 143 (0.333333) | BWH 61 (0.324468) |
| Chi-Squared p-value = 0.996 > 0.05 | BWH 134 (0.312354) | AUB 59 (0.313830) |
| | RMMSC 117 (0.272727) | RMMSC 52 (0.276596) |
| | UMASS 35 (0.081585) | UMASS 16 (0.085106) |
| Age | count 429.000000 | count 188.000000 |
| t-test p-value = 0.451 > 0.05 | mean 41.615501 | mean 42.466383 |
| | std 12.685738 | std 13.469106 |
| | min 13.420000 | min 17.040000 |
| | 25% 32.560000 | 25% 31.150000 |
| | 50% 40.000000 | 50% 43.140000 |
| | 75% 50.000000 | 75% 50.150000 |
| | max 78.000000 | max 74.000000 |
| Disease Duration | count 428.000000 *(missing 1) | count 188.000000 |
| t-test p-value = 0.752 > 0.05 | mean 9.558983 | mean 9.185151 |
| | std 8.533333 | std 8.833820 |
| | min −0.583333 | min −0.250000 |
| | 25% 2.980000 | 25% 2.540041 |
| | 50% 7.415000 | 50% 7.020000 |
| | 75% 13.844610 | 75% 12.545517 |
| | max 46.962355 | max 41.771389 |
| Sex | F 302 (0.703963) | F 134 (0.712766) |
| z-test p-value = 0.117 > 0.05 | M 127 (0.296037) | M 54 (0.287234) |

TABLE 26
Biomarker pairs, triplets, or quadruplets that are predictive of
mild/moderate v. severe multiple sclerosis disability.

| Type | Proteins | AUROC | +/−SD | PPV | +/−SD |
|---|---|---|---|---|---|
| Pairs | CDCP1, GFAP | 0.732 | 0.092 | 0.185 | 0.103 |
| Pairs | APLP1, GFAP | 0.731 | 0.090 | 0.176 | 0.094 |
| Pairs | GFAP, CXCL13 | 0.729 | 0.091 | 0.158 | 0.059 |
| Pairs | MOG, GFAP | 0.727 | 0.112 | 0.184 | 0.102 |
| Pairs | OPG, GFAP | 0.725 | 0.126 | 0.188 | 0.112 |
| Triplets | CDCP1, APLP1, | 0.764 | 0.079 | 0.179 | 0.052 |
| | GFAP | | | | |
| Triplets | CDCP1, MOG, | 0.755 | 0.100 | 0.194 | 0.112 |
| | GFAP | | | | |
| Triplets | APLP1, GFAP, | 0.750 | 0.090 | 0.185 | 0.076 |
| | CXCL13 | | | | |
| Triplets | CDCP1, GFAP, | 0.749 | 0.086 | 0.180 | 0.042 |
| | SERPINA9 | | | | |
| Triplets | MOG, GFAP, | 0.748 | 0.104 | 0.190 | 0.068 |
| | CXCL13 | | | | |
| Quadruplets | CDCP1, CCL20, | 0.779 | 0.074 | 0.188 | 0.058 |
| | APLP1, GFAP | | | | |
| Quadruplets | CDCP1, APLP1, | 0.770 | 0.074 | 0.184 | 0.034 |
| | GFAP, CXCL13 | | | | |
| Quadruplets | CDCP1, CCL20, | 0.768 | 0.092 | 0.188 | 0.076 |
| | MOG, GFAP | | | | |
| Quadruplets | CDCP1, GFAP, | 0.765 | 0.073 | 0.194 | 0.062 |
| | APLP1, SERPINA9 | | | | |
| Quadruplets | CDCP1, MOG, | 0.765 | 0.088 | 0.178 | 0.071 |
| | GFAP, APLP1 | | | | |
| Pairs | CDCP1, OPG | 0.689 | 0.135 | 0.155 | 0.067 |
| Pairs | CDCP1, | 0.671 | 0.087 | 0.132 | 0.042 |
| | SERPINA9 | | | | |
| Pairs | OPG, TNFRSF10A | 0.671 | 0.126 | 0.134 | 0.051 |
| Pairs | OPG, MOG | 0.666 | 0.110 | 0.125 | 0.031 |
| Pairs | CDCP1, MOG | 0.661 | 0.091 | 0.128 | 0.056 |
| Triplets | CDCP1, MOG, | 0.689 | 0.118 | 0.132 | 0.050 |
| | OPG | | | | |
| Triplets | CDCP1, | 0.688 | 0.111 | 0.143 | 0.038 |
| | SERPINA9, OPG | | | | |
| Triplets | CDCP1, OPG, | 0.687 | 0.104 | 0.135 | 0.032 |
| | CXCL13 | | | | |
| Triplets | CDCP1, CXCL9, | 0.687 | 0.135 | 0.154 | 0.072 |
| | OPG | | | | |
| Triplets | CDCP1, FLRT2, | 0.687 | 0.137 | 0.153 | 0.073 |
| | OPG | | | | |
| Quadruplets | CDCP1, MOG, | 0.696 | 0.110 | 0.143 | 0.038 |
| | OPG, CXCL13 | | | | |
| Quadruplets | CDCP1, MOG, | 0.690 | 0.123 | 0.132 | 0.050 |
| | TNFRSF10A, OPG | | | | |
| Quadruplets | CDCP1, CXCL9, | 0.690 | 0.111 | 0.146 | 0.040 |
| | SERPINA9, OPG | | | | |
| Quadruplets | CDCP1, CNTN2, | 0.690 | 0.111 | 0.137 | 0.035 |
| | SERPINA9, OPG | | | | |
| Quadruplets | CDCP1, | 0.689 | 0.109 | 0.139 | 0.033 |
| | SERPINA9, CD6, | | | | |
| | OPG | | | | |

TABLE 27
Biomarker pairs, triplets, or quadruplets that are predictive of multiple sclerosis disease progression.

| | | Pearson's | | Pearson's | | Spearman's | | Spearman's | | Adj. | |
|---|---|---|---|---|---|---|---|---|---|---|---|
| Type | Proteins | R | +/−SD | R-sq | +/− SD | R | +/− SD | R-sq | +/−SD | R-sq | +/−SD |
| Pairs* | GFAP, MOG | 0.334 | 0.188 | 0.147 | 0.138 | 0.286 | 0.173 | 0.112 | 0.104 | 0.132 | 0.141 |
| Pairs* | GFAP, APLP1 | 0.344 | 0.141 | 0.138 | 0.102 | 0.311 | 0.161 | 0.123 | 0.111 | 0.123 | 0.104 |
| Pairs* | OPG, GFAP | 0.321 | 0.179 | 0.135 | 0.123 | 0.265 | 0.172 | 0.100 | 0.094 | 0.120 | 0.125 |
| Pairs* | TNFRSF10A, | 0.313 | 0.164 | 0.125 | 0.107 | 0.261 | 0.177 | 0.099 | 0.104 | 0.110 | 0.109 |
| | GFAP | | | | | | | | | | |
| Pairs* | GFAP, CDCP1 | 0.293 | 0.169 | 0.115 | 0.097 | 0.248 | 0.172 | 0.091 | 0.084 | 0.099 | 0.099 |
| Pairs | GFAP, APLP1 | 0.445 | 0.136 | 0.216 | 0.113 | 0.413 | 0.128 | 0.187 | 0.111 | 0.203 | 0.115 |
| Pairs | GFAP, NEFL | 0.425 | 0.145 | 0.202 | 0.115 | 0.377 | 0.152 | 0.165 | 0.114 | 0.188 | 0.117 |
| Pairs | CNTN2, GFAP | 0.421 | 0.151 | 0.200 | 0.116 | 0.375 | 0.139 | 0.160 | 0.104 | 0.186 | 0.118 |
| Pairs | GH, GFAP | 0.420 | 0.147 | 0.198 | 0.115 | 0.366 | 0.154 | 0.157 | 0.116 | 0.184 | 0.117 |
| Pairs | CXCL9, GFAP | 0.422 | 0.144 | 0.199 | 0.110 | 0.371 | 0.135 | 0.156 | 0.100 | 0.185 | 0.112 |
| Triplets* | OPG, GFAP, | 0.363 | 0.204 | 0.173 | 0.165 | 0.303 | 0.186 | 0.126 | 0.113 | 0.152 | 0.169 |
| | MOG | | | | | | | | | | |
| Triplets* | OPG, GFAP, | 0.372 | 0.156 | 0.162 | 0.121 | 0.330 | 0.172 | 0.138 | 0.127 | 0.141 | 0.124 |
| | APLP1 | | | | | | | | | | |
| Triplets* | GFAP, | 0.350 | 0.171 | 0.152 | 0.118 | 0.300 | 0.167 | 0.118 | 0.094 | 0.130 | 0.121 |
| | TNFRSF10A, | | | | | | | | | | |
| | MOG | | | | | | | | | | |
| Triplets* | CXCL9, OPG, | 0.356 | 0.148 | 0.149 | 0.118 | 0.331 | 0.115 | 0.123 | 0.081 | 0.127 | 0.122 |
| | GFAP | | | | | | | | | | |
| Triplets* | GFAP, | 0.357 | 0.143 | 0.148 | 0.112 | 0.319 | 0.160 | 0.128 | 0.121 | 0.126 | 0.115 |
| | TNFRSF10A, | | | | | | | | | | |
| | APLP1 | | | | | | | | | | |
| Triplets | GFAP, APLP1, | 0.446 | 0.137 | 0.217 | 0.120 | 0.422 | 0.144 | 0.199 | 0.130 | 0.197 | 0.123 |
| | NEFL | | | | | | | | | | |
| Triplets | GFAP, | 0.439 | 0.124 | 0.208 | 0.102 | 0.423 | 0.123 | 0.194 | 0.108 | 0.188 | 0.104 |
| | CXCL13, | | | | | | | | | | |
| | APLP1 | | | | | | | | | | |
| Triplets | GFAP, FLRT2, | 0.435 | 0.127 | 0.205 | 0.102 | 0.416 | 0.122 | 0.188 | 0.107 | 0.184 | 0.105 |
| | APLP1 | | | | | | | | | | |
| Triplets | CXCL9, | 0.440 | 0.137 | 0.213 | 0.116 | 0.410 | 0.141 | 0.188 | 0.119 | 0.192 | 0.119 |
| | GFAP, APLP1 | | | | | | | | | | |
| Triplets | GH, GFAP, | 0.441 | 0.136 | 0.213 | 0.115 | 0.409 | 0.141 | 0.187 | 0.121 | 0.192 | 0.118 |
| | APLP1 | | | | | | | | | | |
| Quadruplets* | CXCL9, OPG, | 0.389 | 0.173 | 0.181 | 0.155 | 0.360 | 0.141 | 0.150 | 0.105 | 0.153 | 0.160 |
| | GFAP, MOG | | | | | | | | | | |
| Quadruplets* | CNTN2, OPG, | 0.366 | 0.200 | 0.174 | 0.160 | 0.314 | 0.180 | 0.131 | 0.109 | 0.145 | 0.165 |
| | GFAP, MOG | | | | | | | | | | |
| Quadruplets* | CXCL9, OPG, | 0.395 | 0.130 | 0.173 | 0.109 | 0.364 | 0.134 | 0.151 | 0.110 | 0.144 | 0.113 |
| | GFAP, APLP1 | | | | | | | | | | |
| Quadruplets* | OPG, GFAP, | 0.363 | 0.198 | 0.171 | 0.167 | 0.309 | 0.170 | 0.124 | 0.109 | 0.142 | 0.173 |
| | PRTG, MOG | | | | | | | | | | |
| Quadruplets* | OPG, GFAP, | 0.358 | 0.207 | 0.171 | 0.167 | 0.299 | 0.186 | 0.124 | 0.112 | 0.142 | 0.173 |
| | OPN, MOG | | | | | | | | | | |
| Quadruplets | GFAP, | 0.441 | 0.123 | 0.210 | 0.106 | 0.430 | 0.138 | 0.204 | 0.125 | 0.182 | 0.109 |
| | CXCL13, | | | | | | | | | | |
| | APLP1, NEFL | | | | | | | | | | |
| Quadruplets | GFAP, FLRT2, | 0.438 | 0.131 | 0.209 | 0.110 | 0.422 | 0.142 | 0.198 | 0.127 | 0.181 | 0.114 |
| | APLP1, NEFL | | | | | | | | | | |
| Quadruplets | OPN, GFAP, | 0.440 | 0.142 | 0.214 | 0.123 | 0.420 | 0.146 | 0.197 | 0.131 | 0.186 | 0.128 |
| | APLP1, NEFL | | | | | | | | | | |
| Quadruplets | CXCL9, | 0.441 | 0.139 | 0.214 | 0.123 | 0.416 | 0.154 | 0.197 | 0.133 | 0.187 | 0.127 |
| | GFAP, APLP1, | | | | | | | | | | |
| | NEFL | | | | | | | | | | |
| Quadruplets | GFAP, | 0.429 | 0.116 | 0.197 | 0.091 | 0.427 | 0.120 | 0.197 | 0.108 | 0.169 | 0.094 |
| | CXCL13, | | | | | | | | | | |
| | FLRT2, | | | | | | | | | | |
| | APLP1 | | | | | | | | | | |
| Pairs* | OPG, NEFL | 0.225 | 0.162 | 0.077 | 0.078 | 0.159 | 0.133 | 0.043 | 0.034 | 0.061 | 0.080 |
| Pairs* | OPG, OPN | 0.212 | 0.155 | 0.069 | 0.073 | 0.129 | 0.117 | 0.030 | 0.025 | 0.053 | 0.074 |
| Pairs* | OPG, FLRT2 | 0.219 | 0.140 | 0.068 | 0.070 | 0.143 | 0.107 | 0.032 | 0.026 | 0.052 | 0.071 |
| Pairs* | OPG, MOG | 0.215 | 0.146 | 0.068 | 0.077 | 0.146 | 0.123 | 0.036 | 0.030 | 0.052 | 0.078 |
| Pairs* | CXCL9, OPG | 0.235 | 0.098 | 0.065 | 0.050 | 0.205 | 0.071 | 0.047 | 0.03 | 0.049 | 0.050 |
| Pairs | GH, NEFL | 0.257 | 0.128 | 0.083 | 0.072 | 0.329 | 0.148 | 0.130 | 0.097 | 0.067 | 0.073 |
| Pairs | CXCL13, | 0.245 | 0.099 | 0.070 | 0.055 | 0.331 | 0.122 | 0.125 | 0.085 | 0.054 | 0.056 |
| | NEFL | | | | | | | | | | |
| Pairs | APLP1, NEFL | 0.241 | 0.135 | 0.077 | 0.077 | 0.291 | 0.176 | 0.116 | 0.098 | 0.061 | 0.078 |
| Pairs | CCL20, NEFL | 0.213 | 0.103 | 0.056 | 0.045 | 0.295 | 0.143 | 0.108 | 0.090 | 0.040 | 0.046 |
| Pairs | CXCL9, NEFL | 0.229 | 0.127 | 0.068 | 0.066 | 0.287 | 0.148 | 0.104 | 0.088 | 0.052 | 0.067 |
| Triplets* | OPG, MOG, | 0.243 | 0.174 | 0.089 | 0.099 | 0.179 | 0.173 | 0.062 | 0.049 | 0.066 | 0.101 |
| | NEFL | | | | | | | | | | |
| Triplets* | OPG, FLRT2, | 0.228 | 0.161 | 0.078 | 0.080 | 0.167 | 0.131 | 0.045 | 0.036 | 0.054 | 0.082 |
| | NEFL | | | | | | | | | | |
| Triplets* | CXCL9, OPG, | 0.248 | 0.121 | 0.076 | 0.061 | 0.222 | 0.096 | 0.059 | 0.043 | 0.052 | 0.062 |
| | NEFL | | | | | | | | | | |
| Triplets* | OPG, CDCP1, | 0.216 | 0.170 | 0.076 | 0.081 | 0.143 | 0.152 | 0.043 | 0.034 | 0.051 | 0.083 |
| | NEFL | | | | | | | | | | |
| Triplets* | OPG, OPN, | 0.216 | 0.168 | 0.075 | 0.080 | 0.150 | 0.138 | 0.041 | 0.034 | 0.051 | 0.082 |
| | NEFL | | | | | | | | | | |
| Triplets | GH, APLP1, | 0.247 | 0.131 | 0.078 | 0.072 | 0.303 | 0.166 | 0.119 | 0.094 | 0.054 | 0.073 |
| | NEFL | | | | | | | | | | |
| Triplets | GH, CXCL13, | 0.249 | 0.111 | 0.074 | 0.061 | 0.314 | 0.131 | 0.116 | 0.086 | 0.050 | 0.062 |
| | NEFL | | | | | | | | | | |
| Triplets | GH, CDCP1, | 0.290 | 0.066 | 0.089 | 0.037 | 0.304 | 0.139 | 0.112 | 0.077 | 0.065 | 0.038 |
| | NEFL | | | | | | | | | | |
| Triplets | CXCL13, | 0.231 | 0.107 | 0.065 | 0.057 | 0.303 | 0.130 | 0.108 | 0.083 | 0.040 | 0.058 |
| | CCL20, NEFL | | | | | | | | | | |
| Triplets | GH, CCL20, | 0.225 | 0.111 | 0.063 | 0.051 | 0.295 | 0.140 | 0.107 | 0.087 | 0.039 | 0.053 |
| | NEFL | | | | | | | | | | |
| Quadruplets* | CDCP1, | 0.166 | 0.080 | 0.034 | 0.025 | 0.132 | 0.128 | 0.034 | 0.031 | 0.000 | 0.026 |
| | CXCL13. | | | | | | | | | | |
| | MOG, NEFL | | | | | | | | | | |
| Quadruplets* | CD6, CXCL9, | 0.145 | 0.110 | 0.033 | 0.038 | 0.153 | 0.078 | 0.030 | 0.025 | −0.001 | 0.039 |
| | CXCL13, | | | | | | | | | | |
| | NEFL | | | | | | | | | | |
| Quadruplets* | CXCL9, | 0.124 | 0.103 | 0.026 | 0.029 | 0.135 | 0.069 | 0.023 | 0.020 | −0.008 | 0.030 |
| | CXCL13, | | | | | | | | | | |
| | MOG, NEFL | | | | | | | | | | |
| Quadruplets* | CD6, CDCP1, | 0.139 | 0.080 | 0.026 | 0.022 | 0.118 | 0.125 | 0.030 | 0.029 | −0.008 | 0.023 |
| | CXCL13, | | | | | | | | | | |
| | NEFL | | | | | | | | | | |
| Quadruplets* | CD6, CXCL9, | 0.091 | 0.081 | 0.015 | 0.012 | 0.111 | 0.046 | 0.014 | 0.011 | −0.020 | 0.013 |
| | CXCL13, | | | | | | | | | | |
| | MOG | | | | | | | | | | |
| Quadruplets | CDCP1, | 0.282 | 0.058 | 0.083 | 0.032 | 0.267 | 0.133 | 0.089 | 0.071 | 0.051 | 0.033 |
| | CXCL13, | | | | | | | | | | |
| | MOG, NEFL | | | | | | | | | | |
| Quadruplets | CD6, CDCP1, | 0.277 | 0.047 | 0.079 | 0.026 | 0.261 | 0.136 | 0.086 | 0.067 | 0.047 | 0.027 |
| | CXCL13, | | | | | | | | | | |
| | NEFL | | | | | | | | | | |
| Quadruplets | CXCL9, | 0.208 | 0.117 | 0.057 | 0.058 | 0.264 | 0.128 | 0.086 | 0.086 | 0.024 | 0.060 |
| | CXCL13, | | | | | | | | | | |
| | MOG, NEFL | | | | | | | | | | |
| Quadruplets | CD6, CXCL9, | 0.200 | 0.075 | 0.046 | 0.028 | 0.210 | 0.101 | 0.055 | 0.037 | 0.012 | 0.029 |
| | CXCL13, | | | | | | | | | | |
| | NEFL | | | | | | | | | | |
| Quadruplets | CD6, CDCP1, | 0.199 | 0.072 | 0.045 | 0.030 | 0.175 | 0.100 | 0.041 | 0.039 | 0.011 | 0.031 |
| | CXCL13, | | | | | | | | | | |
| | MOG | | | | | | | | | | |

*indicates that values were transformed or demographically adjusted.

1. A method for predicting multiple sclerosis disease progression in a subject, the method comprising:
obtaining or having obtained a dataset comprising expression levels of a plurality of biomarkers, wherein the plurality of biomarkers comprise one or more biomarkers in at least one group selected from group 1, group 2, and group 3,
wherein group 1 comprises one or more of biomarker 1, biomarker 2, biomarker 3, biomarker 4, biomarker 5, biomarker 6, biomarker 7, and biomarker 8,
wherein biomarker 1 is GFAP, NEFL, OPN, CXCL9, MOG, or CHI3L1
wherein biomarker 2 is CDCP1, IL-18BP, IL-18, GFAP, or MSR1,
wherein biomarker 3 is MOG, CADM3, KLK6, BCAN, OMG, or GFAP,
wherein biomarker 4 is CXCL13, NOS3, or MMP-2,
wherein biomarker 5 is OPG, TFF3, or ENPP2,
wherein biomarker 6 is APLP1, SEZ6L, BCAN, DPP6, NCAN, or KLK6,
wherein biomarker 7 is VCAN, TINAGL1, CANT1, NECTIN2, MMP-9, or NPDC1,
wherein biomarker 8 is NEFL, MOG, CADM3, or GFAP, and
wherein group 2 comprises one or more of biomarker 9, biomarker 10, biomarker 11, biomarker 12, biomarker 13, biomarker 14, biomarker 15, biomarker 16, and biomarker 17,
wherein biomarker 9 is CXCL9, CXCL10, IL-12B, CXCL11, or GFAP,
wherein biomarker 10 is TNFRSF10A, TNFRSF11A, SPON2, CHI3L1, or IFI30,
wherein biomarker 11 is CCL20, CCL3, or TWEAK,
wherein biomarker 12 is TNFSF13B, CXCL16, ALCAM, or IL-18,
wherein biomarker 13 is OPN, OMD, MEPE, or GFAP,
wherein biomarker 14 is SERPINA9, TNFRSF9, or CNTN4,
wherein biomarker 15 is CD6, CD5, CRTAM, CD244, or TNFRSF9,
wherein biomarker 16 is FLRT2, DDR1, NTRK2, CDH6, MMP-2,
wherein biomarker 17 is CNTN2, DPP6, GDNFR-alpha-3, or SCARF2, and
wherein group 3 comprises one or more of biomarker 18, biomarker 19, biomarker 20, and biomarker 21,
wherein biomarker 18 is COL4A1, IL-6, Notch 3, or PCDH17,
wherein biomarker 19 is GH, GH2, or IGFBP-1,
wherein biomarker 20 is IL-12B, IL12A, or CXCL9, and
wherein biomarker 21 is PRTG, NTRK2, NTRK3, or CNTN4, and
generating a prediction of multiple sclerosis disease progression by applying a predictive model to the expression levels of the plurality of biomarkers.

2. The method of claim 1, wherein the plurality of biomarkers comprise each biomarker in group 1, wherein biomarker 1 is GFAP, wherein biomarker 2 is CDCP1, wherein biomarker 3 is MOG, wherein biomarker 4 is CXCL13, wherein biomarker 5 is OPG, wherein biomarker 6 is APLP1, wherein biomarker 7 is VCAN, and wherein biomarker 8 is NEFL.

3. The method of claim 2, wherein a performance of the predictive model is characterized by a correlation coefficient (R²) of at least 0.31.

4. The method of claim 2, wherein a performance of the predictive model is characterized by an AUROC of at least 0.77.

5. The method of claim 2, wherein a performance of the predictive model is characterized by a PPV of at least 0.19.

6. The method of claim 2, wherein the plurality of biomarkers further comprise each biomarker in group 2, wherein biomarker 9 is CXCL9, wherein biomarker 10 is TNFRSF10A, wherein biomarker 11 is CCL20, wherein biomarker 12 is TNFSF13B, wherein biomarker 13 is OPN, wherein biomarker 14 is SERPINA9, wherein biomarker 15 is CD6, wherein biomarker 16 is FLRTs, and wherein biomarker 17 is CNTN2.

7. The method of claim 6, wherein a performance of the predictive model is characterized by a correlation coefficient (R²) of at least 0.35.

8. The method of claim 6, wherein a performance of the predictive model is characterized by an AUROC of at least 0.76.

9. The method of claim 6, wherein a performance of the predictive model is characterized by a PPV of at least 0.19.

10. The method of any one of claims 6-9, wherein the plurality of biomarkers further comprise each biomarker in group 3, wherein biomarker 18 is COL4A1, wherein biomarker 19 is GH, wherein biomarker 20 is IL-12B, and wherein biomarker 21 is PRTG.

11. The method of claim 10, wherein a performance of the predictive model is characterized by a correlation coefficient (R²) of at least 0.36.

12. The method of claim 10, wherein a performance of the predictive model is characterized by an AUROC of at least 0.74.

13. The method of claim 10, wherein a performance of the predictive model is characterized by a PPV of at least 0.19.

14. The method of claim 1, wherein the plurality of biomarkers comprises one or more biomarkers in group 1, wherein the one or more biomarkers in group 1 comprises GFAP.

15. The method of claim 14, wherein a performance of the predictive model is characterized by an AUROC of at least 0.70.

16. The method of claim 14 or 15, wherein a performance of the predictive model is characterized by an AUROC between 0.72 and 0.78.

17. The method of claim 14, wherein a performance of the predictive model is characterized by a Pearson's R² coefficient of at least 0.10.

18. The method of claim 14 or 15, wherein a performance of the predictive model is characterized by an Pearson's R² coefficient between 0.11 and 0.22.

19. The method of claim 1, wherein the plurality of biomarkers does not include GFAP.

20. The method of claim 19, wherein a performance of the predictive model is characterized by an AUROC of at least 0.65.

21. The method of claim 19 or 20, wherein a performance of the predictive model is characterized by an AUROC between 0.66 and 0.70.

22. The method of claim 14, wherein a performance of the predictive model is characterized by a Pearson's R² coefficient of at least 0.10.

23. The method of claim 14 or 15, wherein a performance of the predictive model is characterized by an Pearson's R² coefficient between 0.015 and 0.090.

24. The method of any one of claims 1-23, wherein the prediction of multiple sclerosis disease progression is a measure of brain parenchymal fraction value.

25. The method of any one of claims 1-23, wherein the prediction of multiple sclerosis disease progression is a measure of expanded disability status scale (EDSS) score.

26. The method of claim 25, wherein an expanded disability status scale (EDSS) score less than 6 indicates a mild/moderate MS disease progression and a EDSS score greater than or equal to 6 indicates a severe MS disease progression.

27. The method of any one of claims 1-23, wherein the prediction of multiple sclerosis disease progression is a measure of a patient determined disease steps (PDDS) score.

28. The method of claim 27, wherein the prediction of multiple sclerosis disease progression is a patient determined disease steps (PDDS) score differentiating between severe and mild/moderate MS disease progression.

29. The method of claim 28, wherein a patient determined disease steps (PDDS) score less than or equal to 4 indicates a mild/moderate MS disease progression and a PDDS score greater than 4 indicates a severe MS disease progression.

30. The method of claim 1-23, wherein the prediction of multiple sclerosis disease progression is a PRO measurement information system (PROMIS) score.

31. The method of claim 1-23, wherein the prediction of multiple sclerosis disease progression is a Multiple Sclerosis Rating Scale, Revised (MSRS-R) score.

32. A method for predicting multiple sclerosis disease progression in a subject, the method comprising:
obtaining or having obtained a dataset comprising expression levels of a plurality of biomarkers comprising at least one of:
one or more neuroaxonal integrity biomarkers selected from a group consisting of CNTN2, FLRT2, NEFL, PRTG, SERPINA9, OPG, GFAP, and TNFRSF10A;
one or more neuroinflammation biomarkers selected from a group consisting of CCL20, GH, TNFRSF10A, CXCL9, CXCL13, IL-12B, CD6, and TNFSF13B;
one or more immune modulation biomarkers selected from a group consisting of CDCP1, CD6, CXCL9, CXCL13, IL-12B, and TNFSF13B; or
one or more myelination biomarkers selected from a group consisting of MOG, APLP1, and OPN; and
generating a prediction of multiple sclerosis disease progression by applying a predictive model to the expression levels of the plurality of biomarkers.

33. A method for predicting multiple sclerosis disease progression in a subject, the method comprising:
obtaining or having obtained a dataset comprising expression levels of a plurality of biomarkers comprising at least one of:
one or more neuroaxonal integrity biomarkers selected from a group consisting of CNTN2, FLRT2, NEFL, PRTG, SERPINA9, OPG, GFAP, and TNFRSF10A;
one or more neuroinflammation biomarkers selected from a group consisting of CCL20, GH, TNFRSF10A, CXCL9, CXCL13, IL-12B, CD6, and TNFSF13B;
one or more immune modulation biomarkers selected from a group consisting of CDCP1, CD6, CXCL9, CXCL13, IL-12B, and TNFSF13B;
one or more myelination biomarkers selected from a group consisting of MOG, APLP1, and OPN; or
one or more cerebrovascular function biomarkers selected from a group consisting of COL4A1, VCAN, GFAP, and CD6; and
generating a prediction of multiple sclerosis disease progression by applying a predictive model to the expression levels of the plurality of biomarkers.

34. The method of claim 32 or 33, wherein the one or more neuroaxonal integrity biomarkers comprise NEFL, OPG, and GFAP, wherein the one or more neuroinflammation biomarkers comprise CXCL13, and CXCL9, wherein the one or more immune modulation biomarkers comprise CDCP1, and wherein the one or more myelination biomarkers comprise MOG and APLP1.

35. The method of claim 34, wherein the one or more neuroaxonal integrity biomarkers further comprise SERPINA9, FLRT2, and CNTN2, wherein the one or more neuroinflammation biomarkers further comprise CCL20, CXCL9, TNFRSF10A, and CD6, wherein the one or more immune modulation biomarkers further comprise TNFSF13B, and wherein the one or more myelination biomarkers further comprise OPN.

36. The method of claim 35, wherein the one or more neuroaxonal integrity biomarkers further comprise PRTG, and wherein the one or more immune modulation biomarkers further comprise IL-12B.

37. The method of claim 36, wherein a performance of the predictive model is characterized by a correlation coefficient (R²) of at least 0.35.

38. The method of claim 36, wherein a performance of the predictive model is characterized by an AUROC of at least 0.74.

39. The method of claim 36, wherein a performance of the predictive model is characterized by a PPV of at least 0.17.

40. The method of claim 32, wherein the plurality of biomarkers comprises one or more neuroaxonal integrity biomarkers, wherein the one or more neuroaxonal integrity biomarkers comprises GFAP.

41. The method of claim 40, wherein a performance of the predictive model is characterized by an AUROC of at least 0.70.

42. The method of claim 40 or 41, wherein a performance of the predictive model is characterized by an AUROC between 0.72 and 0.78.

43. The method of claim 40, wherein a performance of the predictive model is characterized by a Pearson's R² coefficient of at least 0.10.

44. The method of claim 40 or 41, wherein a performance of the predictive model is characterized by an Pearson's R² coefficient between 0.11 and 0.22.

45. The method of claim 32, wherein the plurality of biomarkers does not include GFAP.

46. The method of claim 45, wherein a performance of the predictive model is characterized by an AUROC of at least 0.65.

47. The method of claim 45 or 46, wherein a performance of the predictive model is characterized by an AUROC between 0.66 and 0.70.

48. The method of claim 45, wherein a performance of the predictive model is characterized by a Pearson's R² coefficient of at least 0.10.

49. The method of claim 45 or 46, wherein a performance of the predictive model is characterized by an Pearson's R² coefficient between 0.015 and 0.090.

50. The method of any one of claims 32-49, wherein the prediction of multiple sclerosis disease progression is a brain parenchymal fraction value.

51. The method of any one of claims 32-49, wherein the prediction of multiple sclerosis disease progression is an expanded disability status scale (EDSS) score.

52. The method of claim 51, wherein an expanded disability status scale (EDSS) score less than 6 indicates a mild/moderate MS disease progression and a EDSS score greater than or equal to 6.0 indicates a severe MS disease progression.

53. The method of any one of claims 32-49, wherein the prediction of multiple sclerosis disease progression is a patient determined disease steps (PDDS) score.

54. The method of claim 53, wherein the prediction of multiple sclerosis disease progression is a patient determined disease steps (PDDS) score differentiating between severe and mild/moderate MS disease progression.

55. The method of claim 54, wherein a patient determined disease steps (PDDS) score less than or equal to 4 indicates a mild/moderate MS disease progression and a PDDS score greater than 4 indicates a severe MS disease progression.

56. The method of claim 32-49, wherein the prediction of multiple sclerosis disease progression is a PRO measurement information system (PROMIS) score.

57. The method of claim 32-49, wherein the prediction of multiple sclerosis disease progression is a Multiple Sclerosis Rating Scale, Revised (MSRS-R) score.

58. A method for predicting multiple sclerosis disease progression in a subject, the method comprising:
obtaining or having obtained a dataset comprising expression levels of a plurality of biomarkers, wherein the plurality of biomarkers comprises two or more of: GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG; and
generating a prediction of multiple sclerosis disease progression by applying a predictive model to the expression levels of the plurality of biomarkers.

59. The method of claim 58, wherein the plurality of biomarkers comprise each of GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG.

60. The method of claim 59, wherein a performance of the predictive model is characterized by a correlation coefficient (R²) of at least 0.35.

61. The method of claim 59, wherein a performance of the predictive model is characterized by an AUROC of at least 0.74.

62. The method of claim 59, wherein a performance of the predictive model is characterized by a PPV of at least 0.17.

63. The method of claim 58, wherein the plurality of biomarkers comprises GFAP.

64. The method of claim 63, wherein the plurality of biomarkers further comprises CDCP1.

65. The method of claim 63, wherein the plurality of biomarkers further comprises APLP1.

66. The method of claim 63, wherein the plurality of biomarkers further comprises CXCL13.

67. The method of claim 63, wherein the plurality of biomarkers further comprises MOG.

68. The method of claim 63, wherein the plurality of biomarkers further comprises OPG.

69. The method of claim 63, wherein the plurality of biomarkers further comprises CDCP1 and APLP1.

70. The method of claim 63, wherein the plurality of biomarkers further comprises MOG and CDCP1.

71. The method of claim 63, wherein the plurality of biomarkers further comprises APLP1 and CXCL 13.

72. The method of claim 63, wherein the plurality of biomarkers further comprises CDCP1 and SERPINA9.

73. The method of claim 63, wherein the plurality of biomarkers further comprises MOG and CXCL13.

74. The method of claim 63, wherein the plurality of biomarkers further comprises CDCP1, CCL20, and APLP1.

75. The method of claim 63, wherein the plurality of biomarkers further comprises CDCP1, APLP1 and CXCL13.

76. The method of claim 63, wherein the plurality of biomarkers further comprises CDCP1, CCL20, and MOG.

77. The method of claim 63, wherein the plurality of biomarkers further comprises CDCP1, APLP1, and SERPINA9.

78. The method of claim 63, wherein the plurality of biomarkers further comprises CDCP1, MOG, and APLP1.

79. The method of any one of claims 63-78, wherein a performance of the predictive model is characterized by an AUROC of at least 0.70.

80. The method of any one of claims 63-79, wherein a performance of the predictive model is characterized by an AUROC between 0.72 and 0.78.

81. The method of claim 63, wherein the plurality of biomarkers further comprises MOG.

82. The method of claim 63, wherein the plurality of biomarkers further comprises APLP1.

83. The method of claim 63, wherein the plurality of biomarkers further comprises OPG.

84. The method of claim 63, wherein the plurality of biomarkers further comprises TNFRSF10A.

85. The method of claim 63, wherein the plurality of biomarkers further comprises CDCP1.

86. The method of claim 63, wherein the plurality of biomarkers further comprises APLP1.

87. The method of claim 63, wherein the plurality of biomarkers further comprises NEFL.

88. The method of claim 63, wherein the plurality of biomarkers further comprises CNTN2.

89. The method of claim 63, wherein the plurality of biomarkers further comprises GH.

90. The method of claim 63, wherein the plurality of biomarkers further comprises CXCL9.

91. The method of claim 63, wherein the plurality of biomarkers further comprises OPG and MOG.

92. The method of claim 63, wherein the plurality of biomarkers further comprises OPG and APLP1.

93. The method of claim 63, wherein the plurality of biomarkers further comprises TNFRSF10A and MOG.

94. The method of claim 63, wherein the plurality of biomarkers further comprises CXCL9 and OPG.

95. The method of claim 63, wherein the plurality of biomarkers further comprises TNFRSF10A and APLP1.

96. The method of claim 63, wherein the plurality of biomarkers further comprises APLP1 and NEFL.

97. The method of claim 63, wherein the plurality of biomarkers further comprises CXCL13 and APLP1.

98. The method of claim 63, wherein the plurality of biomarkers further comprises FLRT2 and APLP1.

99. The method of claim 63, wherein the plurality of biomarkers further comprises CXCL9 and APLP1.

100. The method of claim 63, wherein the plurality of biomarkers further comprises GH and APLP1.

101. The method of claim 63, wherein the plurality of biomarkers further comprises CXCL9, OPG, and MOG.

102. The method of claim 63, wherein the plurality of biomarkers further comprises CNTN2, OPG, and MOG.

103. The method of claim 63, wherein the plurality of biomarkers further comprises CXCL9, OPG, and APLP1.

104. The method of claim 63, wherein the plurality of biomarkers further comprises OPG, PRTG, and MOG.

105. The method of claim 63, wherein the plurality of biomarkers further comprises OPG, OPN, and MOG.

106. The method of claim 63, wherein the plurality of biomarkers further comprises CXCL13, APLP1, and NEFL.

107. The method of claim 63, wherein the plurality of biomarkers further comprises FLRT2, APLP1, and NEFL.

108. The method of claim 63, wherein the plurality of biomarkers further comprises OPN, APLP1, and NEFL.

109. The method of claim 63, wherein the plurality of biomarkers further comprises CXCL9, APLP1, and NEFL.

110. The method of claim 63, wherein the plurality of biomarkers further comprises CXCL13, FLRT2, and APLP1.

111. The method of any one of claims 81-110, wherein a performance of the predictive model is characterized by a Pearson's R² coefficient of at least 0.10.

112. The method of any one of claims 81-111, wherein a performance of the predictive model is characterized by an Pearson's R² coefficient between 0.11 and 0.22.

113. The method of claim 58, wherein the plurality of biomarkers does not include GFAP.

114. The method of claim 113, wherein the plurality of biomarkers comprises CDCP1 and OPG.

115. The method of claim 113, wherein the plurality of biomarkers comprises CDCP1 and SERPINA9.

116. The method of claim 113, wherein the plurality of biomarkers comprises OPG and TNFRSF10A.

117. The method of claim 113, wherein the plurality of biomarkers comprises OPG and MOG.

118. The method of claim 113, wherein the plurality of biomarkers comprises CDCP1 and MOG.

119. The method of claim 113, wherein the plurality of biomarkers comprises CDCP1, MOG, and OPG.

120. The method of claim 113, wherein the plurality of biomarkers comprises CDCP1, SERPIN A9, and OPG.

121. The method of claim 113, wherein the plurality of biomarkers comprises CDCP1, OPG, and CXCL13.

122. The method of claim 113, wherein the plurality of biomarkers comprises CDCP1, CXCL9, and OPG.

123. The method of claim 113, wherein the plurality of biomarkers comprises CDCP1, FLRT2, and OPG.

124. The method of claim 113, wherein the plurality of biomarkers comprises CDCP1, MOG, OPG, and CXCL13.

125. The method of claim 113, wherein the plurality of biomarkers comprises CDCP1, MOG, TNFRSF10A, and OPG.

126. The method of claim 113, wherein the plurality of biomarkers comprises CDCP1, CXCL9, SERPINA9, and OPG,

127. The method of claim 113, wherein the plurality of biomarkers comprises CDCP1, CNTN2, SERPINA9, and OPG.

128. The method of claim 113, wherein the plurality of biomarkers comprises CDCP1, SERPINA9, CD6, and OPG.

129. The method of any one of claims 113-128, wherein a performance of the predictive model is characterized by an AUROC of at least 0.65.

130. The method of any one of claims 113-129, wherein a performance of the predictive model is characterized by an AUROC between 0.66 and 0.70.

131. The method of claim 113, wherein the plurality of biomarkers comprises OPG and NEFL.

132. The method of claim 113, wherein the plurality of biomarkers comprises OPG and OPN.

133. The method of claim 113, wherein the plurality of biomarkers comprises OPG and FLRT2.

134. The method of claim 113, wherein the plurality of biomarkers comprises OPG and MOG.

135. The method of claim 113, wherein the plurality of biomarkers comprises CXCL9 and OPG.

136. The method of claim 113, wherein the plurality of biomarkers comprises GH and NEFL.

137. The method of claim 113, wherein the plurality of biomarkers comprises CXCL13 and NEFL.

138. The method of claim 113, wherein the plurality of biomarkers comprises APLP1 and NEFL.

139. The method of claim 113, wherein the plurality of biomarkers comprises CCL20 and NEFL.

140. The method of claim 113, wherein the plurality of biomarkers comprises CXCL9 and NEFL.

141. The method of claim 113, wherein the plurality of biomarkers comprises OPG, MOG, and NEFL.

142. The method of claim 113, wherein the plurality of biomarkers comprises OPG, FLRT2, and NEFL.

143. The method of claim 113, wherein the plurality of biomarkers comprises CXCL9, OPG, and NEFL.

144. The method of claim 113, wherein the plurality of biomarkers comprises OPG, CDCP1, and NEFL.

145. The method of claim 113, wherein the plurality of biomarkers comprises OPG, OPN, and NEFL.

146. The method of claim 113, wherein the plurality of biomarkers comprises GH, APLP1, and NEFL.

147. The method of claim 113, wherein the plurality of biomarkers comprises GH, CXCL13, and NEFL.

148. The method of claim 113, wherein the plurality of biomarkers comprises GH, CDCP1, and NEFL.

149. The method of claim 113, wherein the plurality of biomarkers comprises CXCL13, CCL20, and NEFL.

150. The method of claim 113, wherein the plurality of biomarkers comprises GH, CCL20, and NEFL.

151. The method of claim 113, wherein the plurality of biomarkers comprises CDCP1, CXCL13, MOG, and NEFL.

152. The method of claim 113, wherein the plurality of biomarkers comprises CD6, CXCL9, CXCL13, and NEFL.

153. The method of claim 113, wherein the plurality of biomarkers comprises CXCL9, CXCL13, MOG, and NEFL.

154. The method of claim 113, wherein the plurality of biomarkers comprises CD6, CDCP1, CXCL13, and NEFL.

155. The method of claim 113, wherein the plurality of biomarkers comprises CD6, CXCL9, CXCL13, and MOG.

156. The method of claim 113, wherein the plurality of biomarkers comprises CDCP1, CXCL13, MOG, and NEFL.

157. The method of claim 113, wherein the plurality of biomarkers comprises CD6, CDCP1, CXCL13, and NEFL.

158. The method of claim 113, wherein the plurality of biomarkers comprises CXCL9, CXCL13, MOG, and NEFL.

159. The method of claim 113, wherein the plurality of biomarkers comprises CD6, CXCL9, CXCL13, and NEFL.

160. The method of claim 113, wherein the plurality of biomarkers comprises CD6, CDCP1, CXCL13, and MOG.

161. The method of claim 131-160, wherein a performance of the predictive model is characterized by a Pearson's R² coefficient of at least 0.10.

162. The method of claim 131-161, wherein a performance of the predictive model is characterized by an Pearson's R² coefficient between 0.015 and 0.090.

163. The method of any one of claims 58-162, wherein the prediction of multiple sclerosis disease progression is a brain parenchymal fraction value.

164. The method of any one of claims 58-162, wherein the prediction of multiple sclerosis disease progression is an expanded disability status scale (EDSS) score.

165. The method of claim 164, wherein an expanded disability status scale (EDSS) score less than or equal to 6 indicates a mild/moderate MS disease progression and a EDSS score greater than 6.5 indicates a severe MS disease progression.

166. The method of any one of claims 58-162, wherein the prediction of multiple sclerosis disease progression is a patient determined disease steps (PDDS) score.

167. The method of claim 166, wherein the prediction of multiple sclerosis disease progression is a patient determined disease steps (PDDS) score differentiating between severe and mild/moderate MS disease progression.

168. The method of claim 167, wherein a patient determined disease steps (PDDS) score less than or equal to 4 indicates a mild/moderate MS disease progression and a PDDS score greater than 4 indicates a severe MS disease progression.

169. The method of claim 58-162, wherein the prediction of multiple sclerosis disease progression is a PRO measurement information system (PROMIS) score.

170. The method of claim 58-162, wherein the prediction of multiple sclerosis disease progression is a Multiple Sclerosis Rating Scale, Revised (MSRS-R) score.

171. The method of any one of claims 1-170, wherein generating the prediction of multiple sclerosis disease progression by applying the predictive model to the expression levels of the plurality of biomarkers further comprises applying the predictive model to one or more subject attributes of the subject, wherein subject attributes comprise any of age, sex, and disease duration.

172. The method of any one of claims 1-171, wherein generating the prediction of multiple sclerosis disease progression comprises comparing a score outputted by the predictive model to a reference score.

173. The method of claim 172, wherein the reference score corresponds to any of:
A) an EDSS score;
B) a brain parenchymal fraction value;
C) a PDDS score;
D) a PROMIS score; or
E) a MSRS-R score.

174. The method of claim 173, wherein the reference score further corresponds to a mild/moderate MS disease progression or a severe MS disease progression.

175. The method of any one of claims 1-174, wherein the expression levels of the plurality of biomarkers is determined from a test sample obtained from the subject.

176. The method of claim 175, wherein the test sample is a blood or serum sample.

177. The method of claim 175 or 176, wherein the subject has multiple sclerosis, is suspected of having multiple sclerosis, or was previously diagnosed with multiple sclerosis.

178. The method of any one of claims 1-177, wherein obtaining or having obtained the dataset comprises performing an immunoassay to determine the expression levels of the plurality of biomarkers.

179. The method of claim 178, wherein the immunoassay is a Proximity Extension Assay (PEA) or LUMINEX xMAP Multiplex Assay.

180. The method of claim 178 or 179, wherein performing the immunoassay comprises contacting a test sample with a plurality of reagents comprising antibodies.

181. The method of claim 180, wherein the antibodies comprise one of monoclonal and polyclonal antibodies.

182. The method of claim 180, wherein the antibodies comprise both monoclonal and polyclonal antibodies.

183. The method of any one of claims 1-182, further comprising:
selecting a therapy for administering to the subject based on the prediction of multiple sclerosis disease progression.

184. The method of any one of claims 1-182, further comprising:
determining a therapeutic efficacy of a therapy previously administered to the subject based on the prediction of multiple sclerosis disease progression.

185. The method of claim 184, wherein determining the therapeutic efficacy of the therapy comprises comparing the prediction to a prior prediction determined for the subject at a prior timepoint

186. The method of claim 185, wherein determining the therapeutic efficacy of the therapy comprises determining that the therapy exhibits efficacy responsive to a difference between the prediction and the prior prediction.

187. The method of claim 185, wherein determining the therapeutic efficacy of the therapy comprises determining that the therapy lacks efficacy responsive to a lack of difference between the prediction and the prior prediction.

188. A non-transitory computer readable medium for predicting multiple sclerosis disease progression in a subject, the non-transitory computer readable medium comprising instructions that, when executed by a processor, cause the processor to:
obtain a dataset comprising expression levels of a plurality of biomarkers, wherein the plurality of biomarkers comprise one or more biomarkers in at least one group selected from group 1, group 2, and group 3,
wherein group 1 comprises one or more of biomarker 1, biomarker 2, biomarker 3, biomarker 4, biomarker 5, biomarker 6, biomarker 7, and biomarker 8,
wherein biomarker 1 is GFAP, NEFL, OPN, CXCL9, MOG, or CHI3L1
wherein biomarker 2 is CDCP1, IL-18BP, IL-18, GFAP, or MSR1,
wherein biomarker 3 is MOG, CADM3, KLK6, BCAN, OMG, or GFAP,
wherein biomarker 4 is CXCL13, NOS3, or MMP-2,
wherein biomarker 5 is OPG, TFF3, or ENPP2,
wherein biomarker 6 is APLP1, SEZ6L, BCAN, DPP6, NCAN, or KLK6,
wherein biomarker 7 is VCAN, TINAGL1, CANT1, NECTIN2, MMP-9, or NPDC1,
wherein biomarker 8 is NEFL, MOG, CADM3, or GFAP, and
wherein group 2 comprises one or more of biomarker 9, biomarker 10, biomarker 11, biomarker 12, biomarker 13, biomarker 14, biomarker 15, biomarker 16, and biomarker 17,
wherein biomarker 9 is CXCL9, CXCL10, IL-12B, CXCL11, or GFAP,
wherein biomarker 10 is TNFRSF10A, TNFRSF11A, SPON2, CHI3L1, or IFI30,
wherein biomarker 11 is CCL20, CCL3, or TWEAK,
wherein biomarker 12 is TNFSF13B, CXCL16, ALCAM, or IL-18,
wherein biomarker 13 is OPN, OMD, MEPE, or GFAP,
wherein biomarker 14 is SERPINA9, TNFRSF9, or CNTN4,
wherein biomarker 15 is CD6, CD5, CRTAM, CD244, or TNFRSF9,
wherein biomarker 16 is FLRT2, DDR1, NTRK2, CDH6, MMP-2,
wherein biomarker 17 is CNTN2, DPP6, GDNFR-alpha-3, or SCARF2, and
wherein group 3 comprises one or more of biomarker 18, biomarker 19, biomarker 20, and biomarker 21,
wherein biomarker 18 is COL4A1, IL-6, Notch 3, or PCDH17,
wherein biomarker 19 is GH, GH2, or IGFBP-1,
wherein biomarker 20 is IL-12B, IL12A, or CXCL9, and
wherein biomarker 21 is PRTG, NTRK2, NTRK3, or CNTN4, and
generate a prediction of multiple sclerosis disease progression by applying a predictive model to the expression levels of the plurality of biomarkers.

189. The non-transitory computer readable medium of claim 188, wherein the plurality of biomarkers comprise each biomarker in group 1, wherein biomarker 1 is GFAP, wherein biomarker 2 is CDCP1, wherein biomarker 3 is MOG, wherein biomarker 4 is CXCL13, wherein biomarker 5 is OPG, wherein biomarker 6 is APLP1, wherein biomarker 7 is VCAN, and wherein biomarker 8 is NEFL.

190. The non-transitory computer readable medium of claim 189, wherein a performance of the predictive model is characterized by a correlation coefficient (R²) of at least 0.31.

191. The non-transitory computer readable medium of claim 189, wherein a performance of the predictive model is characterized by an AUROC of at least 0.77.

192. The non-transitory computer readable medium of claim 189, wherein a performance of the predictive model is characterized by a PPV of at least 0.19.

193. The non-transitory computer readable medium of claim 189, wherein the plurality of biomarkers further comprise each biomarker in group 2, wherein biomarker 9 is CXCL9, wherein biomarker 10 is TNFRSF10A, wherein biomarker 11 is CCL20, wherein biomarker 12 is TNFSF13B, wherein biomarker 13 is OPN, wherein biomarker 14 is SERPINA9, wherein biomarker 15 is CD6, wherein biomarker 16 is FLRTs, and wherein biomarker 17 is CNTN2.

194. The non-transitory computer readable medium of claim 193, wherein a performance of the predictive model is characterized by a correlation coefficient (R²) of at least 0.35.

195. The non-transitory computer readable medium of claim 193, wherein a performance of the predictive model is characterized by an AUROC of at least 0.76.

196. The non-transitory computer readable medium of claim 193, wherein a performance of the predictive model is characterized by a PPV of at least 0.19.

197. The non-transitory computer readable medium of any one of claims 193-196, wherein the plurality of biomarkers further comprise each biomarker in group 3, wherein biomarker 18 is COL4A1, wherein biomarker 19 is GH, wherein biomarker 20 is IL-12B, and wherein biomarker 21 is PRTG.

198. The non-transitory computer readable medium of claim 197, wherein a performance of the predictive model is characterized by a correlation coefficient (R²) of at least 0.36.

199. The non-transitory computer readable medium of claim 197, wherein a performance of the predictive model is characterized by an AUROC of at least 0.74.

200. The non-transitory computer readable medium of claim 197, wherein a performance of the predictive model is characterized by a PPV of at least 0.19.

201. The non-transitory computer readable medium of claim 188, wherein the plurality of biomarkers comprises one or more biomarkers in group 1, wherein the one or more biomarkers in group 1 comprises GFAP.

202. The non-transitory computer readable medium of claim 201, wherein a performance of the predictive model is characterized by an AUROC of at least 0.70.

203. The non-transitory computer readable medium of claim 201 or 202, wherein a performance of the predictive model is characterized by an AUROC between 0.72 and 0.78.

204. The non-transitory computer readable medium of claim 201, wherein a performance of the predictive model is characterized by a Pearson's R² coefficient of at least 0.10.

205. The non-transitory computer readable medium of claim 201 or 202, wherein a performance of the predictive model is characterized by an Pearson's R² coefficient between 0.11 and 0.22.

206. The non-transitory computer readable medium of claim 188, wherein the plurality of biomarkers does not include GFAP.

207. The non-transitory computer readable medium of claim 206, wherein a performance of the predictive model is characterized by an AUROC of at least 0.65.

208. The non-transitory computer readable medium of claim 206 or 207, wherein a performance of the predictive model is characterized by an AUROC between 0.66 and 0.70.

209. The non-transitory computer readable medium of claim 206, wherein a performance of the predictive model is characterized by a Pearson's R² coefficient of at least 0.10.

210. The non-transitory computer readable medium of claim 206 or 209, wherein a performance of the predictive model is characterized by an Pearson's R² coefficient between 0.015 and 0.090.

211. The non-transitory computer readable medium of any one of claims 188-210, wherein the prediction of multiple sclerosis disease progression is a measure of brain parenchymal fraction value.

212. The non-transitory computer readable medium of any one of claims 188-210, wherein the prediction of multiple sclerosis disease progression is a measure of expanded disability status scale (EDSS) score.

213. The non-transitory computer readable medium of claim 212, wherein an expanded disability status scale (EDSS) score less than 6 indicates a mild/moderate MS disease progression and a EDSS score greater than or equal to 6 indicates a severe MS disease progression.

214. The non-transitory computer readable medium of any one of claims 188-210, wherein the prediction of multiple sclerosis disease progression is a measure of a patient determined disease steps (PDDS) score.

215. The non-transitory computer readable medium of claim 214, wherein the prediction of multiple sclerosis disease progression is a patient determined disease steps (PDDS) score differentiating between severe and mild/moderate MS disease progression.

216. The non-transitory computer readable medium of claim 215, wherein a patient determined disease steps (PDDS) score less than or equal to 4 indicates a mild/moderate MS disease progression and a PDDS score greater than 4 indicates a severe MS disease progression.

217. The non-transitory computer readable medium of any one of claims 188-210, wherein the prediction of multiple sclerosis disease progression is a PRO measurement information system (PROMIS) score.

218. The non-transitory computer readable medium of any one of claims 188-210, wherein the prediction of multiple sclerosis disease progression is a Multiple Sclerosis Rating Scale, Revised (MSRS-R) score.

219. A non-transitory computer readable medium for predicting multiple sclerosis disease progression in a subject, the non-transitory computer readable medium comprising instructions that, when executed by a processor, cause the processor to:
obtaining or having obtained a dataset comprising expression levels of a plurality of biomarkers comprising at least one of:
one or more neuroaxonal integrity biomarkers selected from a group consisting of CNTN2, FLRT2, NEFL, PRTG, SERPINA9, OPG, GFAP, and TNFRSF10A;
one or more neuroinflammation biomarkers selected from a group consisting of CCL20, GH, TNFRSF10A, CXCL9, CXCL13, IL-12B, CD6, and TNFSF13B;
one or more immune modulation biomarkers selected from a group consisting of CDCP1, CD6, CXCL9, CXCL13, IL-12B, and TNFSF13B; or
one or more myelination biomarkers selected from a group consisting of MOG, APLP1, and OPN; and
generating a prediction of multiple sclerosis disease progression by applying a predictive model to the expression levels of the plurality of biomarkers.

220. A non-transitory computer readable medium for predicting multiple sclerosis disease progression in a subject, the non-transitory computer readable medium comprising instructions that, when executed by a processor, cause the processor to:
obtain a dataset comprising expression levels of a plurality of biomarkers comprising at least one of:
one or more neuroaxonal integrity biomarkers selected from a group consisting of CNTN2, FLRT2, NEFL, PRTG, SERPINA9, OPG, GFAP, and TNFRSF10A;
one or more neuroinflammation biomarkers selected from a group consisting of CCL20, GH, TNFRSF10A, CXCL9, CXCL13, IL-12B, CD6, and TNFSF13B;
one or more immune modulation biomarkers selected from a group consisting of CDCP1, CD6, CXCL9, CXCL13, IL-12B, and TNFSF13B;
one or more myelination biomarkers selected from a group consisting of MOG, APLP1, and OPN; or
one or more cerebrovascular function biomarkers selected from a group consisting of COL4A1, VCAN, GFAP, and CD6; and
generate a prediction of multiple sclerosis disease progression by applying a predictive model to the expression levels of the plurality of biomarkers.

221. The non-transitory computer readable medium of claim 219 or 220, wherein the one or more neuroaxonal integrity biomarkers comprise NEFL, OPG, and GFAP, wherein the one or more neuroinflammation biomarkers comprise CXCL13, and CXCL9, wherein the one or more immune modulation biomarkers comprise CDCP1, and wherein the one or more myelination biomarkers comprise MOG and APLP1.

222. The non-transitory computer readable medium of claim 221, wherein the one or more neuroaxonal integrity biomarkers further comprise SERPINA9, FLRT2, and CNTN2, wherein the one or more neuroinflammation biomarkers further comprise CCL20, CXCL9, TNFRSF10A, and CD6, wherein the one or more immune modulation biomarkers further comprise TNFSF13B, and wherein the one or more myelination biomarkers further comprise OPN.

223. The non-transitory computer readable medium of claim 222, wherein the one or more neuroaxonal integrity biomarkers further comprise PRTG, and wherein the one or more immune modulation biomarkers further comprise IL-12B.

224. The non-transitory computer readable medium of claim 223, wherein a performance of the predictive model is characterized by a correlation coefficient (R²) of at least 0.35.

225. The non-transitory computer readable medium of claim 223, wherein a performance of the predictive model is characterized by an AUROC of at least 0.74.

226. The non-transitory computer readable medium of claim 223, wherein a performance of the predictive model is characterized by a PPV of at least 0.17.

227. The non-transitory computer readable medium of claim 219, wherein the plurality of biomarkers comprises one or more neuroaxonal integrity biomarkers, wherein the one or more neuroaxonal integrity biomarkers comprises GFAP.

228. The non-transitory computer readable medium of claim 227, wherein a performance of the predictive model is characterized by an AUROC of at least 0.70.

229. The non-transitory computer readable medium of claim 227 or 228, wherein a performance of the predictive model is characterized by an AUROC between 0.72 and 0.78.

230. The non-transitory computer readable medium of claim 227, wherein a performance of the predictive model is characterized by a Pearson's R² coefficient of at least 0.10.

231. The non-transitory computer readable medium of claim 227 or 230, wherein a performance of the predictive model is characterized by an Pearson's R² coefficient between 0.11 and 0.22.

232. The non-transitory computer readable medium of claim 219, wherein the plurality of biomarkers does not include GFAP.

233. The non-transitory computer readable medium of claim 232, wherein a performance of the predictive model is characterized by an AUROC of at least 0.65.

234. The non-transitory computer readable medium of claim 232 or 233, wherein a performance of the predictive model is characterized by an AUROC between 0.66 and 0.70.

235. The non-transitory computer readable medium of claim 232, wherein a performance of the predictive model is characterized by a Pearson's R² coefficient of at least 0.10.

236. The non-transitory computer readable medium of claim 232 or 235, wherein a performance of the predictive model z is characterized by an Pearson's R² coefficient between 0.015 and 0.090.

237. The non-transitory computer readable medium of any one of claims 219-236, wherein the prediction of multiple sclerosis disease progression is a brain parenchymal fraction value.

238. The non-transitory computer readable medium of any one of claims 219-236, wherein the prediction of multiple sclerosis disease progression is an expanded disability status scale (EDSS) score.

239. The non-transitory computer readable medium of claim 238, wherein an expanded disability status scale (EDSS) score less than 6 indicates a mild/moderate MS disease progression and a EDSS score greater than or equal to 6.0 indicates a severe MS disease progression.

240. The non-transitory computer readable medium of any one of claims 219-236, wherein the prediction of multiple sclerosis disease progression is a patient determined disease steps (PDDS) score.

241. The non-transitory computer readable medium of claim 240, wherein the prediction of multiple sclerosis disease progression is a patient determined disease steps (PDDS) score differentiating between severe and mild/moderate MS disease progression.

242. The non-transitory computer readable medium of claim 241, wherein a patient determined disease steps (PDDS) score less than or equal to 4 indicates a mild/moderate MS disease progression and a PDDS score greater than 4 indicates a severe MS disease progression.

243. The non-transitory computer readable medium of claim 219-236, wherein the prediction of multiple sclerosis disease progression is a PRO measurement information system (PROMIS) score.

244. The non-transitory computer readable medium of claim 219-236, wherein the prediction of multiple sclerosis disease progression is a Multiple Sclerosis Rating Scale, Revised (MSRS-R) score.

245. A non-transitory computer readable medium for predicting multiple sclerosis disease progression in a subject, the non-transitory computer readable medium comprising instructions that, when executed by a processor, cause the processor to:
obtain a dataset comprising expression levels of a plurality of biomarkers, wherein the plurality of biomarkers comprises two or more of: GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG; and
generate a prediction of multiple sclerosis disease progression by applying a predictive model to the expression levels of the plurality of biomarkers.

246. The non-transitory computer readable medium of claim 245, wherein the plurality of biomarkers comprise each of GFAP, CDCP1, MOG, CXCL13, OPG, APLP1, VCAN, NEFL, CXCL9, TNFRSF10A, CCL20/MIP 3-α, TNFSF13B, CD6, SERPINA9, FLRT2, OPN, CNTN2, COL4A1, GH, IL-12B, and PRTG.

247. The non-transitory computer readable medium of claim 246, wherein a performance of the predictive model is characterized by a correlation coefficient (R²) of at least 0.35.

248. The non-transitory computer readable medium of claim 247, wherein a performance of the predictive model is characterized by an AUROC of at least 0.74.

249. The non-transitory computer readable medium of claim 247, wherein a performance of the predictive model is characterized by a PPV of at least 0.17.

250. The non-transitory computer readable medium of claim 245, wherein the plurality of biomarkers comprises GFAP.

251. The non-transitory computer readable medium of claim 250, wherein the plurality of biomarkers further comprises CDCP1.

252. The non-transitory computer readable medium of claim 250, wherein the plurality of biomarkers further comprises APLP1.

253. The non-transitory computer readable medium of claim 250, wherein the plurality of biomarkers further comprises CXCL13.

254. The non-transitory computer readable medium of claim 250, wherein the plurality of biomarkers further comprises MOG.

255. The non-transitory computer readable medium of claim 250, wherein the plurality of biomarkers further comprises OPG.

256. The non-transitory computer readable medium of claim 250, wherein the plurality of biomarkers further comprises CDCP1 and APLP1.

257. The non-transitory computer readable medium of claim 250, wherein the plurality of biomarkers further comprises MOG and CDCP1.

258. The non-transitory computer readable medium of claim 250, wherein the plurality of biomarkers further comprises APLP1 and CXCL13.

259. The non-transitory computer readable medium of claim 250, wherein the plurality of biomarkers further comprises CDCP1 and SERPINA9.

260. The non-transitory computer readable medium of claim 250, wherein the plurality of biomarkers further comprises MOG and CXCL13.

261. The non-transitory computer readable medium of claim 250, wherein the plurality of biomarkers further comprises CDCP1, CCL20, and APLP1.

262. The non-transitory computer readable medium of claim 250, wherein the plurality of biomarkers further comprises CDCP1, APLP1 and CXCL13.

263. The non-transitory computer readable medium of claim 250, wherein the plurality of biomarkers further comprises CDCP1, CCL20, and MOG.

264. The non-transitory computer readable medium of claim 250, wherein the plurality of biomarkers further comprises CDCP1, APLP1, and SERPINA9.

265. The non-transitory computer readable medium of claim 250, wherein the plurality of biomarkers further comprises CDCP1, MOG, and APLP1.

266. The non-transitory computer readable medium of any one of claims 250-265, wherein a performance of the predictive model is characterized by an AUROC of at least 0.70.

267. The non-transitory computer readable medium of any one of claims 250-266, wherein a performance of the predictive model is characterized by an AUROC between 0.72 and 0.78.

268. The non-transitory computer readable medium of claim 250, wherein the plurality of biomarkers further comprises MOG.

269. The non-transitory computer readable medium of claim 250, wherein the plurality of biomarkers further comprises APLP1.

270. The non-transitory computer readable medium of claim 250, wherein the plurality of biomarkers further comprises OPG.

271. The non-transitory computer readable medium of claim 250, wherein the plurality of biomarkers further comprises TNFRSF10A.

272. The non-transitory computer readable medium of claim 250, wherein the plurality of biomarkers further comprises CDCP1.

273. The non-transitory computer readable medium of claim 250, wherein the plurality of biomarkers further comprises APLP1.

274. The non-transitory computer readable medium of claim 250, wherein the plurality of biomarkers further comprises NEFL.

275. The non-transitory computer readable medium of claim 250, wherein the plurality of biomarkers further comprises CNTN2.

276. The non-transitory computer readable medium of claim 250, wherein the plurality of biomarkers further comprises GH.

277. The non-transitory computer readable medium of claim 250, wherein the plurality of biomarkers further comprises CXCL9.

278. The non-transitory computer readable medium of claim 250, wherein the plurality of biomarkers further comprises OPG and MOG.

279. The non-transitory computer readable medium of claim 250, wherein the plurality of biomarkers further comprises OPG and APLP1.

280. The non-transitory computer readable medium of claim 250, wherein the plurality of biomarkers further comprises TNFRSF10A and MOG.

281. The non-transitory computer readable medium of claim 250, wherein the plurality of biomarkers further comprises CXCL9 and OPG.

282. The non-transitory computer readable medium of claim 250, wherein the plurality of biomarkers further comprises TNFRSF10A and APLP1.

283. The non-transitory computer readable medium of claim 250, wherein the plurality of biomarkers further comprises APLP1 and NEFL.

284. The non-transitory computer readable medium of claim 250, wherein the plurality of biomarkers further comprises CXCL13 and APLP1.

285. The non-transitory computer readable medium of claim 250, wherein the plurality of biomarkers further comprises FLRT2 and APLP1.

286. The non-transitory computer readable medium of claim 250, wherein the plurality of biomarkers further comprises CXCL9 and APLP1.

287. The non-transitory computer readable medium of claim 250, wherein the plurality of biomarkers further comprises GH and APLP1.

288. The non-transitory computer readable medium of claim 250, wherein the plurality of biomarkers further comprises CXCL9, OPG, and MOG.

289. The non-transitory computer readable medium of claim 250, wherein the plurality of biomarkers further comprises CNTN2, OPG, and MOG.

290. The non-transitory computer readable medium of claim 250, wherein the plurality of biomarkers further comprises CXCL9, OPG, and APLP1.

291. The non-transitory computer readable medium of claim 250, wherein the plurality of biomarkers further comprises OPG, PRTG, and MOG.

292. The non-transitory computer readable medium of claim 250, wherein the plurality of biomarkers further comprises OPG, OPN, and MOG.

293. The non-transitory computer readable medium of claim 250, wherein the plurality of biomarkers further comprises CXCL13, APLP1, and NEFL.

294. The non-transitory computer readable medium of claim 250, wherein the plurality of biomarkers further comprises FLRT2, APLP1, and NEFL.

295. The non-transitory computer readable medium of claim 250, wherein the plurality of biomarkers further comprises OPN, APLP1, and NEFL.

296. The non-transitory computer readable medium of claim 250, wherein the plurality of biomarkers further comprises CXCL9, APLP1, and NEFL.

297. The non-transitory computer readable medium of claim 250, wherein the plurality of biomarkers further comprises CXCL13, FLRT2, and APLP1.

298. The non-transitory computer readable medium of any one of claims 268-297, wherein a performance of the predictive model is characterized by a Pearson's R² coefficient of at least 0.10.

299. The non-transitory computer readable medium of any one of claims 268-298, wherein a performance of the predictive model is characterized by an Pearson's R² coefficient between 0.11 and 0.22.

300. The non-transitory computer readable medium of claim 245, wherein the plurality of biomarkers does not include GFAP.

301. The non-transitory computer readable medium of claim 300, wherein the plurality of biomarkers comprises CDCP1 and OPG.

302. The non-transitory computer readable medium of claim 300, wherein the plurality of biomarkers comprises CDCP1 and SERPINA9.

303. The non-transitory computer readable medium of claim 300, wherein the plurality of biomarkers comprises OPG and TNFRSF10A.

304. The non-transitory computer readable medium of claim 300, wherein the plurality of biomarkers comprises OPG and MOG.

305. The non-transitory computer readable medium of claim 300, wherein the plurality of biomarkers comprises CDCP1 and MOG.

306. The non-transitory computer readable medium of claim 300, wherein the plurality of biomarkers comprises CDCP1, MOG, and OPG.

307. The non-transitory computer readable medium of claim 300, wherein the plurality of biomarkers comprises CDCP1, SERPIN A9, and OPG.

308. The non-transitory computer readable medium of claim 300, wherein the plurality of biomarkers comprises CDCP1, OPG, and CXCL13.

309. The non-transitory computer readable medium of claim 300, wherein the plurality of biomarkers comprises CDCP1, CXCL9, and OPG.

310. The non-transitory computer readable medium of claim 300, wherein the plurality of biomarkers comprises CDCP1, FLRT2, and OPG.

311. The non-transitory computer readable medium of claim 300, wherein the plurality of biomarkers comprises CDCP1, MOG, OPG, and CXCL13.

312. The non-transitory computer readable medium of claim 300, wherein the plurality of biomarkers comprises CDCP1, MOG, TNFRSF10A, and OPG.

313. The non-transitory computer readable medium of claim 300, wherein the plurality of biomarkers comprises CDCP1, CXCL9, SERPINA9, and OPG,

314. The non-transitory computer readable medium of claim 300, wherein the plurality of biomarkers comprises CDCP1, CNTN2, SERPINA9, and OPG.

315. The non-transitory computer readable medium of claim 300, wherein the plurality of biomarkers comprises CDCP1, SERPINA9, CD6, and OPG.

316. The non-transitory computer readable medium of any one of claims 300-315, wherein a performance of the predictive model is characterized by an AUROC of at least 0.65.

317. The non-transitory computer readable medium of any one of claims 300-316, wherein a performance of the predictive model is characterized by an AUROC between 0.66 and 0.70.

318. The non-transitory computer readable medium of claim 300, wherein the plurality of biomarkers comprises OPG and NEFL.

319. The non-transitory computer readable medium of claim 300, wherein the plurality of biomarkers comprises OPG and OPN.

320. The non-transitory computer readable medium of claim 300, wherein the plurality of biomarkers comprises OPG and FLRT2.

321. The non-transitory computer readable medium of claim 300, wherein the plurality of biomarkers comprises OPG and MOG.

322. The non-transitory computer readable medium of claim 300, wherein the plurality of biomarkers comprises CXCL9 and OPG.

323. The non-transitory computer readable medium of claim 300, wherein the plurality of biomarkers comprises GH and NEFL.

324. The non-transitory computer readable medium of claim 300, wherein the plurality of biomarkers comprises CXCL13 and NEFL.

325. The non-transitory computer readable medium of claim 300, wherein the plurality of biomarkers comprises APLP1 and NEFL.

326. The non-transitory computer readable medium of claim 300, wherein the plurality of biomarkers comprises CCL20 and NEFL.

327. The non-transitory computer readable medium of claim 300, wherein the plurality of biomarkers comprises CXCL9 and NEFL.

328. The non-transitory computer readable medium of claim 300, wherein the plurality of biomarkers comprises OPG, MOG, and NEFL.

329. The non-transitory computer readable medium of claim 300, wherein the plurality of biomarkers comprises OPG, FLRT2, and NEFL.

330. The non-transitory computer readable medium of claim 300, wherein the plurality of biomarkers comprises CXCL9, OPG, and NEFL.

331. The non-transitory computer readable medium of claim 300, wherein the plurality of biomarkers comprises OPG, CDCP1, and NEFL.

332. The non-transitory computer readable medium of claim 300, wherein the plurality of biomarkers comprises OPG, OPN, and NEFL.

333. The non-transitory computer readable medium of claim 300, wherein the plurality of biomarkers comprises GH, APLP1, and NEFL.

334. The non-transitory computer readable medium of claim 300, wherein the plurality of biomarkers comprises GH, CXCL13, and NEFL.

335. The non-transitory computer readable medium of claim 300, wherein the plurality of biomarkers comprises GH, CDCP1, and NEFL.

336. The non-transitory computer readable medium of claim 300, wherein the plurality of biomarkers comprises CXCL13, CCL20, and NEFL.

337. The non-transitory computer readable medium of claim 300, wherein the plurality of biomarkers comprises GH, CCL20, and NEFL.

338. The non-transitory computer readable medium of claim 300, wherein the plurality of biomarkers comprises CDCP1, CXCL13, MOG, and NEFL.

339. The non-transitory computer readable medium of claim 300, wherein the plurality of biomarkers comprises CD6, CXCL9, CXCL13, and NEFL.

340. The non-transitory computer readable medium of claim 300, wherein the plurality of biomarkers comprises CXCL9, CXCL13, MOG, and NEFL.

341. The non-transitory computer readable medium of claim 300, wherein the plurality of biomarkers comprises CD6, CDCP1, CXCL13, and NEFL.

342. The non-transitory computer readable medium of claim 300, wherein the plurality of biomarkers comprises CD6, CXCL9, CXCL13, and MOG.

343. The non-transitory computer readable medium of claim 300, wherein the plurality of biomarkers comprises CDCP1, CXCL13, MOG, and NEFL.

344. The non-transitory computer readable medium of claim 300, wherein the plurality of biomarkers comprises CD6, CDCP1, CXCL13, and NEFL.

345. The non-transitory computer readable medium of claim 300, wherein the plurality of biomarkers comprises CXCL9, CXCL13, MOG, and NEFL.

346. The non-transitory computer readable medium of claim 300, wherein the plurality of biomarkers comprises CD6, CXCL9, CXCL13, and NEFL.

347. The non-transitory computer readable medium of claim 300, wherein the plurality of biomarkers comprises CD6, CDCP1, CXCL13, and MOG.

348. The non-transitory computer readable medium of claim 318-347, wherein a performance of the predictive model is characterized by a Pearson's R² coefficient of at least 0.10.

349. The non-transitory computer readable medium of claim 318-348, wherein a performance of the predictive model is characterized by an Pearson's R² coefficient between 0.015 and 0.090.

350. The non-transitory computer readable medium of any one of claims 245-349, wherein the prediction of multiple sclerosis disease progression is a brain parenchymal fraction value.

351. The non-transitory computer readable medium of any one of claims 245-349, wherein the prediction of multiple sclerosis disease progression is an expanded disability status scale (EDSS) score.

352. The non-transitory computer readable medium of claim 351, wherein an expanded disability status scale (EDSS) score less than or equal to 6 indicates a mild/moderate MS disease progression and a EDSS score greater than 6.5 indicates a severe MS disease progression.

353. The non-transitory computer readable medium of any one of claims 245-349, wherein the prediction of multiple sclerosis disease progression is a patient determined disease steps (PDDS) score.

354. The non-transitory computer readable medium of claim 353, wherein the prediction of multiple sclerosis disease progression is a patient determined disease steps (PDDS) score differentiating between severe and mild/moderate MS disease progression.

355. The non-transitory computer readable medium of claim 354, wherein a patient determined disease steps (PDDS) score less than or equal to 4 indicates a mild/moderate MS disease progression and a PDDS score greater than 4 indicates a severe MS disease progression.

356. The non-transitory computer readable medium of any one of claims 245-349, wherein the prediction of multiple sclerosis disease progression is a PRO measurement information system (PROMIS) score.

357. The non-transitory computer readable medium of any one of claims 245-349, wherein the prediction of multiple sclerosis disease progression is a Multiple Sclerosis Rating Scale, Revised (MSRS-R) score.

358. The non-transitory computer readable medium of any one of claims 188-357, wherein generating the prediction of multiple sclerosis disease progression by applying the predictive model to the expression levels of the plurality of biomarkers further comprises applying the predictive model to one or more subject attributes of the subject, wherein subject attributes comprise any of age, sex, and disease duration.

359. The non-transitory computer readable medium of any one of claims 188-358, wherein generating the prediction of multiple sclerosis disease progression comprises comparing a score outputted by the predictive model to a reference score.

360. The non-transitory computer readable medium of claim 359, wherein the reference score corresponds to any of:
A) an EDSS score;
B) a brain parenchymal fraction value;
C) a PDDS score;
D) a PROMIS score; or
E) a MSRS-R score.

361. The non-transitory computer readable medium of claim 360, wherein the reference score further corresponds to a mild/moderate MS disease progression or a severe MS disease progression.

362. The non-transitory computer readable medium of any one of claims 188-361, wherein the expression levels of the plurality of biomarkers is determined from a test sample obtained from the subject.

363. The non-transitory computer readable medium of claim 362, wherein the test sample is a blood or serum sample.

364. The non-transitory computer readable medium of claim 362 or 363, wherein the subject has multiple sclerosis, is suspected of having multiple sclerosis, or was previously diagnosed with multiple sclerosis.

365. The non-transitory computer readable medium of any one of claims 188-364, wherein obtaining or having obtained the dataset comprises performing an immunoassay to determine the expression levels of the plurality of biomarkers.

366. The non-transitory computer readable medium of claim 365, wherein the immunoassay is a Proximity Extension Assay (PEA) or LUMINEX xMAP Multiplex Assay.

367. The non-transitory computer readable medium of claim 365 or 366, wherein performing the immunoassay comprises contacting a test sample with a plurality of reagents comprising antibodies.

368. The non-transitory computer readable medium of claim 367, wherein the antibodies comprise one of monoclonal and polyclonal antibodies.

369. The non-transitory computer readable medium of claim 367, wherein the antibodies comprise both monoclonal and polyclonal antibodies.

370. The non-transitory computer readable medium of any one of claims 188-369, further comprising instructions that, when executed by a processor, cause the processor to:
select a therapy for administering to the subject based on the prediction of multiple sclerosis disease progression.

371. The non-transitory computer readable medium of any one of claims 188-370, further comprising instructions that, when executed by a processor, cause the processor to:
determine a therapeutic efficacy of a therapy previously administered to the subject based on the prediction of multiple sclerosis disease progression.

372. The non-transitory computer readable medium of claim 371, wherein the instructions that cause the processor to determine the therapeutic efficacy of the therapy further comprises instructions that, when executed by the processor, cause the processor to compare the prediction to a prior prediction determined for the subject at a prior timepoint

373. The non-transitory computer readable medium of claim 372, wherein the instructions that cause the processor to determine the therapeutic efficacy of the therapy further comprises instructions that, when executed by the processor, cause the processor to determine that the therapy exhibits efficacy responsive to a difference between the prediction and the prior prediction.

374. The non-transitory computer readable medium of claim 372, wherein the instructions that cause the processor to determine the therapeutic efficacy of the therapy further comprises instructions that, when executed by the processor, cause the processor to determine that the therapy lacks efficacy responsive to a lack of difference between the prediction and the prior prediction.
